SEQUENCE LISTING (352 sequences)

SEQ ID NO: 1 - DNA, 438 bp, Homo sapiens
  source 1..438: mol_type = genomic DNA
  CDS 1..438: protein_id = 339; translation = MNLGLSLIFLALILKGVQCQVQLQQSGAGLLKPSETLSLTCAVS GGSFSGYYWTWIRQRPGKGLEWIGEINHRGSTDYNPSLKSRVTMSIDTSKSQFSLNLK SVTAADTAVYYCARAAWGSCYDGTCYPAEYFQYWGQGTLVTVSS
atgaacctcgggctcagtttgattttccttgccctcattttaaaaggtgtccagtgtcaggtacagctgcagcagtcaggcgcaggattattgaagccttcggagaccctttccctcacctgcgctgtgtctggtgggtccttcagtggttactactggacctggatccgccagcgcccagggaaggggctggagtggattggagaaatcaatcatcgtggaagcaccgattacaacccgtccctcaagagtcgagtcaccatgtcaatagacacgtccaagagccagttctccctgaatttgaaatctgtgaccgccgcggacacggctgtgtattactgtgcgagagccgcctgggggtcttgttatgatgggacctgctaccccgctgaatacttccaatactggggccagggaaccctggtcaccgtctcctca

SEQ ID NO: 2 - protein, 146 aa, Homo sapiens
MNLGLSLIFLALILKGVQCQVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSS

SEQ ID NO: 3 - protein, 127 aa, synthetic construct
  REGION 1..127: note = Description of the artificial sequence: amino acid sequence of VH of MOG01 excluding signal sequence
QVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSS

SEQ ID NO: 4 - protein, 5 aa, synthetic construct
  REGION 1..5: note = Description of the artificial sequence: amino acid sequence of HCDR1 of MOG01
GYYWT

SEQ ID NO: 5 - protein, 16 aa, synthetic construct
  REGION 1..16: note = Description of the artificial sequence: amino acid sequence of HCDR2 of MOG01
EINHRGSTDYNPSLKS

SEQ ID NO: 6 - protein, 19 aa, synthetic construct
  REGION 1..19: note = Description of the artificial sequence: amino acid sequence of HCDR3 of MOG01
AAWGSCYDGTCYPAEYFQY

SEQ ID NO: 7 - DNA, 390 bp, Homo sapiens
  source 1..390: mol_type = genomic DNA
  CDS 1..390: protein_id = 340; translation = MKLPVRLLVLMFWIPASSSQSALTQPASVSGSPGQSITISCTGT SRDVGGYNYVSWYQQHPGKAPKLMIYDVNNRPSGVSNRFSGSKSGNTASLTISGLQAE DEADYFCSSYTSSSTPVVFGGGTKLTVL
atgaagttgcctgttaggctgttggtgctgatgttctggattcctgcttccagcagtcagtctgccctgactcagcctgcctccgtgtctgggtctcctggacagtcgatcaccatctcctgcactggaaccagccgtgacgttggtggttataactatgtctcctggtaccaacaacacccaggcaaagcccccaaactcatgatttatgatgtcaataatcggccctcaggggtttctaatcggttctctggctccaagtctggcaacacggcctccctgaccatctctgggctccaggctgaggacgaggctgattatttctgcagctcatatacaagcagtagcacccctgtggtattcggcggtgggaccaagctgaccgtccta

SEQ ID NO: 8 - protein, 130 aa, Homo sapiens
MKLPVRLLVLMFWIPASSSQSALTQPASVSGSPGQSITISCTGTSRDVGGYNYVSWYQQHPGKAPKLMIYDVNNRPSGVSNRFSGSKSGNTASLTISGLQAEDEADYFCSSYTSSSTPVVFGGGTKLTVL

SEQ ID NO: 9 - protein, 111 aa, synthetic construct
  REGION 1..111: note = Description of the artificial sequence: amino acid sequence of VL of MOG01 excluding signal sequence
QSALTQPASVSGSPGQSITISCTGTSRDVGGYNYVSWYQQHPGKAPKLMIYDVNNRPSGVSNRFSGSKSGNTASLTISGLQAEDEADYFCSSYTSSSTPVVFGGGTKLTVL

SEQ ID NO: 10 - protein, 14 aa, synthetic construct
  REGION 1..14: note = Description of the artificial sequence: amino acid sequence of LCDR1 of MOG01
TGTSRDVGGYNYVS

SEQ ID NO: 11 - protein, 7 aa, synthetic construct
  REGION 1..7: note = Description of the artificial sequence: amino acid sequence of LCDR2 of MOG01
DVNNRPS

SEQ ID NO: 12 - protein, 11 aa, synthetic construct
  REGION 1..11: note = Description of the artificial sequence: amino acid sequence of LCDR3 of MOG01
SSYTSSSTPVV

SEQ ID NO: 13 - DNA, 423 bp, Homo sapiens
  source 1..423: mol_type = genomic DNA
  CDS 1..423: protein_id = 341; translation = MNLGLSLIFLALILKGVQCQVQLQESGPGLVKSSETLSLTCAVS GHSISSAYYWGWIRQPPGKGLEWLGSIYHSGNTYYNPSLKSRVTISVDTSKNQFSLRL TSVTAADTAVYYCARGRGYSGYDSGMDVWGQGTTVTVSS
atgaacctcgggctcagtttgattttccttgccctcattttaaaaggtgtccagtgtcaggtgcagctgcaggagtcgggcccaggactggtgaagtcttcggagaccctgtccctcacctgcgctgtctctggtcactccatcagcagtgcttactactggggctggatccggcagcccccagggaaggggctggagtggcttgggagtatttatcatagtgggaacacctactacaacccgtccctcaagagtcgagtcaccatatcagtagacacgtccaagaaccagttctccctgaggctgacctctgtgaccgccgcagacacggccgtgtattactgtgcgagagggcgtggatatagtggctacgatagcggtatggacgtctggggccaagggaccacggtcaccgtctcctca

SEQ ID NO: 14 - protein, 141 aa, Homo sapiens
MNLGLSLIFLALILKGVQCQVQLQESGPGLVKSSETLSLTCAVSGHSISSAYYWGWIRQPPGKGLEWLGSIYHSGNTYYNPSLKSRVTISVDTSKNQFSLRLTSVTAADTAVYYCARGRGYSGYDSGMDVWGQGTTVTVSS

SEQ ID NO: 15 - protein, 122 aa, synthetic construct
  REGION 1..122: note = Description of the artificial sequence: amino acid sequence of VH of MOG09 excluding signal sequence
QVQLQESGPGLVKSSETLSLTCAVSGHSISSAYYWGWIRQPPGKGLEWLGSIYHSGNTYYNPSLKSRVTISVDTSKNQFSLRLTSVTAADTAVYYCARGRGYSGYDSGMDVWGQGTTVTVSS

SEQ ID NO: 16 - protein, 6 aa, synthetic construct
  REGION 1..6: note = Description of the artificial sequence: amino acid sequence of HCDR1 of MOG09
SAYYWG

SEQ ID NO: 17 - protein, 16 aa, synthetic construct
  REGION 1..16: note = Description of the artificial sequence: amino acid sequence of HCDR2 of MOG09
SIYHSGNTYYNPSLKS

SEQ ID NO: 18 - protein, 13 aa, synthetic construct
  REGION 1..13: note = Description of the artificial sequence: amino acid sequence of HCDR3 of MOG09
GRGYSGYDSGMDV

SEQ ID NO: 19 - DNA, 387 bp, Homo sapiens
  source 1..387: mol_type = genomic DNA
  CDS 1..387: protein_id = 342; translation = MKLPVRLLVLMFWIPASSSSYVLTQPPSASGTPGQRVTISCSGT SSNIGINSVNWYQQLPGMAPKLVIYSRDQRPSGVPDRFSGSQSGTSASLAINGLQSED EADYWCSTWDDSLNGWVFGGGTKLTVL
atgaagttgcctgttaggctgttggtgctgatgttctggattcctgcttccagcagttcctatgtgctgactcagccaccctcagcgtctgggacccccgggcagagggtcaccatctcttgttctggaaccagctccaacatcggaatcaatagtgtaaactggtatcaacagctcccaggaatggcccccaaactcgtcatctacagtagggatcagcggccctcaggggtccctgaccgattctctggctcccagtctggcacctcagcctccctggccatcaatggcctccagtctgaggatgaggctgattattggtgttcaacatgggatgacagcctgaatggttgggtgttcggcggagggaccaagctgaccgtccta

SEQ ID NO: 20 - protein, 129 aa, Homo sapiens
MKLPVRLLVLMFWIPASSSSYVLTQPPSASGTPGQRVTISCSGTSSNIGINSVNWYQQLPGMAPKLVIYSRDQRPSGVPDRFSGSQSGTSASLAINGLQSEDEADYWCSTWDDSLNGWVFGGGTKLTVL

SEQ ID NO: 21 - protein, 110 aa, synthetic construct
  REGION 1..110: note = Description of the artificial sequence: amino acid sequence of VL of MOG09 excluding signal sequence
SYVLTQPPSASGTPGQRVTISCSGTSSNIGINSVNWYQQLPGMAPKLVIYSRDQRPSGVPDRFSGSQSGTSASLAINGLQSEDEADYWCSTWDDSLNGWVFGGGTKLTVL

SEQ ID NO: 22 - protein, 13 aa, synthetic construct
  REGION 1..13: note = Description of the artificial sequence: amino acid sequence of LCDR1 of MOG09
SGTSSNIGINSVN

SEQ ID NO: 23 - protein, 7 aa, synthetic construct
  REGION 1..7: note = Description of the artificial sequence: amino acid sequence of LCDR2 of MOG09
SRDQRPS

SEQ ID NO: 24 - protein, 11 aa, synthetic construct
  REGION 1..11: note = Description of the artificial sequence: amino acid sequence of LCDR3 of MOG09
STWDDSLNGWV

SEQ ID NO: 25 - DNA, 414 bp, Homo sapiens
  source 1..414: mol_type = genomic DNA
  CDS 1..414: protein_id = 343; translation = MNLGLSLIFLALILKGVQCQVQLVQSGAEVKKPGASVKVSCQAS GYTFTGDYIHWVRQAPGQGLEYLGWINPDRGFTYYTQKFQGRVTMTRDTSSNTAYMEL SSLRSDDTAMYYCTRENPRAYFFDLWGQGTLVTVSS
atgaacctcgggctcagtttgattttccttgccctcattttaaaaggtgtccagtgtcaggtgcagctggtgcaatctggggctgaggtgaagaagcctggggcctcagtgaaggtctcctgccaggcttctggatacacgttcaccggcgactatattcactgggtgcgacaggcccctggacaagggctggaatacttgggatggatcaaccctgacaggggtttcacatactatacacagaagtttcagggcagggtcaccatgacccgggacacgtccagcaacacagcctacatggagctgagcagcctgagatctgacgacacggccatgtattactgtacgagagagaaccctcgcgcgtacttctttgacctctggggccagggaaccctggtcaccgtctcctca

SEQ ID NO: 26 - protein, 138 aa, Homo sapiens
MNLGLSLIFLALILKGVQCQVQLVQSGAEVKKPGASVKVSCQASGYTFTGDYIHWVRQAPGQGLEYLGWINPDRGFTYYTQKFQGRVTMTRDTSSNTAYMELSSLRSDDTAMYYCTRENPRAYFFDLWGQGTLVTVSS

SEQ ID NO: 27 - protein, 119 aa, synthetic construct
  REGION 1..119: note = Description of the artificial sequence: amino acid sequence of VH of MOG14 excluding signal sequence
QVQLVQSGAEVKKPGASVKVSCQASGYTFTGDYIHWVRQAPGQGLEYLGWINPDRGFTYYTQKFQGRVTMTRDTSSNTAYMELSSLRSDDTAMYYCTRENPRAYFFDLWGQGTLVTVSS

SEQ ID NO: 28 - protein, 5 aa, synthetic construct
  REGION 1..5: note = Description of the artificial sequence: amino acid sequence of HCDR1 of MOG14
GDYIH

SEQ ID NO: 29 - protein, 17 aa, synthetic construct
  REGION 1..17: note = Description of the artificial sequence: amino acid sequence of HCDR2 of MOG14
WINPDRGFTYYTQKFQG

SEQ ID NO: 30 - protein, 10 aa, synthetic construct
  REGION 1..10: note = Description of the artificial sequence: amino acid sequence of HCDR3 of MOG14
ENPRAYFFDL

SEQ ID NO: 31 - DNA, 381 bp, Homo sapiens
  source 1..381: mol_type = genomic DNA
  CDS 1..381: protein_id = 344; translation = MKLPVRLLVLMFWIPASSSEIVLTQSPGTLSLSPGERATLSCRA SQSISGSYVTWYQQKPGQAPRLLIYATSNRAIGIPDKFSGGGSGRDFTLTINRLEPED FAVYYCQQSVSSPYTFGQGTKVEIK
atgaagttgcctgttaggctgttggtgctgatgttctggattcctgcttccagcagtgaaatagtgttgacgcagtctccaggcaccctgtctttgtctccaggggaaagagccactctctcctgcagggccagtcagagtattagcggcagctacgtgacctggtaccagcagaagcctggccaggctcccaggctcctcatctatgctacatccaatagggccattggcatcccagacaagttcagtggcggtgggtctgggagagacttcactctcaccatcaacagactggagcctgaagattttgcagtgtattactgtcagcagagtgttagttctccgtacacttttggccaggggaccaaggtggaaatcaaa

SEQ ID NO: 32 - protein, 127 aa, Homo sapiens
MKLPVRLLVLMFWIPASSSEIVLTQSPGTLSLSPGERATLSCRASQSISGSYVTWYQQKPGQAPRLLIYATSNRAIGIPDKFSGGGSGRDFTLTINRLEPEDFAVYYCQQSVSSPYTFGQGTKVEIK

SEQ ID NO: 33 - protein, 108 aa, synthetic construct
  REGION 1..108: note = Description of the artificial sequence: amino acid sequence of VL of MOG14 excluding signal sequence
EIVLTQSPGTLSLSPGERATLSCRASQSISGSYVTWYQQKPGQAPRLLIYATSNRAIGIPDKFSGGGSGRDFTLTINRLEPEDFAVYYCQQSVSSPYTFGQGTKVEIK

SEQ ID NO: 34 - protein, 12 aa, synthetic construct
  REGION 1..12: note = Description of the artificial sequence: amino acid sequence of LCDR1 of MOG14
RASQSISGSYVT

SEQ ID NO: 35 - protein, 7 aa, synthetic construct
  REGION 1..7: note = Description of the artificial sequence: amino acid sequence of LCDR2 of MOG14
ATSNRAI

SEQ ID NO: 36 - protein, 9 aa, synthetic construct
  REGION 1..9: note = Description of the artificial sequence: amino acid sequence of LCDR3 of MOG14
QQSVSSPYT

SEQ ID NO: 37 - DNA, 411 bp, synthetic construct
  misc_feature 1..411: note = Description of the artificial sequence: sequence of VHH of iMOG_3Rim1_S32 including signal sequence
  source 1..411: mol_type = other DNA
  CDS 1..411: protein_id = 345; translation = MYRMQLLSCIALSLALVTNSQVQLVESGGGLVQTGGSLRLSCAA SGSMFSTMGWFRQAPGNQRELVAIMSSGGTANYADSVKGRFTISGDNVKNTVTLQMNS LNPEDTAVYYCRFTGWVKSSFSTYWGQGTQVTVSS
atgtacaggatgcaactcctgtcttgcattgcactaagtcttgcacttgtcacgaattcgcaggtgcagctcgtggagtctgggggaggcttggtgcagactggggggtctctgagactctcctgtgcagcctctggaagcatgttcagtaccatgggctggttccgccaggctccagggaaccagcgcgagttggtcgccattatgtcatccggtggtaccgcaaactatgcagactctgtgaagggccgattcaccatctccggagacaacgtcaagaacacggtgactctccaaatgaacagcctgaatccagaggacacagccgtctattattgtaggtttaccggttgggtcaagagttcgttctctacgtactggggccaggggacccaggtcaccgtctcctca

SEQ ID NO: 38 - protein, 137 aa, synthetic construct
  REGION 1..137: note = Synthetic Construct
MYRMQLLSCIALSLALVTNSQVQLVESGGGLVQTGGSLRLSCAASGSMFSTMGWFRQAPGNQRELVAIMSSGGTANYADSVKGRFTISGDNVKNTVTLQMNSLNPEDTAVYYCRFTGWVKSSFSTYWGQGTQVTVSS

SEQ ID NO: 39 - protein, 117 aa, synthetic construct
  REGION 1..117: note = Description of the artificial sequence: amino acid sequence of VHH of iMOG_3Rim1_S32 excluding signal sequence
QVQLVESGGGLVQTGGSLRLSCAASGSMFSTMGWFRQAPGNQRELVAIMSSGGTANYADSVKGRFTISGDNVKNTVTLQMNSLNPEDTAVYYCRFTGWVKSSFSTYWGQGTQVTVSS

SEQ ID NO: 40 - skipped

SEQ ID NO: 41 - protein, 16 aa, synthetic construct
  REGION 1..16: note = Description of the artificial sequence: amino acid sequence of CDR2 of iMOG_3Rim1_S32
IMSSGGTANYADSVKG

SEQ ID NO: 42 - protein, 11 aa, synthetic construct
  REGION 1..11: note = Description of the artificial sequence: amino acid sequence of CDR3 of iMOG_3Rim1_S32
TGWVKSSFSTY

SEQ ID NO: 43 - DNA, 36 bp, synthetic construct
  misc_feature 1..36: note = Description of the artificial sequence: primer1
  source 1..36: mol_type = other DNA
ggattcctgcttccagcagtcagtctgccctgactc

SEQ ID NO: 44 - DNA, 36 bp, synthetic construct
  misc_feature 1..36: note = Description of the artificial sequence: primer2
  source 1..36: mol_type = other DNA
ggcggccttgggctgacctaggacggtcagcttggt

SEQ ID NO: 45 - DNA, 80 bp, synthetic construct
  misc_feature 1..80: note = Description of the artificial sequence: primer3
  source 1..80: mol_type = other DNA
acgccatcacagatctgcctcttcaaaatgaagttgcctgttaggctgttggtgctgatgttctggattcctgcttccag

SEQ ID NO: 46 - DNA, 36 bp, synthetic construct
  misc_feature 1..36: note = Description of the artificial sequence: primer4
  source 1..36: mol_type = other DNA
aaaaggtgtccagtgtcaggtacagctgcagcagtc

SEQ ID NO: 47 - DNA, 32 bp, synthetic construct
  misc_feature 1..32: note = primer5
  source 1..32: mol_type = other DNA
gccccttggtgctagctgaggagacggtgacc

SEQ ID NO: 48 - DNA, 80 bp, synthetic construct
  misc_feature 1..80: note = Description of the artificial sequence: primer6
  source 1..80: mol_type = other DNA
acacagacccgtcgacccctcaccatgaacctcgggctcagtttgattttccttgccctcattttaaaaggtgtccagtg

SEQ ID NO: 49 - DNA, 36 bp, synthetic construct
  misc_feature 1..36: note = Description of the artificial sequence: primer7
  source 1..36: mol_type = other DNA
ggattcctgcttccagcagttcctatgtgctgactc

SEQ ID NO: 50 - DNA, 36 bp, synthetic construct; identical to SEQ ID NO: 44
  misc_feature 1..36: note = Description of the artificial sequence: primer8
  source 1..36: mol_type = other DNA

SEQ ID NO: 51 - DNA, 36 bp, synthetic construct
  misc_feature 1..36: note = Description of the artificial sequence: primer9
  source 1..36: mol_type = other DNA
aaaaggtgtccagtgtcaggtgcagctgcaggagtc

SEQ ID NO: 52 - DNA, 32 bp, synthetic construct; identical to SEQ ID NO: 47
  misc_feature 1..32: note = Description of the artificial sequence: primer10
  source 1..32: mol_type = other DNA

SEQ ID NO: 53 - DNA, 40 bp, synthetic construct
  misc_feature 1..40: note = Description of the artificial sequence: primer11
  source 1..40: mol_type = other DNA
ggattcctgcttccagcagtgaaatagtgttgacgcagtc

SEQ ID NO: 54 - DNA, 36 bp, synthetic construct
  misc_feature 1..36: note = Description of the artificial sequence: primer12
  source 1..36: mol_type = other DNA
gtgcagccaccgtacgtttgatttccaccttggtcc

SEQ ID NO: 55 - DNA, 36 bp, synthetic construct
  misc_feature 1..36: note = Description of the artificial sequence: primer13
  source 1..36: mol_type = other DNA
aaaaggtgtccagtgtcaggtgcagctggtgcaatc

SEQ ID NO: 56 - DNA, 32 bp, synthetic construct; identical to SEQ ID NO: 47
  misc_feature 1..32: note = Description of the artificial sequence: primer14
  source 1..32: mol_type = other DNA

SEQ ID NO: 57 - DNA, 40 bp, synthetic construct
  misc_feature 1..40: note = Description of the artificial sequence: primer15
  source 1..40: mol_type = other DNA
tgcacttgtcacgaattcgcaggtgcagctcgtggagtct

SEQ ID NO: 58 - DNA, 45 bp, synthetic construct
  misc_feature 1..45: note = Description of the artificial sequence: primer16
  source 1..45: mol_type = other DNA
accatatttggactcagatctggccgctgaggagacggtgacctg

SEQ ID NO: 59 - DNA, 30 bp, synthetic construct
  misc_feature 1..30: note = Description of the artificial sequence: primer17
  source 1..30: mol_type = other DNA
acagtctcctcagctagcaccaaggggcca

SEQ ID NO: 60 - DNA, 36 bp, synthetic construct
  misc_feature 1..36: note = Description of the artificial sequence: primer18
  source 1..36: mol_type = other DNA
ctgctgcagctgtacctgggaccctcctcctccgga

SEQ ID NO: 61 - DNA, 33 bp, synthetic construct
  misc_feature 1..33: note = Description of the artificial sequence: primer19
  source 1..33: mol_type = other DNA
ggaggaggagggtcccaggtacagctgcagcag

SEQ ID NO: 62 - DNA, 33 bp, synthetic construct
  misc_feature 1..33: note = Description of the artificial sequence: primer20
  source 1..33: mol_type = other DNA
aagcggccgcctggatcctcataggacggtcag

SEQ ID NO: 63 - DNA, 39 bp, synthetic construct
  misc_feature 1..39: note = Description of the artificial sequence: primer21
  source 1..39: mol_type = other DNA
tcagtcataatgtctagaggagacatccagatgacccag

SEQ ID NO: 64 - DNA, 37 bp, synthetic construct
  misc_feature 1..37: note = Description of the artificial sequence: primer22
  source 1..37: mol_type = other DNA
gggcggccttgggctgacctttgatctccaccttggt

SEQ ID NO: 65 - DNA, 41 bp, synthetic construct
  misc_feature 1..41: note = Description of the artificial sequence: primer23
  source 1..41: mol_type = other DNA
cagcctttcctggtatacttagtgaggtgcagttggtggag

SEQ ID NO: 66 - DNA, 36 bp, synthetic construct
  misc_feature 1..36: note = Description of the artificial sequence: primer24
  source 1..36: mol_type = other DNA
tggccccttggtgctagccgaggagacggtgaccag

SEQ ID NO: 67 - DNA, 738 bp, Rattus norvegicus
  source 1..738: mol_type = genomic DNA
  CDS 1..738: protein_id = 346; translation = MAGVWSLSLPSCLLSLLLLLQLSRSYAGQFRVIGPGHPIRALVG DEAELPCRISPGKNATGMEVGWYRSPFSRVVHLYRNGKDQDAEQAPEYRGRTELLKES IGEGKVALRIQNVRFSDEGGYTCFFRDHSYQEEAAVELKVEDPFYWINPGVLALIALV PMLLLQVSVGLVFLFLQHRLRGKLRAEVENLHRTFDPHFLRVPCWKITLFVIVPVLGP LVALIICYNWLHRRLAGQFLEELRNPF
atggccggtgtgtggagcctttctctgcccagctgcctcctgtccctgctcctcctcctccagttgtcacgcagctacgcaggacagttcagagtgatagggccagggcatcccatccgggctttagttggggatgaagcagaactgccgtgccgtatatctcctgggaagaatgccacgggcatggaggtggggtggtaccgttctcccttttcaagagtggttcatctgtaccgaaatggcaaggaccaagacgcagagcaagcgcctgaataccggggacgcacagagcttctgaaagagtctatcggcgagggaaaggttgccctcaggatccagaacgtgaggttctcggatgaaggaggctacacatgcttcttcagagaccactcctaccaagaagaagccgccgtggagttgaaagtagaagatcccttctactggatcaaccctggcgtgctggctctcattgcccttgtgcctatgctgctcctgcaggtctctgtaggccttgtattcctcttcctgcagcacagactgagaggaaaactccgtgcagaagtcgagaatctccatcggacttttgatcctcacttcctgagagtgccctgctggaagataacactgtttgttattgtccctgttcttggacccctggttgctttgatcatctgctacaactggctgcaccgaagactggcaggacagtttcttgaagagctaagaaaccccttttga

SEQ ID NO: 68 - protein, 245 aa, Rattus norvegicus
MAGVWSLSLPSCLLSLLLLLQLSRSYAGQFRVIGPGHPIRALVGDEAELPCRISPGKNATGMEVGWYRSPFSRVVHLYRNGKDQDAEQAPEYRGRTELLKESIGEGKVALRIQNVRFSDEGGYTCFFRDHSYQEEAAVELKVEDPFYWINPGVLALIALVPMLLLQVSVGLVFLFLQHRLRGKLRAEVENLHRTFDPHFLRVPCWKITLFVIVPVLGPLVALIICYNWLHRRLAGQFLEELRNPF

SEQ ID NO: 69 - DNA, 1167 bp, synthetic construct
  misc_feature 1..1167: note = Description of the artificial sequence: sequence of rMOG-FLAG-Fc
  source 1..1167: mol_type = other DNA
  CDS 1..1167: protein_id = 347; translation = MAGVWSLSLPSCLLSLLLLLQLSRSYAGQFRVIGPGHPIRALVG DEAELPCRISPGKNATGMEVGWYRSPFSRVVHLYRNGKDQDAEQAPEYRGRTELLKES IGEGKVALRIQNVRFSDEGGYTCFFRDHSYQEEAAVELKVEDPFYWSRADYKDDDDKT SDKTHTCPPCPAPELLGGPSVFLFPPKPKDTLMISRTPEVTCVVVDVSHEDPEVKFNW YVDGVEVHNAKTKPREEQYNSTYRVVSVLTVLHQDWLNGKEYKCKVSNKALPAPIEKT ISKAKGQPREPQVYTLPPSRDELTKNQVSLTCLVKGFYPSDIAVEWESNGQPENNYKT TPPVLDSDGSFFLYSKLTVDKSRWQQGNVFSCSVMHEALHNHYTQKSLSLSPGK
atggccggtgtgtggagcctttctctgcccagctgcctcctgtccctgctcctcctcctccagttgtcacgcagctacgcaggacagttcagagtgatagggccagggcatcccatccgggctttagttggggatgaagcagaactgccgtgccgtatatctcctgggaagaatgccacgggcatggaggtggggtggtaccgttctcccttttcaagagtggttcatctgtaccgaaatggcaaggaccaagacgcagagcaagcgcctgaataccggggacgcacagagcttctgaaagagtctatcggcgagggaaaggttgccctcaggatccagaacgtgaggttctcggatgaaggaggctacacatgcttcttcagagaccactcctaccaagaagaagccgccgtggagttgaaagtagaagatcccttctactggtctagagcagactacaaggacgacgatgacaagactagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 70 - protein, 388 aa, synthetic construct
  REGION 1..388: note = Synthetic Construct
MAGVWSLSLPSCLLSLLLLLQLSRSYAGQFRVIGPGHPIRALVGDEAELPCRISPGKNATGMEVGWYRSPFSRVVHLYRNGKDQDAEQAPEYRGRTELLKESIGEGKVALRIQNVRFSDEGGYTCFFRDHSYQEEAAVELKVEDPFYWSRADYKDDDDKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - DNA, 1137 bp, synthetic construct
  misc_feature 1..1137: note = Description of the artificial sequence: sequence of rMOG-GST
  source 1..1137: mol_type = other DNA
  CDS 1..1137: protein_id = 348; translation = MAGVWSLSLPSCLLSLLLLLQLSRSYAGQFRVIGPGHPIRALVG DEAELPCRISPGKNATGMEVGWYRSPFSRVVHLYRNGKDQDAEQAPEYRGRTELLKES IGEGKVALRIQNVRFSDEGGYTCFFRDHSYQEEAAVELKVEDPFYWGTLEVLFQGPMS PILGYWKIKGLVQPTRLLLEYLEEKYEEHLYERDEGDKWRNKKFELGLEFPNLPYYID GDVKLTQSMAIIRYIADKHNMLGGCPKERAEISMLEGAVLDIRYGVSRIAYSKDFETL KVDFLSKLPEMLKMFEDRLCHKTYLNGDHVTHPDFMLYDALDVVLYMDPMCLDAFPKL VCFKKRIEAIPQIDKYLKSSKYIAWPLQGWQATFGGGDHPPKSD
atggccggtgtgtggagcctttctctgcccagctgcctcctgtccctgctcctcctcctccagttgtcacgcagctacgcaggacagttcagagtgatagggccagggcatcccatccgggctttagttggggatgaagcagaactgccgtgccgtatatctcctgggaagaatgccacgggcatggaggtggggtggtaccgttctcccttttcaagagtggttcatctgtaccgaaatggcaaggaccaagacgcagagcaagcgcctgaataccggggacgcacagagcttctgaaagagtctatcggcgagggaaaggttgccctcaggatccagaacgtgaggttctcggatgaaggaggctacacatgcttcttcagagaccactcctaccaagaagaagccgccgtggagttgaaagtagaagatcccttctactggggtaccctggaagttctgttccaggggcccatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggattga

SEQ ID NO: 72 - protein, 378 aa, synthetic construct
  REGION 1..378: note = Synthetic Construct
MAGVWSLSLPSCLLSLLLLLQLSRSYAGQFRVIGPGHPIRALVGDEAELPCRISPGKNATGMEVGWYRSPFSRVVHLYRNGKDQDAEQAPEYRGRTELLKESIGEGKVALRIQNVRFSDEGGYTCFFRDHSYQEEAAVELKVEDPFYWGTLEVLFQGPMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 73 - DNA, 744 bp, Mus musculus
  source 1..744: mol_type = genomic DNA
  CDS 1..744: protein_id = 349; translation = MACLWSFSWPSCFLSLLLLLLLQLSCSYAGQFRVIGPGYPIRAL VGDEAELPCRISPGKNATGMEVGWYRSPFSRVVHLYRNGKDQDAEQAPEYRGRTELLK ETISEGKVTLRIQNVRFSDEGGYTCFFRDHSYQEEAAMELKVEDPFYWVNPGVLTLIA LVPTILLQVSVGLVFLFLQHRLRGKLRAEVENLHRTFDPHFLRVPCWKITLFVIVPVL GPLVALIICYNWLHRRLAGQFLEELRNPF
atggcctgtttgtggagcttctcttggcccagctgcttcctctcccttctcctcctccttctcctccagttgtcatgcagctatgcaggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtgctgactctcatcgcacttgtgcctacgatcctcctgcaggtctctgtaggccttgtattcctcttcctgcagcacagactgagaggaaaacttcgtgcagaagtagagaatctccatcggacttttgatcctcacttcctgagggtgccctgctggaagataacactgtttgttattgtgcctgttcttggacccctggttgccttgatcatctgctacaactggctgcaccgaagactggcaggacagtttcttgaagagctaagaaaccccttttga

SEQ ID NO: 74 - protein, 247 aa, Mus musculus
MACLWSFSWPSCFLSLLLLLLLQLSCSYAGQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVEDPFYWVNPGVLTLIALVPTILLQVSVGLVFLFLQHRLRGKLRAEVENLHRTFDPHFLRVPCWKITLFVIVPVLGPLVALIICYNWLHRRLAGQFLEELRNPF

SEQ ID NO: 75 - DNA, 744 bp, Macaca fascicularis
  source 1..744: mol_type = genomic DNA
  CDS 1..744: protein_id = 350; translation = MASLSRPSLPSCLCSFLLLLLLQVSSSYAGQFRVIGPRQPIRAL VGDEVELPCRISPGKNATGMEVGWYRPPFSRVVHLYRNGRDQDGEQAPEYRGRTELLK DAIGEGKVTLRIRNVRFSDEGGFTCFFRDHSYQEEAAIELKVEDPFYWVSPAVLVLLA VLPVLLLQITVGLVFLCLQYRLRGKLRAEIENLHRTFDPHFLRVPCWKITLFVIVPVL GPLVALIICYNWLHRRLAGQFLEELRNPF
atggcaagcttatcaagaccctctctgcccagctgcctctgctccttcctcctcctcctgctcctccaagtgtcttccagctacgcaggacagttcagagtgataggaccaagacaacctatccgggctctggtcggtgatgaagtggaattgccatgtcgcatatctcctgggaagaacgctacaggcatggaagtgggatggtaccggccccccttctctagggtggttcatctctacagaaatggcagggaccaagatggagagcaagcacctgaatatcggggccggacagagctgctgaaagacgctattggtgagggaaaggtgactctcaggatccggaatgtaaggttctcagatgaaggaggtttcacctgcttcttccgagatcattcttaccaagaggaggcagcaatagaattgaaagtggaagatcccttctactgggtcagccctgcagtgctggttctcctcgcggtgctgcctgtgctccttctgcagatcactgtcggcctcgtcttcctctgcctgcagtatagactgagaggaaaacttcgagcagagatagagaatctccaccggacttttgatccccactttctgagggtgccctgctggaagataaccctgtttgtaattgtgccggttcttggacccctggttgccttgatcatctgctacaactggctacatcgaagactagcagggcaattccttgaagagctaagaaaccctttctga

SEQ ID NO: 76 - protein, 247 aa, Macaca fascicularis
MASLSRPSLPSCLCSFLLLLLLQVSSSYAGQFRVIGPRQPIRALVGDEVELPCRISPGKNATGMEVGWYRPPFSRVVHLYRNGRDQDGEQAPEYRGRTELLKDAIGEGKVTLRIRNVRFSDEGGFTCFFRDHSYQEEAAIELKVEDPFYWVSPAVLVLLAVLPVLLLQITVGLVFLCLQYRLRGKLRAEIENLHRTFDPHFLRVPCWKITLFVIVPVLGPLVALIICYNWLHRRLAGQFLEELRNPF

SEQ ID NO: 77 - DNA, 744 bp, Homo sapiens
  source 1..744: mol_type = genomic DNA
  CDS 1..744: protein_id = 351; translation = MASLSRPSLPSCLCSFLLLLLLQVSSSYAGQFRVIGPRHPIRAL VGDEVELPCRISPGKNATGMEVGWYRPPFSRVVHLYRNGKDQDGDQAPEYRGRTELLK DAIGEGKVTLRIRNVRFSDEGGFTCFFRDHSYQEEAAMELKVEDPFYWVSPGVLVLLA VLPVLLLQITVGLVFLCLQYRLRGKLRAEIENLHRTFDPHFLRVPCWKITLFVIVPVL GPLVALIICYNWLHRRLAGQFLEELRNPF
atggcaagcttatcaagaccctctctgcccagctgcctctgctccttcctcctcctcctcctcctccaagtgtcttccagctatgcagggcagttcagagtgataggaccaagacaccctatccgggctctggtcggggatgaagtggaattgccatgtcgcatatctcctgggaagaacgctacaggcatggaggtggggtggtaccgcccccccttctctagggtggttcatctctacagaaatggcaaggaccaagatggagaccaggcacctgaatatcggggccggacagagctgctgaaagatgctattggtgagggaaaggtgactctcaggatccggaatgtaaggttctcagatgaaggaggtttcacctgcttcttccgagatcattcttaccaagaggaggcagcaatggaattgaaagtagaagatcctttctactgggtgagccctggagtgctggttctcctcgcggtgctgcctgtgctcctcctgcagatcactgttggcctcgtcttcctctgcctgcagtacagactgagaggaaaacttcgagcagagatagagaatctccaccggacttttgatccccactttctgagggtgccctgctggaagataaccctgtttgtaattgtgccggttcttggacccttggttgccttgatcatctgctacaactggctacatcgaagactagcagggcaattccttgaagagctacgaaatcccttctga

SEQ ID NO: 78 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFDPHFLRVPCWKITLFVIVPVLGPLVALIICYNWLHRRLAGQFLEELRNPF

SEQ ID NO: 79 - DNA, 33 bp, synthetic construct
  misc_feature 1..33: note = Description of the artificial sequence: primer25
  source 1..33: mol_type = other DNA
gagacgccatcacagatcatcccaccatgtaca

SEQ ID NO: 80 - DNA, 33 bp, synthetic construct
  misc_feature 1..33: note = Description of the artificial sequence: primer26
  source 1..33: mol_type = other DNA
gtaaccgttaacggatcctcatttacccagaga

SEQ ID NO: 81 - DNA, 36 bp, synthetic construct
  misc_feature 1..36: note = Description of the artificial sequence: primer27
  source 1..36: mol_type = other DNA
accaaggtggagatcaaacgtacggtggctgcacca

SEQ ID NO: 82 - DNA, 36 bp, synthetic construct
  misc_feature 1..36: note = Description of the artificial sequence: primer28
  source 1..36: mol_type = other DNA
cggccacacgttgaattctcaacactctcccctgtt

SEQ ID NO: 83 - DNA, 36 bp, synthetic construct
  misc_feature 1..36: note = Description of the artificial sequence: primer29
  source 1..36: mol_type = other DNA
gattcctgcttccagcagtcagtttgtgctttctca

SEQ ID NO: 84 - DNA, 36 bp, synthetic construct
  misc_feature 1..36: note = Description of the artificial sequence: primer30
  source 1..36: mol_type = other DNA
ggcggccttgggctgacctaggacagtgagcttggt

SEQ ID NO: 85 - DNA, 36 bp, synthetic construct
  misc_feature 1..36: note = Description of the artificial sequence: primer31
  source 1..36: mol_type = other DNA
ttaaaaggtgtccagtgtgaggtgcagctggtggaa

SEQ ID NO: 86 - DNA, 36 bp, synthetic construct
  misc_feature 1..36: note = Description of the artificial sequence: primer32
  source 1..36: mol_type = other DNA
tggccccttggtgctagctgaggagactgtgaccat

SEQ ID NO: 87 - DNA, 38 bp, synthetic construct
  misc_feature 1..38: note = Description of the artificial sequence: primer33
  source 1..38: mol_type = other DNA
tcagtcataatgtctagaggacagtttgtgctttctca

SEQ ID NO: 88 - DNA, 36 bp, synthetic construct
  misc_feature 1..36: note = Description of the artificial sequence: primer34
  source 1..36: mol_type = other DNA
cggccacacgttgaattctcatgaacattctgtagg

SEQ ID NO: 89 - DNA, 41 bp, synthetic construct
  misc_feature 1..41: note = Description of the artificial sequence: primer35
  source 1..41: mol_type = other DNA
cagcctttcctggtatacttagtgaggtgcagctggtggaa

SEQ ID NO: 90 - DNA, 30 bp, synthetic construct; identical to SEQ ID NO: 59
  misc_feature 1..30: note = Description of the artificial sequence: primer36
  source 1..30: mol_type = other DNA

SEQ ID NO: 91 - DNA, 35 bp, synthetic construct
  misc_feature 1..35: note = Description of the artificial sequence: primer37
  source 1..35: mol_type = other DNA
tccaccagctgcacctcggaccctcctcctccgga

SEQ ID NO: 92 - DNA, 33 bp, synthetic construct
  misc_feature 1..33: note = Description of the artificial sequence: primer38
  source 1..33: mol_type = other DNA
ggaggaggagggtccgaggtgcagctggtggaa

SEQ ID NO: 93 - DNA, 36 bp, synthetic construct
  misc_feature 1..36: note = Description of the artificial sequence: primer39
  source 1..36: mol_type = other DNA
aagcggccgcctggatcctcataggacagtgagctt

SEQ ID NO: 94 - DNA, 33 bp, synthetic construct
  misc_feature 1..33: note = Description of the artificial sequence: primer40
  source 1..33: mol_type = other DNA
agacgccatcacagatctgcctcttcaaaatga

SEQ ID NO: 95 - DNA, 36 bp, synthetic construct
  misc_feature 1..36: note = Description of the artificial sequence: primer41
  source 1..36: mol_type = other DNA
tggtgcagccaccgtacgtttgatttccagcttggt

SEQ ID NO: 96 - DNA, 33 bp, synthetic construct
  misc_feature 1..33: note = Description of the artificial sequence: primer42
  source 1..33: mol_type = other DNA
gaacacagacccgtcgacccctcaccatgaacc

SEQ ID NO: 97 - DNA, 36 bp, synthetic construct
  misc_feature 1..36: note = Description of the artificial sequence: primer43
  source 1..36: mol_type = other DNA
tggccccttggtgctagcggaggagactgtgagagt

SEQ ID NO: 98 - DNA, 2640 bp, synthetic construct
  misc_feature 1..2640: note = Description of the artificial sequence: sequence of hHER2-GST
  source 1..2640: mol_type = other DNA
  CDS 1..2640: protein_id = 352; translation = MELAALCRWGLLLALLPPGAASTQVCTGTDMKLRLPASPETHLD MLRHLYQGCQVVQGNLELTYLPTNASLSFLQDIQEVQGYVLIAHNQVRQVPLQRLRIV RGTQLFEDNYALAVLDNGDPLNNTTPVTGASPGGLRELQLRSLTEILKGGVLIQRNPQ LCYQDTILWKDIFHKNNQLALTLIDTNRSRACHPCSPMCKGSRCWGESSEDCQSLTRT VCAGGCARCKGPLPTDCCHEQCAAGCTGPKHSDCLACLHFNHSGICELHCPALVTYNT DTFESMPNPEGRYTFGASCVTACPYNYLSTDVGSCTLVCPLHNQEVTAEDGTQRCEKC SKPCARVCYGLGMEHLREVRAVTSANIQEFAGCKKIFGSLAFLPESFDGDPASNTAPL QPEQLQVFETLEEITGYLYISAWPDSLPDLSVFQNLQVIRGRILHNGAYSLTLQGLGI SWLGLRSLRELGSGLALIHHNTHLCFVHTVPWDQLFRNPHQALLHTANRPEDECVGEG LACHQLCARGHCWGPGPTQCVNCSQFLRGQECVEECRVLQGLPREYVNARHCLPCHPE CQPQNGSVTCFGPEADQCVACAHYKDPPFCVARCPSGVKPDLSYMPIWKFPDEEGACQ PCPINCTHSCVDLDDKGCPAEQRASGTLEVLFQGPMSPILGYWKIKGLVQPTRLLLEY LEEKYEEHLYERDEGDKWRNKKFELGLEFPNLPYYIDGDVKLTQSMAIIRYIADKHNM LGGCPKERAEISMLEGAVLDIRYGVSRIAYSKDFETLKVDFLSKLPEMLKMFEDRLCH KTYLNGDHVTHPDFMLYDALDVVLYMDPMCLDAFPKLVCFKKRIEAIPQIDKYLKSSK YIAWPLQGWQATFGGGDHPPKSD
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagcggtaccctggaagttctgttccaggggcccatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggattga

SEQ ID NO: 99 - protein, 879 aa, synthetic construct
  REGION 1..879: note = Synthetic Construct
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASGTLEVLFQGPMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 100 - DNA, 1182 bp, synthetic construct
  misc_feature 1..1182: note = Description of the artificial sequence: base sequence of hMOG-FLAG-Fc including signal sequence
  source 1..1182: mol_type = other DNA
atggcaagcttatcaagaccctctctgcccagctgcctctgctccttcctcctcctcctcctcctccaagtgtcttccagctatgcagggcagttcagagtgataggaccaagacaccctatccgggctctggtcggggatgaagtggaattgccatgtcgcatatctcctgggaagaacgctacaggcatggaggtggggtggtaccgcccccccttctctagggtggttcatctctacagaaatggcaaggaccaagatggagaccaggcacctgaatatcggggccggacagagctgctgaaagatgctattggtgagggaaaggtgactctcaggatccggaatgtaaggttctcagatgaaggaggtttcacctgcttcttccgagatcattcttaccaagaggaggcagcaatggaattgaaagtagaagatcctttctactgggtgagccctggatctagagcagactacaaggacgacgatgacaagactagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 101 - protein, 394 aa, synthetic construct
  REGION 1..394: note = Description of the artificial sequence: amino acid sequence of hMOG-FLAG-Fc including signal sequence
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGSRADYKDDDDKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - DNA, 1182 bp, synthetic construct
  misc_feature 1..1182: note = The Description of the artificial sequence: base sequence of mMOG-FLAG-Fc including signal sequence
  source 1..1182: mol_type = other DNA
atggcctgtttgtggagcttctcttggcccagctgcttcctctcccttctcctcctccttctcctccagttgtcatgcagctatgcaggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggttctagagcagactacaaggacgacgatgacaagactagtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 103 - protein, 394 aa, synthetic construct
  REGION 1..394: note = Description of the artificial sequence: amino acid sequence of mMOG-FLAG-Fc including signal sequence
MACLWSFSWPSCFLSLLLLLLLQLSCSYAGQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVEDPFYWVNPGSRADYKDDDDKTSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - DNA, 1152 bp, synthetic construct
  misc_feature 1..1152: note = Description of the artificial sequence: base sequence of hMOG-GST including signal sequence
  source 1..1152: mol_type = other DNA
atggcaagcttatcaagaccctctctgcccagctgcctctgctccttcctcctcctcctcctcctccaagtgtcttccagctatgcagggcagttcagagtgataggaccaagacaccctatccgggctctggtcggggatgaagtggaattgccatgtcgcatatctcctgggaagaacgctacaggcatggaggtggggtggtaccgcccccccttctctagggtggttcatctctacagaaatggcaaggaccaagatggagaccaggcacctgaatatcggggccggacagagctgctgaaagatgctattggtgagggaaaggtgactctcaggatccggaatgtaaggttctcagatgaaggaggtttcacctgcttcttccgagatcattcttaccaagaggaggcagcaatggaattgaaagtagaagatcctttctactgggtgagccctggaggtaccctggaagttctgttccaggggcccatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggat

SEQ ID NO: 105 - protein, 384 aa, synthetic construct
  REGION 1..384: note = Description of the artificial sequence: amino acid sequence of hMOG-GST including signal sequence
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGGTLEVLFQGPMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 106 - DNA, 1152 bp, synthetic construct
  misc_feature 1..1152: note = Description of the artificial sequence: base sequence of mMOG-GST including signal sequence
  source 1..1152: mol_type = other DNA
atggcctgtttgtggagcttctcttggcccagctgcttcctctcccttctcctcctccttctcctccagttgtcatgcagctatgcaggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtggtaccctggaagttctgttccaggggcccatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggat

SEQ ID NO: 107 - protein, 384 aa, synthetic construct
  REGION 1..384: note = Description of the artificial sequence: amino acid sequence of mMOG-GST including signal sequence
MACLWSFSWPSCFLSLLLLLLLQLSCSYAGQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVEDPFYWVNPGGTLEVLFQGPMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 108 - DNA, 1371 bp, synthetic construct
  misc_feature 1..1371: note = Description of the artificial sequence: base sequence of the antibody sequence of pCI-AVM-hLG4PE (R409K / S354C / T366W) -FLAG tag excluding signal sequence
  source 1..1371: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatgccaggaggagatgaccaagaaccaggtcagcctgtggtgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtaaagactacaaggacgacgatgacaag

SEQ ID NO: 109 - protein, 457 aa, synthetic construct
  REGION 1..457: note = Description of the artificial sequence: amino acid sequence of the antibody sequence of pCI-AVM-hLG4PE (R409K / S354C / T366W) -FLAG tag excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGKDYKDDDDK

SEQ ID NO: 110 - DNA, 1380 bp, synthetic construct
  misc_feature 1..1380: note = Description of the artificial sequence: base sequence of the antibody sequence of pCI-MOG01-hLG4PE (R409K / Y349C / T366S / L368A / Y407V) -His tag excluding signal sequence
  source 1..1380: mol_type = other DNA
caggtacagctgcagcagtcaggcgcaggattattgaagccttcggagaccctttccctcacctgcgctgtgtctggtgggtccttcagtggttactactggacctggatccgccagcgcccagggaaggggctggagtggattggagaaatcaatcatcgtggaagcaccgattacaacccgtccctcaagagtcgagtcaccatgtcaatagacacgtccaagagccagttctccctgaatttgaaatctgtgaccgccgcggacacggctgtgtattactgtgcgagagccgcctgggggtcttgttatgatgggacctgctaccccgctgaatacttccaatactggggccagggaaccctggtcaccgtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtgcaccctgcccccatcccaggaggagatgaccaagaaccaggtcagcctgtcctgcgcggtcaaaggcttctaccccagcgacatcgccgtggagtgggagagcaatgggcagccggagaacaactacaagaccacgcctcccgtgctggactccgacggctccttcttcctcgtcagcaagctaaccgtggacaagagcaggtggcaggaggggaatgtcttctcatgctccgtgatgcatgaggctctgcacaaccactacacacagaagagcctctccctgtctctgggtaaacaccaccaccaccaccac

SEQ ID NO: 111 - protein, 460 aa, synthetic construct
  REGION 1..460: note = Description of the artificial sequence: amino acid sequence of the antibody sequence of pCI-MOG01-hLG4PE (R409K / Y349C / T366S / L368A / Y407V) -His tag excluding signal sequence
QVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLGKHHHHHH

SEQ ID NO: 112 - DNA, 2043 bp, synthetic construct
  misc_feature 1..2043: note = Description of the artificial sequence: base sequence of the antibody sequence of pCI-AVM-hLG4PE (R409K) -linker-MOG01 VL-CL excluding signal sequence
  source 1..2043: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtaaaggaggaggagggtccggaggaggagggtccggaggaggagggtcccagtctgccctgactcagcctgcctccgtgtctgggtctcctggacagtcgatcaccatctcctgcactggaaccagccgtgacgttggtggttataactatgtctcctggtaccaacaacacccaggcaaagcccccaaactcatgatttatgatgtcaataatcggccctcaggggtttctaatcggttctctggctccaagtctggcaacacggcctccctgaccatctctgggctccaggctgaggacgaggctgattatttctgcagctcatatacaagcagtagcacccctgtggtattcggcggtgggaccaagctgaccgtcctaggtcagcccaaggccgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 113 - protein, 681 aa, synthetic construct
  REGION 1..681: note = Description of the artificial sequence: amino acid sequence of the antibody sequence of pCI-AVM-hLG4PE (R409K) -linker-MOG01 VL-CL excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGKGGGGSGGGGSGGGGSQSALTQPASVSGSPGQSITISCTGTSRDVGGYNYVSWYQQHPGKAPKLMIYDVNNRPSGVSNRFSGSKSGNTASLTISGLQAEDEADYFCSSYTSSSTPVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 114 - DNA, 675 bp, synthetic construct
  misc_feature 1..675: note = Description of the artificial sequence: base sequence of antibody sequence of pCI-MOG01 VH-CH excluding signal sequence
  source 1..675: mol_type = other DNA
caggtacagctgcagcagtcaggcgcaggattattgaagccttcggagaccctttccctcacctgcgctgtgtctggtgggtccttcagtggttactactggacctggatccgccagcgcccagggaaggggctggagtggattggagaaatcaatcatcgtggaagcaccgattacaacccgtccctcaagagtcgagtcaccatgtcaatagacacgtccaagagccagttctccctgaatttgaaatctgtgaccgccgcggacacggctgtgtattactgtgcgagagccgcctgggggtcttgttatgatgggacctgctaccccgctgaatacttccaatactggggccagggaaccctggtcaccgtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagtt

SEQ ID NO: 115 - protein, 225 aa, synthetic construct
  REGION 1..225: note = Description of the artificial sequence: amino acid sequence of antibody sequence of pCI-MOG01 VH-CH excluding signal sequence
QVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 116 - DNA, 2181 bp, synthetic construct
  misc_feature 1..2181: note = Description of the artificial sequence: base sequence of antibody sequence of pCI-AVM-hLG4PE (R409K / S354C / T366W) -linker-MOG01 scFv-FLAG tag excluding signal sequence
  source 1..2181: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatgccaggaggagatgaccaagaaccaggtcagcctgtggtgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtggaggaagcggaggaggagggtccggaggaggagggtccggaggaggagggtcccaggtacagctgcagcagtcaggcgcaggattattgaagccttcggagaccctttccctcacctgcgctgtgtctggtgggtccttcagtggttactactggacctggatccgccagcgcccagggaaggggctggagtggattggagaaatcaatcatcgtggaagcaccgattacaacccgtccctcaagagtcgagtcaccatgtcaatagacacgtccaagagccagttctccctgaatttgaaatctgtgaccgccgcggacacggctgtgtattactgtgcgagagccgcctgggggtcttgttatgatgggacctgctaccccgctgaatacttccaatactggggccagggaaccctggtcaccgtctcctcaggaggcggtggcagcggtgggcgcgcctcgggcggaggtggttcacagtctgccctgactcagcctgcctccgtgtctgggtctcctggacagtcgatcaccatctcctgcactggaaccagccgtgacgttggtggttataactatgtctcctggtaccaacaacacccaggcaaagcccccaaactcatgatttatgatgtcaataatcggccctcaggggtttctaatcggttctctggctccaagtctggcaacacggcctccctgaccatctctgggctccaggctgaggacgaggctgattatttctgcagctcatatacaagcagtagcacccctgtggtattcggcggtgggaccaagctgaccgtcctagactacaaggacgacgatgacaag

SEQ ID NO: 117 - protein, 727 aa, synthetic construct
  REGION 1..727: note = Description of the artificial sequence: amino acid sequence of the antibody sequence of pCI-AVM-hLG4PE (R409K / S354C / T366W) -linker-MOG01 scFv-FLAG tag excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGGGSGGGGSGGGGSGGGGSQVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSSGGGGSGGRASGGGGSQSALTQPASVSGSPGQSITISCTGTSRDVGGYNYVSWYQQHPGKAPKLMIYDVNNRPSGVSNRFSGSKSGNTASLTISGLQAEDEADYFCSSYTSSSTPVVFGGGTKLTVLDYKDDDDK

SEQ ID NO: 118 - DNA, 1365 bp, synthetic construct
  misc_feature 1..1365: note = Description of the artificial sequence: base sequence of the antibody sequence of pCI-AVM-hLG4PE (R409K / Y349C / T366S / L368A / Y407V) -His tag excluding signal sequence
  source 1..1365: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtgcaccctgcccccatcccaggaggagatgaccaagaaccaggtcagcctgtcctgcgcggtcaaaggcttctaccccagcgacatcgccgtggagtgggagagcaatgggcagccggagaacaactacaagaccacgcctcccgtgctggactccgacggctccttcttcctcgtcagcaagctaaccgtggacaagagcaggtggcaggaggggaatgtcttctcatgctccgtgatgcatgaggctctgcacaaccactacacacagaagagcctctccctgtctctgggtaaacaccaccaccaccaccac

SEQ ID NO: 119 - protein, 455 aa, synthetic construct
  REGION 1..455: note = Description of the artificial sequence: amino acid sequence of the antibody sequence of pCI-AVM-hLG4PE (R409K / Y349C / T366S / L368A / Y407V) -His tag excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLGKHHHHHH

SEQ ID NO: 120 - DNA, 2151 bp, synthetic construct
  misc_feature 1..2151: note = Description of the artificial sequence: base sequence of the antibody sequence of pCI-AVM-hLG4PE (R409K) _MOG01 scFv2 excluding signal sequence
  source 1..2151: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtaaaggaggaggagggtccggaggaggagggtccggaggaggagggtcccaggtacagctgcagcagtcaggcgcaggattattgaagccttcggagaccctttccctcacctgcgctgtgtctggtgggtccttcagtggttactactggacctggatccgccagcgcccagggaaggggctggagtggattggagaaatcaatcatcgtggaagcaccgattacaacccgtccctcaagagtcgagtcaccatgtcaatagacacgtccaagagccagttctccctgaatttgaaatctgtgaccgccgcggacacggctgtgtattactgtgcgagagccgcctgggggtcttgttatgatgggacctgctaccccgctgaatacttccaatactggggccagggaaccctggtcaccgtctcctcaggaggcggtggcagcggtgggcgcgcctcgggcggaggtggttcacagtctgccctgactcagcctgcctccgtgtctgggtctcctggacagtcgatcaccatctcctgcactggaaccagccgtgacgttggtggttataactatgtctcctggtaccaacaacacccaggcaaagcccccaaactcatgatttatgatgtcaataatcggccctcaggggtttctaatcggttctctggctccaagtctggcaacacggcctccctgaccatctctgggctccaggctgaggacgaggctgattatttctgcagctcatatacaagcagtagcacccctgtggtattcggcggtgggaccaagctgaccgtccta

SEQ ID NO: 121 - protein, 717 aa, synthetic construct
  REGION 1..717: note = Description of the artificial sequence: amino acid sequence of antibody sequence of pCI-AVM-hLG4PE (R409K) _MOG 01 scFv 2 excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGKGGGGSGGGGSGGGGSQVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSSGGGGSGGRASGGGGSQSALTQPASVSGSPGQSITISCTGTSRDVGGYNYVSWYQQHPGKAPKLMIYDVNNRPSGVSNRFSGSKSGNTASLTISGLQAEDEADYFCSSYTSSSTPVVFGGGTKLTVL

SEQ ID NO: 122 - DNA, 2157 bp, synthetic construct
  misc_feature 1..2157: note = Description of the artificial sequence: base sequence of antibody sequence of pCI-AVM-hLG4PE (R409K) _MOG01 scFv3 excluding signal sequence
  source 1..2157: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtggaggaagcggaggaggagggtccggaggaggagggtccggaggaggagggtcccagtctgccctgactcagcctgcctccgtgtctgggtctcctggacagtcgatcaccatctcctgcactggaaccagccgtgacgttggtggttataactatgtctcctggtaccaacaacacccaggcaaagcccccaaactcatgatttatgatgtcaataatcggccctcaggggtttctaatcggttctctggctccaagtctggcaacacggcctccctgaccatctctgggctccaggctgaggacgaggctgattatttctgcagctcatatacaagcagtagcacccctgtggtattcggcggtgggaccaagctgaccgtcctaggaggcggtggcagcggtgggcgcgcctcgggcggaggtggttcacaggtacagctgcagcagtcaggcgcaggattattgaagccttcggagaccctttccctcacctgcgctgtgtctggtgggtccttcagtggttactactggacctggatccgccagcgcccagggaaggggctggagtggattggagaaatcaatcatcgtggaagcaccgattacaacccgtccctcaagagtcgagtcaccatgtcaatagacacgtccaagagccagttctccctgaatttgaaatctgtgaccgccgcggacacggctgtgtattactgtgcgagagccgcctgggggtcttgttatgatgggacctgctaccccgctgaatacttccaatactggggccagggaaccctggtcaccgtctcctca

SEQ ID NO: 123 - protein, 719 aa, synthetic construct
  REGION 1..719: note = Description of the artificial sequence: amino acid sequence of the antibody sequence of pCI-AVM-hLG4PE (R409K) _MOG 01 scFv 3 excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGSGGGGSGGGGSGGGGSQSALTQPASVSGSPGQSITISCTGTSRDVGGYNYVSWYQQHPGKAPKLMIYDVNNRPSGVSNRFSGSKSGNTASLTISGLQAEDEADYFCSSYTSSSTPVVFGGGTKLTVLGGGGSGGRASGGGGSQVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSS

SEQ ID NO: 124 - DNA, 2175 bp, synthetic construct
  misc_feature 1..2175: note = Description of the artificial sequence: base sequence of the antibody sequence of pCI-AVM-hLG4PE (R409K) _MOG 01 scFv 4 excluding signal sequence
  source 1..2175: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtggaggaggagggtccggaggaggagggtccggtggaggtgggtcccaggtacagctgcagcagtcaggcgcaggattattgaagccttcggagaccctttccctcacctgcgctgtgtctggtgggtccttcagtggttactactggacctggatccgccagcgcccagggaaggggctggagtggattggagaaatcaatcatcgtggaagcaccgattacaacccgtccctcaagagtcgagtcaccatgtcaatagacacgtccaagagccagttctccctgaatttgaaatctgtgaccgccgcggacacggctgtgtattactgtgcgagagccgcctgggggtcttgttatgatgggacctgctaccccgctgaatacttccaatactggggccagggaaccctggtcaccgtctcctcagctagcaccggaggcggtggcagcggaggaggagggtccggtgggggcggctcgggcggaggtggttcacagtctgccctgactcagcctgcctccgtgtctgggtctcctggacagtcgatcaccatctcctgcactggaaccagccgtgacgttggtggttataactatgtctcctggtaccaacaacacccaggcaaagcccccaaactcatgatttatgatgtcaataatcggccctcaggggtttctaatcggttctctggctccaagtctggcaacacggcctccctgaccatctctgggctccaggctgaggacgaggctgattatttctgcagctcatatacaagcagtagcacccctgtggtattcggcggtgggaccaagctgaccgtcctaggt

SEQ ID NO: 125 - protein, 725 aa, synthetic construct
  REGION 1..725: note = Description of the artificial sequence: amino acid sequence of the antibody sequence of pCI-AVM-hLG4PE (R409K) _MOG 01 scFv 4 excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSQVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSSASTGGGGSGGGGSGGGGSGGGGSQSALTQPASVSGSPGQSITISCTGTSRDVGGYNYVSWYQQHPGKAPKLMIYDVNNRPSGVSNRFSGSKSGNTASLTISGLQAEDEADYFCSSYTSSSTPVVFGGGTKLTVLG

SEQ ID NO: 126 - DNA, 2175 bp, synthetic construct
  misc_feature 1..2175: note = Description of the artificial sequence: base sequence of antibody sequence of pCI-AVM-hLG4PE (R409K) _MOG 01 scFv 5 excluding signal sequence
  source 1..2175: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtggaggaggagggtccggaggaggagggtccggtggaggtgggtcccagtctgccctgactcagcctgcctccgtgtctgggtctcctggacagtcgatcaccatctcctgcactggaaccagccgtgacgttggtggttataactatgtctcctggtaccaacaacacccaggcaaagcccccaaactcatgatttatgatgtcaataatcggccctcaggggtttctaatcggttctctggctccaagtctggcaacacggcctccctgaccatctctgggctccaggctgaggacgaggctgattatttctgcagctcatatacaagcagtagcacccctgtggtattcggcggtgggaccaagctgaccgtcctaggtggaggcggtggcagcggaggaggagggtccggtgggggcggctcgggcggaggtggttcacaggtacagctgcagcagtcaggcgcaggattattgaagccttcggagaccctttccctcacctgcgctgtgtctggtgggtccttcagtggttactactggacctggatccgccagcgcccagggaaggggctggagtggattggagaaatcaatcatcgtggaagcaccgattacaacccgtccctcaagagtcgagtcaccatgtcaatagacacgtccaagagccagttctccctgaatttgaaatctgtgaccgccgcggacacggctgtgtattactgtgcgagagccgcctgggggtcttgttatgatgggacctgctaccccgctgaatacttccaatactggggccagggaaccctggtcaccgtctcctcagctagcacc

SEQ ID NO: 127 - protein, 725 aa, synthetic construct
  REGION 1..725: note = Description of the artificial sequence: amino acid sequence of the antibody sequence of pCI-AVM-hLG4PE (R409K) _MOG01 scFv5 excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSQSALTQPASVSGSPGQSITISCTGTSRDVGGYNYVSWYQQHPGKAPKLMIYDVNNRPSGVSNRFSGSKSGNTASLTISGLQAEDEADYFCSSYTSSSTPVVFGGGTKLTVLGGGGGSGGGGSGGGGSGGGGSQVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSSAST

SEQ ID NO: 128 - DNA, 2148 bp, synthetic construct
  misc_feature 1..2148: note = Description of the artificial sequence: base sequence of the antibody sequence of pCI-AVM-hLG4PE (R409K) _MOG01 scFv6 excluding signal sequence
  source 1..2148: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtggaggaggagggtccggaggaggagggtccggtggaggtgggtcccagtctgccctgactcagcctgcctccgtgtctgggtctcctggacagtcgatcaccatctcctgcactggaaccagccgtgacgttggtggttataactatgtctcctggtaccaacaacacccaggcaaagcccccaaactcatgatttatgatgtcaataatcggccctcaggggtttctaatcggttctctggctccaagtctggcaacacggcctccctgaccatctctgggctccaggctgaggacgaggctgattatttctgcagctcatatacaagcagtagcacccctgtggtattcggcggtgggaccaagctgaccgtcctaggaggcggtggcagcggtgggcgcgcctcgggcggaggtggttcacaggtacagctgcagcagtcaggcgcaggattattgaagccttcggagaccctttccctcacctgcgctgtgtctggtgggtccttcagtggttactactggacctggatccgccagcgcccagggaaggggctggagtggattggagaaatcaatcatcgtggaagcaccgattacaacccgtccctcaagagtcgagtcaccatgtcaatagacacgtccaagagccagttctccctgaatttgaaatctgtgaccgccgcggacacggctgtgtattactgtgcgagagccgcctgggggtcttgttatgatgggacctgctaccccgctgaatacttccaatactggggccagggaaccctggtcaccgtctcctca

SEQ ID NO: 129 - protein, 716 aa, synthetic construct
  REGION 1..716: note = Description of the artificial sequence: amino acid sequence of the antibody sequence of pCI-AVM-hLG4PE (R409K) _MOG 01 scFv 6 excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSQSALTQPASVSGSPGQSITISCTGTSRDVGGYNYVSWYQQHPGKAPKLMIYDVNNRPSGVSNRFSGSKSGNTASLTISGLQAEDEADYFCSSYTSSSTPVVFGGGTKLTVLGGGGSGGRASGGGGSQVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSS

SEQ ID NO: 130 - DNA, 2172 bp, synthetic construct
  misc_feature 1..2172: note = Description of the artificial sequence: base sequence of the antibody sequence of pCI-AVM-hLG4PE (R409K) _MOG01 scFv7 excluding signal sequence
  source 1..2172: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtggaggaagcggaggaggagggtccggaggaggagggtccggaggaggagggtcccagtctgccctgactcagcctgcctccgtgtctgggtctcctggacagtcgatcaccatctcctgcactggaaccagccgtgacgttggtggttataactatgtctcctggtaccaacaacacccaggcaaagcccccaaactcatgatttatgatgtcaataatcggccctcaggggtttctaatcggttctctggctccaagtctggcaacacggcctccctgaccatctctgggctccaggctgaggacgaggctgattatttctgcagctcatatacaagcagtagcacccctgtggtattcggcggtgggaccaagctgaccgtcctaggaggcggtggcagcggaggaggagggtccggtgggggcggctcgggcggaggtggttcacaggtacagctgcagcagtcaggcgcaggattattgaagccttcggagaccctttccctcacctgcgctgtgtctggtgggtccttcagtggttactactggacctggatccgccagcgcccagggaaggggctggagtggattggagaaatcaatcatcgtggaagcaccgattacaacccgtccctcaagagtcgagtcaccatgtcaatagacacgtccaagagccagttctccctgaatttgaaatctgtgaccgccgcggacacggctgtgtattactgtgcgagagccgcctgggggtcttgttatgatgggacctgctaccccgctgaatacttccaatactggggccagggaaccctggtcaccgtctcctca

SEQ ID NO: 131 - protein, 724 aa, synthetic construct
  REGION 1..724: note = Description of the artificial sequence: amino acid sequence of the antibody sequence of pCI-AVM-hLG4PE (R409K) _MOG01 scFv7 excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGSGGGGSGGGGSGGGGSQSALTQPASVSGSPGQSITISCTGTSRDVGGYNYVSWYQQHPGKAPKLMIYDVNNRPSGVSNRFSGSKSGNTASLTISGLQAEDEADYFCSSYTSSSTPVVFGGGTKLTVLGGGGSGGGGSGGGGSGGGGSQVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSS

SEQ ID NO: 132 - DNA, 2169 bp, synthetic construct
  misc_feature 1..2169: note = Description of the artificial sequence: base sequence of the antibody sequence of pCI-AVM-hLG4PE (R409K) _MOG 01 scFv 8 excluding signal sequence
  source 1..2169: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtggaggaagcggaggaggagggtccggaggaggagggtccggaggaggagggtcccagtctgccctgactcagcctgcctccgtgtctgggtctcctggacagtcgatcaccatctcctgcactggaaccagccgtgacgttggtggttataactatgtctcctggtaccaacaacacccaggcaaagcccccaaactcatgatttatgatgtcaataatcggccctcaggggtttctaatcggttctctggctccaagtctggcaacacggcctccctgaccatctctgggctccaggctgaggacgaggctgattatttctgcagctcatatacaagcagtagcacccctgtggtattcggcggtgggaccaagctgaccgtcctaggtggaggcggtggcagcggtgggcgcgcctcgggcggaggtggttcacaggtacagctgcagcagtcaggcgcaggattattgaagccttcggagaccctttccctcacctgcgctgtgtctggtgggtccttcagtggttactactggacctggatccgccagcgcccagggaaggggctggagtggattggagaaatcaatcatcgtggaagcaccgattacaacccgtccctcaagagtcgagtcaccatgtcaatagacacgtccaagagccagttctccctgaatttgaaatctgtgaccgccgcggacacggctgtgtattactgtgcgagagccgcctgggggtcttgttatgatgggacctgctaccccgctgaatacttccaatactggggccagggaaccctggtcaccgtctcctcagctagcacc

SEQ ID NO: 133 - protein, 723 aa, synthetic construct
  REGION 1..723: note = Description of the artificial sequence: amino acid sequence of the antibody sequence of pCI-AVM-hLG4PE (R409K) _MOG 01 scFv 8 excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGSGGGGSGGGGSGGGGSQSALTQPASVSGSPGQSITISCTGTSRDVGGYNYVSWYQQHPGKAPKLMIYDVNNRPSGVSNRFSGSKSGNTASLTISGLQAEDEADYFCSSYTSSSTPVVFGGGTKLTVLGGGGGSGGRASGGGGSQVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSSAST

SEQ ID NO: 134 - DNA, 2163 bp, synthetic construct
  misc_feature 1..2163: note = Description of the artificial sequence: base sequence of antibody sequence of pCI-AVM-hLG4PE (R409K) _MOG01 scFv9 excluding signal sequence
  source 1..2163: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtggaggaggagggtccggaggaggagggtccggtggaggtgggtcccagtctgccctgactcagcctgcctccgtgtctgggtctcctggacagtcgatcaccatctcctgcactggaaccagccgtgacgttggtggttataactatgtctcctggtaccaacaacacccaggcaaagcccccaaactcatgatttatgatgtcaataatcggccctcaggggtttctaatcggttctctggctccaagtctggcaacacggcctccctgaccatctctgggctccaggctgaggacgaggctgattatttctgcagctcatatacaagcagtagcacccctgtggtattcggcggtgggaccaagctgaccgtcctaggaggcggtggcagcggaggaggagggtccggtgggggcggctcgggcggaggtggttcacaggtacagctgcagcagtcaggcgcaggattattgaagccttcggagaccctttccctcacctgcgctgtgtctggtgggtccttcagtggttactactggacctggatccgccagcgcccagggaaggggctggagtggattggagaaatcaatcatcgtggaagcaccgattacaacccgtccctcaagagtcgagtcaccatgtcaatagacacgtccaagagccagttctccctgaatttgaaatctgtgaccgccgcggacacggctgtgtattactgtgcgagagccgcctgggggtcttgttatgatgggacctgctaccccgctgaatacttccaatactggggccagggaaccctggtcaccgtctcctca

SEQ ID NO: 135 - protein, 721 aa, synthetic construct
  REGION 1..721: note = Description of the artificial sequence: amino acid sequence of the antibody sequence of pCI-AVM-hLG4PE (R409K) _MOG01 scFv9 excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSQSALTQPASVSGSPGQSITISCTGTSRDVGGYNYVSWYQQHPGKAPKLMIYDVNNRPSGVSNRFSGSKSGNTASLTISGLQAEDEADYFCSSYTSSSTPVVFGGGTKLTVLGGGGSGGGGSGGGGSGGGGSQVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSS

SEQ ID NO: 136 - DNA, 2160 bp, synthetic construct
  misc_feature 1..2160: note = Description of the artificial sequence: base sequence of antibody sequence of pCI-AVM-hLG4PE (R409K) _MOG 01 scFv 10 excluding signal sequence
  source 1..2160: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtggaggaggagggtccggaggaggagggtccggtggaggtgggtcccagtctgccctgactcagcctgcctccgtgtctgggtctcctggacagtcgatcaccatctcctgcactggaaccagccgtgacgttggtggttataactatgtctcctggtaccaacaacacccaggcaaagcccccaaactcatgatttatgatgtcaataatcggccctcaggggtttctaatcggttctctggctccaagtctggcaacacggcctccctgaccatctctgggctccaggctgaggacgaggctgattatttctgcagctcatatacaagcagtagcacccctgtggtattcggcggtgggaccaagctgaccgtcctaggtggaggcggtggcagcggtgggcgcgcctcgggcggaggtggttcacaggtacagctgcagcagtcaggcgcaggattattgaagccttcggagaccctttccctcacctgcgctgtgtctggtgggtccttcagtggttactactggacctggatccgccagcgcccagggaaggggctggagtggattggagaaatcaatcatcgtggaagcaccgattacaacccgtccctcaagagtcgagtcaccatgtcaatagacacgtccaagagccagttctccctgaatttgaaatctgtgaccgccgcggacacggctgtgtattactgtgcgagagccgcctgggggtcttgttatgatgggacctgctaccccgctgaatacttccaatactggggccagggaaccctggtcaccgtctcctcagctagcacc

SEQ ID NO: 137 - protein, 720 aa, synthetic construct
  REGION 1..720: note = Description of the artificial sequence: amino acid sequence of the antibody sequence of pCI-AVM-hLG4PE (R409K) _MOG 01 scFv 10 excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSQSALTQPASVSGSPGQSITISCTGTSRDVGGYNYVSWYQQHPGKAPKLMIYDVNNRPSGVSNRFSGSKSGNTASLTISGLQAEDEADYFCSSYTSSSTPVVFGGGTKLTVLGGGGGSGGRASGGGGSQVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSSAST

SEQ ID NO: 138 - DNA, 2184 bp, synthetic construct
  misc_feature 1..2184: note = Description of the artificial sequence: base sequence of the antibody sequence of pCI-AVM-hLG4PE (R409K) _MOG01 scFv11 excluding signal sequence
  source 1..2184: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtggaggaagcggaggaggagggtccggaggaggagggtccggaggaggagggtcccagtctgccctgactcagcctgcctccgtgtctgggtctcctggacagtcgatcaccatctcctgcactggaaccagccgtgacgttggtggttataactatgtctcctggtaccaacaacacccaggcaaagcccccaaactcatgatttatgatgtcaataatcggccctcaggggtttctaatcggttctctggctccaagtctggcaacacggcctccctgaccatctctgggctccaggctgaggacgaggctgattatttctgcagctcatatacaagcagtagcacccctgtggtattcggcggtgggaccaagctgaccgtcctaggtggaggcggtggcagcggaggaggagggtccggtgggggcggctcgggcggaggtggttcacaggtacagctgcagcagtcaggcgcaggattattgaagccttcggagaccctttccctcacctgcgctgtgtctggtgggtccttcagtggttactactggacctggatccgccagcgcccagggaaggggctggagtggattggagaaatcaatcatcgtggaagcaccgattacaacccgtccctcaagagtcgagtcaccatgtcaatagacacgtccaagagccagttctccctgaatttgaaatctgtgaccgccgcggacacggctgtgtattactgtgcgagagccgcctgggggtcttgttatgatgggacctgctaccccgctgaatacttccaatactggggccagggaaccctggtcaccgtctcctcagctagcacc

SEQ ID NO: 139 - protein, 728 aa, synthetic construct
  REGION 1..728: note = Description of the artificial sequence: amino acid sequence of the antibody sequence of pCI-AVM-hLG4PE (R409K) _MOG 01 scFv 11 excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGSGGGGSGGGGSGGGGSQSALTQPASVSGSPGQSITISCTGTSRDVGGYNYVSWYQQHPGKAPKLMIYDVNNRPSGVSNRFSGSKSGNTASLTISGLQAEDEADYFCSSYTSSSTPVVFGGGTKLTVLGGGGGSGGGGSGGGGSGGGGSQVQLQQSGAGLLKPSETLSLTCAVSGGSFSGYYWTWIRQRPGKGLEWIGEINHRGSTDYNPSLKSRVTMSIDTSKSQFSLNLKSVTAADTAVYYCARAAWGSCYDGTCYPAEYFQYWGQGTLVTVSSAST

SEQ ID NO: 140 - DNA, 2040 bp, synthetic construct
  misc_feature 1..2040: note = Description of the artificial sequence: base sequence of the antibody sequence of pCI-AVM-hLG4PE (R409K) -linker-AVMVL-CL excluding signal sequence
  source 1..2040: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtaaaggaggaggagggtccggaggaggagggtccggaggaggagggtcccagtttgtgctttctcagccaaactctgtgtctacgaatctcggaagcacagtcaaactgtcttgcaagcgcagcactggtaacattggaagcaattatgtgagctggtaccagcagcatgagggaagatctcccaccactatgatttatagggatgataagagaccagatggagttcctgacaggttctctggctccattgacagatcttccgactcagccctcctgacaatcaataatgtgcagactgaagatgaagctgactacttctgtcagtcttacagtagtggtattaatattttcggcggtggaaccaagctcactgtcctaggtcagcccaaggccgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 141 - protein, 680 aa, synthetic construct
  REGION 1..680: note = Description of the artificial sequence: amino acid sequence of the antibody sequence of pCI-AVM-hLG4PE (R409K) -linker-AVMVL-CL excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGKGGGGSGGGGSGGGGSQFVLSQPNSVSTNLGSTVKLSCKRSTGNIGSNYVSWYQQHEGRSPTTMIYRDDKRPDGVPDRFSGSIDRSSDSALLTINNVQTEDEADYFCQSYSSGINIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 142 - DNA, 660 bp, synthetic construct
  misc_feature 1..660: note = Description of the artificial sequence: base sequence of antibody sequence of pCI-AVMVH-CH excluding signal sequence
  source 1..660: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagtt

SEQ ID NO: 143 - protein, 220 aa, synthetic construct
  REGION 1..220: note = Description of the artificial sequence: amino acid sequence of antibody sequence of pCI-AVMVH-CH excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 144 - DNA, 2163 bp, synthetic construct
  misc_feature 1..2163: note = Description of the artificial sequence: base sequence of the antibody sequence of pCI-AVM-hLG4PE (R409K / S354C / T366W) -linker-AVMscFv-FLAG tag excluding signal sequence
  source 1..2163: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatgccaggaggagatgaccaagaaccaggtcagcctgtggtgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtggaggaagcggaggaggagggtccggaggaggagggtccggaggaggagggtccgaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcaggaggcggtggcagcggtgggcgcgcctcgggcggaggtggttcacagtttgtgctttctcagccaaactctgtgtctacgaatctcggaagcacagtcaaactgtcttgcaagcgcagcactggtaacattggaagcaattatgtgagctggtaccagcagcatgagggaagatctcccaccactatgatttatagggatgataagagaccagatggagttcctgacaggttctctggctccattgacagatcttccgactcagccctcctgacaatcaataatgtgcagactgaagatgaagctgactacttctgtcagtcttacagtagtggtattaatattttcggcggtggaaccaagctcactgtcctagactacaaggacgacgatgacaag

SEQ ID NO: 145 - protein, 721 aa, synthetic construct
  REGION 1..721: note = Description of the artificial sequence: amino acid sequence of the antibody sequence of pCI-AVM-hLG4PE (R409K / S354C / T366W) -linker-AVMscFv-FLAG tag excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLGGGSGGGGSGGGGSGGGGSEVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSGGGGSGGRASGGGGSQFVLSQPNSVSTNLGSTVKLSCKRSTGNIGSNYVSWYQQHEGRSPTTMIYRDDKRPDGVPDRFSGSIDRSSDSALLTINNVQTEDEADYFCQSYSSGINIFGGGTKLTVLDYKDDDDK

SEQ ID NO: 146 - DNA, 2139 bp, synthetic construct
  misc_feature 1..2139: note = Description of the artificial sequence: base sequence of the antibody sequence of pCI-AVM-hLG4PE (R409K) _AVMscFv3 excluding signal sequence
  source 1..2139: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtggaggaagcggaggaggagggtccggaggaggagggtccggaggaggagggtcccagtttgtgctttctcagccaaactctgtgtctacgaatctcggaagcacagtcaaactgtcttgcaagcgcagcactggtaacattggaagcaattatgtgagctggtaccagcagcatgagggaagatctcccaccactatgatttatagggatgataagagaccagatggagttcctgacaggttctctggctccattgacagatcttccgactcagccctcctgacaatcaataatgtgcagactgaagatgaagctgactacttctgtcagtcttacagtagtggtattaatattttcggcggtggaaccaagctcactgtcctaggaggcggtggcagcggtgggcgcgcctcgggcggaggtggttcagaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctca

SEQ ID NO: 147 - protein, 713 aa, synthetic construct
  REGION 1..713: note = Description of the artificial sequence: amino acid sequence of the antibody sequence of pCI-AVM-hLG4PE (R409K) _AVMscFv3 excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGSGGGGSGGGGSGGGGSQFVLSQPNSVSTNLGSTVKLSCKRSTGNIGSNYVSWYQQHEGRSPTTMIYRDDKRPDGVPDRFSGSIDRSSDSALLTINNVQTEDEADYFCQSYSSGINIFGGGTKLTVLGGGGSGGRASGGGGSEVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSS

SEQ ID NO: 148 - DNA, 2157 bp, synthetic construct
  misc_feature 1..2157: note = Description of the artificial sequence: base sequence of the antibody sequence of pCI-AVM-hLG4PE (R409K) _AVMscFv5 excluding signal sequence
  source 1..2157: mol_type = other DNA
gaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcaccaaggggccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtggaggaggagggtccggaggaggagggtccggtggaggtgggtcccagtttgtgctttctcagccaaactctgtgtctacgaatctcggaagcacagtcaaactgtcttgcaagcgcagcactggtaacattggaagcaattatgtgagctggtaccagcagcatgagggaagatctcccaccactatgatttatagggatgataagagaccagatggagttcctgacaggttctctggctccattgacagatcttccgactcagccctcctgacaatcaataatgtgcagactgaagatgaagctgactacttctgtcagtcttacagtagtggtattaatattttcggcggtggaaccaagctcactgtcctaggtggaggcggtggcagcggaggaggagggtccggtgggggcggctcgggcggaggtggttcagaggtgcagctggtggaatctgggggaggcttagtgcagcctggaagatccctgaaactctcctgtgcagcctcaggattcactttcagtaactatgccatggcttgggtccgccgggctccaacgaagggtctggagtgggtcgcatccattagtaatggtggtggtaacacttactatcgcgactccgtgaagggccgattcactatctccagagatgatgcaaaaaacaccctatacctgcaaatggacagtctgaggtctgaggacacggccacttattactgtgcaagacacgggaattatatatattatgggtccttctttgattactggggccaaggagtcatggtcacagtctcctcagctagcacc

SEQ ID NO: 149 - protein, 719 aa, synthetic construct
  REGION 1..719: note = Description of the artificial sequence: amino acid sequence of the antibody sequence of pCI-AVM-hLG4PE (R409K) _AVMscFv5 excluding signal sequence
EVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSQFVLSQPNSVSTNLGSTVKLSCKRSTGNIGSNYVSWYQQHEGRSPTTMIYRDDKRPDGVPDRFSGSIDRSSDSALLTINNVQTEDEADYFCQSYSSGINIFGGGTKLTVLGGGGGSGGGGSGGGGSGGGGSEVQLVESGGGLVQPGRSLKLSCAASGFTFSNYAMAWVRRAPTKGLEWVASISNGGGNTYYRDSVKGRFTISRDDAKNTLYLQMDSLRSEDTATYYCARHGNYIYYGSFFDYWGQGVMVTVSSAST

SEQ ID NO: 150 - DNA, 1887 bp, synthetic construct
  misc_feature 1..1887: note = Description of the artificial sequence: base sequence of Acid Sphingomyelinase (ASM)
  source 1..1887: mol_type = other DNA
atgccccgctacggagcgtcactccgccagagctgccccaggtccggccgggagcagggacaagacgggaccgccggagcccccggactcctttggatgggcctggtgctggcgctggcgctggcgctggcgctggctctgtctgactctcgggttctctgggctccggcagaggctcaccctctttctccccaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcatacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccaggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgc

SEQ ID NO: 151 - DNA, 369 bp, synthetic construct
  misc_feature 1..369: note = Description of the artificial sequence: base sequence encoding VH excluding signal sequence of MOG 301
  source 1..369: mol_type = other DNA
caggtgcagctggtgcagtctggagctgaggtgaagaagcctggggcctcagtgaaggtctcctgcaaggcttctggttacagctttaccagctatggtatcaactgggtgcgacaggccccaggacaagggcttgagtggatgggatggatcagcgcttacaatggttacacaaactatgcacagaagctccagggcagagtcaccatgaccagagacacatccacgcgcacagcctacatggagctgaggagcctgagatctgacgacacggccgtgtattactgtgcgagagagtacgatattttgactggttattccgatgcttttgatatctggggccaagggaccctggtcactgtctcctca

SEQ ID NO: 152 - protein, 123 aa, synthetic construct
  REGION 1..123: note = The Description of the artificial sequence: amino acid sequence of VH excluding signal sequence of MOG 301
QVQLVQSGAEVKKPGASVKVSCKASGYSFTSYGINWVRQAPGQGLEWMGWISAYNGYTNYAQKLQGRVTMTRDTSTRTAYMELRSLRSDDTAVYYCAREYDILTGYSDAFDIWGQGTLVTVSS

SEQ ID NO: 153 - protein, 5 aa, synthetic construct
  REGION 1..5: note = The Description of the artificial sequence: amino acid sequence of HCDR1 of MOG 301
SYGIN

SEQ ID NO: 154 - protein, 17 aa, synthetic construct
  REGION 1..17: note = The Description of the artificial sequence: amino acid sequence of HCDR2 of MOG 301
WISAYNGYTNYAQKLQG

SEQ ID NO: 155 - protein, 14 aa, synthetic construct
  REGION 1..14: note = The Description of the artificial sequence: amino acid sequence of HCDR3 of MOG 301
EYDILTGYSDAFDI

SEQ ID NO: 156 - DNA, 324 bp, synthetic construct
  misc_feature 1..324: note = Description of the artificial sequence: base sequence encoding VL excluding signal sequence of MOG 301
  source 1..324: mol_type = other DNA
gaaatagtgatgacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgctcactttcggcggagggaccaaggtggaaatcaaa

SEQ ID NO: 157 - protein, 108 aa, synthetic construct
  REGION 1..108: note = Description of the artificial sequence: amino acid sequence of VL excluding signal sequence of MOG 301
EIVMTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIK

SEQ ID NO: 158 - protein, 12 aa, synthetic construct
  REGION 1..12: note = The Description of the artificial sequence: amino acid sequence of LCDR 1 of MOG 301
RASQSVSSSYLA

SEQ ID NO: 159 - protein, 7 aa, synthetic construct
  REGION 1..7: note = The Description of the artificial sequence: amino acid sequence of LCDR 2 of MOG 301
GASSRAT

SEQ ID NO: 160 - protein, 9 aa, synthetic construct
  REGION 1..9: note = The Description of the artificial sequence: amino acid sequence of LCDR 3 of MOG 301
QQYGSSPLT

SEQ ID NO: 161 - DNA, 366 bp, synthetic construct
  misc_feature 1..366: note = The Description of the artificial sequence: base sequence encoding VH excluding signal sequence of MOG 303
  source 1..366: mol_type = other DNA
gaggtgcagctggtggagtctgggggaggcttggtacagcctggggggtccctgagactctcctgtgcagcctctggattcacctttagcagctatgccatgggctgggtccgccaggctccagggaaggggctggaatgggtctcagctgttagtggtagtggtggtagcacatactacgcagactccgtgaagggccggttcaccatctccagagacaattccaagaacacgctgtatctgcaaatgaacagcctgagagccgaggacacggccgtatattactgtgcgaaaggaggatacgatattttgactggttacttctttgactactggggccagggaaccacggtcactgtctcctca

SEQ ID NO: 162 - protein, 122 aa, synthetic construct
  REGION 1..122: note = The Description of the artificial sequence: amino acid sequence of VH excluding signal sequence of MOG303
EVQLVESGGGLVQPGGSLRLSCAASGFTFSSYAMGWVRQAPGKGLEWVSAVSGSGGSTYYADSVKGRFTISRDNSKNTLYLQMNSLRAEDTAVYYCAKGGYDILTGYFFDYWGQGTTVTVSS

SEQ ID NO: 163 - protein, 5 aa, synthetic construct
  REGION 1..5: note = The Description of the artificial sequence: amino acid sequence of HCDR1 of MOG303
SYAMG

SEQ ID NO: 164 - protein, 17 aa, synthetic construct
  REGION 1..17: note = The Description of the artificial sequence: amino acid sequence of HCDR2 of MOG303
AVSGSGGSTYYADSVKG

SEQ ID NO: 165 - protein, 13 aa, synthetic construct
  REGION 1..13: note = The Description of the artificial sequence: amino acid sequence of HCDR3 of MOG303
GGYDILTGYFFDY

SEQ ID NO: 166 - DNA, 321 bp, synthetic construct
  misc_feature 1..321: note = The Description of the artificial sequence: base sequence encoding VL excluding signal sequence of MOG 303
  source 1..321: mol_type = other DNA
gccatccagatgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccattcactttcggccctgggacacgactggagattaaa

SEQ ID NO: 167 - protein, 107 aa, synthetic construct
  REGION 1..107: note = The Description of the artificial sequence: amino acid sequence of VL excluding signal sequence of MOG 303
AIQMTQSPSSLSASVGDRVTITCRASQGISSALAWYQQKPGKAPKLLIYDASSLESGVPSRFSGSGSGTDFTLTISSLQPEDFATYYCQQFNSYPFTFGPGTRLEIK

SEQ ID NO: 168 - protein, 11 aa, synthetic construct
  REGION 1..11: note = The Description of the artificial sequence: amino acid sequence of LCDR 1 of MOG 303
RASQGISSALA

SEQ ID NO: 169 - protein, 7 aa, synthetic construct
  REGION 1..7: note = The Description of the artificial sequence: amino acid sequence of LCDR 2 of MOG 303
DASSLES

SEQ ID NO: 170 - protein, 9 aa, synthetic construct
  REGION 1..9: note = The Description of the artificial sequence: amino acid sequence of LCDR 3 of MOG 303
QQFNSYPFT

SEQ ID NO: 171 - DNA, 381 bp, synthetic construct
  misc_feature 1..381: note = The Description of the artificial sequence: base sequence encoding VH (excluding signal sequence) of MOG 307
  source 1..381: mol_type = other DNA
cgggtcaccttgagggagtctggtcctacgctggtgaaacccacacagaccctcacgctgacctgcaccttctctgggttctcactcagcactagtggagtgggtgtgggctggatccgtcagcccccaggaaaggccctggagtggcttgcactcattttttgggatgatgatagtcactacagcccatctctgaagagcaggctcaccatcaccaaggacacctccaaaaaccaggtggtccttacaatgaccaacatggaccctgtggacacagccacatattactgtgcacggtattactttggttcggggagttatttccctagctactggtacttcgatctctggggccgtggcaccctggtcactgtctcctca

SEQ ID NO: 172 - protein, 127 aa, synthetic construct
  REGION 1..127: note = The Description of the artificial sequence: amino acid sequence of VH excluding signal sequence of MOG 307
RVTLRESGPTLVKPTQTLTLTCTFSGFSLSTSGVGVGWIRQPPGKALEWLALIFWDDDSHYSPSLKSRLTITKDTSKNQVVLTMTNMDPVDTATYYCARYYFGSGSYFPSYWYFDLWGRGTLVTVSS

SEQ ID NO: 173 - protein, 7 aa, synthetic construct
  REGION 1..7: note = The Description of the artificial sequence: amino acid sequence of HCDR1 of MOG 307
TSGVGVG

SEQ ID NO: 174 - protein, 16 aa, synthetic construct
  REGION 1..16: note = The Description of the artificial sequence: amino acid sequence of HCDR2 of MOG 307
LIFWDDDSHYSPSLKS

SEQ ID NO: 175 - protein, 17 aa, synthetic construct
  REGION 1..17: note = The Description of the artificial sequence: amino acid sequence of HCDR3 of MOG 307
YYFGSGSYFPSYWYFDL

SEQ ID NO: 176 - DNA, 321 bp, synthetic construct
  misc_feature 1..321: note = The Description of the artificial sequence: base sequence encoding VL excluding signal sequence of MOG 307
  source 1..321: mol_type = other DNA
gaaatagtgatgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgacgttcggccaagggaccaaggtggagatcaaa

SEQ ID NO: 177 - protein, 107 aa, synthetic construct
  REGION 1..107: note = The Description of the artificial sequence: amino acid sequence of VL excluding signal sequence of MOG 307
EIVMTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 178 - protein, 11 aa, synthetic construct
  REGION 1..11: note = The Description of the artificial sequence: amino acid sequence of LCDR 1 of MOG 307
RASQSVSSYLA

SEQ ID NO: 179 - protein, 7 aa, synthetic construct
  REGION 1..7: note = The Description of the artificial sequence: amino acid sequence of LCDR 2 of MOG 307
DASNRAT

SEQ ID NO: 180 - protein, 9 aa, synthetic construct
  REGION 1..9: note = The Description of the artificial sequence: amino acid sequence of LCDR 3 of MOG 307
QQRSNWPPT

SEQ ID NO: 181 - DNA, 360 bp, synthetic construct
  misc_feature 1..360: note = The Description of the artificial sequence: base sequence encoding VH excluding signal sequence of MOG 310
  source 1..360: mol_type = other DNA
caggtgcagctggtgcaatctggggctgaggtgaagaagcctgggtcctcggtgaaggtctcctgcaaggcttctggaggcaccttcagcagatatgctatcagctgggtgcgacaggcccctggacaagggcttgagtggatgggagggatcatccctatgtttagtacagcaaactacgcacagaagttccagggcagagtcacgattaccgcggacgaatccacgagcacagcctacatggagctgagcagcctgagatctgaggacacggccgtgtattactgtgcgagagattgggcagtggctggtatgggctttaactactggggccagggaaccctggtcactgtctcctca

SEQ ID NO: 182 - protein, 120 aa, synthetic construct
  REGION 1..120: note = The Description of the artificial sequence: amino acid sequence of VH excluding signal sequence of MOG 310
QVQLVQSGAEVKKPGSSVKVSCKASGGTFSRYAISWVRQAPGQGLEWMGGIIPMFSTANYAQKFQGRVTITADESTSTAYMELSSLRSEDTAVYYCARDWAVAGMGFNYWGQGTLVTVSS

SEQ ID NO: 183 - protein, 5 aa, synthetic construct
  REGION 1..5: note = The Description of the artificial sequence: amino acid sequence of HCDR1 of MOG 310
RYAIS

SEQ ID NO: 184 - protein, 17 aa, synthetic construct
  REGION 1..17: note = The Description of the artificial sequence: amino acid sequence of HCDR2 of MOG 310
GIIPMFSTANYAQKFQG

SEQ ID NO: 185 - protein, 11 aa, synthetic construct
  REGION 1..11: note = The Description of the artificial sequence: amino acid sequence of HCDR3 of MOG 310
DWAVAGMGFNY

SEQ ID NO: 186 - DNA, 324 bp, synthetic construct
  misc_feature 1..324: note = The Description of the artificial sequence: base sequence encoding VL excluding signal sequence of MOG 310
  source 1..324: mol_type = other DNA
gaaattgtgttgacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtacacttttggccaggggaccaaagtggatatcaaa

SEQ ID NO: 187 - protein, 108 aa, synthetic construct
  REGION 1..108: note = The Description of the artificial sequence: amino acid sequence of VL excluding signal sequence of MOG 310
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKVDIK

SEQ ID NO: 188 - protein, 12 aa, synthetic construct
  REGION 1..12: note = The Description of the artificial sequence: amino acid sequence of LCDR1 of MOG 310
RASQSVSSSYLA

SEQ ID NO: 189 - protein, 7 aa, synthetic construct
  REGION 1..7: note = The Description of the artificial sequence: amino acid sequence of LCDR 2 of MOG 310
GASSRAT

SEQ ID NO: 190 - protein, 9 aa, synthetic construct
  REGION 1..9: note = The Description of the artificial sequence: amino acid sequence of LCDR3 of MOG 310
QQYGSSPYT

SEQ ID NO: 191 - DNA, 354 bp, synthetic construct
  misc_feature 1..354: note = The Description of the artificial sequence: base sequence encoding VH excluding signal sequence of MOG 312
  source 1..354: mol_type = other DNA
caggtgcagctacagcagtggggcgcaggactgttgaagccttcggagaccctgtccctcacctgcgctgtctatggtgggtccttaagtggttactactggagctggatccgccagcccccagggaaggggctggagtggattggggatatcactcatagtggaagcaccaactacaacccgtccctcaagagtcgagtcaccatgtcagttgacacgtccaaaaaccagttctccctgaacctgaactctgtgaccgccgcggacacggctgtgtattactgtgcgagaagggggataggagctgctgtctttgacctctggggccagggaaccctggtcactgtctcctca

SEQ ID NO: 192 - protein, 118 aa, synthetic construct
  REGION 1..118: note = The Description of the artificial sequence: amino acid sequence of VH excluding signal sequence of MOG 312
QVQLQQWGAGLLKPSETLSLTCAVYGGSLSGYYWSWIRQPPGKGLEWIGDITHSGSTNYNPSLKSRVTMSVDTSKNQFSLNLNSVTAADTAVYYCARRGIGAAVFDLWGQGTLVTVSS

SEQ ID NO: 193 - protein, 5 aa, synthetic construct
  REGION 1..5: note = The Description of the artificial sequence: amino acid sequence of HCDR1 of MOG 312
GYYWS

SEQ ID NO: 194 - protein, 16 aa, synthetic construct
  REGION 1..16: note = The Description of the artificial sequence: amino acid sequence of HCDR2 of MOG 312
DITHSGSTNYNPSLKS

SEQ ID NO: 195 - protein, 10 aa, synthetic construct
  REGION 1..10: note = The Description of the artificial sequence: amino acid sequence of HCDR3 of MOG 312
RGIGAAVFDL

SEQ ID NO: 196 - DNA, 324 bp, synthetic construct
  misc_feature 1..324: note = The Description of the artificial sequence: base sequence encoding VL excluding signal sequence of MOG 312
  source 1..324: mol_type = other DNA
gaaatagtgttgacgcagtctccagccaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagcgtagcaactggcctctcactttcggcggagggaccaaggtggagatcaaa

SEQ ID NO: 197 - protein, 108 aa, synthetic construct
  REGION 1..108: note = The Description of the artificial sequence: amino acid sequence of VL excluding signal sequence of MOG 312
EIVLTQSPATLSLSPGERATLSCRASQSVSSSYLAWYQQKPGQAPRLLIYGASSRATGIPDRFSGSGSGTDFTLTISRLEPEDFAVYYCQQRSNWPLTFGGGTKVEIK

SEQ ID NO: 198 - protein, 12 aa, synthetic construct
  REGION 1..12: note = The Description of the artificial sequence: amino acid sequence of LCDR 1 of MOG 312
RASQSVSSSYLA

SEQ ID NO: 199 - protein, 7 aa, synthetic construct
  REGION 1..7: note = The Description of the artificial sequence: amino acid sequence of LCDR 2 of MOG 312
GASSRAT

SEQ ID NO: 200 - protein, 9 aa, synthetic construct
  REGION 1..9: note = The Description of the artificial sequence: amino acid sequence of LCDR 3 of MOG 312
QQRSNWPLT

SEQ ID NO: 201 - DNA, 366 bp, synthetic construct
  misc_feature 1..366: note = The Description of the artificial sequence: base sequence encoding VH excluding signal sequence of MOG 326
  source 1..366: mol_type = other DNA
gaggtccagctggtacagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagggtattacgatattttgactggttccttctttgactactggggccagggaaccctggtcactgtctcctca

SEQ ID NO: 202 - protein, 122 aa, synthetic construct
  REGION 1..122: note = The Description of the artificial sequence: amino acid sequence of VH excluding signal sequence of MOG 326
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGYYDILTGSFFDYWGQGTLVTVSS

SEQ ID NO: 203 - protein, 5 aa, synthetic construct
  REGION 1..5: note = The Description of the artificial sequence: amino acid sequence of HCDR1 of MOG 326
SYYMH

SEQ ID NO: 204 - protein, 17 aa, synthetic construct
  REGION 1..17: note = The Description of the artificial sequence: amino acid sequence of HCDR2 of MOG 326
IINPSGGSTSYAQKFQG

SEQ ID NO: 205 - protein, 13 aa, synthetic construct
  REGION 1..13: note = The Description of the artificial sequence: amino acid sequence of HCDR3 of MOG 326
GYYDILTGSFFDY

SEQ ID NO: 206 - DNA, 321 bp, synthetic construct
  misc_feature 1..321: note = The Description of the artificial sequence: base sequence encoding VL excluding signal sequence of MOG 326
  source 1..321: mol_type = other DNA
gacatcgtgatgacccagtctccatcctccctgtctgcatctgtaggagacagagtcaccatcacttgccgggcaagtcagggcattagcagtgctttagtctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttaccctctcactttcggcggagggaccaaagtggatatcaaa

SEQ ID NO: 207 - protein, 107 aa, synthetic construct
  REGION 1..107: note = The Description of the artificial sequence: amino acid sequence of VL excluding signal sequence of MOG 326
DIVMTQSPSSLSASVGDRVTITCRASQGISSALVWYQQKPGKAPKLLIYDASSLESGVPSRFSGSGSGTDFTLTISSLQPEDFATYYCQQFNSYPLTFGGGTKVDIK

SEQ ID NO: 208 - protein, 11 aa, synthetic construct
  REGION 1..11: note = The Description of the artificial sequence: amino acid sequence of LCDR 1 of MOG 326
RASQGISSALV

SEQ ID NO: 209 - protein, 7 aa, synthetic construct
  REGION 1..7: note = The Description of the artificial sequence: amino acid sequence of LCDR 2 of MOG 326
DASSLES

SEQ ID NO: 210 - protein, 9 aa, synthetic construct
  REGION 1..9: note = The Description of the artificial sequence: amino acid sequence of LCDR 3 of MOG 326
QQFNSYPLT

SEQ ID NO: 211 - DNA, 357 bp, synthetic construct
  misc_feature 1..357: note = The Description of the artificial sequence: base sequence encoding VH excluding signal sequence of MOG 329
  source 1..357: mol_type = other DNA
caggtgcagctggtggagtctgggggaggcttggtacagcctggggggtccctgagactctcctgtgcagcctctggattcgcctttcgcaactatgccatgaactgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtttattactgtgcgagagactacggtggtatctccccctttgactactggggccagggaaccctggtcactgtctcctca

SEQ ID NO: 212 - protein, 119 aa, synthetic construct
  REGION 1..119: note = The Description of the artificial sequence: amino acid sequence of VH excluding signal sequence of MOG 329
QVQLVESGGGLVQPGGSLRLSCAASGFAFRNYAMNWVRQAPGKGLEWVSAISGSGGSTYYADSVKGRFTISRDNSKNTLYLQMNSLRAEDTAVYYCARDYGGISPFDYWGQGTLVTVSS

SEQ ID NO: 213 - protein, 5 aa, synthetic construct
  REGION 1..5: note = The Description of the artificial sequence: amino acid sequence of HCDR1 of MOG 329
NYAMN

SEQ ID NO: 214 - protein, 17 aa, synthetic construct
  REGION 1..17: note = The Description of the artificial sequence: amino acid sequence of HCDR2 of MOG 329
AISGSGGSTYYADSVKG

SEQ ID NO: 215 - protein, 10 aa, synthetic construct
  REGION 1..10: note = The Description of the artificial sequence: amino acid sequence of HCDR3 of MOG 329
DYGGISPFDY

SEQ ID NO: 216 - DNA, 321 bp, synthetic construct
  misc_feature 1..321: note = The Description of the artificial sequence: base sequence encoding VL excluding signal sequence of MOG 329
  source 1..321: mol_type = other DNA
gacatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttaccctcacacttttggccaggggaccaaagtggatatcaaa

SEQ ID NO: 217 - protein, 107 aa, synthetic construct
  REGION 1..107: note = The Description of the artificial sequence: amino acid sequence of VL excluding signal sequence of MOG 329
DIQLTQSPSSLSASVGDRVTITCRASQGISSALAWYQQKPGKAPKLLIYDASSLESGVPSRFSGSGSGTDFTLTISSLQPEDFATYYCQQFNSYPHTFGQGTKVDIK

SEQ ID NO: 218 - protein, 11 aa, synthetic construct
  REGION 1..11: note = The Description of the artificial sequence: amino acid sequence of LCDR 1 of MOG 329
RASQGISSALA

SEQ ID NO: 219 - protein, 7 aa, synthetic construct
  REGION 1..7: note = The Description of the artificial sequence: amino acid sequence of LCDR 2 of MOG 329
DASSLES

SEQ ID NO: 220 - protein, 9 aa, synthetic construct
  REGION 1..9: note = The Description of the artificial sequence: amino acid sequence of LCDR 3 of MOG 329
QQFNSYPHT

SEQ ID NO: 221 - DNA, 372 bp, synthetic construct
  misc_feature 1..372: note = The Description of the artificial sequence: base sequence encoding VH excluding signal sequence of MOG446
  source 1..372: mol_type = other DNA
caggtgcagctggtggagtctgggggaggtgtggtacggccgggggggtccctgagactctcctgtgcagcctctggattcacctttgatgattatggcatgagctgggtccgccaagctccagggaaggggctggagtgggtctctaatattaattggaatggtgatagcacaggttatgtagactctgtgaagggccgattcaccatctccagagacaacgccaagaactccctgtatctgcaaatgaacagtctgagagccgaggacacggccttgtattactgtgcgagagcgaggacctattactatgtttcggggaggtactactttgactactggggccagggaaccctggtcactgtctcctca

SEQ ID NO: 222 - protein, 124 aa, synthetic construct
  REGION 1..124: note = The Description of the artificial sequence: amino acid sequence of VH excluding signal sequence of MOG446
QVQLVESGGGVVRPGGSLRLSCAASGFTFDDYGMSWVRQAPGKGLEWVSNINWNGDSTGYVDSVKGRFTISRDNAKNSLYLQMNSLRAEDTALYYCARARTYYYVSGRYYFDYWGQGTLVTVSS

SEQ ID NO: 223 - protein, 5 aa, synthetic construct
  REGION 1..5: note = The Description of the artificial sequence: amino acid sequence of HCDR1 of MOG446
DYGMS

SEQ ID NO: 224 - protein, 17 aa, synthetic construct
  REGION 1..17: note = The Description of the artificial sequence: amino acid sequence of HCDR2 of MOG446
NINWNGDSTGYVDSVKG

SEQ ID NO: 225 - protein, 15 aa, synthetic construct
  REGION 1..15: note = The Description of the artificial sequence: amino acid sequence of HCDR3 of MOG446
ARTYYYVSGRYYFDY

SEQ ID NO: 226 - DNA, 321 bp, synthetic construct
  misc_feature 1..321: note = The Description of the artificial sequence: base sequence encoding VL excluding signal sequence of MOG446
  source 1..321: mol_type = other DNA
gacatccagatgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgctcactttcggcggagggaccaaggtggaaatcaaa

SEQ ID NO: 227 - protein, 107 aa, synthetic construct
  REGION 1..107: note = The Description of the artificial sequence: amino acid sequence of VL excluding signal sequence of MOG446
DIQMTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIK

SEQ ID NO: 228 - protein, 11 aa, synthetic construct
  REGION 1..11: note = The Description of the artificial sequence: amino acid sequence of LCDR1 of MOG446
RASQGISSALA

SEQ ID NO: 229 - protein, 7 aa, synthetic construct
  REGION 1..7: note = The Description of the artificial sequence: amino acid sequence of LCDR2 of MOG446
DASSLES

SEQ ID NO: 230 - protein, 9 aa, synthetic construct
  REGION 1..9: note = The Description of the artificial sequence: amino acid sequence of LCDR3 of MOG446
QQFNSYPLT

SEQ ID NO: 231 - DNA, 366 bp, synthetic construct
  misc_feature 1..366: note = The Description of the artificial sequence: base sequence encoding VH excluding signal sequence of MOG456
  source 1..366: mol_type = other DNA
gaggtgcagctggtggagtctgggggaggcctggtcaagcctggggggtccctgagactctcctgtgcagcctctggattcaccttcagtagctatagcatgcactgggtccgccaggctccagggaaggggctggagtgggtctcatccattggtagtaggagtcgttacatatactacgcagactcagtgaagggccgattcaccatctccagagacaacgccaagaactcactgtatctgcaaatgaacagcctgagagccgaggacacggctgtgtattactgtgcgaaagggtattacgatattttgactggttctctctttgactactggggccagggaaccctggtcactgtctcctca

SEQ ID NO: 232 - protein, 122 aa, synthetic construct
  REGION 1..122: note = The Description of the artificial sequence: amino acid sequence of VH excluding signal sequence of MOG 456
EVQLVESGGGLVKPGGSLRLSCAASGFTFSSYSMHWVRQAPGKGLEWVSSIGSRSRYIYYADSVKGRFTISRDNAKNSLYLQMNSLRAEDTAVYYCAKGYYDILTGSLFDYWGQGTLVTVSS

SEQ ID NO: 233 - protein, 5 aa, synthetic construct
  REGION 1..5: note = The Description of the artificial sequence: amino acid sequence of HCDR1 of MOG456
SYSMH

SEQ ID NO: 234 - protein, 17 aa, synthetic construct
  REGION 1..17: note = The Description of the artificial sequence: amino acid sequence of HCDR2 of MOG456
SIGSRSRYIYYADSVKG

SEQ ID NO: 235 - protein, 13 aa, synthetic construct
  REGION 1..13: note = The Description of the artificial sequence: amino acid sequence of HCDR3 of MOG 456
GYYDILTGSLFDY

SEQ ID NO: 236 - DNA, 321 bp, synthetic construct
  misc_feature 1..321: note = The Description of the artificial sequence: base sequence encoding VL excluding signal sequence of MOG456
  source 1..321: mol_type = other DNA
gacatcgtgatgacccagtctccatcctcactgtctgcatctgtaggagacagagtcaccatcacttgtcgggcgagtcagggtattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgtggacgttcggccaagggaccaaggtggagatcaaa

SEQ ID NO: 237 - protein, 107 aa, synthetic construct
  REGION 1..107: note = The Description of the artificial sequence: amino acid sequence of VL excluding signal sequence of MOG456
DIVMTQSPSSLSASVGDRVTITCRASQGISSALAWYQQKPGKAPKLLIYDASSLESGVPSRFSGSGSGTDFTLTISSLQPEDFATYYCQQFNSYPWTFGQGTKVEIK

SEQ ID NO: 238 - protein, 11 aa, synthetic construct
  REGION 1..11: note = The Description of the artificial sequence: amino acid sequence of LCDR 1 of MOG 456
RASQGISSALA

SEQ ID NO: 239 - protein, 7 aa, synthetic construct
  REGION 1..7: note = The Description of the artificial sequence: amino acid sequence of LCDR 2 of MOG 456
DASSLES

SEQ ID NO: 240 - protein, 9 aa, synthetic construct
  REGION 1..9: note = The Description of the artificial sequence: amino acid sequence of LCDR 3 of MOG 456
QQFNSYPWT

SEQ ID NO: 241 - DNA, 357 bp, synthetic construct
  misc_feature 1..357: note = The Description of the artificial sequence: base sequence encoding VH excluding signal sequence of MOG 473
  source 1..357: mol_type = other DNA
caggtgcagctggtggagtctgggggaggcctggtcaagcctggggggtccctgagactctcctgtgcagcctctggattcaccttcagtagctatagcatgcactgggtccgccaggctccagggaaggggctggagtgggtttcggacattagtactaatagtagaaccagaaactatgcagactctgtgaagggccgattcaccatctccagagacaatgccaagaactcagtgtatctgcaaatgcacagcctgagggacgaggacacggctgtgtactactgtgcgagagactacggtggtatctattactttgactattggggccagggaaccctggtcactgtctcctca

SEQ ID NO: 242 - protein, 119 aa, synthetic construct
  REGION 1..119: note = The Description of the artificial sequence: amino acid sequence of VH excluding signal sequence of MOG 473
QVQLVESGGGLVKPGGSLRLSCAASGFTFSSYSMHWVRQAPGKGLEWVSDISTNSRTRNYADSVKGRFTISRDNAKNSVYLQMHSLRDEDTAVYYCARDYGGIYYFDYWGQGTLVTVSS

SEQ ID NO: 243 - protein, 5 aa, synthetic construct
  REGION 1..5: note = The Description of the artificial sequence: amino acid sequence of HCDR1 of MOG 473
SYSMH

SEQ ID NO: 244 - protein, 17 aa, synthetic construct
  REGION 1..17: note = The Description of the artificial sequence: amino acid sequence of HCDR2 of MOG 473
DISTNSRTRNYADSVKG

SEQ ID NO: 245 - protein, 10 aa, synthetic construct
  REGION 1..10: note = The Description of the artificial sequence: amino acid sequence of HCDR3 of MOG 473
DYGGIYYFDY

SEQ ID NO: 246 - DNA, 324 bp, synthetic construct
  misc_feature 1..324: note = The Description of the artificial sequence: base sequence encoding VL excluding signal sequence of MOG 473
  source 1..324: mol_type = other DNA
gaaatagtgatgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgtacacttttggccaggggaccaagctggagatcaaa

SEQ ID NO: 247 - protein, 108 aa, synthetic construct
  REGION 1..108: note = The Description of the artificial sequence: amino acid sequence of VL excluding signal sequence of MOG 473
EIVMTQSPATLSLSPGERATLSCRASQSVSSYLAWYQQKPGQAPRLLIYDASNRATGIPARFSGSGSGTDFTLTISSLEPEDFAVYYCQQRSNWPPYTFGQGTKLEIK

SEQ ID NO: 248 - protein, 11 aa, synthetic construct
  REGION 1..11: note = The Description of the artificial sequence: amino acid sequence of LCDR1 of MOG 473
RASQSVSSYLA

SEQ ID NO: 249 - protein, 7 aa, synthetic construct
  REGION 1..7: note = The Description of the artificial sequence: amino acid sequence of LCDR 2 of MOG 473
DASNRAT

SEQ ID NO: 250 - protein, 10 aa, synthetic construct
  REGION 1..10: note = The Description of the artificial sequence: amino acid sequence of LCDR 3 of MOG 473
QQRSNWPPYT

SEQ ID NO: 251 - DNA, 369 bp, synthetic construct
  misc_feature 1..369: note = The Description of the artificial sequence: base sequence encoding VH excluding signal sequence of MOG426
  source 1..369: mol_type = other DNA
caggtccagctggtacagtctggagctgaggtgaagaagcctggggcctcagtgaaggtctcctgcaaggcttctggttacagctttaacagctatggtatcaactgggtgcgacaggcccctggacaagggcttgagtggatgggatggatcagcgcttacactggtaaaacaagttatgcacagaaggtccagggcagagtcaccatgaccacagacagatccacgagcacagcctacatggagctgaggagcctgagatctgacgacacggccatgtattactgtgcgagagagtacgatattttgactggttattccgatgcttttgatacctggggccaagggacaatggtcaccgtctcttca

SEQ ID NO: 252 - protein, 123 aa, synthetic construct
  REGION 1..123: note = The Description of the artificial sequence: amino acid sequence of VH excluding signal sequence of MOG426
QVQLVQSGAEVKKPGASVKVSCKASGYSFNSYGINWVRQAPGQGLEWMGWISAYTGKTSYAQKVQGRVTMTTDRSTSTAYMELRSLRSDDTAMYYCAREYDILTGYSDAFDTWGQGTMVTVSS

SEQ ID NO: 253 - DNA, 321 bp, synthetic construct
  misc_feature 1..321: note = The Description of the artificial sequence: base sequence encoding VL excluding signal sequence of MOG426
  source 1..321: mol_type = other DNA
gaaattgtgttgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtggcaactggccgctcactttcggcggagggaccaagctggagatcaaa

SEQ ID NO: 254 - protein, 107 aa, synthetic construct
  REGION 1..107: note = The Description of the artificial sequence: amino acid sequence of VL excluding signal sequence of MOG426
EIVLTQSPATLSLSPGERATLSCRASQSVSSYLAWYQQKPGQAPRLLIYDASNRATGIPARFSGSGSGTDFTLTISSLEPEDFAVYYCQQRGNWPLTFGGGTKLEIK

SEQ ID NO: 255 - DNA, 369 bp, synthetic construct
  misc_feature 1..369: note = The Description of the artificial sequence: base sequence encoding VH excluding signal sequence of MOG428
  source 1..369: mol_type = other DNA
gaggtccagctggtacagtctggagctgaggtgaagaagcctggggcctcagtgaaggtctcctgcaaggcttctggttacagctttaacagctatggtatcaactgggtgcgacaggcccctggacaagggcttgagtggatgggatggatcagcgcttacactggtaaaacaagttatgcacagaaggtccagggcagagtcaccatgaccacagacagatccacgagcacagcctacatggagctgaggagcctgagatctgacgacacggccatgtattactgtgcgagagagtacgatattttgactggttattccgatgcttttgatacctggggccaagggacaatggtcaccgtctcttca

SEQ ID NO: 256 - protein, 123 aa, synthetic construct
  REGION 1..123: note = The Description of the artificial sequence: amino acid sequence of VH excluding signal sequence of MOG428
EVQLVQSGAEVKKPGASVKVSCKASGYSFNSYGINWVRQAPGQGLEWMGWISAYTGKTSYAQKVQGRVTMTTDRSTSTAYMELRSLRSDDTAMYYCAREYDILTGYSDAFDTWGQGTMVTVSS

SEQ ID NO: 257 - DNA, 324 bp, synthetic construct
  misc_feature 1..324: note = The Description of the artificial sequence: base sequence encoding VL excluding signal sequence of MOG428
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcggacgttcggccaagggaccaagctggagatcaaa

SEQ ID NO: 258 - protein, 108 aa, synthetic construct
  REGION 1..108: note = The Description of the artificial sequence: amino acid sequence of VL excluding signal sequence of MOG428
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKLEIK

SEQ ID NO: 259 - DNA, 366 bp, synthetic construct
  misc_feature 1..366: note = The Description of the artificial sequence: base sequence encoding VH excluding signal sequence of MOG 313
  source 1..366: mol_type = other DNA
caggtgcagctggtggagtctgggggaggcttggtacagcctggggggtccctgagactctcctgtgcagcctctggattcacctttagcagctatgccatgagctgggtccgccaggctccagggaaggggctggagtgggtctcagttattagtggtagtggtggtagcacatactacgcagactccgtgaagggccggttcaccatctccagagacaattccaagaacacgctgtatctgcaaatgaacagcctgagagccgaggacacggccgtatattactgtgcgaaagcatattacgatattttgactggttccctctttgactcctggggccagggaaccctggtcactgtctcctca

SEQ ID NO: 260 - protein, 122 aa, synthetic construct
  REGION 1..122: note = The Description of the artificial sequence: amino acid sequence of VH excluding signal sequence of MOG 313
QVQLVESGGGLVQPGGSLRLSCAASGFTFSSYAMSWVRQAPGKGLEWVSVISGSGGSTYYADSVKGRFTISRDNSKNTLYLQMNSLRAEDTAVYYCAKAYYDILTGSLFDSWGQGTLVTVSS

SEQ ID NO: 261 - DNA, 321 bp, synthetic construct
  misc_feature 1..321: note = The Description of the artificial sequence: base sequence encoding VL excluding signal sequence of MOG 313
  source 1..321: mol_type = other DNA
gacatcgtgatgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttaccctctcactttcggcggagggaccaaggtggaaatcaaa

SEQ ID NO: 262 - protein, 107 aa, synthetic construct
  REGION 1..107: note = The Description of the artificial sequence: amino acid sequence of VL excluding signal sequence of MOG
DIVMTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIK

SEQ ID NO: 263 - DNA, 366 bp, synthetic construct
  misc_feature 1..366: note = The Description of the artificial sequence: base sequence encoding VH excluding signal sequence of MOG 314
  source 1..366: mol_type = other DNA
caggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagaggtagtagcacatactacgcagactccgtgaagggccggttcaccatctccagagacaattccaagaacacgctgtatctgcaaatgaacagcctgagagccgaggacacggccgtatattactgtgcgaaaggctattacgatattttgactggttccttctttgactactggggccagggaaccctggtcaccgtctcctca

SEQ ID NO: 264 - protein, 122 aa, synthetic construct
  REGION 1..122: note = The Description of the artificial sequence: amino acid sequence of VH excluding signal sequence of MOG 314
QVQLVESGGGLVQPGGSLRLSCAASGFTFSSYAMSWVRQAPGKGLEWVSAISGRGSSTYYADSVKGRFTISRDNSKNTLYLQMNSLRAEDTAVYYCAKGYYDILTGSFFDYWGQGTLVTVSS

SEQ ID NO: 265 - DNA, 321 bp, synthetic construct
  misc_feature 1..321: note = The Description of the artificial sequence: base sequence encoding VL excluding signal sequence of MOG 314
  source 1..321: mol_type = other DNA
gacatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgtacacttttggccaggggaccaagctggagatcaaa

SEQ ID NO: 266 - protein, 107 aa, synthetic construct
  REGION 1..107: note = The Description of the artificial sequence: amino acid sequence of VL excluding signal sequence of MOG 314
DIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 267 - DNA, 366 bp, synthetic construct
  misc_feature 1..366: note = The Description of the artificial sequence: base sequence encoding VH excluding signal sequence of MOG 315
  source 1..366: mol_type = other DNA
gaggtgcagctggtggagtctgggggaggcttggtacagcctggggggtccctgagactctcctgtgcagcctctggattcacctttagcacctatgccatgagctgggtccgccaggctccagggaaggggctggagtgggtctcagctattagtggtagtggtgttagcacatactacgcagactccgtgaagggccggttcaccctctccagagacaattccaagaacacgctgtatctgcaaatgaacagcctgagagccgaggacacggccgtatattactgtgcgaaagcgtattacgatattttgactggtaatttccttgactactggggccagggaaccctggtcactgtctcctca

SEQ ID NO: 268 - protein, 122 aa, synthetic construct
  REGION 1..122: note = The Description of the artificial sequence: amino acid sequence of VH excluding signal sequence of MOG 315
EVQLVESGGGLVQPGGSLRLSCAASGFTFSTYAMSWVRQAPGKGLEWVSAISGSGVSTYYADSVKGRFTLSRDNSKNTLYLQMNSLRAEDTAVYYCAKAYYDILTGNFLDYWGQGTLVTVSS

SEQ ID NO: 269 - DNA, 321 bp, synthetic construct
  misc_feature 1..321: note = The Description of the artificial sequence: base sequence encoding VL excluding signal sequence of MOG 315
  source 1..321: mol_type = other DNA
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgctcactttcggcggagggaccaaggtggaaatcaaa

SEQ ID NO: 270 - protein, 107 aa, synthetic construct [sequence omitted]

SEQ ID NO: 271 - DNA, 366 bp, synthetic construct
  misc_feature 1..366: note = The Description of the artificial sequence: base sequence encoding VH excluding signal sequence of MOG331
  source 1..366: mol_type = other DNA
gaggtgcagctggtggagtccgggggaggcttggtatagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccgattcaccatctccagagacaattccaagaacacactgtatctgcaaatgaacagcctgagagccgaggacacggccgtatattactgtgcgaaagcgtattacgatattttgactggttccttctttgactactggggccagggaaccctggtcactgtctcctca

SEQ ID NO: 272 - protein, 122 aa, synthetic construct
  REGION 1..122: note = The Description of the artificial sequence: amino acid sequence of VH excluding signal sequence of MOG331
EVQLVESGGGLVYPGGSLRLSCAASGFTFSSYAMSWVRQAPGKGLEWVSAISGSGGSTYYADSVKGRFTISRDNSKNTLYLQMNSLRAEDTAVYYCAKAYYDILTGSFFDYWGQGTLVTVSS

SEQ ID NO: 273 - DNA, 321 bp, synthetic construct
  misc_feature 1..321: note = The Description of the artificial sequence: base sequence encoding VL excluding signal sequence of MOG331
  source 1..321: mol_type = other DNA
gaaatagtgatgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagactggagcctgaagattttgcagtgtattactgtcagcagtatggtagctcaccgctcactttcggcggagggaccaagctggagatcaaa

SEQ ID NO: 274 - protein, 107 aa, synthetic construct
  REGION 1..107: note = The Description of the artificial sequence: amino acid sequence of VL excluding signal sequence of MOG331
EIVMTQSPATLSLSPGERATLSCRASQSVSSYLAWYQQKPGQAPRLLIYDASNRATGIPARFSGSGSGTDFTLTISRLEPEDFAVYYCQQYGSSPLTFGGGTKLEIK

SEQ ID NO: 275 - DNA, 366 bp, synthetic construct
  misc_feature 1..366: note = The Description of the artificial sequence: base sequence encoding VH excluding signal sequence of MOG357
  source 1..366: mol_type = other DNA
gaggtgcagctggtggagactgggggaggcttggtacagcctggggggtccctgagactctcctgtgcagcctctggattcacctttagcagctatgccatgagctgggtccgccaggctccagggaaggggctggagtgggtctcagatattaatcatagtggtggtagcacatactacgcagactccgtgaagggccggttcaccatctccagagacaattccaagcacacgctgtatctgcaaatgaacagcctgagagccgaggacacggccgtatattactgtgcgaaagactattacgatattttgactggttccttctttgactactggggccagggaaccctggtcactgtctcctca

SEQ ID NO: 276 - protein, 122 aa, synthetic construct
  REGION 1..122: note = The Description of the artificial sequence: amino acid sequence of VH excluding signal sequence of MOG 357
EVQLVETGGGLVQPGGSLRLSCAASGFTFSSYAMSWVRQAPGKGLEWVSDINHSGGSTYYADSVKGRFTISRDNSKHTLYLQMNSLRAEDTAVYYCAKDYYDILTGSFFDYWGQGTLVTVSS

SEQ ID NO: 277 - DNA, 321 bp, synthetic construct
  misc_feature 1..321: note = The Description of the artificial sequence: base sequence encoding VL excluding signal sequence of MOG 357
  source 1..321: mol_type = other DNA
gacatcgtgatgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgtggacgttcggccaagggaccaaggtggagatcaaa

SEQ ID NO: 278 - protein, 107 aa, synthetic construct; identical to SEQ ID NO: 237
  REGION 1..107: note = The Description of the artificial sequence: amino acid sequence of VL excluding signal sequence of MOG 357

SEQ ID NO: 279 - DNA, 366 bp, synthetic construct
  misc_feature 1..366: note = The Description of the artificial sequence: base sequence encoding VH excluding signal sequence of MOG 476
  source 1..366: mol_type = other DNA
gaggtgcagctggtggagtctgggggaggcttggtacagcctggggggtccctgagactctcctgtgcagcctctggattcacttttagcagctatgccatgaactgggtccgccaggctccagggaaggggctggagtgggtctcagctattagttatagtggtcgtagcacatactacgcagactccgtgaagggccgattcaccatctccagagacaattccaagaacacgctgtatctgcaaatgaacagcctgagagccgaggacacggccgtatattactgtgcgaagggcctttacgatattttgactggtggcggatttgactactggggccagggaaccctggtcaccgtctcttca

SEQ ID NO: 280 - protein, 122 aa, synthetic construct
  REGION 1..122: note = The Description of the artificial sequence: amino acid sequence of VH excluding signal sequence of MOG 476
EVQLVESGGGLVQPGGSLRLSCAASGFTFSSYAMNWVRQAPGKGLEWVSAISYSGRSTYYADSVKGRFTISRDNSKNTLYLQMNSLRAEDTAVYYCAKGLYDILTGGGFDYWGQGTLVTVSS

SEQ ID NO: 281 - DNA, 321 bp, synthetic construct
  misc_feature 1..321: note = The Description of the artificial sequence: base sequence encoding VL excluding signal sequence of MOG 476
  source 1..321: mol_type = other DNA
gacatccagatgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgtccacttttggccaggggacacgactggagattaaa

SEQ ID NO: 282 - protein, 107 aa, synthetic construct
  REGION 1..107: note = The Description of the artificial sequence: amino acid sequence of VL excluding signal sequence of MOG 476
DIQMTQSPSSLSASVGDRVTITCRASQGISSALAWYQQKPGKAPKLLIYDASSLESGVPSRFSGSGSGTDFTLTISSLQPEDFATYYCQQFNSYPSTFGQGTRLEIK

SEQ ID NO: 283 - DNA, 381 bp, synthetic construct
  misc_feature 1..381: note = The Description of the artificial sequence: base sequence encoding VH excluding signal sequence of MOG 323
  source 1..381: mol_type = other DNA
cggatcaccttgagggagtctggtcctacgctggtgaaacccacacagaccctcacgctgacctgcaccttctctgggttctcactcagcactagtggagtgggtgtgggctggatccgtcagcccccaggaaaggccctggagtggcttgcactcattttttgggatgatgatagtcactacagcccatctctgaagagcaggctcaccatcaccaaggacacctccaaaaaccaggtggtccttacaatgaccaacatggaccctgtggacacagccacatattactgtgcacggtattactttggttcggggagttatttccctagctactggtacttcgatctctggggccgtggcaccctggtcaccgtctcctca

SEQ ID NO: 284 - protein, 127 aa, synthetic construct
  REGION 1..127: note = The Description of the artificial sequence: amino acid sequence of VH excluding signal sequence of MOG 323
RITLRESGPTLVKPTQTLTLTCTFSGFSLSTSGVGVGWIRQPPGKALEWLALIFWDDDSHYSPSLKSRLTITKDTSKNQVVLTMTNMDPVDTATYYCARYYFGSGSYFPSYWYFDLWGRGTLVTVSS

SEQ ID NO: 285 - DNA, 321 bp, synthetic construct
  misc_feature 1..321: note = The Description of the artificial sequence: base sequence encoding VL excluding signal sequence of MOG 323
  source 1..321: mol_type = other DNA
gaaatagtgatgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctcccactttcggcggagggaccaagctggagatcaaa

SEQ ID NO: 286 - protein, 107 aa, synthetic construct
  REGION 1..107: note = The Description of the artificial sequence: amino acid sequence of VL excluding signal sequence of MOG 323
EIVMTQSPATLSLSPGERATLSCRASQSVSSYLAWYQQKPGQAPRLLIYDASNRATGIPARFSGSGSGTDFTLTISSLEPEDFAVYYCQQRSNWPPTFGGGTKLEIK

SEQ ID NO: 287 - DNA, 381 bp, synthetic construct
  misc_feature 1..381: note = The Description of the artificial sequence: base sequence encoding VH excluding signal sequence of MOG 341
  source 1..381: mol_type = other DNA
cggatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattgggatgatgataaacactacagcccatctctgaagagcaggctcaccatcaccaaggacacctccaaaaaccaggtggtccttacaatgaccaacatggaccctgtggacacagccacatattactgtgcacggtattactttggttcggggagttattcccctagctactggtacttcgatctctggggccgtggcaccctggtcactgtctcctca

SEQ ID NO: 288 - protein, 127 aa, synthetic construct
  REGION 1..127: note = The Description of the artificial sequence: amino acid sequence of VH excluding signal sequence of MOG 341
RITLKESGPTLVKPTQTLTLTCTFSGFSLSTSGVGVGWIRQPPGKALEWLALIYWDDDKHYSPSLKSRLTITKDTSKNQVVLTMTNMDPVDTATYYCARYYFGSGSYSPSYWYFDLWGRGTLVTVSS

SEQ ID NO: 289 - DNA, 321 bp, synthetic construct
  misc_feature 1..321: note = The Description of the artificial sequence: base sequence encoding VL excluding signal sequence of MOG 341
  source 1..321: mol_type = other DNA
gaaattgtgttgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctcccactttcggcggagggaccaaggtggagatcaaa

SEQ ID NO: 290 - protein, 107 aa, synthetic construct
  REGION 1..107: note = The Description of the artificial sequence: amino acid sequence of VL excluding signal sequence of MOG 341
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGGGTKVEIK

SEQ ID NO: 291 - DNA, 381 bp, synthetic construct
  misc_feature 1..381: note = The Description of the artificial sequence: base sequence encoding VH excluding signal sequence of MOG 354
  source 1..381: mol_type = other DNA
cggatcaccttgagggagtctggtcctacgctggtgaaacccacacagaccctcacgctgacctgcacctcctctgggctctcactcagcactagtggagtgggtgtgggctggatccgtcagcccccaggaaaggccctggagtggcttgcactcattttttgggatgatgatacacactacagcccatctctgaagagcaggctcaccatcaccaaggacacctccaaaaaccaggtggtccttacaatgaccaacatggaccctgtggacacagccacatattactgtgcacggtattactttggttcggggagttatttccctagctactggtacttcgatctctggggccgtggcaccatggtcaccgtctcttca

SEQ ID NO: 292 - protein, 127 aa, synthetic construct
  REGION 1..127: note = The Description of the artificial sequence: amino acid sequence of VH excluding signal sequence of MOG 354
RITLRESGPTLVKPTQTLTLTCTSSGLSLSTSGVGVGWIRQPPGKALEWLALIFWDDDTHYSPSLKSRLTITKDTSKNQVVLTMTNMDPVDTATYYCARYYFGSGSYFPSYWYFDLWGRGTMVTVSS

SEQ ID NO: 293 - DNA, 321 bp, synthetic construct
  misc_feature 1..321: note = The Description of the artificial sequence: base sequence encoding VL excluding signal sequence of MOG 354
  source 1..321: mol_type = other DNA
gaaatagtgatgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgctcactttcggcggagggaccaaggtggaaatcaaa

SEQ ID NO: 294 - protein, 107 aa, synthetic construct
  REGION 1..107: note = The Description of the artificial sequence: amino acid sequence of VL excluding signal sequence of MOG 354
EIVMTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 295 - DNA, 381 bp, synthetic construct
  misc_feature 1..381: note = The Description of the artificial sequence: base sequence encoding VH excluding signal sequence of MOG 355
  source 1..381: mol_type = other DNA
cggatcaccttgagggagtctggtcctacgctggtgaaacccacacagaccctcacgctgacctgcacctcctctgggctctcactcagcactagtggagtgggtgtgggctggatccgtcagcccccaggaaaggccctggagtggcttgcactcattttttgggatgatgatacacactacagcccatctctgaagagcaggctcaccatcaccaaggacacctccaaaaaccaggtggtccttacaatgaccaacatggaccctgtggacacagccacatattactgtgcacggtattactttggttcggggagttatttccctagctactggtacttcgatctctggggccgtggcaccctggtcactgtctcctca

SEQ ID NO: 296 - protein, 127 aa, synthetic construct
  REGION 1..127: note = The Description of the artificial sequence: amino acid sequence of VH excluding signal sequence of MOG 355
RITLRESGPTLVKPTQTLTLTCTSSGLSLSTSGVGVGWIRQPPGKALEWLALIFWDDDTHYSPSLKSRLTITKDTSKNQVVLTMTNMDPVDTATYYCARYYFGSGSYFPSYWYFDLWGRGTLVTVSS

SEQ ID NO: 297 - DNA, 321 bp, synthetic construct
  misc_feature 1..321: note = The Description of the artificial sequence: base sequence encoding VL excluding signal sequence of MOG 355
  source 1..321: mol_type = other DNA
gaaatagtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgctcactttcggcggagggaccaagctggagatcaaa

SEQ ID NO: 298 - protein, 107 aa, synthetic construct
  REGION 1..107: note = The Description of the artificial sequence: amino acid sequence of VL excluding signal sequence of MOG 355
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKLEIK

SEQ ID NO: 299 - DNA, 360 bp, synthetic construct
  misc_feature 1..360: note = The Description of the artificial sequence: base sequence encoding VH excluding signal sequence of MOG 308
  source 1..360: mol_type = other DNA
gaggtgcagctggtgcagtccggggctgaggtgaggaagtctgggtcctcggtgaaggtctcctgcaaggcttctggaggcaccttcagcagatatgctatcagctgggtgcgacaggcccctggacaagggcttgagtggatgggagggatcatccctatgtttagtacgacaaactacgcacagaagttccagggcagagtcacgattaccgcggacgaatccacgagcacagcctacatggaactgagcagcctgagatctgaggacacggccgtgtattactgtgcgagagattgggcagtggctggtatggggtttgcctactggggccagggaaccctggtcaccgtctcctca

SEQ ID NO: 300 - protein, 120 aa, synthetic construct
  REGION 1..120: note = The Description of the artificial sequence: amino acid sequence of VH excluding signal sequence of MOG 308
EVQLVQSGAEVRKSGSSVKVSCKASGGTFSRYAISWVRQAPGQGLEWMGGIIPMFSTTNYAQKFQGRVTITADESTSTAYMELSSLRSEDTAVYYCARDWAVAGMGFAYWGQGTLVTVSS

SEQ ID NO: 301 - DNA, 321 bp, synthetic construct
  misc_feature 1..321: note = The Description of the artificial sequence: base sequence encoding VL excluding signal sequence of MOG 308
  source 1..321: mol_type = other DNA
gaaatagtgatgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgacgttcggccaagggaccaaagtggatatcaaa

SEQ ID NO: 302 - protein, 107 aa, synthetic construct
  REGION 1..107: note = The Description of the artificial sequence: amino acid sequence of VL excluding signal sequence of MOG 308
EIVMTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVDIK

SEQ ID NO: 303 - DNA, 360 bp, synthetic construct
  misc_feature 1..360: note = The Description of the artificial sequence: base sequence encoding VH excluding signal sequence of MOG 316
  source 1..360: mol_type = other DNA
gaagtgcagctggtgcagtccggggctgaggtgaagaagcctgggtcctcggtgaaggtctcctgcaaggcttctggaggcaccttcagcagatatgctatcagctgggtgcgacaggcccctggacaagggcttgagtggatgggagggatcatccctatgtttaatacagcaaactacgcacagaagttccagggcagagtcacgattaccgcggacgaatccacgagcacagcctacatggagctgagcagcctgagatctgaggacacggccgtgtattactgtgcgagagattgggcagtggctggtatggggtttaactactggggccagggaaccctggtcaccgtctcctca

SEQ ID NO: 304 - protein, 120 aa, synthetic construct
  REGION 1..120: note = The Description of the artificial sequence: amino acid sequence of VH excluding signal sequence of MOG 316
EVQLVQSGAEVKKPGSSVKVSCKASGGTFSRYAISWVRQAPGQGLEWMGGIIPMFNTANYAQKFQGRVTITADESTSTAYMELSSLRSEDTAVYYCARDWAVAGMGFNYWGQGTLVTVSS

SEQ ID NO: 305 - DNA, 321 bp, synthetic construct
  misc_feature 1..321: note = The Description of the artificial sequence: base sequence encoding VL excluding signal sequence of MOG 316
  source 1..321: mol_type = other DNA
gacatccagatgacccagtctccatcctccctgtctgcatctgtaggagacagagtcaccatcacttgccgggcaagtcagggcattaacagtgctttagcctggtatcagcagaaaccagggaaagctcctaagctcctgatctatgctgcatccagtttgcaaagtggggtcccatcaaggttcagcggcagtggatctgggacagatttcactctcaccatcagcagcctgcagcctgaagattttgcaacttattactgccaacagtataatagttacccgatcaccttcggccaagggacacgactggagattaaa

SEQ ID NO: 306 - protein, 107 aa, synthetic construct
  REGION 1..107: note = The Description of the artificial sequence: amino acid sequence of VL excluding signal sequence of MOG 316
DIQMTQSPSSLSASVGDRVTITCRASQGINSALAWYQQKPGKAPKLLIYAASSLQSGVPSRFSGSGSGTDFTLTISSLQPEDFATYYCQQYNSYPITFGQGTRLEIK

SEQ ID NO: 307 - DNA, 360 bp, synthetic construct
  misc_feature 1..360: note = The Description of the artificial sequence: base sequence encoding VH excluding signal sequence of MOG 319
  source 1..360: mol_type = other DNA
caggtgcagctggtgcaatctggggctgaggtgaagaagcctgggtcctcggtgaaggtctcctgcaaggcttctggaggcaccttcagcagatatgctatcagctgggtgcgacaggcccctggacaagggcttgagtggatgggagggatcatccctatgtttagtacagcaaactacgcacagaagttccagggcagagtcacgattaccgcggacgaatccacgagcacagcctacatggagctgagcagcctgagatctgacgacacggccgtgtattactgtgcgagagattgggcagtggctggtatgggctttaactactggggccagggaaccctggtcactgtctcctca

SEQ ID NO: 308 - protein, 120 aa, synthetic construct
  REGION 1..120: note = The Description of the artificial sequence: amino acid sequence of VH excluding signal sequence of MOG 319
QVQLVQSGAEVKKPGSSVKVSCKASGGTFSRYAISWVRQAPGQGLEWMGGIIPMFSTANYAQKFQGRVTITADESTSTAYMELSSLRSDDTAVYYCARDWAVAGMGFNYWGQGTLVTVSS

SEQ ID NO: 309 - DNA, 324 bp, synthetic construct
  misc_feature 1..324: note = The Description of the artificial sequence: base sequence encoding VL excluding signal sequence of MOG 319
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccattcactttcggccctgggaccaaggtggaaatcaaa

SEQ ID NO: 310 - protein, 108 aa, synthetic construct
  REGION 1..108: note = The Description of the artificial sequence: amino acid sequence of VL excluding signal sequence of MOG 319
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVEIK

SEQ ID NO: 311 - DNA, 360 bp, synthetic construct
  misc_feature 1..360: note = The Description of the artificial sequence: base sequence encoding VH excluding signal sequence of MOG 320
  source 1..360: mol_type = other DNA
caggtgcagctggtgcagtctggggctgaggtgaagaagcctgggtcctcggtgaaggtctcctgcaaggcttctggaggcaccttcagcagatatgctatcaactgggtgcgacaggcccctggacaagggcttgagtggatgggagggatcatccctatgtttagtacagtaaattacgcacagaagttccagggcagagtcacgattaccgcggacgaatccacgagcacagcctacatggagctgagcagcctgagatctgaggacacggccgtgtattactgtgcgagagattgggcagtggctggtatggggtttgactactggggccagggaaccctggtcaccgtctcctca

SEQ ID NO: 312 - protein, 120 aa, synthetic construct
  REGION 1..120: note = The Description of the artificial sequence: amino acid sequence of VH excluding signal sequence of MOG 320
QVQLVQSGAEVKKPGSSVKVSCKASGGTFSRYAINWVRQAPGQGLEWMGGIIPMFSTVNYAQKFQGRVTITADESTSTAYMELSSLRSEDTAVYYCARDWAVAGMGFDYWGQGTLVTVSS

SEQ ID NO: 313 - DNA, 324 bp, synthetic construct
  misc_feature 1..324: note = The Description of the artificial sequence: base sequence encoding VL excluding signal sequence of MOG 320
  source 1..324: mol_type = other DNA
gaaatagtgttgacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccattcactttcggccctgggaccaaagtggatatcaaa

SEQ ID NO: 314 - protein, 108 aa, synthetic construct
  REGION 1..108: note = The Description of the artificial sequence: amino acid sequence of VL excluding signal sequence of MOG 320
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIK

SEQ ID NO: 315 - DNA, 360 bp, synthetic construct
  misc_feature 1..360: note = FThe Description of the artificial sequence: base sequence encoding VH excluding signal sequence of MOG 338
  source 1..360: mol_type = other DNA
caggtccagctggtgcagtctggggctgaggtgaagaagcctgggtcctcggtgaaggtctcctgcaaggcttctggaggcaccttcagcagatatgctatcagctgggtgcgacaggcccctggacaagggcttgagtggatgggagggatcatccctatgtttaatacagcaaactacgcacagaagttccagggcagagtcacgattaccgcggacgaatccacgagcacagcctacatggagctgagcagcctgagatctgaggacacggccgtgtattactgtgcgagagattgggcagtggctggtatggggtttgacccctggggccagggaaccctggtcactgtctcctca

SEQ ID NO: 316 - protein, 120 aa, synthetic construct
  REGION 1..120: note = The Description of the artificial sequence: amino acid sequence of VH excluding signal sequence of MOG 338
QVQLVQSGAEVKKPGSSVKVSCKASGGTFSRYAISWVRQAPGQGLEWMGGIIPMFNTANYAQKFQGRVTITADESTSTAYMELSSLRSEDTAVYYCARDWAVAGMGFDPWGQGTLVTVSS

SEQ ID NO: 317 - DNA, 321 bp, synthetic construct
  misc_feature 1..321: note = The Description of the artificial sequence: base sequence encoding VL excluding signal sequence of MOG 338
  source 1..321: mol_type = other DNA
gaaattgtgttgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtaacaactggcctctcactttcggcggagggaccaagctggagatcaaa

SEQ ID NO: 318 - protein, 107 aa, synthetic construct
  REGION 1..107: note = The Description of the artificial sequence: amino acid sequence of VL excluding signal sequence of MOG 338
EIVLTQSPATLSLSPGERATLSCRASQSVSSYLAWYQQKPGQAPRLLIYDASNRATGIPARFSGSGSGTDFTLTISSLEPEDFAVYYCQQRNNWPLTFGGGTKLEIK

SEQ ID NO: 319 - DNA, 360 bp, synthetic construct
  misc_feature 1..360: note = The Description of the artificial sequence: base sequence encoding VH excluding signal sequence of MOG352
  source 1..360: mol_type = other DNA
gaggtgcagctggtgcagtccggggctgaggtgaagaagcctgggtcctcggtgaaggtctcctgcaaggcttctggaggcaccttcagcagatatgctatcaactgggtgcgacaggcccctggacaagggcttgagtggatgggagggatcatccctatgtttagtacagtaaactacgcacagaagttccagggcagagtcacgattaccgcggacgaatccacgagcacagcctacatggagctgagcagcctgagatctgaggacacggccgtgtattactgtgcgagagattgggcagtggctggtatggggtttgactactggggccagggaaccctggtcactgtctcctca

SEQ ID NO: 320 - protein, 120 aa, synthetic construct
  REGION 1..120: note = The Description of the artificial sequence: amino acid sequence of VH excluding signal sequence of MOG352
EVQLVQSGAEVKKPGSSVKVSCKASGGTFSRYAINWVRQAPGQGLEWMGGIIPMFSTVNYAQKFQGRVTITADESTSTAYMELSSLRSEDTAVYYCARDWAVAGMGFDYWGQGTLVTVSS

SEQ ID NO: 321 - DNA, 321 bp, synthetic construct
  misc_feature 1..321: note = The Description of the artificial sequence: base sequence encoding VL excluding signal sequence of MOG352
  source 1..321: mol_type = other DNA
gccatccagatgacccagtctccatcttccgtgtctgcatctgtaggagacagagtcaccatcacttgtcgggcgagtcagggtattagcagctggttagcctggtatcagcataaaccagggaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttacccgctcactttcggcggagggaccaaagtggatatcaaa

SEQ ID NO: 322 - protein, 107 aa, synthetic construct
  REGION 1..107: note = The Description of the artificial sequence: amino acid sequence of VL excluding signal sequence of MOG 352
AIQMTQSPSSVSASVGDRVTITCRASQGISSWLAWYQHKPGKAPKSLIYAASSLQSGVPSRFSGSGSGTDFTLTISSLQPEDFATYYCQQYNSYPLTFGGGTKVDIK

SEQ ID NO: 323 - DNA, 360 bp, synthetic construct
  misc_feature 1..360: note = The Description of the artificial sequence: base sequence encoding VH excluding signal sequence of MOG 359
  source 1..360: mol_type = other DNA
gaggtccagctggtgcagtctggggctgaggtgaagaagcctgggtcctcggtgaaggtctcctgcaaggcttctggaggcaccttcagcagatatgctatcagctgggtgcgacaggcccctggacaagggcttgagtggatgggagggatcatccctatgtttaatacagcaaactacgcacagaagttccagggcagagtcacgattaccgcggacgaatccacgagcacagcctacatggagctgagcagcctgagatctgaggacacggccgtgtattactgtgcgagagattgggcagtggctggtatggggtttaactactggggccagggaaccctggtcactgtctcctca

SEQ ID NO: 324 - protein, 120 aa, synthetic construct; identical to SEQ ID NO: 304
  REGION 1..120: note = The Description of the artificial sequence: amino acid sequence of VH excluding signal sequence of MOG 359

SEQ ID NO: 325 - DNA, 324 bp, synthetic construct
  misc_feature 1..324: note = The Description of the artificial sequence: base sequence encoding VL excluding signal sequence of MOG 359
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgctcactttcggcggagggaccaagctggagatcaaa

SEQ ID NO: 326 - protein, 108 aa, synthetic construct
  REGION 1..108: note = The Description of the artificial sequence: amino acid sequence of VL excluding signal sequence of MOG 359
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKLEIK

SEQ ID NO: 327 - DNA, 360 bp, synthetic construct
  misc_feature 1..360: note = The Description of the artificial sequence: base sequence encoding VH excluding signal sequence of MOG 478
  source 1..360: mol_type = other DNA
gaggtccagctggtacagtctggggctgaggtgaagaagcctgggtcctcggtgaaggtctcctgcaaggcttctggaggcaccttcagcagatatgctatcagctgggtgcgacaggcccctggacaagggcttgagtggatgggagggatcatccctatgtttgctacagcaaactacgcacagaagttccaggccagagtcacgattaccgcggacgaaaccacgagcacagcctacatggagctgagcagcctgagatctgaggacacgggcgtgtattactgtgcgagagattgggcagtggctgctatggggtttgcccactggggccagggaaccctggtcactgtctcctca

SEQ ID NO: 328 - protein, 120 aa, synthetic construct
  REGION 1..120: note = The Description of the artificial sequence: amino acid sequence of VH excluding signal sequence of MOG 478
EVQLVQSGAEVKKPGSSVKVSCKASGGTFSRYAISWVRQAPGQGLEWMGGIIPMFATANYAQKFQARVTITADETTSTAYMELSSLRSEDTGVYYCARDWAVAAMGFAHWGQGTLVTVSS

SEQ ID NO: 329 - DNA, 324 bp, synthetic construct
  misc_feature 1..324: note = The Description of the artificial sequence: base sequence encoding VL excluding signal sequence of MOG 478
  source 1..324: mol_type = other DNA
gaaatagtgatgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgatcaccttcggccaagggacacgactggagattaaa

SEQ ID NO: 330 - protein, 108 aa, synthetic construct
  REGION 1..108: note = The Description of the artificial sequence: amino acid sequence of VL excluding signal sequence of MOG 478
EIVMTQSPGTLSLSPGERATLSCRASQSVSSSYLAWYQQKPGQAPRLLIYGASSRATGIPDRFSGSGSGTDFTLTISRLEPEDFAVYYCQQYGSSPITFGQGTRLEIK

SEQ ID NO: 331 - DNA, 357 bp, synthetic construct
  misc_feature 1..357: note = The Description of the artificial sequence: base sequence encoding VH excluding signal sequence of MOG 470
  source 1..357: mol_type = other DNA
caggtgcagctggtggagtctgggggaggcttggtacagccgggggggtccctgagactctcctgtgcagcctctggattcacctttagcagctatgccatgaactgggtccgccaggctccagggaaggggctggagtgggtctcagctattagtggtagtggtggtagcacatactacgcagactccgtgaagggccggttcaccatctccagagacaattccaagaacacgctgtatctgcaagtgaacagcctgagagccgaggacacggccgtttattactgtgcgagagactacggtggtatctccccctttgactactggggccagggaaccctggtcaccgtctcctca

SEQ ID NO: 332 - protein, 119 aa, synthetic construct
  REGION 1..119: note = The Description of the artificial sequence: amino acid sequence of VH excluding signal sequence of MOG 470
QVQLVESGGGLVQPGGSLRLSCAASGFTFSSYAMNWVRQAPGKGLEWVSAISGSGGSTYYADSVKGRFTISRDNSKNTLYLQVNSLRAEDTAVYYCARDYGGISPFDYWGQGTLVTVSS

SEQ ID NO: 333 - DNA, 321 bp, synthetic construct
  misc_feature 1..321: note = The Description of the artificial sequence: base sequence encoding VL excluding signal sequence of MOG 470
  source 1..321: mol_type = other DNA
gaaatagtgatgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagactggagcctgaagattttgcagtgtattactgtcagcagtatggtagctcaccgtacacttttggccaggggacacgactggagattaaa

SEQ ID NO: 334 - protein, 107 aa, synthetic construct
  REGION 1..107: note = The Description of the artificial sequence: amino acid sequence of VL excluding signal sequence of MOG 470
EIVMTQSPATLSLSPGERATLSCRASQSVSSYLAWYQQKPGQAPRLLIYDASNRATGIPARFSGSGSGTDFTLTISRLEPEDFAVYYCQQYGSSPYTFGQGTRLEIK

SEQ ID NO: 335 - DNA, 366 bp, synthetic construct
  misc_feature 1..366: note = The Description of the artificial sequence: base sequence encoding VH excluding signal sequence of MOG 418
  source 1..366: mol_type = other DNA
caggtgtagctggtgcagtctgggggaggcctggtcaagcctggggggtccctgagactctcctgtgcagcctctggattcaccttcagtagctatagcatgcactgggtccgccaggctccagggaaggggctggagtgggtctcatccattagtcatagtagtagttacatatcctacgcagactcagtgaagggccgattcaccatctccagagacaacgccaagaattcactgtatctgcaaatgaacagcctgagagccgaggacacggctgtgtattactgtgcgaaagggtattacgatattttgactggttctctctttgactactggggccagggaaccctggtcaccgtctcctca

SEQ ID NO: 336 - protein, 122 aa, synthetic construct
  REGION 1..122: note = The Description of the artificial sequence: amino acid sequence of VH excluding signal sequence of MOG 418
QVYLVQSGGGLVKPGGSLRLSCAASGFTFSSYSMHWVRQAPGKGLEWVSSISHSSSYISYADSVKGRFTISRDNAKNSLYLQMNSLRAEDTAVYYCAKGYYDILTGSLFDYWGQGTLVTVSS

SEQ ID NO: 337 - DNA, 321 bp, synthetic construct
  misc_feature 1..321: note = The Description of the artificial sequence: base sequence encoding VL excluding signal sequence of MOG 418
  source 1..321: mol_type = other DNA
gaaattgtgttgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagactggagcctgaagattttgcagtgtattactgtcagcagtatggtagctcaccattcactttcggccctgggaccaaagtggatatcaaa

SEQ ID NO: 338 - protein, 107 aa, synthetic construct
  REGION 1..107: note = The Description of the artificial sequence: amino acid sequence of VL excluding signal sequence of MOG 418
EIVLTQSPATLSLSPGERATLSCRASQSVSSYLAWYQQKPGQAPRLLIYDASNRATGIPARFSGSGSGTDFTLTISRLEPEDFAVYYCQQYGSSPFTFGPGTKVDIK

SEQ ID NO: 339 - protein, 146 aa, Homo sapiens; identical to SEQ ID NO: 2

SEQ ID NO: 340 - protein, 130 aa, Homo sapiens; identical to SEQ ID NO: 8

SEQ ID NO: 341 - protein, 141 aa, Homo sapiens; identical to SEQ ID NO: 14

SEQ ID NO: 342 - protein, 129 aa, Homo sapiens; identical to SEQ ID NO: 20

SEQ ID NO: 343 - protein, 138 aa, Homo sapiens; identical to SEQ ID NO: 26

SEQ ID NO: 344 - protein, 127 aa, Homo sapiens; identical to SEQ ID NO: 32

SEQ ID NO: 345 - protein, 137 aa, synthetic construct; identical to SEQ ID NO: 38

SEQ ID NO: 346 - protein, 245 aa, Rattus norvegicus; identical to SEQ ID NO: 68

SEQ ID NO: 347 - protein, 388 aa, synthetic construct; identical to SEQ ID NO: 70

SEQ ID NO: 348 - protein, 378 aa, synthetic construct; identical to SEQ ID NO: 72

SEQ ID NO: 349 - protein, 247 aa, Mus musculus; identical to SEQ ID NO: 74

SEQ ID NO: 350 - protein, 247 aa, Macaca fascicularis; identical to SEQ ID NO: 76

SEQ ID NO: 351 - protein, 247 aa, Homo sapiens; identical to SEQ ID NO: 78

SEQ ID NO: 352 - protein, 879 aa, synthetic construct; identical to SEQ ID NO: 99

(19) United States
(12) Patent Application Publication
(10) Pub. No.: US 2026/0092110 A1
(43) Pub. Date: Apr. 2, 2026

(54) ANTIBODY WHICH BINDS TO MYELIN OLIGODENDROCYTE GLYCOPROTEIN

(71) Applicants: Kyowa Kirin Co., Ltd., Tokyo (JP); KAGOSHIMA UNIVERSITY, Kagoshima (JP)

(72) Inventors: Nobuaki TAKAHASHI, Tokyo (JP); Ryosuke NAKANO, Tokyo (JP); Sayaka MAEDA, Tokyo (JP); Yuji ITO, Kagoshima (JP)

(73) Assignees: Kyowa Kirin Co., Ltd., Tokyo (JP); KAGOSHIMA UNIVERSITY, Kagoshima (JP)

(21) Appl. No.: 19/175,567

(22) Filed: Apr. 10, 2025

Related U.S. Application Data
(63) Continuation of application No. 18/363,113, filed on Aug. 1, 2023, now Pat. No. 12,297,268.
Application No. 18/363,113 is a continuation of application No. 17/399,354, filed on Aug. 11, 2021, now abandoned.
Application No. 17/399,354 is a division of application No. 16/473,482, now Pat. No. 11,117,963.

(30) Foreign Application Priority Data
Dec. 26, 2016 (JP) 2016-251106

(51) Int. Cl. C07K 16/28; A61K 47/10; A61K 47/36; C12N 5/12; G01N 33/563

(52) U.S. Cl. CPC C07K 16/2803; A61K 47/10; A61K 47/36; C12N 5/12; G01N 33/563; C07K 2317/24; C07K 2317/31; C07K 2317/565

(57) ABSTRACT

The invention relates to an antibody which binds to myelin oligodendrocyte glycoprotein (MOG), an antibody fragment thereof, a hybridoma which produces the antibody or the antibody fragment, a nucleic acid containing a nucleotide sequence which encodes the antibody or the antibody fragment, a transformant cell containing a vector containing the nucleic acid, a method for producing the antibody or the antibody fragment, a composition containing the antibody or the antibody fragment and a method for detecting or measuring an antigen that is present in the brain, a method for diagnosing or treating a brain disease, a method for improving the property of an antibody of accumulating in the brain and a method for increasing the amount of an antibody in the brain which use the antibody or the antibody fragment.

### CROSS REFERENCE TO RELATED APPLICATIONS

This application is a continuation of U.S. patent application Ser. No. 18/363,113 filed on Aug. 1, 2023 which is a continuation of U.S. patent application Ser. No. 17/399,354 filed on Aug. 11, 2021, which is a divisional application of U.S. patent application Ser. No. 16/473,482 filed on Jun. 25, 2019, now U.S. Pat. No. 11,117,963, which is a national stage application of International Application No. PCT/JP2017/046445 filed on Dec. 25, 2017.

### SEQUENCE LISTING

A sequence listing in electronic (XML file) format is filed with this application and incorporated herein by reference. The name of the ASCII text file is “Sequence_Listing-0515”; the file was created on Apr. 10, 2025; the size of the file is 570,775 bytes.

### TECHNICAL FIELD

The present invention relates to an antibody which binds to myelin oligodendrocyte glycoprotein (MOG), an antibody fragment thereof, a hybridoma which produces the antibody or the antibody fragment, a nucleic acid containing a nucleotide sequence which encodes the antibody or the antibody fragment, a transformant cell containing a vector containing the nucleic acid, a method for producing the antibody or the antibody fragment, a composition containing the antibody or the antibody fragment and a method for detecting or measuring an antigen that is present in the brain, a method for diagnosing or treating a brain disease, a method for improving the property of an antibody of accumulating in the brain and a method for increasing the amount of an antibody in the brain which use the antibody or the antibody fragment.

### BACKGROUND ART

Since the approval of a mouse anti-CD3 antibody, muromonab-CD3 (OKT3) as the first antibody drug by FDA in 1986, many antibody drugs have been developed. In 1994, a chimeric antibody, abciximab, in which a variable region of a mouse antibody and a constant region of a human antibody are linked to reduce the antigenicity of the mouse antibody, has been approved.

To further reduce the antigenicity, humanized antibody technique in which a complementarity determining region (CDR below), which plays an important role in binding to an antigen, of a variable region of a mouse antibody is inserted to the frame work region (FR below) of a human antibody has been developed, and a humanized anti-CD20 antibody, dacizumab has been approved in 1997.

Moreover, phage display technique using a human antibody sequence library has been used, and a fully human anti-TNF α antibody, adalimumab, which is the first antibody using the phage display technique that has been approved, has been approved in 2002. Sixty or more antibody drugs targeting antigens such as CD20, CD52, TNF α, HER2 and EGFR have already been approved (NPL 1).

In this manner, antibodies are a widely recognized drug format. Most of the antibody drugs that have been approved so far are those for cancers and immune diseases, which account for about 75% or more of all the antibody drugs.

The importance of biologics such as antibodies is increasing also in the treatment of central nervous system diseases, and it is reported that a monoclonal antibody to amyloid p is studied in Alzheimer's disease and that neurotrophic factors (brain-derived neurotrophic factor BDNF and glial-derived neurotrophic factor GDNF) having neuroprotective effect exhibit neuroprotective effect in central nervous system diseases in an animal model (NPL 2).

However, when an antibody is peripherally administered, the amount sent to the central nervous system is lower than those to the other organs, and the antibody migration rate (the ratio of the concentration in the cerebrospinal fluid (CSF) to the serum concentration) is reported to be 0.1-0.3% (NPLs 3-5).

A reason why the drug delivery amount decreases in the central nervous system including the brain and the bone marrow is the mechanism which is called the blood brain barrier (BBB) and which limits the transportation of a substance between the tissue liquids of the blood and the brain. The blood brain barrier has a physical/nonspecific control mechanism due to the intercellular adhesion of the vascular endothelial cells and a substrate-specific efflux mechanism due to efflux transporters. The blood brain barrier protects the central nervous system from foreign matters or drugs and plays an important role in maintaining the homeostasis.

However, due to the existence of the blood brain barrier, the effective concentration of drug administration is not easily obtained in the central nervous system, and the drug development is difficult. For example, although enzyme replacement therapy is conducted by intravenously administering α-L-iduronidase to Hurler syndrome (mucopolysaccharidosis-I) or iduronate-2-sulfatase to Hunter syndrome (mucopolysaccharidosis-II), the enzymes do not pass through the blood brain barrier due to their high molecular weights, and no efficacy on conditions in the central nervous system has been found (NPLs 6-9). Moreover, it is reported that side effects such as production of a neutralizing antibody are caused because a certain amount of a recombinant enzyme is continuously administered regularly (NPL 10).

Moreover, attempts to directly administer biologics into the medullary cavity or the brain have been made to increase the concentration in the brain. For example, a method of administering iduronate-2-sulfatase into the brain of patients with Hunter syndrome (mucopolysaccharidosis-II) to prevent the progress of brain disorder of the patients is reported (PTL 1). However, direct administration into the medullary cavity or the brain is highly invasive (NPL 11).

Therefore, various delivery techniques have been studied to increase the concentrations of substances with high molecular weights such as biologics in the brain. For example, methods for allowing a substance with a high molecular weight to pass through the blood brain barrier through endocytosis by binding the substance and a membrane protein which is expressed in the brain vascular endothelial cells and by forming a complex of the substance with a high molecular weight and the membrane protein are reported.

Most of the reported techniques use receptor-mediated transcytosis (RMT below), and the target receptors expressed in the brain vascular endothelium are, for example, transferrin receptors, insulin receptors, insulin-like growth factor receptors and the low-density lipoprotein receptor family (LDLRf).

Techniques for passing the blood brain barrier through a transferrin receptor by producing a fused protein of an anti-transferrin receptor antibody and a nerve growth factor are reported. Reported techniques using an anti-transferrin receptor antibody are bispecific antibodies of an anti-transferrin receptor antibody and an anti-beta secretase (BACE1) antibody (PTLs 2 and 3 and NPLs 12 and 13) and fused antibodies obtained by fusing a monovalent anti-transferrin receptor antibody to the carboxyl terminus of an anti-amyloid p antibody (PTL 4 and NPL 14).

It is reported that, regarding the brain delivery using a bispecific antibody of an anti-transferrin receptor antibody and an anti-BACE1 antibody, the amount of the antibody taken into the brain increases to about four times the amount of the control when the antibody is administered to a mouse at 20 mg/kg body weight (NPL 13).

Furthermore, a technique for allowing a drug to pass through the blood brain barrier by encapsulating the drug with a liposome having an anti-transferrin receptor antibody on its surface is reported. It is reported that the amount taken into the rat brain increases to about two to five times when an anti-rat transferrin receptor antibody fused to immunomicelle is used (NPL 9).

Techniques for passing through the blood brain barrier through an insulin receptor by producing a fused protein of a neurotrophic factor, an enzyme or an anti-amyloid antibody fused to the carboxyl terminus of an anti-insulin receptor antibody are reported (NPLs 16-19).

It is reported that when a fused antibody of a labeled anti-human insulin receptor antibody and GDNF is administered to a rhesus monkey, the amount taken into the brain after two hours is about 15 times compared to that of GDNF (NPL 17).

However, because transferrin receptors and insulin receptors are expressed not only in the brain vascular endothelial cells but also in the whole body including the liver and the like, a drug is delivered also to the liver and the like as the amount of the drug delivered to the central nervous system increases in these techniques (NPL 20). Moreover, because the antigen is expressed in the whole body, the half-life of the antibody in the blood is short (NPL 12).

Moreover, it is reported that an antibody (Fc5) to TMEM30A, which is an antigen expressed in the brain vascular endothelial membrane, shows an RMT-like activity (PTL 5 and NPLs 21 and 22). Fc5 is an antibody of a variable domain of heavy chain of heavy chain antibody (VHH below) of a single domain derived from llama, and it is shown in an in vitro BBB model and in a rat in vivo model that the amount of Fc5 fused with human Fc delivered to the brain is higher than that of the control IgG.

It is reported that the CSF exposure of a Fc5-derived single chain antibody (scFv) fused with a metabotropic glutamate receptor type I (mGluRI below) antibody is higher than that of a control single chain antibody fused with a mGluRI antibody in a rat model, but the increase in the amount is around five times (NPL 23).

It is also reported that an IgG antibody is rapidly discharged from the brain to the circulating blood by neonatal Fc receptor (FcRn) (NPLs 24 and 25), and for example, the half-life of IgG in the brain after the administration into the brain is as short as 48 minutes in rats (NPL 24).

MOG is a protein belonging to the immunoglobulin superfamily and constitutes myelin. Whole human MOG consists of 218 amino acids, and human MOG is expressed in the outermost layer of myelin in the central nervous system and plays a role in the cell adhesion and the cell surface interaction (NPLs 26-28).

MOG is considered as a candidate of an autoantigen in inflammatory diseases in which the glial cells in the central nerves are attacked by the autoimmunity, such as multiple sclerosis (MS) (NPLs 29 and 30). It is reported that, although the concentrations of anti-MOG antibodies in the serum are low in MS patients, anti-MOG antibodies are detected also in the central nerves (NPL 29).

As a reason for this, it is reported that the blood brain barrier breaks due to leakage of humoral factors and entry of inflammatory cells in pathological conditions such as MS and that antibodies easily migrate to the central nervous system (NPLs 30 and 31). It is also reported that autoantibodies are produced locally in the central nervous system due to B cells and plasma cells infiltrated to the central nervous system (NPLs 30, 32 and 33).

Experimental autoimmune encephalomyelitis (EAE) and MS have many pathological conditions in common, and thus EAE is a model used for studying the pathological conditions of MS. It is reported that EAE can be induced by immunizing an animal with MOG protein or peptide (NPL 34).

It is also reported that the EAE score deteriorates when an anti-MOG antibody is administered to an animal in which EAE has been induced (NPLs 29 and 35). However, the EAE score reaches its peak one to two days (NPL 29) or four days (NPL 35) after the administration of the antibody, and the deterioration is temporal. On the other hand, it is also reported that EAE does not develop even when an anti-MOG antibody alone is administered to a normal animal (NPLs 36 and 37).

### CITATION LIST

### Patent Literature

PTL 1: International Publication No. 2012/023623
PTL 2: International Publication No. 2016/081640
PTL 3: International Publication No. 2016/081643
PTL 4: International Publication No. 2014/033074
PTL 5: Canadian Patent No. 2623841

### Non Patent Literature

NPL 1: Kyla R R. and Richard C C., Biotechnol Adv, pii: S0734-9750 (16), 30091-X, 2016
NPL 2: Pardridge W M., Bioconjugate Chem., 19, 1327-1338, 2008
NPL 3: Wang W., et al., Clin. pharmacol. Ther., 84, 548-558, 2008
NPL 4: Garg A., et al., AAPSJ., 11, 553-557, 2009
NPL 5: Kaj B., et al., Arch. Neurol., 69 (8), 1002-1010, 2012
NPL 6: Wraith J E. et al., J. Pediatr. 144 (5), 581-588, 2004
NPL 7: Muenzer J. et al., Genet Med. 8 (8), 465-473, 2006
NPL 8: Document attached to intravenous infusion 2.9 mg of Aldurazyme (registered trademark) (July, 2016, 8th edition)
NPL 9: Document attached to intravenous infusion 6 mg of Elaprase (registered trademark) (July, 2016, 6th edition)
NPL 10: Brooks, D. A. et al., Trends Mol. Med. 9, 450-453, 2003
NPL 11: Sorrentino N C. et al., Pediatr Endocrinol Rev. 1, 630-638, 2016
NPL 12: Couch J A., et al., Science Translational Medicine, 5, 183ra57, 2013
NPL 13: Yu Y J., et al., Science Translational Medicine, 6, 261ra154, 2014
NPL 14: Niewoehner J., et al., Neuron. 81, 49-60, 2014
NPL 15: Jun Y, et al., Macromol. Biosci. 12, 1209-1219, 2012
NPL 16: Pardridge W M. and Boado R J., Methods in Enzymology, 503, 269-292, 2012
NPL 17: Boado R J., et al., Drug Metab. Dispos., 37 (12), 2299-2304, 2009
NPL 18: Boado R J., et al., J. Pharmacol. Exp. Ther., 333 (3), 961-969, 2010
NPL 19: Boado R J., et al., Bioconjugate Chem., 1, 97-104, 2012
NPL 20: Yun Zhang. et al., J. Pharmacol. Exp. Ther., 313 (3), 1075-1081, 2005
NPL 21: Abulrob A., et al., J. Neuyrochem., 95 (4), 1201-1214, 2005
NPL 22: Farrington G K., et al., FASEB J., 28, 4764-4778, 2014
NPL 23: Webster C I., et al., FASEB J., 30, 1927-1940, 2016
NPL 24: Zhang Y, et al., J. Neuroimmunol., 114(1-2), 168-172, 2001
NPL 25: Philip R C., et al., Brain Research, 1534, 13-21, 2013
NPL 26: Brunner C., et al., J. Neurochem, 52, 296-394, 1989
NPL 27: Pham-Dinh D., et al., Proc. Natl. Acad. Sci. USA, 90, 7990-7994, 1993
NPL 28: Gardinier M V., et al., J. Neurosci. Res., 33, 177-187, 1992
NPL 29: Eduard Urich, et al., PNAS, 103, 18697-18702, 2006
NPL 30: Markus Reindl, et al., Brain, 122, 2047-2056, 1999
NPL 31: Shimizu F., et al., Nihon Rinsho. 72(11), 1949-1954, 2014
NPL 32: Nese Sinmaz., et al., Ann. N. Y Acad. Sci., 1351, 22-38, 2015
NPL 33: F. J. Quintana, Neurology, 78, 532-539, 2012
NPL 34: Ralf Gold., et al., Brain, 129, 1953-1971, 2006
NPL 35: Margaret M., et al., J. Neuroimmunology, 125, 114-124, 2002
NPL 36: G. Locatelli, et al., Nature Neuro Science, 15(4), 543-551, 2012
NPL 37: H J Schluesener, et al., J. Immunol., 139, 4016-4021, 1987

### SUMMARY OF INVENTION

### Technical Problem

Although it is disclosed in NPLs 29 and 35 that an anti-MOG antibody is detected in the brain when the antibody is administered to an EAE model, there is no report on an anti-MOG antibody which can be detected in the brain when the anti-MOG antibody is peripherally administered to a normal animal.

The invention relates to a myelin oligodendrocyte glycoprotein (MOG)-binding molecule which binds to MOG and methods using the molecule. Specifically, an object is to provide an antibody which binds to MOG, an antibody fragment thereof, a hybridoma which produces the antibody or the antibody fragment, a nucleic acid containing a nucleotide sequence which encodes the antibody or the antibody fragment, a transformant cell containing a vector containing the nucleic acid, a method for producing the antibody or the antibody fragment, a composition containing the antibody or the antibody fragment and a method for detecting or measuring an antigen that is present in the brain, a method for diagnosing or treating a brain disease, a method for improving the property of an antibody of accumulating in the brain and a method for increasing the amount of an antibody in the brain which use the antibody or the antibody fragment.

### Solution to Problem

As means for solving the problems, the invention provides a MOG-binding molecule which binds to MOG and methods using the molecule and specifically provides an antibody or an antibody fragment thereof.

That is, the invention relates to (1) to (22) below.

(1) An antibody which binds to myelin oligodendrocyte glycoprotein (referred to as MOG below) or an antibody fragment thereof.

(2) The antibody or the antibody fragment according to (1), wherein the antibody has a property of accumulating in a brain.

(3) The antibody or the antibody fragment according to (1) or (2), wherein the antibody is selected from the group consisting of (a) to (r) below,
(a) an antibody in which the amino acid sequences of complementarity determining regions (CDRs below) 1 to 3 of a heavy chain variable region (referred to as VH below) contain the amino acid sequences of SEQ ID NOs: 4, 5 and 6, respectively, and in which the amino acid sequences of CDRs 1 to 3 of a light chain variable region (VL) contain the amino acid sequences of SEQ ID NOs: 10, 11 and 12, respectively,
(b) an antibody in which the amino acid sequences of CDRs 1 to 3 of VH contain the amino acid sequences of SEQ ID NOs: 16, 17 and 18, respectively, and in which the amino acid sequences of CDRs 1 to 3 of VL contain the amino acid sequences of SEQ ID NOs: 22, 23 and 24, respectively,
(c) an antibody in which the amino acid sequences of CDRs 1 to 3 of VH contain the amino acid sequences of SEQ ID NOs: 28, 29 and 30, respectively, and in which the amino acid sequences of CDRs 1 to 3 of VL contain the amino acid sequences of SEQ ID NOs: 34, 35 and 36, respectively,
(d) an antibody fragment in which the amino acid sequences of CDRs 1 to 3 of a heavy chain variable region of a heavy chain antibody (referred to as VHH below) contain the amino acid sequences of SEQ ID NOs: 40, 41 and 42, respectively,
(e) an antibody in which the amino acid sequences of CDRs 1 to 3 of VH contain the amino acid sequences of SEQ ID NOs: 153, 154 and 155, respectively, and in which the amino acid sequences of CDRs 1 to 3 of VL contain the amino acid sequences of SEQ ID NOs: 158, 159 and 160, respectively,
(f) an antibody in which the amino acid sequences of CDRs 1 to 3 of VH contain the amino acid sequences of SEQ ID NOs: 163, 164 and 165, respectively, and in which the amino acid sequences of CDRs 1 to 3 of VL contain the amino acid sequences of SEQ ID NOs: 168, 169 and 170, respectively,
(g) an antibody in which the amino acid sequences of CDRs 1 to 3 of VH contain the amino acid sequences of SEQ ID NOs: 173, 174 and 175, respectively, and in which the amino acid sequences of CDRs 1 to 3 of VL contain the amino acid sequences of SEQ ID NOs: 178, 179 and 180, respectively,
(h) an antibody in which the amino acid sequences of CDRs 1 to 3 of VH contain the amino acid sequences of SEQ ID NOs: 183, 184 and 185, respectively, and in which the amino acid sequences of CDRs 1 to 3 of VL contain the amino acid sequences of SEQ ID NOs: 188, 189 and 190, respectively,
(i) an antibody in which the amino acid sequences of CDRs 1 to 3 of VH contain the amino acid sequences of SEQ ID NOs: 193, 194 and 195, respectively, and in which the amino acid sequences of CDRs 1 to 3 of VL contain the amino acid sequences of SEQ ID NOs: 198, 199 and 200, respectively,
(j) an antibody in which the amino acid sequences of CDRs 1 to 3 of VH contain the amino acid sequences of SEQ ID NOs: 203, 204 and 205, respectively, and in which the amino acid sequences of CDRs 1 to 3 of VL contain the amino acid sequences of SEQ ID NOs: 208, 209 and 210, respectively,
(k) an antibody in which the amino acid sequences of CDRs 1 to 3 of VH contain the amino acid sequences of SEQ ID NOs: 213, 214 and 215, respectively, and in which the amino acid sequences of CDRs 1 to 3 of VL contain the amino acid sequences of SEQ ID NOs: 218, 219 and 220, respectively,
(l) an antibody in which the amino acid sequences of CDRs 1 to 3 of VH contain the amino acid sequences of SEQ ID NOs: 223, 224 and 225, respectively, and in which the amino acid sequences of CDRs 1 to 3 of VL contain the amino acid sequences of SEQ ID NOs: 228, 229 and 230, respectively,
(m) an antibody in which the amino acid sequences of CDRs 1 to 3 of VH contain the amino acid sequences of SEQ ID NOs: 233, 234 and 235, respectively, and in which the amino acid sequences of CDRs 1 to 3 of VL contain the amino acid sequences of SEQ ID NOs: 238, 239 and 240, respectively,
(n) an antibody in which the amino acid sequences of CDRs 1 to 3 of VH contain the amino acid sequences of SEQ ID NOs: 243, 244 and 245, respectively, and in which the amino acid sequences of CDRs 1 to 3 of VL contain the amino acid sequences of SEQ ID NOs: 248, 249 and 250, respectively,
(o) an antibody which competes in binding to MOG with at least one of the antibodies described in (a) to (n),
(p) an antibody which binds to an epitope containing an epitope to which any one of the antibodies described in (a) to (n) binds,
(q) an antibody which binds to the same epitope as an epitope to which any one of the antibodies described in (a) to (n) binds and
(r) an antibody which contains an amino acid sequence having homology of 85% or higher to the amino acid sequence of any one of the antibodies described in (a) to (n).

(4) The antibody or the antibody fragment according to any one of (1) to (3), wherein the antibody is selected from the group consisting of (a) to (n), (o1) to (o22) and (p) below,
(a) an antibody in which the amino acid sequence of VH contains the amino acid sequence of SEQ ID NO: 3 and in which the amino acid sequence of VL contains the amino acid sequence of SEQ ID NO: 9,
(b) an antibody in which the amino acid sequence of VH contains the amino acid sequence of SEQ ID NO: 15 and in which the amino acid sequence of VL contains the amino acid sequence of SEQ ID NO: 21,
(c) an antibody in which the amino acid sequence of VH contains the amino acid sequence of SEQ ID NO: 27 and in which the amino acid sequence of VL contains the amino acid sequence of SEQ ID NO: 33,
(d) an antibody fragment in which the amino acid sequence of VHH contains the amino acid sequence of SEQ ID NO: 39,
(e) an antibody in which the amino acid sequence of VH contains the amino acid sequence of SEQ ID NO: 152 and in which the amino acid sequence of VL contains the amino acid sequence of SEQ ID NO: 157,
(f) an antibody in which the amino acid sequence of VH contains the amino acid sequence of SEQ ID NO: 162 and in which the amino acid sequence of VL contains the amino acid sequence of SEQ ID NO: 167,
(g) an antibody in which the amino acid sequence of VH contains the amino acid sequence of SEQ ID NO: 172 and in which the amino acid sequence of VL contains the amino acid sequence of SEQ ID NO: 177,
(h) an antibody in which the amino acid sequence of VH contains the amino acid sequence of SEQ ID NO: 182 and in which the amino acid sequence of VL contains the amino acid sequence of SEQ ID NO: 187,
(i) an antibody in which the amino acid sequence of VH contains the amino acid sequence of SEQ ID NO: 192 and in which the amino acid sequence of VL contains the amino acid sequence of SEQ ID NO: 197,
(j) an antibody in which the amino acid sequence of VH contains the amino acid sequence of SEQ ID NO: 202 and in which the amino acid sequence of VL contains the amino acid sequence of SEQ ID NO: 207,
(k) an antibody in which the amino acid sequence of VH contains the amino acid sequence of SEQ ID NO: 212 and in which the amino acid sequence of VL contains the amino acid sequence of SEQ ID NO: 217,
(l) an antibody in which the amino acid sequence of VH contains the amino acid sequence of SEQ ID NO: 222 and in which the amino acid sequence of VL contains the amino acid sequence of SEQ ID NO: 227,
(m) an antibody in which the amino acid sequence of VH contains the amino acid sequence of SEQ ID NO: 232 and in which the amino acid sequence of VL contains the amino acid sequence of SEQ ID NO: 237,
(n) an antibody in which the amino acid sequence of VH contains the amino acid sequence of SEQ ID NO: 242 and in which the amino acid sequence of VL contains the amino acid sequence of SEQ ID NO: 247,
(o1) an antibody in which the amino acid sequence of VH contains the amino acid sequence of SEQ ID NO: 252 and in which the amino acid sequence of VL contains the amino acid sequence of SEQ ID NO: 254,
(o2) an antibody in which the amino acid sequence of VH contains the amino acid sequence of SEQ ID NO: 256 and in which the amino acid sequence of VL contains the amino acid sequence of SEQ ID NO: 258,
(o3) an antibody in which the amino acid sequence of VH contains the amino acid sequence of SEQ ID NO: 260 and in which the amino acid sequence of VL contains the amino acid sequence of SEQ ID NO: 262,
(o4) an antibody in which the amino acid sequence of VH contains the amino acid sequence of SEQ ID NO: 264 and in which the amino acid sequence of VL contains the amino acid sequence of SEQ ID NO: 266,
(o5) an antibody in which the amino acid sequence of VH contains the amino acid sequence of SEQ ID NO: 268 and in which the amino acid sequence of VL contains the amino acid sequence of SEQ ID NO: 270,
(o6) an antibody in which the amino acid sequence of VH contains the amino acid sequence of SEQ ID NO: 272 and in which the amino acid sequence of VL contains the amino acid sequence of SEQ ID NO: 274,
(o7) an antibody in which the amino acid sequence of VH contains the amino acid sequence of SEQ ID NO: 276 and in which the amino acid sequence of VL contains the amino acid sequence of SEQ ID NO: 278,
(o8) an antibody in which the amino acid sequence of VH contains the amino acid sequence of SEQ ID NO: 280 and in which the amino acid sequence of VL contains the amino acid sequence of SEQ ID NO: 282,
(o9) an antibody in which the amino acid sequence of VH contains the amino acid sequence of SEQ ID NO: 284 and in which the amino acid sequence of VL contains the amino acid sequence of SEQ ID NO: 286,
(o10) an antibody in which the amino acid sequence of VH contains the amino acid sequence of SEQ ID NO: 288 and in which the amino acid sequence of VL contains the amino acid sequence of SEQ ID NO: 290,
(o11) an antibody in which the amino acid sequence of VH contains the amino acid sequence of SEQ ID NO: 292 and in which the amino acid sequence of VL contains the amino acid sequence of SEQ ID NO: 294,
(o12) an antibody in which the amino acid sequence of VH contains the amino acid sequence of SEQ ID NO: 296 and in which the amino acid sequence of VL contains the amino acid sequence of SEQ ID NO: 298,
(o13) an antibody in which the amino acid sequence of VH contains the amino acid sequence of SEQ ID NO: 300 and in which the amino acid sequence of VL contains the amino acid sequence of SEQ ID NO: 302,
(o14) an antibody in which the amino acid sequence of VH contains the amino acid sequence of SEQ ID NO: 304 and in which the amino acid sequence of VL contains the amino acid sequence of SEQ ID NO: 306,
(o15) an antibody in which the amino acid sequence of VH contains the amino acid sequence of SEQ ID NO: 308 and in which the amino acid sequence of VL contains the amino acid sequence of SEQ ID NO: 310,
(o16) an antibody in which the amino acid sequence of VH contains the amino acid sequence of SEQ ID NO: 312 and in which the amino acid sequence of VL contains the amino acid sequence of SEQ ID NO: 314,
(o17) an antibody in which the amino acid sequence of VH contains the amino acid sequence of SEQ ID NO: 316 and in which the amino acid sequence of VL contains the amino acid sequence of SEQ ID NO: 318,
(o18) an antibody in which the amino acid sequence of VH contains the amino acid sequence of SEQ ID NO: 320 and in which the amino acid sequence of VL contains the amino acid sequence of SEQ ID NO: 322,
(o19) an antibody in which the amino acid sequence of VH contains the amino acid sequence of SEQ ID NO: 324 and in which the amino acid sequence of VL contains the amino acid sequence of SEQ ID NO: 326,
(o20) an antibody in which the amino acid sequence of VH contains the amino acid sequence of SEQ ID NO: 328 and in which the amino acid sequence of VL contains the amino acid sequence of SEQ ID NO: 330,
(o21) an antibody in which the amino acid sequence of VH contains the amino acid sequence of SEQ ID NO: 332 and in which the amino acid sequence of VL contains the amino acid sequence of SEQ ID NO: 334,
(o22) an antibody in which the amino acid sequence of VH contains the amino acid sequence of SEQ ID NO: 336 and in which the amino acid sequence of VL contains the amino acid sequence of SEQ ID NO: 338 and
(p) an antibody which contains an amino acid sequence having homology of 85% or higher to the amino acid sequence of any one of the antibodies described in (a) to (n) and (o1) to (o22).

(5) The antibody or the antibody fragment according to any one of (1) to (4), wherein the antibody or the antibody fragment is a bispecific antibody.

(6) The bispecific antibody according to (5), wherein the bispecific antibody binds to MOG and an antigen that is present in a brain.

(7) The bispecific antibody according to (5) or (6), wherein the bispecific antibody contains an antigen binding site which binds to MOG and an antigen binding site which binds to an antigen that is present in a brain.

(8) The antibody fragment according to any one of (1) to (7) which is selected from the group consisting of Fab, Fab′, F(ab′)₂, a single chain antibody (scFv), a dimerized V region (diabody), a disulfide-stabilized V region (dsFv), VHH and, a peptide containing CDR.

(9) The antibody and the antibody fragment according to any one of (1) to (8), wherein the antibody is a genetically recombinant antibody.

(10) The antibody and the antibody fragment according to any one of (1) to (9), wherein the antibody is selected from the group consisting of a mouse antibody, a rat antibody, a rabbit antibody, an alpaca antibody, a camel antibody, a llama antibody, a chimeric antibody, a humanized antibody, and a human antibody.

(11) A fused antibody or a fused antibody fragment which is obtained by binding at least one selected from the group consisting of (a) to (c) below to the antibody which binds to MOG according to any one of (1) to (10) or the antibody fragment thereof,
(a) a hydrophilic polymer,
(b) an amphipathic polymer, and
(c) a functional molecule.

(12) A hybridoma which produces the antibody according to any one of (1) to (11).

(13) A nucleic acid which contains a nucleotide sequence which encodes the antibody according to any one of (1) to (11).

(14) A transformant cell which contains a vector containing the nucleic acid according to (13).

(15) A method for producing the antibody or the antibody fragment according to any one of (1) to (11), including culturing the hybridoma according to (12) or the transformant cell according to (14) and collecting the antibody or the antibody fragment according to any one of (1) to (11) from a culture solution.

(16) A composition which contains the antibody or the antibody fragment according to any one of (1) to (11).

(17) The composition according to (16), wherein the composition is a composition for detecting or measuring an antigen that is present in a brain.

(18) The composition according to (16), wherein the composition is a composition for diagnosing or treating a brain disease.

(19) A method for detecting or measuring an antigen that is present in a brain using the antibody or the antibody fragment according to any one of (1) to (11) or the composition according to (16).

(20) A method for diagnosing or treating a brain disease using the antibody or the antibody fragment according to any one of (1) to (11) or the composition according to (16).

(21) A method for improving the property of accumulating in the brain of an antibody, an antibody fragment thereof, a fused antibody or a fused antibody fragment using the antibody, the antibody fragment, the fused antibody or the fused antibody fragment according to any one of (1) to (11) or the composition according to (16).

(22) A method for increasing the amount of an antibody, the amount of an antibody fragment thereof, the amount of a fused antibody or the amount of a fused antibody fragment in a brain using the antibody, the antibody fragment, the fused antibody or the fused antibody fragment according to any one of (1) to (11) or the composition according to (16).

### Advantageous Effects of Invention

The MOG-binding molecule of the invention improves the property of the binding molecule itself of accumulating in the brain by specifically binding to MOG, and the MOG-binding molecule can also be applied to the treatment of brain diseases because another molecule which is modified by the MOG-binding molecule is delivered and kept in the brain. A specific MOG-binding molecule of the invention is an antibody. The antibody or the antibody fragment of the invention relates to an antibody which binds to MOG in the brain and thus has a property of accumulating in the brain. Accordingly, the antibody or the antibody fragment of the invention can be used for a composition for detecting or measuring an antigen that is present in the brain (MOG, or MOG and another antigen that is present in the brain), a composition for diagnosing a brain disease and a pharmaceutical composition for treating a brain disease.

### BRIEF DESCRIPTION OF DRAWINGS

FIG. 1 shows the results of analysis by ELISA of the affinities to rMOG-FLAG_Fc of phage clones displaying scFv that binds to MOG. The vertical axis shows the absorbance relative to rMOG-FLAG_Fc, and the horizontal axis shows the names of the scFv antibodies displayed by the phage clones.

FIG. 2 shows the results of analysis using a flow cytometer of the affinities of anti-MOG antibodies to BEK cells, rat MOG/HEK cells, mouse MOG/HEK cells, cynomolgus monkey MOG/HEK cells or human MOG/HEK cells. The vertical axis shows the number of cells, and the horizontal axis shows the fluorescence intensity. A dotted line indicates a histogram of the affinity of an anti-AVM antibody used as a negative control, and a solid line indicates a histogram of the affinity of a MOG antibody.

FIGS. 3A and 3B show the results of evaluation of the rat brain migration properties of anti-MOG antibodies. FIG. 3A shows the antibody concentrations in the serum four days after administering the antibodies to rats. The vertical axis shows the antibody concentration (ng/mL), and the horizontal axis shows the administered antibodies. FIG. 3B shows the antibody concentrations in the brain tissues four days after administering the antibodies to rats. The vertical axis shows the antibody amount per brain weight (ng/g brain), and the horizontal axis shows the administered antibodies. In both figures, the white bars show the results of an anti-AVM antibody used as a negative control, and the black bars show the results of the anti-MOG antibodies.

FIGS. 4A and 4B show the results of evaluation of the rat brain migration property of an anti-MOG antibody. FIG. 4A shows the antibody concentrations in the serum four days and 10 days after administering the antibodies to rats. The vertical axis shows the antibody concentration (ng/mL), and the horizontal axis shows the period (day) after the administration of the antibodies. FIG. 4B shows the antibody concentrations in the brain tissues four days and 10 days after administering the antibodies to rats. The vertical axis shows the antibody amount per brain weight (ng/g brain), and the horizontal axis shows the period (day) after the administration of the antibodies. In both figures, the plots with white diamond markers show the results of an anti-AVM antibody used as a negative control. The plots with white square markers show the results of an anti-transferrin receptor antibody, OX26 antibody, and the plots with black triangle markers show the results of the anti-MOG antibody, MOG01 antibody.

FIG. 5 shows the results of analysis using a flow cytometer of the affinities of bispecific antibodies to HEK293F cells, rat MOG/HEK293F cells or human MOG/HEK293F cells. The vertical axis shows the number of cells, and the horizontal axis shows the fluorescence intensity. A dotted line indicates a histogram of the affinity of an anti-AVM antibody used as a negative control, and a solid line indicates a histogram of the affinity of a bispecific antibody.

FIG. 6 shows the results of analysis using a flow cytometer of the affinities of bispecific antibodies to human breast cancer cell line, SK-BR-3. The vertical axis shows the number of cells, and the horizontal axis shows the fluorescence intensity. A dotted line indicates a histogram of the affinity of an anti-AVM antibody used as a negative control, and a solid line indicates a histogram of the affinity of a bispecific antibody.

FIGS. 7A and 7B show the results of evaluation of the rat brain migration properties of bispecific antibodies which bind to MOG. FIG. 7A shows the antibody concentrations in the serum 10 days after administering the antibodies to rats. The vertical axis shows the antibody concentration (ng/mL), and the horizontal axis shows the bispecific antibodies used. FIG. 7B shows the antibody concentrations in the brain tissues 10 days after administering the antibodies to rats. The vertical axis shows the antibody amount per brain weight (ng/g brain), and the horizontal axis shows the bispecific antibodies used.

FIGS. 8A and 8B show the results of evaluation of the mouse brain migration property of an anti-MOG01 antibody. FIG. 8A shows the antibody concentrations in the serum 3, 6, 10, 14, 21 and 28 days after administering the antibodies to mice. The vertical axis shows the antibody concentration (ng/mL), and the horizontal axis shows the time (day). FIG. 8B shows the antibody concentrations in the brain tissues 3, 6, 10, 14, 21 and 28 days after administering the antibodies to mice. The vertical axis shows the antibody concentration (ng/g brain), and the horizontal axis shows the time (day). In both figures, the plots with white circle markers show the results of an anti-AVM antibody used as a negative control, and the plots with black square markers show the results of MOG01 scFv-hG4PE.

FIGS. 9A to 9C show the results of evaluation of the mouse brain migration property imaging of an anti-MOG01 antibody. FIG. 9A shows the measurement data of the fluorescence intensities in the brain six days after administering an Alexa FluorR 488-labeled anti-AVM antibody as a negative control and an Alexa FluorR 488-labeled anti-MOG01 antibody to mice, and FIG. 9B shows the measurement data of the fluorescence intensities in the brain after 14 days. FIG. 9C shows the values obtained by correcting the fluorescence amounts in the brain after six days and 14 days using the fluorescence intensities of the administered antibodies. The vertical axis shows the fluorescence amount in brain/fluorescence amount of administered antibody (%), and the horizontal axis shows the administered antibodies.

FIGS. 10A to 10C show the structures of bispecific antibodies which bind to AVM and MOG. FIG. 10A shows the structure of AVM-MOG01 IgG4PE(R409K) antibody, and FIG. 10B shows the structure of AVM IgG4PE(R409K)_MOG01 Fab antibody. FIG. 10C shows the structure of AVM IgG4PE(R409K)_MOG01sscFv antibody.

FIGS. 11A and 11B show the structures of bispecific antibodies which bind to AVM and MOG. FIG. 11A shows the structure of AVM IgG4PE(R409K)_MOG01dscFv antibody, AVM IgG4PE(R409K)_MOG01dscFv2 antibody and AVM IgG4PE(R409K)_MOG01dscFv4 antibody, and FIG. 11B shows the structure of AVM IgG4PE(R409K)_MOG01dscFv3 antibody and AVM IgG4PE(R409K)_MOG01dscFv5 antibody to AVM IgG4PE(R409K)_MOG01dscFv11antibody.

FIGS. 12A to 12C show the results of analysis using a flow cytometer of the affinities of bispecific antibodies to human MOG/L929 cells. The vertical axis shows the average fluorescence intensity, and the horizontal axis shows the antibody concentration. In FIG. 12A, the plot with white circle markers shows the results of AVM IgG4PE(R409K) antibody (negative control), and the plot with black square markers shows the results of AVM-MOG01 IgG4PE(R409K) antibody. In FIG. 12B, the plot with white circle markers shows the results of AVM IgG4PE(R409K)_AVMsscFv antibody (negative control), and the plot with black square markers shows the results of AVM IgG4PE(R409K)_MOG01sscFv antibody. In FIG. 12C, the plot with white circle markers shows the results of AVM IgG4PE(R409K)_AVM Fab antibody (negative control), and the plot with black square markers shows the results of AVM IgG4PE(R409K)_MOG01 Fab antibody.

FIGS. 13A and 13B show the results of analysis using a flow cytometer of the affinities of bispecific antibodies to human MOG/L929 cells. The vertical axis shows the average fluorescence intensity, and the horizontal axis shows the antibody concentration. In FIG. 13A, the plot with white square markers shows the results of AVM IgG4PE(R409K)_MOG01dscFv antibody, the plot with white circle markers shows the results of AVM IgG4PE(R409K)_MOG01dscFv2 antibody, and the plot with white triangle markers shows the results of AVM IgG4PE(R409K)_MOG01dscFv4 antibody. In FIG. 13B, the plot with white diamond markers shows the results of AVM IgG4PE(R409K)_MOG01dscFv3 antibody, the plot with black diamond markers shows the results of AVM IgG4PE(R409K)_MOG01dscFv5 antibody, the plot with white circle markers shows the results of AVM IgG4PE(R409K)_MOG01dscFv6 antibody, the plot with black circle markers shows the results of AVM IgG4PE(R409K)_MOG01dscFv7 antibody, the plot with white triangle markers shows the results of AVM IgG4PE(R409K)_MOG01dscFv8 antibody, the plot with black triangle markers shows the results of AVM IgG4PE(R409K)_MOG01dscFv9 antibody, the plot with white square markers shows the results of AVM IgG4PE(R409K)_MOG01dscFvlO antibody, and the plot with black square markers shows the results of AVM IgG4PE(R409K)_MOG01dscFv11 antibody.

FIGS. 14A and 14B show the results of evaluation of the mouse brain migration properties of bispecific antibodies. The vertical axis shows the antibody concentration, and the horizontal axis shows the bispecific antibodies used. FIGS. 14A and 14B show the antibody concentrations in the serum and in the brain tissues, respectively, 10 days after the administration of AVM IgG4PE(R409K) antibody (negative control) and AVM-MOG01 IgG4PE(R409K) antibody.

FIGS. 15A and 15B show the results of evaluation of the mouse brain migration properties of bispecific antibodies. The vertical axis shows the antibody concentration, and the horizontal axis shows the bispecific antibodies used. FIGS. 15A and 15B show the antibody concentrations in the serum and in the brain tissues, respectively, 10 days after the administration of AVM IgG4PE(R409K) AVMsscFv antibody (negative control) and AVM IgG4PE(R409K)_MOG01sscFv antibody.

FIGS. 16A and 16B show the results of evaluation of the mouse brain migration properties of bispecific antibodies. The vertical axis shows the antibody concentration, and the horizontal axis shows the bispecific antibodies used. FIGS. 16A and 16B show the antibody concentrations in the serum and in the brain tissues, respectively, 10 days after the administration of AVM IgG4PE(R409K)_AVM Fab antibody (negative control) and AVM IgG4PE(R409K)_MOG01 Fab antibody.

FIGS. 17A to 17D show the results of evaluation of the mouse brain migration properties of bispecific antibodies. The vertical axis shows the antibody concentration, and the horizontal axis shows the bispecific antibodies used. The negative control corresponding to AVM IgG4PE(R409K)_MOG01dscFv antibody is AVM IgG4PE(R409K)_AVMdscFv antibody, and the negative control corresponding to AVM IgG4PE(R409K)_MOG01dscFv3 antibody is AVM IgG4PE(R409K)_AVMdscFv3 antibody. The negative control corresponding to AVM IgG4PE(R409K)_MOG01dscFv5 antibody is AVM IgG4PE(R409K)_AVMdscFv5 antibody. FIG. 17A shows the antibody concentrations in the serum 10 days after the administration of the antibodies. FIG. 17B shows the antibody concentrations in the brain tissues 10 days after the administration of the antibodies. FIG. 17C shows the antibody concentrations in the serum 28 days after the administration of the antibodies. FIG. 17D shows the antibody concentrations in the brain tissues 28 days after the administration of the antibodies.

FIG. 18 shows the amino acid sequences of scFv of clones similar to a MOG antibody and shows clones similar to MOG301 antibody.

FIG. 19 shows the amino acid sequences of scFv of clones similar to a MOG antibody and shows clones similar to MOG303 antibody.

FIG. 20 shows the amino acid sequences of scFv of clones similar to a MOG antibody and shows clones similar to MOG307 antibody.

FIG. 21 shows the amino acid sequences of scFv of clones similar to a MOG antibody and shows clones similar to MOG310 antibody.

FIGS. 22A and 22B show the amino acid sequences of scFv of clones similar to MOG antibodies. FIG. 22A shows a clone similar to MOG329 antibody, and FIG. 22B shows a clone similar to MOG456 antibody.

FIG. 23 shows the results of analysis using a flow cytometer of the affinities of anti-MOG antibodies to Expi293F cells. The vertical axis shows the number of cells, and the horizontal axis shows the fluorescence intensity. A dotted line indicates a histogram of the affinity of an anti-AVM antibody used as a negative control, and a solid line indicates a histogram of the affinity of a MOG antibody.

FIG. 24 shows the results of analysis using a flow cytometer of the affinities of anti-MOG antibodies to mouse MOG/Expi293F cells. The vertical axis shows the number of cells, and the horizontal axis shows the fluorescence intensity. A dotted line indicates a histogram of the affinity of an anti-AVM antibody used as a negative control, and a solid line indicates a histogram of the affinity of a MOG antibody.

FIG. 25 shows the results of analysis using a flow cytometer of the affinities of anti-MOG antibodies to human MOG/Expi293F cells. The vertical axis shows the number of cells, and the horizontal axis shows the fluorescence intensity. A dotted line indicates a histogram of the affinity of an anti-AVM antibody used as a negative control, and a solid line indicates a histogram of the affinity of a MOG antibody.

FIG. 26 shows the results of analysis using a flow cytometer of the affinity of an enzyme-fused antibody, MOG01 IgG4PE(R409K)-ASM to human MOG/L929 cells. The vertical axis shows the average fluorescence intensity, and the horizontal axis shows the antibody concentration.

FIG. 27 shows the results of analysis by ELISA method of the affinities of anti-ASM antibodies (manufactured by LSBio) to MOG01 IgG4PE(R409K)-ASM and AVM IgG4PE(R409K)-ASM. The vertical axis shows the absorbance, and the horizontal axis shows the names of the immobilized antibodies. MOG01 IgG4PE and AVM IgG4PE were used as negative controls. The bars shaded with thin lines show the data of the anti-ASM antibodies at 5 μg/mL, and the bars shaded with thick lines show the data of the anti-ASM antibodies at 1 μg/mL. The white bars show the data of the anti-ASM antibodies at 0.2 μg/mL.

FIGS. 28A and 28B show the results of evaluation of the mouse brain migration properties of enzyme-fused antibodies, MOG01 IgG4PE(R409K)-ASM and AVM IgG4PE(R409K)-ASM. The vertical axis shows the antibody concentration, and the horizontal axis shows the enzyme-fused antibodies used. FIG. 28A shows the antibody concentrations in the serum 10 days after the administration of the antibodies. FIG. 28B shows the antibody concentrations in the brain tissues 10 days after the administration of the antibodies.

### DESCRIPTION OF EMBODIMENTS

The invention relates to an antigen-binding molecule which binds to myelin-oligodendrocyte glycoprotein (referred to as MOG below). More specifically, the invention relates to an antibody which binds to MOG or an antibody fragment thereof.

The MOG-binding molecule of the invention may be a molecule of any state as long as the molecule specifically binds to MOG and accumulates in the brain, and the MOG-binding molecule may be any of molecules such as proteins, nucleic acids and synthetic organic low-molecular-weight compounds/high-molecular-weight compounds. Specifically, the MOG-binding molecule may be any of recombinant proteins, antibodies, aptamers, low-molecular-weight compounds obtained by screening low-molecular-weight molecules and the like, but an antibody and an antibody fragment thereof are preferable. The MOG-binding molecule is preferably a molecule which binds to an extracellular region of MOG.

MOG is a protein belonging to the immunoglobulin superfamily and constitutes myelin. Whole human MOG, for example, consists of 218 amino acids, and human MOG is expressed in the outermost layer of myelin in the central nervous system and plays a role in the cell adhesion and the cell surface interaction.

The kinds of animal of MOG to which the MOG-binding molecule of the invention binds are mouse, rat, cynomolgus monkey, human and/or the like but are not particularly limited to these kinds, and an appropriate animal kind can be selected depending on the use of the antibody. For example, when the antibody of the invention is used for a pharmaceutical use for humans, the antibody is preferably an antibody which binds to at least human MOG.

In the invention, human MOG is a polypeptide which contains the amino acid sequence of SEQ ID NO: 78 or the amino acid sequence of NCBI accession No. AAB08088, a polypeptide which has the amino acid sequence of SEQ ID NO: 78 or the amino acid sequence of NCBI accession No. AAB08088, wherein one or more amino acids are deleted, substituted or added, and which has a function of human MOG, a polypeptide which has an amino acid sequence having homology of 60% or higher, preferably 80% or higher, further preferably 90% or higher, most preferably 95% or higher to the amino acid sequence of SEQ ID NO: 78 or the amino acid sequence of NCBI accession No. AAB08088 and which has a function of human MOG or the like.

The polypeptide which has the amino acid sequence of SEQ ID NO: 78 or the amino acid sequence of NCBI accession No. AAB08088, wherein one or more amino acids are deleted, substituted or added, can be obtained by introducing a site-specific mutation for example to DNA that encodes a polypeptide containing the amino acid sequence of SEQ ID NO: 78 using the site-directed mutagenesis [Molecular Cloning, A Laboratory Manual, Second Edition, Cold Spring Harbor Laboratory Press (1989), Current Protocols in Molecular Biology, John Wiley & Sons (1987-1997), Nucleic acids Research, 10, 6487 (1982), Proc. Natl. Acad. Sci. USA, 79, 6409 (1982), Gene, 34, 315 (1985), Nucleic Acids Research, 13, 4431 (1985) and Proc. Natl. Acad. Sci. USA, 82, 488 (1985)] or the like.

The number of amino acids that are deleted, substituted or added is not particularly limited but is preferably one to tens, for example, 1 to 20, more preferably one to a few, for example, one to five amino acids.

The same applies to the amino acid sequence of mouse MOG [SEQ ID NO: 74 and NCBI accession No. NP_034944], the amino acid sequence of rat MOG [SEQ ID NO: 68 and NCBI accession No. AAA41628] and the amino acid sequence of cynomolgus monkey MOG [SEQ ID NO: 76 and NCBI accession No. NP_001271785].

Genes which encode human MOG are the nucleotide sequence of SEQ ID NO: 77 and the nucleotide sequence of NCBI accession No. U64564. A gene containing DNA which has the nucleotide sequence of SEQ ID NO: 77 or the nucleotide sequence of NCBI accession No. U64564, wherein one or more bases are deleted, substituted or added, and which encodes a polypeptide having a function of MOG, a gene containing DNA which has a nucleotide sequence having homology of at least 60% or higher to the nucleotide sequence of SEQ ID NO: 77 or the nucleotide sequence of NCBI accession No. U64564, preferably a nucleotide sequence having homology of 80% or higher or further preferably a nucleotide sequence having homology of 95% or higher and which encodes a polypeptide having a function of MOG, a gene which contains DNA that hybridizes with DNA containing the nucleotide sequence of SEQ ID NO: 77 or the nucleotide sequence of NCBI accession No. U64564 under stringent conditions and which encodes a polypeptide having a function of MOG and another gene are also included as the genes that encode MOG in the invention.

The DNA that hybridizes under stringent conditions means hybridizable DNA that is obtained by a colony hybridization method, a plaque hybridization method, a southern blot hybridization method, a DNA microarray method or the like using DNA containing the nucleotide sequence of SEQ ID NO: 77 or the nucleotide sequence of NCBI accession No. U64564 as a probe.

Specifically, it is possible to exemplify DNA that can be identified by washing a filter or a glass slide under the condition of 65° C. using a SSC solution of the concentration of 0.1 to 2 times (the composition of the SSC solution with the concentration of 1 time is 150 mmol/L sodium chloride and 15 mmol/L sodium citrate), after performing hybridization [Molecular Cloning, A Laboratory Manual, Second Edition, Cold Spring Harbor Laboratory Press (1989), Current Protocols in Molecular Biology, John Wiley & Sons (1987-1997) and DNA Cloning 1: Core Techniques, A Practical Approach, Second Edition, Oxford University, (1995)] at 65° C. in the presence of 0.7 to 1.0 mol/L sodium chloride using a filter or a glass slide on which DNA derived from a hybridized colony or plaque or a PCR product or DNA oligo having the sequence is fixed.

Examples of the hybridizable DNA include DNA having homology of at least 60% or higher to the nucleotide sequence of SEQ ID NO: 77 or the nucleotide sequence of NCBI accession No. U64564, preferably DNA having homology of 80% or higher and further preferably DNA having homology of 95% or higher.

The same applies to the nucleotide sequence of mouse MOG [SEQ ID NO: 73 and NCBI accession No. NM_010814], the nucleotide sequence of rat MOG [SEQ ID NO: 67 and NCBI accession No. M99485] and the nucleotide sequence of cynomolgus monkey MOG [SEQ ID NO: 75 and NCBI accession No. NM_001284856].

The function of MOG is involvement in the cell adhesion, the cell surface interaction and the like on myelin.

Genetic polymorphism is often recognized in a nucleotide sequence of a gene that encodes a protein of a eukaryote. The genes that encode MOG in the invention also include genes in which small scale mutations arise in the nucleotide sequences by such polymorphism in the genes used in the invention.

A value of homology in the invention may be a value calculated using a homology detection program known to those skilled in the art unless particularly specified. Regarding a nucleotide sequence, there are a value calculated using a default parameter of BLAST [J. Mol. Biol., 215, 403 (1990)] and the like. Regarding an amino acid sequence, there are a value calculated using a default parameter of BLAST2 [Nucleic Acids Res., 25, 3389 (1997), Genome Res., 7, 649 (1997) and http://www.ncbi.nlm.nih.gov/Education/BLASTinfo/information3.htmL] and the like.

Regarding the default parameters, G (Cost to open gap) is 5 for a nucleotide sequence and 11 for an amino acid sequence, −E (Cost to extend gap) is 2 for a nucleotide sequence and 1 for an amino acid sequence, −q (Penalty for nucleotide mismatch) is −3, −r (reward for nucleotide match) is 1, −e (expect value) is 10, −W (wordsize) is 11 residues for a nucleotide sequence and 3 residues for an amino acid sequence, −y [Dropoff (X) for blast extensions in bits] is 20 for the blastn and 7 for programs other than the blastn, −X (X dropoff value for gapped alignment in bits) is 15, and −Z (final X dropoff value for gapped alignment in bits) is 50 for the blastn and 25 for programs other than the blastn (http://www.ncbi.nlm.nih.gov/blast/htmL/blastcgihelp.htmL).

A polypeptide containing a partial sequence of the amino acid sequence of any of the MOG kinds can be produced by a method known to those skilled in the art. Specifically, the polypeptide can be produced by deleting a part of DNA that encodes the amino acid sequence of any of the MOG kinds and culturing a transformant into which an expression vector including the DNA has been introduced. In addition, a polypeptide having the amino acid sequence of any of the MOG kinds in which one or more amino acids are deleted, substituted or added can be obtained by the same method as above.

Furthermore, a polypeptide that has the amino acid sequence of any of the MOG kinds or a polypeptide having the amino acid sequence of any of the MOG kinds in which one or more amino acids are deleted, substituted or added can be produced also using a chemical synthesis method such as a fluorenylmethyloxycarbonyl (Fmoc) method or a t-butyloxycarbonyl (tBoc) method.

In the invention, the extracellular region of human MOG is the amino acid sequence of from position 30 to position 154 or from position 232 to position 247 in the amino acid sequence of SEQ ID NO: 78 or NCBI accession No. AAB08088 and is preferably the amino acid sequence of from position 30 to position 154.

The extracellular region of mouse MOG is the amino acid sequence of from position 30 to position 157 or from position 232 to position 247 in the amino acid sequence of SEQ ID NO: 74 or NCBI accession No. NP_034944 and is preferably the amino acid sequence of from position 30 to position 157. The extracellular region of rat MOG is the amino acid sequence of from position 28 to position 155 or from position 230 to 245 in the amino acid sequence of SEQ ID NO: 68 or NCBI accession No. AAA41628 and is preferably the amino acid sequence of from position 28 to position 155.

The extracellular region of cynomolgus monkey MOG is the amino acid sequence of from position 30 to position 154 or from position 232 to position 247 in the amino acid sequence of SEQ ID NO: 76 or NCBI accession No. NP_001271785 and is preferably the amino acid sequence of from position 30 to position 154.

That the antibody of the invention binds to an extracellular region of MOG can be confirmed by measuring the affinity of the antibody of the invention to MOG-expressing cells or a recombinant MOG protein using ELISA, flow cytometry, surface plasmon resonance method or the like. Moreover, binding of the antibody can be confirmed also using a combination of known immunological detection methods [Monoclonal Antibodies-Principles and practice, Third edition, Academic Press (1996), Antibodies-A Laboratory Manual, Cold Spring Harbor Laboratory (1988) and A manual for monoclonal antibody experiments, Kodansha scientific books (1987)] and the like.

The MOG-binding molecule of the invention is a molecule which specifically binds to MOG in the brain and which thus has a property of accumulating in the brain, and for example, the antibody is an antibody which binds to MOG in the brain and which thus has a property of accumulating in the brain. Moreover, the antibody of the invention is an antibody which passes through the blood brain barrier in the brain from the peripheral part, migrates to the brain and binds to MOG in the brain when peripherally administered to an animal and which thus has a property of accumulating in the brain. The antibody of the invention is preferably an antibody having an excellent property of accumulating in the brain or an antibody having an improved property of accumulating in the brain.

In the invention, the property of accumulating in the brain is a property of a subject of accumulating in the brain when the subject is administered to an animal to be tested. That is, the property means that the concentration in the brain (or the amount in the brain) of the subject increases or that the subject exists at a certain detectable concentration due to at least any one cause selected from an increase in the migration into the brain, an increase in the accumulation in the brain, a decrease in the migration to outside from the brain, a decrease in the discharge to outside from the brain and a decrease in the decomposition in the brain.

In the invention, that the property of accumulating in the brain is excellent, that the property of accumulating in the brain is high or that the property of accumulating in the brain is improved means that the concentration in the brain (or the amount in the brain) of the subject increases or that the subject exists in the brain at a certain concentration (amount) which is detectable for a long time when the subject is administered to an animal to be tested, as compared to a control after a same period (day) after the administration.

The phenomena are caused by at least any one of an increase in the migration of the subject into the brain, an increase in the accumulation in the brain, a decrease in the migration to outside from the brain, a decrease in the discharge to outside from the brain and a decrease in the decomposition in the brain as compared to a control.

In the invention, that the property of accumulating in the brain is excellent, that the property of accumulating in the brain is high or that the property of accumulating in the brain is improved means, for example, that, when the subject is administered to an animal to be tested, the concentration (amount) of the subject in the brain is higher than that of a control 1 to 10 days after the administration, preferably 2 to 10 days or 3 to 10 days, more preferably 4 to 10 days after the administration or that the peak of the concentration in the brain (or the amount in the brain) of the subject is seen on day 4 or later after the administration, preferably on day 5 or later, day 6 or later, day 7 or later, day 8 or later or day 9 or later, more preferably on day 10 or later after the administration.

The antibody having an excellent property of accumulating in the brain, the antibody having a high property of accumulating in the brain or the antibody having an improved property of accumulating in the brain may be any antibody as long as the antibody is an antibody whose antibody concentration (antibody amount) in the brain is higher than that of a control antibody or an antibody having the characteristic of existing in the brain for a long time.

Examples include antibodies having the characteristic that the migration rate to the brain and/or the accumulation rate in the brain are higher than those of a control antibody, the characteristic that the migration rate to outside from the brain, the discharge rate and/or the decomposition rate in the brain are lower and the characteristic that the migration rate to the brain and/or the accumulation rate in the brain are higher than the migration rate to outside from the brain, the discharge rate and/or the decomposition rate in the brain.

Accordingly, the antibody or the antibody fragment of the invention is, for example an antibody or an antibody fragment thereof whose antibody concentration (or the antibody amount) in the brain is higher than that of a control antibody after a same period (day) after the administration when the antibody or the antibody fragment is administered to an animal or an antibody or an antibody fragment thereof which can exist in the brain for a long time.

The change in the antibody concentration (or the antibody amount) in the brain may be any change, and examples include a case in which the antibody concentration gradually decreases after the antibody concentration in the brain once reaches its peak during the measurement period, a case in which after the antibody concentration in the brain reaches its peak, the antibody concentration is maintained, a case in which the antibody concentration in the brain continues to increase after the administration of the antibody or another case.

The antibody or the antibody fragment of the invention is, for example, an antibody whose antibody concentration or antibody amount in the brain is higher than that of a control antibody on day 4 or day 10 after the administration to a rat, an antibody whose antibody concentration or antibody amount in the brain is maintained or increases from day 4 to day 10 after the administration to a rat, an antibody whose existence in the brain can be clearly confirmed on day 10 or later after the administration to a rat or another antibody but is not limited to these examples.

The control antibody may be any antibody as long as the control antibody is an antibody of the same species or subclass as that of the antibody to be tested, but for example, an anti-avermectin (AVM) antibody and the like can be used.

In the invention, the term “in the brain” is, for example, in the brain parenchyma, in a cerebral ventricle, in the cerebrospinal fluid or the like but is not limited to these examples.

In the invention, the method for administering an antibody to an animal is, for example, intravenous administration, intraventricular administration, intraperitoneal administration, subcutaneous administration, intradermal administration, nasal administration, intrathecal administration or the like but is not limited to these methods.

In the invention, the method for measuring the property of an antibody of accumulating in the brain is, for example, a method of collecting brain tissues several days after administering the antibody to an animal, measuring the antibody concentration of the supernatant obtained after homogenization and centrifugation and calculating the antibody amount per unit brain weight, a method of detecting the antibody using a known immunological method using the collected brain tissues, a method of administering the antibody which has been labeled to an animal and detecting the antibody by an in vivo imaging system sequentially or another method.

The antibody of the invention may be an antibody selected from the group consisting of (a) to (q) below,
(a) an antibody in which the amino acid sequences of CDRs 1 to 3 of VH are the amino acid sequences of SEQ ID NOs: 4, 5 and 6, respectively, and in which the amino acid sequences of CDRs 1 to 3 of VL contain the amino acid sequences of SEQ ID NOs: 10, 11 and 12, respectively,
(b) an antibody in which the amino acid sequences of CDRs 1 to 3 of VH are the amino acid sequences of SEQ ID NOs: 16, 17 and 18, respectively, and in which the amino acid sequences of CDRs 1 to 3 of VL contain the amino acid sequences of SEQ ID NOs: 22, 23 and 24, respectively,
(c) an antibody in which the amino acid sequences of CDRs 1 to 3 of VH are the amino acid sequences of SEQ ID NOs: 28, 29 and 30, respectively, and in which the amino acid sequences of CDRs 1 to 3 of VL contain the amino acid sequences of SEQ ID NOs: 34, 35 and 36, respectively,
(d) an antibody fragment in which the amino acid sequences of CDRs 1 to 3 of VHH contain the amino acid sequences of SEQ ID NOs: 40, 41 and 42, respectively, (e) an antibody in which the amino acid sequences of CDRs 1 to 3 of VH contain the amino acid sequences of SEQ ID NOs: 153, 154 and 155, respectively, and in which the amino acid sequences of CDRs 1 to 3 of VL contain the amino acid sequences of SEQ ID NOs: 158, 159 and 160, respectively,
(f) an antibody in which the amino acid sequences of CDRs 1 to 3 of VH contain the amino acid sequences of SEQ ID NOs: 163, 164 and 165, respectively, and in which the amino acid sequences of CDRs 1 to 3 of VL contain the amino acid sequences of SEQ ID NOs: 168, 169 and 170, respectively,
(g) an antibody in which the amino acid sequences of CDRs 1 to 3 of VH contain the amino acid sequences of SEQ ID NOs: 173, 174 and 175, respectively, and in which the amino acid sequences of CDRs 1 to 3 of VL contain the amino acid sequences of SEQ ID NOs: 178, 179 and 180, respectively,
(h) an antibody in which the amino acid sequences of CDRs 1 to 3 of VH contain the amino acid sequences of SEQ ID NOs: 183, 184 and 185, respectively, and in which the amino acid sequences of CDRs 1 to 3 of VL contain the amino acid sequences of SEQ ID NOs: 188, 189 and 190, respectively,
(i) an antibody in which the amino acid sequences of CDRs 1 to 3 of VH contain the amino acid sequences of SEQ ID NOs: 193, 194 and 195, respectively, and in which the amino acid sequences of CDRs 1 to 3 of VL contain the amino acid sequences of SEQ ID NOs: 198, 199 and 200, respectively,
(j) an antibody in which the amino acid sequences of CDRs 1 to 3 of VH contain the amino acid sequences of SEQ ID NOs: 203, 204 and 205, respectively, and in which the amino acid sequences of CDRs 1 to 3 of VL contain the amino acid sequences of SEQ ID NOs: 208, 209 and 210, respectively,
(k) an antibody in which the amino acid sequences of CDRs 1 to 3 of VH contain the amino acid sequences of SEQ ID NOs: 213, 214 and 215, respectively, and in which the amino acid sequences of CDRs 1 to 3 of VL contain the amino acid sequences of SEQ ID NOs: 218, 219 and 220, respectively,
(l) an antibody in which the amino acid sequences of CDRs 1 to 3 of VH contain the amino acid sequences of SEQ ID NOs: 223, 224 and 225, respectively, and in which the amino acid sequences of CDRs 1 to 3 of VL contain the amino acid sequences of SEQ ID NOs: 228, 229 and 230, respectively,
(m) an antibody in which the amino acid sequences of CDRs 1 to 3 of VH contain the amino acid sequences of SEQ ID NOs: 233, 234 and 235, respectively, and in which the amino acid sequences of CDRs 1 to 3 of VL contain the amino acid sequences of SEQ ID NOs: 238, 239 and 240, respectively,
(n) an antibody in which the amino acid sequences of CDRs 1 to 3 of VH contain the amino acid sequences of SEQ ID NOs: 243, 244 and 245, respectively, and in which the amino acid sequences of CDRs 1 to 3 of VL contain the amino acid sequences of SEQ ID NOs: 248, 249 and 250, respectively,
(o) an antibody which competes in binding to MOG with at least one of the antibodies described in (a) to (n),
(p) an antibody which binds to an epitope containing an epitope to which any one of the antibodies described in (a) to (n) binds and
(q) an antibody which binds to the same epitope as an epitope to which any one of the antibodies described in (a) to (n) binds.

As the antibody of the invention, an antibody having amino acid sequences of CDRs 1 to 3 of VH and CDRs 1 to 3 of VL of an antibody having homology of 85% or higher, preferably 90% or higher to the amino acid sequences of CDRs 1 to 3 of VH and CDRs 1 to 3 of VL of any one of the antibodies described in (a) to (n) is included. The homology of 90% or higher is more preferably homology of 91%, 92%, 93%, 94%, 95%, 96%, 97%, 98% or 99% or the like.

In the invention, embodiments of the antibodies described in (a) to (n) include human anti-MOG monoclonal antibodies, MOG01 antibody, MOG09 antibody and MOG14 antibody and an alpaca anti-MOG monoclonal VHH antibody, iMOG-3Rim1-S32 antibody. In addition, embodiments include a human chimeric antibody of iMOG-3Rim1-S32, a humanized antibody of iMOG-3Rim1-S32 and the like.

In the invention, the antibody of (o) is a second antibody which inhibits binding of a first antibody and MOG, wherein the antibody described in any of (a) to (n) is the first antibody.

In the invention, the antibody of (p) is a second antibody which binds to a second epitope containing a first epitope, wherein the antibody described in any of (a) to (n) is a first antibody, and the epitope to which the first antibody binds is the first epitope.

Moreover, the antibody of (q) of the invention is a second antibody which binds to a first epitope, wherein the antibody described in any of (a) to (n) is a first antibody, and the epitope to which the first antibody binds is the first epitope.

Furthermore, the antibody of the invention may be specifically an antibody selected from the group consisting of (a) to (n) and (o1) to (o22) below,
(a) an antibody in which the amino acid sequence of VH is the amino acid sequence of SEQ ID NO: 3 and in which the amino acid sequence of VL contains the amino acid sequence of SEQ ID NO: 9,
(b) an antibody in which the amino acid sequence of VH is the amino acid sequence of SEQ ID NO: 15 and in which the amino acid sequence of VL contains the amino acid sequence of SEQ ID NO: 21,
(c) an antibody in which the amino acid sequence of VH is the amino acid sequence of SEQ ID NO: 27 and in which the amino acid sequence of VL contains the amino acid sequence of SEQ ID NO: 33,
(d) an antibody fragment in which the amino acid sequence of VHH contains the amino acid sequence of SEQ ID NO: 39,
(e) an antibody in which the amino acid sequence of VH contains the amino acid sequence of SEQ ID NO: 152 and in which the amino acid sequence of VL contains the amino acid sequence of SEQ ID NO: 157,
(f) an antibody in which the amino acid sequence of VH contains the amino acid sequence of SEQ ID NO: 162 and in which the amino acid sequence of VL contains the amino acid sequence of SEQ ID NO: 167,
(g) an antibody in which the amino acid sequence of VH contains the amino acid sequence of SEQ ID NO: 172 and in which the amino acid sequence of VL contains the amino acid sequence of SEQ ID NO: 177,
(h) an antibody in which the amino acid sequence of VH contains the amino acid sequence of SEQ ID NO: 182 and in which the amino acid sequence of VL contains the amino acid sequence of SEQ ID NO: 187,
(i) an antibody in which the amino acid sequence of VH contains the amino acid sequence of SEQ ID NO: 192 and in which the amino acid sequence of VL contains the amino acid sequence of SEQ ID NO: 197,
(j) an antibody in which the amino acid sequence of VH contains the amino acid sequence of SEQ ID NO: 202 and in which the amino acid sequence of VL contains the amino acid sequence of SEQ ID NO: 207,
(k) an antibody in which the amino acid sequence of VH contains the amino acid sequence of SEQ ID NO: 212 and in which the amino acid sequence of VL contains the amino acid sequence of SEQ ID NO: 217,
(l) an antibody in which the amino acid sequence of VH contains the amino acid sequence of SEQ ID NO: 222 and in which the amino acid sequence of VL contains the amino acid sequence of SEQ ID NO: 227,
(m) an antibody in which the amino acid sequence of VH contains the amino acid sequence of SEQ ID NO: 232 and in which the amino acid sequence of VL contains the amino acid sequence of SEQ ID NO: 237,
(n) an antibody in which the amino acid sequence of VH contains the amino acid sequence of SEQ ID NO: 242 and in which the amino acid sequence of VL contains the amino acid sequence of SEQ ID NO: 247,
(o1) an antibody in which the amino acid sequence of VH contains the amino acid sequence of SEQ ID NO: 252 and in which the amino acid sequence of VL contains the amino acid sequence of SEQ ID NO: 254,
(o2) an antibody in which the amino acid sequence of VH contains the amino acid sequence of SEQ ID NO: 256 and in which the amino acid sequence of VL contains the amino acid sequence of SEQ ID NO: 258,
(o3) an antibody in which the amino acid sequence of VH contains the amino acid sequence of SEQ ID NO: 260 and in which the amino acid sequence of VL contains the amino acid sequence of SEQ ID NO: 262,
(o4) an antibody in which the amino acid sequence of VH contains the amino acid sequence of SEQ ID NO: 264 and in which the amino acid sequence of VL contains the amino acid sequence of SEQ ID NO: 266,
(o5) an antibody in which the amino acid sequence of VH contains the amino acid sequence of SEQ ID NO: 268 and in which the amino acid sequence of VL contains the amino acid sequence of SEQ ID NO: 270,
(o6) an antibody in which the amino acid sequence of VH contains the amino acid sequence of SEQ ID NO: 272 and in which the amino acid sequence of VL contains the amino acid sequence of SEQ ID NO: 274,
(o7) an antibody in which the amino acid sequence of VH contains the amino acid sequence of SEQ ID NO: 276 and in which the amino acid sequence of VL contains the amino acid sequence of SEQ ID NO: 278,
(o8) an antibody in which the amino acid sequence of VH contains the amino acid sequence of SEQ ID NO: 280 and in which the amino acid sequence of VL contains the amino acid sequence of SEQ ID NO: 282,
(o9) an antibody in which the amino acid sequence of VH contains the amino acid sequence of SEQ ID NO: 284 and in which the amino acid sequence of VL contains the amino acid sequence of SEQ ID NO: 286,
(o10) an antibody in which the amino acid sequence of VH contains the amino acid sequence of SEQ ID NO: 288 and in which the amino acid sequence of VL contains the amino acid sequence of SEQ ID NO: 290,
(o11) an antibody in which the amino acid sequence of VH contains the amino acid sequence of SEQ ID NO: 292 and in which the amino acid sequence of VL contains the amino acid sequence of SEQ ID NO: 294,
(o12) an antibody in which the amino acid sequence of VH contains the amino acid sequence of SEQ ID NO: 296 and in which the amino acid sequence of VL contains the amino acid sequence of SEQ ID NO: 298,
(o13) an antibody in which the amino acid sequence of VH contains the amino acid sequence of SEQ ID NO: 300 and in which the amino acid sequence of VL contains the amino acid sequence of SEQ ID NO: 302,
(o14) an antibody in which the amino acid sequence of VH contains the amino acid sequence of SEQ ID NO: 304 and in which the amino acid sequence of VL contains the amino acid sequence of SEQ ID NO: 306,
(o15) an antibody in which the amino acid sequence of VH contains the amino acid sequence of SEQ ID NO: 308 and in which the amino acid sequence of VL contains the amino acid sequence of SEQ ID NO: 310,
(o16) an antibody in which the amino acid sequence of VH contains the amino acid sequence of SEQ ID NO: 312 and in which the amino acid sequence of VL contains the amino acid sequence of SEQ ID NO: 314,
(o17) an antibody in which the amino acid sequence of VH contains the amino acid sequence of SEQ ID NO: 316 and in which the amino acid sequence of VL contains the amino acid sequence of SEQ ID NO: 318,
(o18) an antibody in which the amino acid sequence of VH contains the amino acid sequence of SEQ ID NO: 320 and in which the amino acid sequence of VL contains the amino acid sequence of SEQ ID NO: 322,
(o19) an antibody in which the amino acid sequence of VH contains the amino acid sequence of SEQ ID NO: 324 and in which the amino acid sequence of VL contains the amino acid sequence of SEQ ID NO: 326,
(o20) an antibody in which the amino acid sequence of VH contains the amino acid sequence of SEQ ID NO: 328 and in which the amino acid sequence of VL contains the amino acid sequence of SEQ ID NO: 330,
(o21) an antibody in which the amino acid sequence of VH contains the amino acid sequence of SEQ ID NO: 332 and in which the amino acid sequence of VL contains the amino acid sequence of SEQ ID NO: 334 and
(o22) an antibody in which the amino acid sequence of VH contains the amino acid sequence of SEQ ID NO: 336 and in which the amino acid sequence of VL contains the amino acid sequence of SEQ ID NO: 338.

As the antibody of the invention, an antibody having amino acid sequences of VH and VL of an antibody having homology of 85% or higher, preferably 90% or higher to the amino acid sequences of VH and VL of any one of the antibodies described in (a) to (n) and (o1) to (o22) is included. The homology of 90% or higher is more preferably homology of 91%, 92%, 93%, 94%, 95%, 96%, 97%, 98% or 99% or the like.

In the invention, embodiments of the antibodies described in (a) to (n) and (o1) to (o22) include human anti-MOG monoclonal antibodies, MOG01 antibody, MOG09 antibody and MOG14 antibody and an alpaca anti-MOG monoclonal VHH antibody, iMOG-3Rim1-S32 antibody. In addition, embodiments include an iMOG-3Rim1-S32 human chimeric antibody, an iMOG-3Rim1-S32 humanized antibody and the like.

In the invention, the EU index refers to the position of an amino acid residue according to Sequences of Proteins of Immunological Interest, Fifth edition (1991). The positions of the amino acid residues shown below are all the positions of the amino acid residues according to the EU index unless particularly described.

An antibody molecule is also called an immunoglobulin (referred to as Ig below), and its basic structure is a tetramer having two polypeptides called heavy chains (referred to as H chains below) and two polypeptides called light chains (referred to as L chains below).

Each H chain is composed of a H chain variable region (also referred to as VH) and a H chain constant region (also referred to as CH) from the N-terminus side, and each L chain is composed of a L chain variable region (also referred to as VL) and a L chain constant region (also referred to as CL) from the N-terminus side.

For CH, α, δ, ε, γ and μ chains are known for each subclass. CH is further composed of a CH1 domain, a hinge domain, a CH2 domain and a CH3 domain from the N-terminus side.

A domain is a functional structural unit which constitutes a polypeptide of an antibody molecule. The CH2 domain and the CH3 domain are together called a Fc (Fragment, crystallizable) region or simply Fc. For CL, C_{λ} chain and C_{κ} chain are known.

The subclasses of antibody in which CH is α, δ, ε, γ and μ chains are called IgA, IgD, IgE, IgG and IgM, respectively. There are sometimes isotypes for a subclass of an antibody depending on the animal. In human, there are isotypes IgA1 and IgA2 for IgA, and there are isotypes IgG1, IgG2, IgG3 and IgG4 for IgG.

The CH1 domain, the hinge domain, the CH2 domain, the CH3 domain and the Fc region in the invention can be identified by the positions of the amino acid residues from the N-terminus according to the EU index.

Specifically, CH1 is identified as the amino acid sequence of from position 118 to position 215 according to the EU index, and the hinge is identified as the amino acid sequence of from position 216 to position 230 according to the EU index. CH2 is identified as the amino acid sequence of from position 231 to position 340 according to the EU index, and CH3 is identified as the amino acid sequence of from position 341 to position 447 according to the EU index. The Fc region is identified as the amino acid sequence of from position 231 to position 447 according to the EU index.

As the antibody of the invention, a polyclonal antibody, a monoclonal antibody and an oligoclonal antibody are all included. A polyclonal antibody is a group of antibody molecules that are secreted by antibody-producing cells of different clones. A monoclonal antibody is an antibody that is secreted by antibody-producing cells of a single clone and recognizes only one epitope (also called an antigenic determinant). The amino acid sequences (primary sequences) of same monoclonal antibodies are the same. An oligoclonal antibody is a group of antibody molecules in which different monoclonal antibodies are mixed.

The monoclonal antibody of the invention may be an antibody that is produced from a hybridoma or a genetically recombinant antibody that is produced by a transformant transformed with an expression vector containing the antibody genes.

The epitope may be a single amino acid sequence, a three-dimensional structure made of an amino acid sequence, an amino acid sequence modified after translation, a three-dimensional structure made of an amino acid sequence modified after translation which the monoclonal antibody recognizes and binds to or the like.

The amino acid sequence modified after translation may be an O-linked glycan in which sugar chains are attached to Tyr and Ser having OH substituents, an N-linked glycan in which sugar chains are attached to Gln and Asn having NH₂ substituents or a tyrosine sulfated amino acid sequence in which a sulfuric acid molecule is attached to Tyr having OH substituents.

The epitope of MOG to which the antibody of the invention binds can be identified by an antibody-binding test using a deletion variant of MOG in which some domains are lost, a mutant in which some domains are replaced with domains derived from another protein, a partial peptide fragment of MOG or the like. The antibody-binding test can also be conducted using cells expressing the deletion variant or the mutant.

Alternatively, the epitope of MOG to which the antibody of the invention binds can also be identified by adding the antibody of the invention to peptide fragments of MOG obtained by decomposition using a protease and conducting epitope mapping using a known mass spectrometry.

As the antibody of the invention, genetically recombinant antibodies of a mouse antibody, a rat antibody, a hamster antibody, a rabbit antibody, a llama antibody, a camel antibody, an alpaca antibody, a chimeric antibody, a humanized antibody (also called a “Complementarity Determining Region (CDR)-inserted antibody”), a human antibody and the like are also included.

In the invention, the chimeric antibody is an antibody in which VH and VL are derived from a different animal kind from that of CH and CL. An antibody composed of VH and VL of an antibody of an animal other than human (a non-human animal) and CH and CL of a human antibody is called a human chimeric antibody, and an antibody composed of VH and VL of an antibody of an animal other than mouse and CH and CL of a mouse antibody is called a mouse chimeric antibody. Other chimeric antibodies are named in the same manner.

As the non-human animal, any animal such as mouse, rat, hamster, rabbit, llama, camel or alpaca can be used as long as a hybridoma can be produced or an antibody phage library can be produced.

A hybridoma is a cell which is obtained by cell fusion of a B cell obtained by immunizing a non-human animal with an antigen and a myeloma cell derived from a mouse or the like and which produces a monoclonal antibody having a desired antigen specificity.

An antibody phage library is a library produced by cloning the genes of immunoglobulin variable regions to a phage and expressing an antigen-binding molecule on its surface. The phages used are M13 phage and the like but are not particularly limited.

The antigen-binding molecule which is displayed on a phage may be in any form but is preferably an antibody fragment such as scFv, Fab or VHH.

In the invention, the antibody phage library may be any library of an immune library, a naive library and a synthetic library.

An immune library is an antibody phage library which is constructed based on the antibody genes derived from lymphocytes of an animal immunized with an antigen or a patient. A naive library is an antibody phage library which is constructed based on the antibody genes derived from lymphocytes of a normal animal or a healthy human. A synthetic library is a library in which CDRs of a V gene in genome DNA or a reconstructed functional V gene are replaced with oligonucleotides that encode any amino acid sequences of appropriate lengths.

As a method for producing a chimeric antibody, a method for producing a human chimeric antibody is described below. Other chimeric antibodies can also be produced by the same method.

A human chimeric antibody can be produced by obtaining cDNAs that encode VH and VL from a hybridoma derived from a non-human animal cell producing a monoclonal antibody, inserting the cDNAs into an expression vector for animal cells having DNA that encodes CH and CL of a human antibody, thereby constructing a human chimeric antibody expression vector, introducing the vector to an animal cell and expressing the antibody.

A human chimeric antibody can also be produced by cloning the genes that encode VH and VL from an antibody phage library derived from a non-human animal, inserting the genes into an expression vector for animal cells having DNA that encodes CH and CL of a human antibody, thereby constructing a human chimeric antibody expression vector, introducing the vector to an animal cell and expressing the antibody.

A humanized antibody is an antibody in which the amino acid sequences of CDRs of VH and VL of an antibody of a non-human animal are implanted to the corresponding CDRs of VH and VL of a human antibody. The region other than the CDRs of VH and VL is called a framework region (referred to as FR below).

A humanized antibody can be produced by constructing cDNA that encodes the amino acid sequence of VH formed from the amino acid sequences of CDRs of VH of an antibody of a non-human animal and the amino acid sequence of FR of VH of any human antibody and cDNA that encodes the amino acid sequence of VL formed from the amino acid sequences of CDRs of VL of an antibody of a non-human animal and the amino acid sequence of FR of VL of any human antibody, inserting the cDNAs to an expression vector for animal cells having DNA that encodes CH and CL of a human antibody, thereby constructing a humanized antibody expression vector, introducing the vector to an animal cell and expressing the antibody.

A human antibody is originally an antibody that naturally exists in the human body, but antibodies obtained from a human antibody phage library and a human antibody-producing transgenic animal and the like are also included.

A human antibody can be obtained by immunizing a mouse having a human immunoglobulin gene (Tomizuka K. et al., Proc Natl Acad Sci USA. 97, 722-7, 2000.) with a desired antigen. A human antibody can be obtained also without immunization by selecting a human antibody having a desired binding activity using a phage display library obtained by amplifying antibody genes from human-derived B cells (Winter G. et al., Annu Rev Immunol. 12:433-55. 1994).

Moreover, a human antibody can be obtained by producing cells which produce a human antibody having a desired binding activity by immortalizing human B cells using EB virus (Rosen A. et al., Nature 267, 52-54.1977).

A human antibody phage library is a library in which antibody fragments such as Fab, scFv and VHH are expressed on the surface of phages by inserting an antibody gene produced from lymphocytes of a human (a healthy individual or a patient) to phage genes. It is possible to collect phages on which antibody fragments having a desired antigen binding activity are expressed using binding activity to a substrate to which an antigen is fixed as an index from the library. The antibody fragments can be further converted to a human antibody molecule formed from two whole H chains and two whole L chains using the genetic engineering technique.

A human antibody-producing transgenic animal is an animal in which a human antibody gene is incorporated into the chromosomes of the host animal. Specifically, a human antibody-producing transgenic animal can be produced by introducing a human antibody gene to mouse ES cells, implanting the ES cells to an early embryo of another mouse and then causing development.

A human antibody can be produced from a human antibody-producing transgenic animal by culturing a human antibody-producing hybridoma obtained by a general hybridoma production method performed for mammals other than human, producing and accumulating the human antibody in the culture and purifying the antibody from the culture.

The antibody of the invention includes a heavy chain antibody composed of heavy chains only. Heavy chain antibodies are an antibody obtained from a Camelidae animal such as llama, camel and alpaca and a genetically recombinant antibody produced based on the antibody.

In the invention, the antibody fragment is a fragment of an antibody which has an antigen binding activity. Examples include Fab, Fab′, F(ab′)₂, single chain Fv (scFv), diabody, dsFv, a peptide containing CDRs, VHH and the like. The antibody fragment of the invention also includes any antibody fragment as long as the antibody fragment contains a partial fragment of an antibody and has a MOG binding activity, such as an antibody fragment obtained by fusing the whole or a part of a constant region or Fc of an antibody to the antibody fragment or an antibody fragment containing a constant region or Fc.

Fab is an antibody fragment which has an antigen binding activity and a molecular weight of approximately fifty thousand and in which about a half of the H chain in the N-terminus side and the entire L chain are linked to each other through disulfide bonds (S—S bonds) (cleaved at the 224th amino acid residue in the H chain), of the fragments obtained by treating IgG antibody with a protease, papain.

F(ab′)₂ is an antibody fragment which has an antigen binding activity and a molecular weight of approximately hundred thousand and which is slightly larger than the one in which Fabs are bound through the S—S bond in the hinge region (cleaved at the 234th amino acid residue in the H chain), of the fragments obtained by treating IgG with a protease, pepsin.

Fab′ is an antibody fragment which has an antigen binding activity and a molecular weight of approximately fifty thousand and in which the S—S bond in the hinge region of the above F(ab′)₂ is cleaved.

scFv is a VH-P-VL or VL-P-VH polypeptide in which one VH and one VL are linked using an appropriate peptide linker (P) such as a linker peptide of any number of connected linkers each having four Gly residues and one Ser residue (G4S) and is an antibody fragment having an antigen binding activity.

Diabody is an antibody fragment in which scFvs having same or different antigen binding specificities form a dimer and is an antibody fragment having a divalent antigen binding activity to a same antigen or specific antigen binding activities to different antigens.

dsFv is a fragment in which polypeptides obtained by substituting one amino acid residue in VH and that in VL with cysteine residues are bound through the S—S bond between the cysteine residues.

A peptide containing CDR is composed of and contains at least one or more regions of CDRs of VH or VL. In a peptide containing CDRs, the CDRs can be bound directly or through an appropriate peptide linker.

Production can be performed by constructing DNA that encodes CDRs of VH and VL of the antibody of the invention, inserting the DNA into an expression vector for a prokaryote or an expression vector for a eukaryote and introducing the expression vector into a prokaryote or a eukaryote for expression. In addition, a peptide containing CDR can also be produced by a chemical synthesis method such as the Fmoc method or the tBoc method.

VHH is a variable region of a heavy chain antibody and is also called a nanobody.

The antibody fragment of the invention includes any antibody fragment as long as the antibody fragment contains any of the antibody fragments described above or a partial fragment thereof and has a MOG binding activity.

In the invention, an antibody having one antigen binding site or an antibody fragment thereof is called a monovalent antibody. The formats of a monovalent antibody are the formats of an antibody having one antigen binding site or an antibody fragment thereof described in International Publication No. 2014/054804, International Publication No. 2011/090754, International Publication No. 2007/048037, International Publication No. 2012/116927 and the like and other formats.

In the invention, an antibody of one molecule which binds to three or more different antigens or epitopes or an antibody fragment thereof is called a multispecific antibody. In the invention, an antibody of one molecule which binds to two different antigens or epitopes or an antibody fragment thereof is called a bispecific antibody.

The formats of a multispecific antibody or a bispecific antibody are the formats described in International Publication No. 2009/131239, International Publication No. 2014/054804, International Publication No. 01/077342, US Patent Application Publication No. 2007/0071675, International Publication No. 2007/024715, Wu et al., [Nature Biotechnology, 2007, 25(11), p. 1290-1297], Labrijn et al., [PNAS 2013, vol. 110, no. 13, p 5145-5150], Jong et al., [http://dx.doi.org/10.1371/journal.pbio.1002344], Kontermann et al., [mAbs 2012, vol. 4, issue 2, p 182-197], Spiess et al., [Molecular Immunology 67 (2015) 95-106], Ridgway et al., [Protein engineering, 1996 vol. 9 no. 7 pp 617-621, International Publication No. 2009/080251, International Publication No. 2010/151792, International Publication No. 2014/033074 and the like and other formats.

The bispecific antibody may be specifically any of the bispecific antibodies described below and the like.
(l) A bispecific antibody in which amino acid modifications S354C/T366W are introduced to CH3 of one of the two heavy chains of an antibody (heavy chain A) and in which amino acid modifications Y349C/T366S/L368A/Y407V are introduced to CH3 of the other heavy chain (heavy chain B).

(2) A bispecific antibody in which an antibody fragment is fused to a C-terminus of an antibody.

(3) A bispecific antibody in which an antibody fragment is fused to a N-terminus of an antibody.

The bispecific antibody described in (1) may be a bispecific antibody in which the antigen binding site containing VH of heavy chain A binds to MOG and in which the antigen binding site containing VH of heavy chain B binds to an antigen that is present in the brain or one in which the bindings are the other way around.

The bispecific antibody described in (2) may be any bispecific antibody of a bispecific antibody in which an antibody fragment is bound to the C-terminus of one of the two heavy chains constituting the antibody and a bispecific antibody in which antibody fragments are bound to both of the two heavy chains. Moreover, an appropriate linker may be between the C-terminus of the heavy chain of the antibody and the antibody fragment.

The antibody fragment(s) that the bispecific antibody described in (2) has is preferably scFv, Fab, VHH or the like but is not particularly limited to these fragments.

The bispecific antibody described in (2) may be a bispecific antibody in which the antigen binding site at the N-terminus binds to MOG and in which the antigen binding site at the C-terminus binds to an antigen that is present in the brain or one in which the bindings are the other way around.

The bispecific antibody described in (3) is a bispecific antibody in which an antibody fragment is bound to the N-terminus of at least any one of the two heavy chains or the light chains constituting the antibody. Moreover, an appropriate linker may be between the N-terminus of the heavy chain and/or the light chain of the antibody and the antibody fragment. The antibody fragment that the bispecific antibody described in (3) has is preferably scFv, Fab, VHH or the like but is not particularly limited to these fragments.

The bispecific antibody described in (3) is a bispecific antibody having a structure VH₁-CH1-VH₂—CH1-hinge-CH2-CH3 from the N-terminus of a heavy chain, a bispecific antibody which has the heavy chain structure and in which VH₁ and VH₂ each form an antigen binding site with VL or the like. The VLs with which VH₁ and VH₂ form antigen binding sites may have a same amino acid sequence or different amino acid sequences.

In the invention, the multispecific antibody or the bispecific antibody may be any antibody as long as the antibody is a multispecific antibody or a bispecific antibody which binds to MOG. Of such antibodies, a multispecific antibody or a bispecific antibody which binds to MOG and an antigen that is present in the brain is preferable, and a multispecific antibody or a bispecific antibody containing an antigen binding site which binds to MOG and an antigen binding site which binds to an antigen that is present in the brain is more preferable.

In the invention, the antigen that is present in the brain is a protein, a sugar chain, a lipid or the like and is preferably a protein of these antigens.

Examples of the protein that is present in the brain include MOG, Prion, 5T4, AFP, ADAM-10, ADAM-12, ADAM17, AFP, AXL, BSG, C5, C5R, CA9, CA72-4, CCL11, CCL2, CCR1, CCR4, CCR5, CCR6, CD2, CD3E, CD4, CD5, CD6, CD8, CD11, CD18, CD19, CD20, CD22, CD24, CD25, CD29, CD30, CD32B, CD33, CD37, CD38, CD40, CD40LG, CD44, CD47, CD52, CD55SC1, CD56, CD66E, CD71, CD72, CD74, CD79a, CD79b, CD80, CD86, CD95, CD98, CD137, CD147, CD138, CD168, CD200, CD248, CD254, CD257, CDH3, CEA, CEACAM1, CEACAM5, CEACAM6, CEACAM8, Claudin3, Claudin4, c-Met, CS-1, CSF2RA, CSPG-4, CTLA4, CRF-1, Cripto, CXCR4, CXCR5, DLL4, DR4, DR5, ED-B, EFNA2, EGFR, EGFRvIII, ETBR, ENPP3, EPCAM, EphA2, ERBB2, ERBB3, ERBB4, FAPα, FAS, FcγRI, FCER2, FGFR1, FGFR2, FGFR3, FGFR4, FLT1, FOLH1, FOLR1, GDF2, GFR, GLP1R, glypican-3, GPNMB, GRP78, HB-EGF, HGF, HLA-DRP, ICAM1, IFNA1, IFNA1, IgE, IgE-Fe, IGF1R, IL10, IL12B, IL13, IL15, IL17A, ILlA, IL1B, IL2RA, IL4, IL5, IL5RA, IL6, IL6R, IL9, IL2Rα, IL2Rβ, IL2Rγ, INSR, ITGA2, ITGA2B2, ITGB3, ITGA4, ITGB7, ITGA5, ITGAL, ITGAV, ITGB3, ITGB2, KDR, LlCAM, mesothelin, MMP14, MMP15, MST1R, MSTN, MUC1, MUC4, MUC16, MUC5AC, myostatin, NECTIN4, NGF, NOTCH, NRG1, NRP, OX40, OX40L, PDGFA, PDGFB, PDGFRA, PDGFRB, PD1, PDL1, PSCA, SLAM7, SLC44A4, TAG-72, TCR, TGFB1, TGFB2, TGFBR, TNF, TNFR, TNFRSF10A, TNFRSF10B, TNFRSF12A, TNFSF13, TNFSF14, TNFSF2, TNFSF7, TRAILR2, TRKA, TRKB, TRKC, VEGF, VEGFR, VLA-4, CGRP, alpha-synuclein, TDP-43, Tau, FUS, Amyloid-beta (Aβ), APP, BACE1, Presenilin, LINGO-1, Nogo, polyQ, androgen receptor, huntingtin, ataxin 1, ataxin 2, RGMA, Phospho-Tau, Phospho-alpha-synuclein and the like, but the protein is not limited to these proteins.

Examples of the sugar chain that is present in the brain include Lewis-x, Lewis-y, CD15 and the like, but the sugar chain is not limited to these sugar chain.

Examples of the lipid that is present in the brain include GD1a, GD2, GD3, GM1, GM2, GM3, phosphatidylserine and the like, but the lipid is not limited to these lipids.

The antibody or the antibody fragment of the invention also includes an antibody containing any amino acid that is modified after translation. Examples of the modification after translation include deletion of the lysine residue at the C-terminus of a H chain (lysine clipping), conversion of the glutamine residue at the N-terminus of a polypeptide into pyroglutamine (puroGlu) and the like [Beck et al, Analytical Chemistry, 85, 715-736(2013)].

An amino acid residue in the Fc region of the antibody or the antibody fragment of the invention may be modified. Examples of the amino acid modification in the Fc region include amino acid modification for stabilizing the antibody or regulating the half-life in the blood and the like. Specific examples of the amino acid modification in the Fc region include those in International Publication No. 2006/033386, International Publication No. 2006/075668, International Publication No. 2011/122011, International Publication No. 2009/125825 and the like.

The antibody or the antibody fragment of the invention also includes a fused antibody or a fused antibody fragment in which an antibody or an antibody fragment is modified. The method for modifying an antibody is not particularly limited, and any method which can modify a desired amino acid residue and a sugar chain can be used.

Examples include chemical modification using chemical reaction [Introduction to Antibody Engineering, Chijinshokan Co., Ltd. (1994); and Kolb et al., Angew Chem Int Ed Engl. 40. 2004-21, 2001], modification by the genetic engineering technique in which a recombinant protein expression vector is introduced to an appropriate host cell for expression using genetic recombination technology and the like.

In the invention, examples of the molecule which modifies the antibody or the antibody fragment include a hydrophilic polymer, an amphipathic polymer, a functional molecule and the like. Examples of the hydrophilic polymer and the amphipathic polymer include a polyoxyalkylene, a molecule containing a polyol or a polysaccharide and the like.

Examples of the polyoxyalkylene include linear or branched chain polyethylene glycol (referred to as PEG below), polypropylene glycol, polypropylene ethylene glycol and the like.

Examples of the molecule containing a polyol or a polysaccharide include homo- or hetero-polysaccharides such as amylose, dextran, pullulan or glycogen composed of linear or branched chain polyglycerol and the like.

The molecular weight of the molecule containing a hydrophilic polymer or an amphipathic polymer is not particularly limited but is preferably 100 Da or more, preferably for example 100 Da to 100 kDa.

Examples of the functional molecule include an antigen-binding molecule, a fragment thereof, a drug, a bioactive peptide, a bioactive protein, a nucleic acid, a radiolabeling compound, a sugar chain, a lipid, a fluorescent compound and the like. A molecule which has double specificity as a result of modification with a functional molecule such as an antigen-binding molecule is a bispecific antibody.

Examples of the antigen-binding molecule include an antibody, a receptor, a ligand and the like.

The fragment of the antigen-binding molecule may be any fragment as long as the fragment is a fragment of the antigen-binding molecule and has an antigen binding activity.

Examples of the drug include anti-cancer drugs such as alkylating agents, nitrosoureas, antimetabolites, antiviral agents, antibiotics, plant alkaloids, topoisomerase inhibitors, tubulin polymerization inhibitors, hormonal therapy agents, hormone antagonists, aromatase inhibitors, P-glycoprotein inhibitors, platinum complex derivatives, M cycle inhibitor or kinase inhibitors [Clinical oncology, Cancer and chemotherapy (1996)], anti-inflammatory agents such as steroids such as hydrocortisone or prednisone, nonsteroidal drugs such as aspirin or indomethacin, immune modulating drugs such as gold thiomalate or penicillamine, immunosuppressive drugs such as cyclophosphamide or azathioprine, antihistamine drugs such as chlorpheniramine maleate or clemastine [Inflammation and anti-inflammatory therapy, Ishiyaku Pub, Inc. (1982)] and the like.

Examples of the anti-cancer drugs include mertansine, emtansine, amifostine (Ethyol), cisplatin, dacarbazine (DTIC), dactinomycin, mechlorethamine (nitrogen mustard), streptozocin, cyclophosphamide, ifosfamide, carmustine (BCNU), lomustine (CCNU), doxorubicin (Adriamycin), epirubicin, gemcitabine (Gemzar), daunorubicin, procarbazine, mitomycin, cytarabine, etoposide, 5-fluorouracil, fluorouracil, vinblastine, vincristine, bleomycin, daunomycin, peplomycin, estramustine, paclitaxel (Taxol), docetaxel (Taxotere), Aldesleukin, asparaginase, busulfan, carboplatin, oxaliplatin, nedaplatin, cladribine, camptothecin, 7-ethyl-10-hydroxycamptothecin (SN38), floxuridine, fludarabine, hydroxyurea, idarubicin, mesna, irinotecan (CPT-11), nogitecan, mitoxantrone, topotecan, leuprolide, megestrol, melphalan, mercaptopurine, hydroxycarbamide, plicamycin, mitotane, pegaspargase, pentostatin, pipobroman, streptozocin, tamoxifen, goserelin, leuprorelin, flutamide, teniposide, testolactone, thioguanine, thiotepa, uracil mustard, vinorelbine, chlorambucil, hydrocortisone, prednisolone, methylprednisolone, vindesine, nimustine, semustine, capecitabine, Tomudex, azacitidine, UFT, oxaloplatin, gefitinib (Iressa), imatinib (STI571), erlotinib, FMS-like tyrosine kinase 3 (Flt3) inhibitor, vascular endothelial growth factor receptor (VEGFR) inhibitor, fibroblast growth factor receptor (FGFR) inhibitor, epidermal growth factor receptor (EGFR) inhibitor such as Tarceva, radicicol, 17-allylamino-17-demethoxygeldanamycin, rapamycin, amsacrine, all-trans retinoic acid, thalidomide, lenalidomide, anastrozole, fadrozole, letrozole, exemestane, gold thiomalate, D-penicillamine, bucillamine, azathioprine, mizoribine, cyclosporine, rapamycin, hydrocortisone, bexarotene (Targretin), tamoxifen, dexamethasone, progestins, estrogens, anastrozole (Arimidex), Leuplin, Aspirin, indomethacin, celecoxib, azathioprine, penicillamine, gold thiomalate, chlorpheniramine maleate, chloropheniramine, clemastine, tretinoin, bexarotene, arsenic, bortezomib, allopurinol, calicheamicin, ibritumomab tiuxetan, targretin, ozogamine, clarithromycin, leucovorin, ketoconazole, aminoglutethimide, suramin, methotrexate, maytansinoid, derivatives thereof and the like.

Examples of the method for binding the drug and the antibody or the antibody fragment include a method of binding the drug to an amino group of the antibody through glutaraldehyde, a method of binding an amino group of the drug to a carboxyl group of the antibody through water-soluble carbodiimide and the like in addition to the above method.

Examples of the bioactive peptide or the bioactive protein include interferon (referred to as IFN below)-α, IFN-β, IFN-γ, interleukin (referred to as IL below)-2, IL-12, IL-15, IL-18, IL-21, IL-23, granulocyte colony stimulating factor (G-CSF), granulocyte-macrophage colony stimulating factor (GM-CSF), macrophage colony stimulating factor (M-CSF), cytokines or growth factors which activate immunocompetent cells such as NK cells, macrophages or neutrophils, proteases such as hydrase, lyase and isomerase, enzymes such as acid sphingomyelinase, toxins including bacterial toxins and phytotoxins such as ricin, diphtheria toxin or ONTAK, antimicrobial peptides having cytomembrane-damaging activity, peptides having cytomembrane-binding affinity or permeability to cytomembrane, derivatives thereof and the like.

The nucleic acid may be any molecule as long as it is a molecule in which nucleotides or molecules having equivalent function to that of nucleotides are polymerized, and examples include siRNA, microRNA, antisense RNA, DNA aptamers and the like.

The radiolabeling compound may be any nuclide that is used for applications for diagnoses or treatment, and examples include ³H, ¹⁴C, ³²P, ³³P, ³⁵S, ⁵¹Cr, ⁵⁷CO, ¹⁸F, ¹⁵³Gd, ¹⁵⁹Gd, ⁶⁴Cu, ⁶⁸Ge, ¹⁶⁶Ho, ¹¹⁵In, ¹¹³In, ¹¹²In, ¹¹¹In, ¹³¹I, ¹²⁵I, ¹²³I, ¹²¹I, ¹⁴⁰La, ¹⁷⁷Lu, ⁵⁴Mn, ⁹⁹Mo, ¹⁰³Pd, ¹⁴²Pr, ¹⁴⁹Pm, ¹⁸⁶Re, ¹⁸⁸Re, ²¹¹At, ¹⁰⁵Rh, ⁹⁷Ru, ¹⁵³Sm, ⁴⁷Sc, ⁷⁵Se, ⁸⁵Sr, ⁹⁹Tc, ²⁰¹Ti, ¹¹³Sn, ¹¹⁷Sn, ¹³³Xe, ¹⁶⁹Yb, ¹⁷⁵Yb, ⁹⁰Y, ⁶⁵Zn and compounds containing the nuclides.

The radiolabeling compound can be directly bound to the antibody by the chloramine T method or the like. In addition, a substance that chelates the radiolabeling compound may be bound to the antibody. Examples of the chelating agent include DOTA, PA-DOTA, TRITA, DTPA and the like, and an antibody modified with the chelating agent and a modified antibody which is labeled with the radiolabeling compound through the chelating agent are also included in the antibody of the invention.

Examples of the sugar chain include monosaccharides, disaccharides or oligosaccharides such as fucose, mannose, glucose, allose, aldose, gulose, idose, galactose, talose, ribose, arabinose, xylose, lyxose, erythose, erythrose, threose, cellobiose, maltose, isomaltose, lactose, lipoarabinomannan, Lewis X trisaccharide and sialyl-Lewis X tetrasaccharide and the like. Moreover, the sugar chain may be a natural product containing a sugar chain known as immunoadjuvant and may be β(1→3) glucan (lentinan or schizophyllan), α-galactosylceramide (KRN7000) or the like.

Examples of the lipid include simple lipids (neutral lipids), which are esters of fatty acids and alcohols and analogues thereof. Examples include fats (for example, triacylglycerol), wax (for example, fatty acid esters of higher alcohols), sterol esters, cholesterol esters, fatty acid esters and the like of vitamins, complex lipids having a polar group such as phosphoric acid, saccharide, sulfuric acid or amine in addition to a fatty acid and an alcohol such as phospholipids (for example, glycerophospholipids, sphingophospholipids and the like) and glycolipids (for example, glyceroglycolipids, sphingoglycolipids and the like), derived lipids which are lipid-soluble compounds of compounds produced by hydrolysis of simple lipids and complex lipids such as fatty acids, higher alcohols, lipid-soluble vitamins, steroids and carbohydrates and the like.

Examples of the fluorescent compound include fluorescent dyes such as fluorescein series like fluorescein isothiocyanate (FITC), rhodamine series, Cy3, Cy5, eosine series, Alexa Fluor series and NBD series, light-emitting substances such as acridinium esters or lophine, fluorescent proteins such as green fluorescent protein (GFP) and the like.

The antibody or the antibody fragment of the invention can be bound to the hydrophilic polymer, the amphipathic polymer or the functional molecule directly or through an appropriate linker. Examples of the linker include esters, disulfides, hydrazones, dipeptides and the like.

When a fused antibody or a fused antibody fragment is produced by modifying the antibody or the antibody fragment of the invention by the genetic engineering technique, a fused antibody or a fused antibody fragment can be produced by linking cDNA encoding a protein to cDNA encoding an antibody to construct DNA that encodes the fused antibody or the fused antibody fragment, inserting the DNA into an expression vector for a prokaryote or a eukaryote, introducing the expression vector into a prokaryote or a eukaryote and expressing the fused antibody or the fused antibody fragment.

The composition of the invention may be any composition as long as the composition contains the antibody or the antibody fragment of the invention. The composition may contain an appropriate carrier or an additive such as a stabilizing agent in addition to the antibody or the antibody fragment.

Examples of the composition of the invention includes a composition for detection or measurement containing the antibody or the antibody fragment of the invention and the like. Examples of the composition of the invention include a pharmaceutical composition (a therapeutic agent) containing the antibody or the antibody fragment of the invention as an active ingredient and the like, and pharmaceutical formulation with a desired dosage form is prepared together with a pharmacologically acceptable carrier.

In the invention, the composition for detection or measurement may be any composition as long as the composition contains the antibody or the antibody fragment of the invention and can detect or measure an antigen to which the antibody or the antibody fragment of the invention specifically binds. The antigen to which the antibody or the antibody fragment of the invention specifically binds is MOG, MOG and an antigen that is present in the brain or the like.

The antibody or the antibody fragment of the invention has a property of binding to MOG in the brain and accumulating in the brain when administered to an animal. Therefore, when the composition for detection or measurement containing the antibody or the antibody fragment is used, the antibody can be maintained in the brain, or the antibody concentration in the brain can be improved. Thus, MOG or MOG and an antigen that is present in the brain can be detected or measured for a long time, and/or MOG or MOG and an antigen that is present in the brain can also be detected or measured with high sensitivity.

For example, when the composition for detection or measurement is a composition containing a bispecific antibody which binds to MOG and an antigen that is present in the brain, MOG and the antigen in the brain, to which the bispecific antibody binds, can be detected or measured for a long time, and/or MOG and the antigen that is present in the brain can be detected or measured with high sensitivity.

Moreover, for example, when the composition for detection or measurement is a composition containing a fused antibody or a fused antibody fragment which is labeled with a radiolabeling compound or a fluorescent dye and which binds to MOG, MOG can be detected or measured for a long time, and/or MOG can be detected or measured with high sensitivity.

The pharmaceutical composition (therapeutic agent) containing the antibody of the invention may be a therapeutic agent for any disease as long as the antigen to which the antibody or the antibody fragment of the invention specifically binds is expressed in the disease but is preferably a therapeutic agent for a brain disease.

Examples of the brain disease include Alzheimer's disease, the prodromal stage of Alzheimer's disease, Huntington disease, Parkinson's disease, brain tumors, multiple sclerosis, muscular dystrophy, amyotrophic lateral sclerosis, multiple system atrophy, progressive supranuclear palsy, nigrostriatal degeneration, olivopontocerebellar atrophy, bulbospinal muscular atrophy, spinocerebellar degeneration, cerebrovascular disease, epilepsy, migraine, hyperactivity disorder, Creutzfeldt-Jakob disease, corticobasal degeneration, lysosomal storage disease, depression, dystonia and the like.

The antibody of the invention has a property of binding to MOG in the brain and accumulating in the brain when administered to an animal. Therefore, when the therapeutic agent containing the antibody or the antibody fragment is used, the antibody or the antibody fragment can be maintained in the brain for a long time, and the antibody concentration in the brain can be improved. Thus, therapeutic effects on the diseases can be exhibited.

For example, when the therapeutic agent is a therapeutic agent containing a bispecific antibody which binds to MOG and an antigen that is present in the brain, a therapeutic effect on a brain disease related to the antigen in the brain, to which the bispecific antibody binds, can be exhibited.

Moreover, for example, when the therapeutic agent is a fused antibody or a fused antibody fragment which is modified with a low molecular weight drug and which binds to MOG, a therapeutic effect on a brain disease targeted by the low molecular weight drug can be exhibited. At this point, the therapeutic effect is preferably higher when the therapeutic agent of the invention is used compared to the effect of the low molecular weight drug alone.

The therapeutic agent containing the antibody or the antibody fragment of the invention may be an agent that contains only the antibody or the antibody fragment as an active ingredient, but the agent is generally desirably mixed with one or more pharmacologically acceptable carriers and provided as medicinal formulation that is produced by any method known in the technical field of pharmaceutical science.

As the route of administration, it is preferable to use the most effective route for the treatment, and examples include oral administration or parenteral administration such as intraoral, airway, intrarectal, subcutaneous, intradermal, intramuscular, intraventricular, intraspinal cord, intranasal, intraperitoneal or intravenous administration. Intravenous or intraventricular administration or the like is particularly preferable. Examples of the form of administration include a spray, a capsule, a tablet, a powder, a granule, a syrup, an emulsion, a suppository, an injection, an ointment, a tape and the like.

The dose or the frequency of administration varies according to the desired therapeutic effect, administration method, treatment period, age, body weight and the like but is usually 10 μg/kg to 20 mg/kg per day for adult.

The invention also includes a method for keeping an antibody in the brain, a method for improving the property of an antibody of accumulating in the brain and a method for increasing the antibody concentration (or the antibody amount) in the brain which use the antibody or the antibody fragment of the invention.

The invention also relates to a peptide which binds to MOG, a nucleic acid containing a nucleotide sequence which encodes the peptide, a transformant cell which contains a vector containing the nucleic acid, a method for producing the peptide including culturing the transformant cell and collecting the peptide from the culture solution, a composition containing the peptide or a method for detecting or measuring an antigen that is present in the brain, a method for diagnosing or treating a brain disease, a method for improving the property of a peptide of accumulating in the brain or a method for increasing the peptide amount in the brain that uses the peptide or the composition.

The peptide of the invention includes a fused peptide obtained by modifying a peptide.

The definitions of the terms related to the peptide which binds to MOG and the like are the same as the definitions of the terms described for the antibody which binds to MOG and the like above unless particularly described.

The method for producing the antibody or the antibody fragment of the invention, the method for treating a disease, the method for diagnosing a disease and the like are specifically explained below.

### 1. Production Method of Antibody

### (1) Preparation of Antigen

MOG as an antigen or a MOG-expressing cell can be obtained by introducing an expression vector containing cDNA that encodes the full length of MOG or a partial length thereof to E. coli, yeast, insect cells, animal cells or the like. In addition, MOG can also be obtained by purifying MOG from an animal cell line, an animal cell or an animal tissue of various kinds and the like in which MOG is expressed in a large amount.

In addition, the animal cell line, the animal cell, the animal tissue and the like can also be used as they are as an antigen. Furthermore, a synthetic peptide having a partial sequence of MOG can be prepared using a chemical synthesis method such as the Fmoc method or the tBoc method and used as an antigen.

A known tag such as FLAG or His may be added to the C-terminus or the N-terminus of MOG or the synthetic peptide having a partial sequence of MOG.

MOG used in the invention can be produced using the method described in Molecular Cloning, A Laboratory Manual, Second Edition, Cold Spring Harbor Laboratory Press (1989), Current Protocols In Molecular Biology, John Wiley & Sons (1987-1997) or the like or another method by expressing DNA that encodes MOG in a host cell for example by the following method.

First, a recombinant vector is produced by inserting the full-length cDNA containing the part that encodes MOG into downstream of a promoter in an appropriate expression vector. A DNA fragment of an appropriate length which contains the part that encodes the polypeptide and which is prepared based on the full-length cDNA may be used in place of the full-length cDNA. Next, a transformant that produces the polypeptide can be obtained by introducing the obtained recombinant vector into a host cell suitable for the expression vector.

As the expression vector, any vector can be used as long as it can replicate autonomously or can be inserted into a chromosome in a host cell to be used and which contains a suitable promoter in the position that enables the transcription of DNA that encodes the polypeptide. As the host cell, any cell, such as a microorganism belonging to the genus Escherichia such as E. coli, yeast, an insect cell or an animal cell, can be used as long as it enables the expression of a target gene.

In a case where a prokaryote such as E. coli is used as a host cell, the expression vector is preferably a vector that can replicate autonomously in the prokaryote and that contains a promoter, a ribosomal binding sequence, DNA containing the part encoding human MOG and a transcription termination sequence. In addition, the transcription termination sequence is not essentially needed for the expression vector, but the transcription termination sequence is preferably placed immediately after the structural gene. Furthermore, the recombinant vector may contain a gene controlling the promoter.

As the expression vector, it is preferable to use a plasmid in which the distance between the Shine-Dalgarno sequence (also called SD sequence) that is a ribosomal binding sequence and the initiation codon is appropriately adjusted (to, for example, 6 to 18 nucleotides).

In addition, regarding the nucleotide sequence of DNA that encodes MOG, a nucleotide can be substituted in a manner that the codon becomes optimum for the expression in a host, which enables the enhancement in the production rate of target MOG.

As the expression vector, any vector can be used as long as it can exhibit its function in a host cell to be used. Examples thereof include pBTrp2, pBTac1 and pBTac2 (manufactured by Roche Diagnostics K.K.), pKK233-2 (manufactured by Pharmacia), pSE280 (manufactured by Invitrogen), pGEMEX-1 (manufactured by Promega Corporation), pQE-8 (manufactured by QIAGEN), pKYP10 (JP-A-S58-110600), pKYP200 [Agricultural Biological Chemistry, 48, 669 (1984)], pLSA1 [Agric. Biol. Chem., 53, 277 (1989)], pGEL1 [Proc. Natl. Acad. Sci. USA, 82, 4306 (1985)], pBluescript II SK(−) (manufactured by Stratagene Corporation), pTrs30 [prepared from Escherichia coli JM109/pTrS30 (FERM BP-5407)], pTrs32 [prepared from Escherichia coli JM109/pTrS32 (FERM BP-5408)], pGHA2 [prepared from Escherichia coli IGHA2 (FERM BP-400), JP-A-S60-221091], pGKA2 [prepared from Escherichia coli IGKA2 (FERM BP-6798), JP-A-S60-221091], pTerm2 (U.S. Pat. Nos. 4,686,191, 4,939,094 and United States Patent No. 160,735), pSupex, pUB110, pTP5, pC194, pEG400 [J. Bacteriol., 172, 2392 (1990)], pGEX (manufactured by Pharmacia), pET System (manufactured by Novagen), pME18SFL3 and the like.

As the promoter, any promoter may be used as long as it can exhibit its function in a host cell to be used. Examples thereof include promoters such as a trp promoter (Ptrp), a lac promoter, a PL promoter, a PR promoter or a T7 promoter, which are derived from E. coli, a phage or the like. In addition, examples thereof also include promoters such as a tandem promoter with two tandemly arrayed Ptrps, a tac promoter, a lacT7 promoter or a let I promoter, which are artificially designed and altered.

Examples of the host cell include E. coli XL1-Blue, E. coli XL2-Blue, E. coli DH1, E. coli MC1000, E. coli KY3276, E. coli W1485, E. coli JM109, E. coli HB101, E. coli No. 49, E. coli W3110, E. coli NY49, E. coli DH5a and the like.

As a method for introducing a recombinant vector into a host cell, any method can be used as long as it is a method by which DNA is introduced into a host cell to be used. Examples thereof include a method using calcium ions [Proc. Natl. Acad. Sci. USA, 69, 2110 (1972), Gene, 17, 107 (1982) and Molecular & General Genetics, 168, 111 (1979)].

In a case of using an animal cell as a host, as the expression vector, any vector can be used as long as it can exhibit its function in the animal cell. Examples thereof include pcDNAI, pCDM8 (manufactured by Funakoshi Co., Ltd.), pAGE107 [JP-A-H3-22979; and Cytotechnology, 3, 133 (1990)], pAS3-3 (JP-A-H2-227075), pCDM8 [Nature, 329, 840 (1987)], pcDNAI/Amp (manufactured by Invitrogen), pcDNA3.1 (manufactured by Invitrogen), pREP4 (manufactured by Invitrogen), pAGE103 [J. Biochemistry, 101, 1307 (1987)], pAGE210, pME18SFL3, pKANTEX93 (International Publication No. 97/10354), N5KGlval (U.S. Pat. No. 6,001,358), INPEP4 (manufactured by Biogen-IDEC), pCI (manufactured by Promega Corporation), a transposon vector (International Publication No. 2010/143698) and the like.

As the promoter, any promoter can be used as long as it can exhibit its function in the animal cell. Examples thereof include a promoter of cytomegalovirus (CMV) immediate early (IE) gene, an early promoter of SV40, a retroviral promoter, a metallothionein promoter, a heat-shock promoter, a SRα promoter, a promoter of Moloney murine leukemia virus or an enhancer. In addition, an enhancer of human CMV IE gene may be used together with the promoter.

Examples of the host cell include a human leukemia cell Namalwa, a monkey cell COS, a Chinese hamster ovary cell CHO [Journal of Experimental Medicine, 108, 945 (1958); Proc. Natl. Acad. Sci. USA, 60, 1275 (1968); Genetics, 55, 513 (1968); Chromosoma, 41, 129 (1973); Methods in Cell Science, 18, 115 (1996); Radiation Research, 148, 260 (1997); Proc. Natl. Acad. Sci. USA, 77, 4216 (1980); Proc. Natl. Acad. Sci., 60, 1275 (1968); Cell, 6, 121 (1975); and Molecular Cell Genetics, Appendix I, II (pp. 883-900)]; a CHO cell which lacks dihydrofolate reductase gene (referred to as dhfr below) (CHO/DG44 cell) [Proc. Natl. Acad. Sci. USA, 77,4216(1980)], CHO-K1 (ATCC CCL-61), DUkXB11 (ATCC CCL-9096), Pro-5 (ATCC CCL-1781), CHO—S(Life Technologies, Cat #11619), Pro-3, a rat myeloma cell YB2/3HL.P2.G11.16Ag.20 (also called YB2/0), a mouse myeloma cell NS0, a mouse myeloma cell SP2/0-Ag14, a Syrian hamster cell BHK, HBT5637 (JP-A-S63-000299) and the like.

As a method for introducing an expression vector into a host cell, any method can be used as long as it is a method by which DNA is introduced into an animal cell. Examples thereof include the electroporation [Cytotechnology, 3, 133 (1990)], the calcium phosphate transfection method (JP-A-H2-227075), the lipofection [Proc. Natl. Acad. Sci. USA, 84, 7413 (1987)] and the like.

MOG can be produced by culturing a transformant derived from a microorganism, an animal cell or the like having the expression vector into which DNA that encodes MOG has been introduced and which is obtained as above in a medium, generating and accumulating MOG in the culture solution and then collecting MOG from the culture solution. A method of culturing the transformant in a medium can be performed according to a usual method used for a host culture.

In a case of expression in the cells derived from a eukaryote, MOG added with sugars or sugar chains can be obtained.

When culturing a microorganism that has been transformed by an expression vector using an inducible promoter, an inducer may be added to the medium if necessary. For example, isopropyl-β-D-thiogalactopyranoside or the like may be added to the medium for a case of culturing a microorganism that has been transformed by an expression vector using a lac promoter, and indoleacrylic acid or the like may be added to the medium for a case of culturing a microorganism that has been transformed by an expression vector using a trp promoter.

Examples of the medium in which the transfectant obtained using an animal cell as a host is cultured include RPMI 1640 Medium [The Journal of the American Medical Association, 199, 519 (1967)], Eagle's MEM Medium [Science, 122, 501 (1952)], Dulbecco's Modified MEM Medium [Virology, 8, 396 (1959)], Medium 199 [Proc. Soc. Exp. Biol. Med., 73, 1 (1950)], Iscove's Modified Dulbecco's Medium (IMDM), which are generally used, or a medium in which fetal bovine serum (FBS) or the like is added to such a medium. Culture is usually performed under the conditions of pH 6 to 8 and 30 to 40° C. in the presence of 5% CO₂ for 1 to 7 days. In addition, during the culture, antibiotics such as kanamycin or penicillin may be added to the medium, if necessary.

Examples of the method for expressing a gene that encodes MOG include a method such as secretory production or fused protein expression [Molecular Cloning, A Laboratory Manual, Second Edition, Cold Spring Harbor Laboratory Press (1989)] in addition to direct expression.

Examples of the method for producing MOG include a method of producing in a host cell, a method of secretion out of a host cell and a method of producing on the outer membrane of a host cell. An appropriate method can be selected by changing the host cell to be used or the structure of MOG to be produced.

In a case where MOG is produced in a host cell or on the outer membrane of a host cell, MOG can be actively secreted outside the host cell using the method by Paulson et al. [J. Biol. Chem., 264, 17619 (1989)], the method by Lowe et al. [Proc. Natl. Acad. Sci., USA, 86, 8227 (1989) and Genes Develop., 4, 1288 (1990)] or the method described in JP-A-H05-336963, International Publication No. 94/23021 or the like. In addition, the amount of production of MOG can also be increased using the gene amplification using dihydrofolate reductase gene or the like (JP-A-H2-227075).

Obtained MOG can be isolated and purified as follows for example. In a case where MOG is expressed in the cells in a dissolved state, the cells are collected by centrifugation after completing culture and suspended in an aquatic buffer solution, followed by crushing of the cells using an ultrasonic crusher, a French press, a Manton Gaulin homogenizer, a Dyno mill or the like, and therefore cell-free extract is obtained. A purified sample can be obtained from a supernatant obtained by centrifugation of the cell-free extract using a method such as a general method for isolation and purification of proteins, that is, a solvent extraction method, a salting-out method using ammonium sulfate or the like, a desalting method, a precipitation method using an organic solvent, anion-exchange chromatography using a resin such as Diethylaminoethyl (DEAE)-Sepharose or DIAION HPA-75 (manufactured by Mitsubishi Chemical Corporation), cation-exchange chromatography using a resin such as S-Sepharose FF (manufactured by Pharmacia), hydrophobic interaction chromatography method using a resin such as Butyl Sepharose or Phenyl Sepharose, a gel filtration method using molecular decoration, affinity chromatography, a chromatofocusing method, electrophoresis such as isoelectric focusing electrophoresis and the like alone or in combination.

In a case where MOG forms an insoluble complex and expressed in the cells, the cells are collected and then crushed in the same manner as above, followed by centrifugation, and then an insoluble complex of MOG is collected as a precipitated fraction. The collected insoluble complex of MOG is solubilized with a protein denaturant. A purified sample of the polypeptide can be obtained by the same method for isolation and purification as above, after returning MOG back to the normal three-dimensional structure through dilution or dialysis of the solubilized solution.

In a case where MOG or a derivative thereof such as a sugar-modified complex is extracellularly secreted, MOG or the derivative thereof such as a sugar-modified complex can be collected in a culture supernatant. By subjecting the culture to procedures using a method such as centrifugation as in the same manner as above, thereby obtaining a soluble fraction, and then using the same method for isolation and purification as above, a purified sample can be obtained from the soluble fraction.

In addition, MOG used in the invention can be produced also by a chemical synthesis method such as the Fmoc method or the tBoc method. MOG can be also chemically synthesized using a peptide synthesizer manufactured by Advanced Chemtech, PerkinElmer, Inc., Pharmacia, Protein Technology Instrument, Inc., Shinseserubega Co., Perceptive, Shimadzu Corporation or the like.

### (2) Immunization of Animal and Preparation of Antibody-Producing Cell for Fusion

By immunizing a 3- to 20-week old animal such as a mouse, a rat, a rabbit or a hamster with the antigen obtained in (1), antibody-producing cells are collected from the spleen, lymph nodes or peripheral blood of the animal. In addition, an animal such as a llama, an alpaca or a camel can also be used as the animal to be immunized.

Immunization is performed by administering the antigen for example together with an appropriate adjuvant such as Freund's complete adjuvant, aluminum hydroxide gel or Bordetella pertussis vaccine subcutaneously, intravenously or intraperitoneally to the animal. In a case where the antigen is a partial peptide, a conjugate of the antigen with a carrier protein such as BSA (bovine serum albumin) or KLH (Keyhole Limpet hemocyanin) is produced and used as an immunogen.

When a mouse or a rat is immunized, the administration of the antigen is performed 5 to 10 times every 1 to 2 weeks after the first administration. On the 3^{rd} to 7^{th} day after each administration, the blood is collected from a venous plexus of the fundus of the eye, and the antibody valency of the serum is measured using an enzyme immunoassay method [Antibodies—A Laboratory Manual, Cold Spring Harbor Laboratory (1988)] or the like. An animal in which the serum exhibited sufficient antibody valency with respect to the antigen used for the immunization is used as a supply source for the antibody-producing cells for fusion.

On the 3^{rd} to 7^{th} day after a final administration of the antigen, tissues including the antibody-producing cells such as the spleen are extracted from the immunized animal, and the antibody-producing cells are collected. In a case of using the spleen cells, the spleen is shredded and loosened, followed by centrifugation, and then erythrocytes are removed. The antibody-producing cells for fusion are thus obtained.

Other animals to be immunized can also be immunized by the same method, and antibody-producing cells can be obtained. Appropriate conditions for the interval of immunizations and the period between the final immunization and the collection of the tissues can be determined depending on the kind of the animal to be immunized.

### (3) Preparation of Myeloma Cells

As the myeloma cells, established cells obtained from a mouse are used, and for example, a 8-azaguanine resistant mouse (BALB/c derived) myeloma cell line, P3-X63Ag8-U1 (P3-U1) [Current Topics in Microbiology and Immunology, 18, 1 (1978)], P3-NS1/1-Ag41 (NS-1) [European J. Immunology, 6, 511 (1976)], SP2/0-Ag14(SP-2) [Nature, 276, 269 (1978)], P3-X63-Ag8653 (653) [J. Immunology, 123, 1548 (1979)], P3-X63-Ag8(X63) [Nature, 256, 495(1975)] or the like is used.

The myeloma cells are subjected to subculturing with a normal medium [RPMI1640 medium supplemented with glutamine, 2-mercaptoethanol, gentamicin, FBS and 8-azaguanine] and subjected to subculturing with a normal medium 3 to 4 days before the cell fusion, and 2×10⁷ or more cells are acquired on the day of the fusion.

(4) Cell Fusion and Preparation of Monoclonal Antibody-Producing Hybridoma The antibody-producing cells for fusion obtained in (2) and the myeloma cells obtained in (3) are thoroughly washed with the Minimum Essential Medium (MEM) or PBS (disodium phosphate 1.83 g, monopotassium phosphate 0.21 g, salt 7.65 g, distilled water 1 liter, pH 7.2), mixed at cell numbers of antibody-producing cells for fusion:myeloma cells of 5:1 to 10:1 and centrifuged, and then the supernatant is removed.

After the precipitated cell clusters are loosened thoroughly, a mixture of polyethylene glycol-1000 (PEG-1000), MEM and dimethylsulfoxide is added thereto while stirring at 37° C. Furthermore, 1 to 2 mL of MEM is added thereto every 1 to 2 minutes for several times, and then MEM is added so that the total amount becomes 50 mL.

After centrifugation, the supernatant is removed. The precipitated cell clusters are loosened gently, and then the cells are suspended gently in the HAT medium [normal medium supplemented with hypoxanthine, thymidine and aminopterin]. This suspension is cultured in a 5% CO₂ incubator at 37° C. for 7 to 14 days.

After culturing, a part of the culture supernatant is taken, and cell clusters which react with MOG and which do not react with antigens other than MOG are selected by a method for selecting a hybridoma such as the binding assay described below. Next, after cloning by the limiting dilution method, a hybridoma which stably shows potent antibody valency is selected as a monoclonal antibody-producing hybridoma.

### (5) Preparation of Purified Monoclonal Antibody

The monoclonal antibody-producing hybridoma obtained in (4) is intraperitoneally injected into an 8- to 10-week old mouse or nude mouse which has been treated by pristane treatment [by intraperitoneally administering 2,6,10,14-tetramethylpentadecane (Pristane) 0.5 mL and breeding for 2 weeks]. In 10 to 21 days, the hybridoma becomes an ascites tumor.

The ascites are collected from this mouse, and the solid is removed by centrifugation. Then, by salting out with 40 to 50% ammonium sulfate and purifying by caprylic acid precipitation method, a DEAE-Sepharose column, a protein A-column or a gel filtration column, an IgG or IgM fraction is collected to obtain a purified monoclonal antibody.

Moreover, the monoclonal antibody-producing hybridoma obtained in (4) is cultured in RPMI1640 medium supplemented with 10% FBS or the like, and then the supernatant is removed by centrifugation. The hybridoma is suspended in Hybridoma SFM medium and cultured for 3 to 7 days.

A purified monoclonal antibody can also be obtained by centrifuging the obtained cell suspension, purifying from the obtained supernatant by a protein A-column or a protein G-column and collecting an IgG fraction. In this regard, 5% Daigo's GF21 can be added to Hybridoma SFM medium.

The subclass of the antibody is determined by the enzyme immunoassay method using a subclass typing kit. The protein mass is determined by the Lowry method or by calculating from the absorbance at 280 nm.

### (6) Selection of Antibody

The antibody is selected for example by measuring the affinity of the antibody to MOG-expressing cells using flow cytometry as shown below. The MOG-expressing cells may be any cells as long as MOG is expressed on the cell surface, and examples include animal cells, an animal cell line, the MOG forcibly-expressing cell line obtained in (1) and the like.

After dispensing the MOG-expressing cells to a plate such as a 96-well plate, the substances to be tested such as serum, culture supernatants of hybridomas or purified antibodies are dispensed as the first antibodies and reacted. The cells after the reaction are thoroughly washed with PBS containing 1 to 10% bovine serum albumin (BSA) (referred to as BSA-PBS below) or the like, and an anti-immunoglobulin antibody labeled with a fluorescent reagent or the like is then dispensed as the second antibody and reacted. After thoroughly washing with BSA-PBS or the like, the fluorescence amounts of the labeled antibody are measured using a flow cytometer, and an antibody which specifically reacts with the MOG-expressing cells is thus selected.

Moreover, the antibody can also be selected by measuring the affinity of the monoclonal antibody to MOG-expressing cells, a MOG protein or the like using ELISA or surface plasmon resonance described below. The MOG protein may be a protein composed of some domains of MOG or a protein to which a tag such as GST is added.

In ELISA, after dispensing the MOG-expressing cells or the MOG protein to a plate such as a 96-well plate, the wells are blocked with BSA-PBS, and the substances to be tested such as serum, culture supernatants of hybridomas or purified antibodies are dispensed as the first antibodies and reacted. Next, after thoroughly washing with PBS or the like, an anti-immunoglobulin antibody labeled with a fluorescent reagent or the like is dispensed as the second antibody and reacted.

Then, after thoroughly washing with PBS or the like, a chromogenic reagent is added. At the end, the chromogenic reaction is stopped with a solution for stopping the reaction, and the absorbances of the wells are measured with a microplate reader. An antibody which specifically reacts with the MOG-expressing cells or the MOG protein is thus selected.

In surface plasmon resonance, using a known protocol, the affinity of an antibody which binds to MOG can be measured by immobilizing the antibody on an appropriate sensor chip and using a MOG protein as the analyte.

Using the affinity of the antibody obtained, an antibody having desired affinity to a MOG protein can be selected. The affinity of an antibody which binds to MOG can also be measured by immobilizing a MOG protein on a sensor chip and using the antibody as the analyte.

In addition, an antibody which competes in binding to MOG with the antibody of the invention can be obtained by adding an antibody to be tested to the assay system using flow cytometry or ELISA described above and reacting. That is, by selecting an antibody which inhibits binding of the antibody of the invention and MOG when the antibody to be tested is added by screening, an antibody that competes with the antibody of the invention in binding to the amino acid sequence of MOG or the three-dimensional structure thereof can be obtained.

An antibody which binds to an epitope containing the epitope to which the antibody of the invention binds can be obtained by identifying the epitope of an antibody obtained by the screening method described above by a known method, producing a synthetic peptide containing the identified epitope, a synthetic peptide which mimics the three-dimensional structure of the epitope or the like and immunizing.

An antibody which binds to the same epitope as the epitope to which the antibody of the invention binds can be obtained by identifying the epitope of an antibody obtained by the screening method described above, producing a partial synthetic peptide of the identified epitope, a synthetic peptide which mimics the three-dimensional structure of the epitope or the like and immunizing.

### (7) Acquisition of Antibody by Phage Display Method

### (7-1) Production Method of Antibody Phage Library

In the invention, as the antibody phage library, an immune library, a naive library and a synthetic library can be used. The methods for producing the libraries are described below.

Lymphocytes derived from an animal immunized by the same method as that of (1) or a patient are collected for an immune library, and lymphocytes derived from a normal animal or a healthy human are collected for a naive library. RNA is extracted from the lymphocytes, and cDNA is synthesized by reverse transcription reaction.

A fragment of an antibody gene amplified by PCR using the cDNA as a template is inserted to a phagemid vector, and E. coli is transformed by the phagemid vector. When the obtained transformant is infected with a helper phage, an antibody phage library of the antibody gene can be obtained.

With respect to a synthetic library, CDR of a V gene in the genome DNA or a reconstructed functional V gene is substituted with an oligonucleotide that encodes any amino acid sequence of an appropriate length, and E. coli is transformed by a phagemid vector into which the V gene has been inserted. When the obtained transformant is infected with a helper phage, an antibody phage library can be obtained.

As the cDNA derived from lymphocytes and the antibody phage library, those which are commercially available can also be used.

As the phagemid vector, pCANTAB 5E (Amersham Pharmacia Biotech Inc.), pUC118/pUC119 vector (TaKaRa), pBlueScript II Phagemid Vector (Agilent Technologies), pKSTV-02 (Miyazaki et al, J. Biochem. 2015; 1) and the like can be used.

As the helper phage, M13K07 helper phage (Invitrogen), VCSM13 Interference Resistant Helper Phage (Agilent Technologies), R408 Interference Resistant Helper Phage (Agilent Technologies) and the like can be used.

A phage vector can also be used for phage display. There are a peptide phage library in which filamentous bacteriophage g3p is a displayed molecule (manufactured by New England Biolabs and the like), a method in which g7p, g8p or g9p is a displayed molecule and the like.

Moreover, phage display using T7 phage can also be used. A display system for T7 phage is T7 Select vector (Novagen) or the like.

### (7-2) Selection of Antibody Phage Clone

An antibody phage clone can be selected from the antibody phage libraries produced in (7-1) using the ELISA method shown below.

MOG is immobilized in an immuno tube, and the tube is blocked with a blocking buffer. The antibody phage libraries produced in (7-1) are added to the wells of the tube and reacted. Next, the wells are washed, and a fluorescently labeled anti-phage antibody is added and reacted. Then, the wells are washed again, and a chromogenic solution is added. Then, the chromogenic reaction is stopped with a solution for stopping the reaction, and the absorbances of the wells are measured with a microplate reader. In this manner, an antibody phage clone which binds to MOG is selected.

### 2. Production of Genetically Recombinant Antibody

As examples for producing a genetically recombinant antibody, methods for producing a human chimeric antibody and a humanized antibody are described below. Genetically recombinant mouse antibody, rat antibody, rabbit antibody, hamster antibody, camel antibody, llama antibody, alpaca antibody and human antibody, chimeric antibodies, a heavy chain antibody and the like can also be produced by the same method.

### (1) Construction of Expression Vector for Genetically Recombinant Antibody

An expression vector for a genetically recombinant antibody is an expression vector for animal cells in which DNA that encodes CH and CL of a human antibody has been incorporated and can be constructed by cloning DNAs that encode CH and CL of a human antibody into an expression vector for animal cells.

As the C region of a human antibody, CH and CL of any human antibody can be used. For example, CH of γ1 subclass and CL of κ class of a human antibody and the like are used. As the DNAs that encode CH and CL of the human antibody, cDNA is used, but chromosomal DNA consisting of exons and introns can also be used.

As the expression vector for animal cells, any vector can be used as long as it is capable of incorporating and expressing a gene that encodes the C region of a human antibody. For example, pAGE107 [Cytotechnol., 3, 133 (1990)], pAGE103 [J. Biochem., 101, 1307 (1987)], pHSG274 [Gene, 27, 223 (1984)], pKCR [Proc. Natl. Acad. Sci. USA, 78, 1527 (1981)], pSG1bd2-4 [Cytotechnol., 4, 173 (1990)], pSE1UK1Sed1-3 [Cytotechnol., 13, 79 (1993)] and the like are used.

The promoter and the enhancer of the expression vector for animal cells are the early promoter of SV40 [J. Biochem., 101, 1307 (1987)], the Moloney murine leukemia virus LTR [Biochem. Biophys. Res. Commun., 149, 960 (1987)] or the promoter of immunoglobulin H chain [Cell, 41, 479 (1985)] and the enhancer [Cell, 33, 717 (1983)] or the like.

As the expression vector for the genetically recombinant antibody, an expression vector for a genetically recombinant antibody of a type in which the antibody H chains and L chains are on the same vector (tandem type) [J. Immunol. Methods, 167, 271 (1994)] is used from the viewpoints of ease of construction of the expression vector for the genetically recombinant antibody, ease of introduction into animal cells, balanced expression levels of the antibody H chains and L chains in animal cells and the like, but a type in which the antibody H chains and L chains are on different vectors can also be used. As the tandem type expression vector for a genetically recombinant antibody, pKANTEX93 (International Publication No. 97/10354), pEE18 [Hybridoma, 17, 559 (1998)] and the like are used.

(2) Acquisition of cDNA Encoding V Region of Antibody Derived from Animal Other than Human and Analysis of Amino Acid Sequence

cDNA that encodes VH and VL of a non-human antibody can be obtained, and the amino acid sequence can be analyzed as follows.

(2-1) when Antibody is Obtained by Hybridoma Method

mRNA is extracted from hybridoma cells producing a non-human antibody, and cDNA is synthesized. The synthesized cDNA is cloned into a vector such as a phage or a plasmid to produce a cDNA library.

Recombinant phages or recombinant plasmids having cDNAs that encode VH or VL are isolated from the libraries using DNAs that encode the C region and the V region of the non-human antibody as probes. The entire nucleotide sequences of VH or VL of the target non-human antibody on the recombinant phages or the recombinant plasmids are determined, and then the entire amino acid sequences of VH or VL are deduced from the nucleotide sequences.

As the animal other than human which produces the hybridoma cells producing the non-human antibody, a mouse, a rat, a hamster, a rabbit, a llama, a camel, an alpaca or the like is used, but any animal can be used as long as hybridoma cells can be produced.

For the preparation of total RNA from hybridoma cells, the guanidine thiocyanate-cesium trifluoroacetate method [Methods in Enzymol., 154, 3 (1987)], a kit such as RNA easy Kit (manufactured by QIAGEN) or the like is used.

To prepare mRNA from total RNA, oligo (dT) immobilized cellulose column chromatography [Molecular Cloning, A Laboratory Manual, Second Edition, Cold Spring Harbor Laboratory Press (1989)], a kit such as Oligo-dT30<Super> mRNA Purification (registered trademark) Kit (manufactured by Takara Bio Inc.) or the like is used. Furthermore, mRNA can also be prepared from hybridoma cells using a kit such as Fast Track mRNA Isolation (registered trademark) Kit (manufactured by Invitrogen) or QuickPrep mRNA Purification (registered trademark) Kit (manufactured by Pharmacia).

For the synthesis of cDNA and the production of a cDNA library, a known method [Molecular Cloning, A Laboratory Manual, Second Edition, Cold Spring Harbor Laboratory Press (1989) and Current Protocols in Molecular Biology, Supplement 1, John Wiley & Sons (1987-1997)], a kit such as SuperScript Plasmid System for cDNA Synthesis and Plasmid Cloning (manufactured by Invitrogen) or ZAP-cDNA Synthesis (registered trademark) Kit (manufactured by Stratagene) or the like is used.

When a cDNA library is produced, any vector capable of incorporating the cDNA can be used as a vector into which the cDNA synthesized using mRNA extracted from the hybridoma cells as a template is incorporated. For example, ZAP Express [Strategies, 5, 58 (1992)], pBluescript II SK (+) [Nucleic Acids Research, 17, 9494 (1989)], λZAPII (manufactured by Stratagene), λgt 10 and λgt 11 [DNA Cloning: A Practical Approach, I, 49 (1985)], Lambda Blue Mid (manufactured by Clontech Laboratories, Inc.), λExCell, pT7T3-18U (manufactured by Pharmacia), pCD2 [Mol. Cell. Biol., 3, 280 (1983)], pUC18 [Gene, 33, 103 (1985)] or the like is used.

Any Escherichia coli can be used as Escherichia coli into which a cDNA library constructed by a phage or a plasmid vector is introduced as long as the cDNA library can be introduced, expressed and maintained. For example, XL1-Blue MRF′ [Strategies, 5, 81 (1992)], C600 [Genetics, 39, 440 (1954)], Y1088, Y1090 [Science, 222, 778 (1983)], NM522 [J. Mol. Biol., 166, 1 (1983)], K802 [J. Mol. Biol., 16, 118 (1966)], JM105 [Gene, 38, 275 (1985)] or the like is used.

For the selection of the cDNA clone that encodes VH or VL of the non-human antibody from the cDNA libraries, a colony hybridization method using an isotope- or fluorescently labeled probe, the plaque hybridization method [Molecular Cloning, A Laboratory Manual, Second Edition, Cold Spring Harbor Laboratory Press (1989)] or the like is used.

In addition, the cDNA that encodes VH or VL can also be prepared by preparing primers and performing the polymerase chain reaction method [referred to as PCR method below, Molecular Cloning, A Laboratory Manual, Second Edition, Cold Spring Harbor Laboratory Press (1989) and Current Protocols in Molecular Biology, Supplement 1, John Wiley & Sons (1987-1997)] using the cDNA synthesized from mRNA or a cDNA library as a template.

The selected cDNA is cleaved with an appropriate restriction enzyme or the like and then cloned into a plasmid such as pBluescript SK (−) (manufactured by Stratagene), and the nucleotide sequence of the cDNA is determined by a commonly used nucleotide sequence analysis method or the like. For the nucleotide sequence analysis method, for example, after performing a reaction such as the dideoxy method [Proc. Natl. Acad. Sci. USA, 74, 5463 (1977)], an automatic nucleotide sequence analyzer such as ABI PRISM3700 (manufactured by PE Biosystems) or A.L.F. DNA sequencer (manufactured by Pharmacia) or the like is used.

### (2-2) When Antibody is Obtained by Phage Display Method

The entire nucleotide sequences of VH or VL are determined from the plasmid vectors of the selected phage clones using DNAs that encode the vector region or the V region as probes, and then the entire amino acid sequences of VH or VL can be deduced from the nucleotide sequences.

In both of the hybridoma method and the phage display method, by deducing the entire amino acid sequences of VH and VL from the determined nucleotide sequences and comparing with the entire amino acid sequences of VH and VL of a known antibody [Sequences of Proteins of Immunological Interest, US Dept. Health and Human Services (1991)], it is confirmed whether the obtained cDNA encodes the complete amino acid sequences of VH and VL of an antibody containing a secretion signal sequence.

Regarding the complete amino acid sequences of VH and VL of the antibody containing a secretion signal sequence, by comparing with the entire amino acid sequences of VH and VL of a known antibody [Sequences of Proteins of Immunological Interest, US Dept. Health and Human Services (1991)], the length of the secretion signal sequence and the N-terminus amino acid sequence can be deduced, and the subgroup to which they belong can be found.

In addition, the amino acid sequences of the CDRs of VH and VL can also be determined by comparing with the amino acid sequences of VH and VL of a known antibody [Sequences of Proteins of Immunological Interest, US Dept. Health and Human Services (1991)].

Furthermore, using the obtained complete amino acid sequences of VH and VL, it is possible to confirm whether the complete amino acid sequences of VH and VL are new by carrying out homology search by the BLAST method [J. Mol. Biol., 215, 403 (1990)] or the like using any database such as SWISS-PROT or PIR-Protein.

### (3) Construction of Human Chimeric Antibody Expression Vector

By cloning cDNAs that encode VH and VL of a non-human antibody in the upstream of the respective genes that encode CH and CL of a human antibody in the expression vector for a genetically recombinant antibody obtained in (1), a human chimeric antibody expression vector can be constructed.

In order to link the 3′ terminus sides of the cDNAs that encode VH or VL of the non-human antibody with the respective 5′ terminus sides of CH or CL of the human antibody, cDNAs of VH and VL in which the nucleotide sequences of the linking parts are designed to encode an appropriate amino acid and to become an appropriate restriction enzyme recognition sequence are produced.

The produced cDNAs of VH and VL are cloned in the upstream of the respective genes that encode CH or CL of the human antibody in the expression vector for a genetically recombinant antibody obtained in (1) in a manner that they are expressed in an appropriate form, and therefore a human chimeric antibody expression vector is constructed.

In addition, each of the cDNAs that encode VH or VL of the non-human antibody can be amplified by the PCR method using synthetic DNA having an appropriate restriction enzyme recognition sequence at both ends and cloned into the expression vector for a genetically recombinant antibody obtained in (1).

(4) Construction of cDNA Encoding V Region of Humanized Antibody

A cDNA that encodes VH or VL of a humanized antibody can be constructed as follows.

Amino acid sequences of the FRs of VH and VL of a human antibody for the insertion of the amino acid sequences of the CDRs of VH and VL of a non-human antibody are selected. Any amino acid sequences derived from a human antibody can be used as the selected amino acid sequences of the FRs.

For example, an amino acid sequence of FR of a human antibody registered in a database such as Protein Data Bank, a common amino acid sequence of the subgroups of FR of a human antibody [Sequences of Proteins of Immunological Interest, US Dept. Health and Human Services (1991)] or the like is used. In order to suppress a decrease in binding activity of the antibody, an amino acid sequence of FR having as high homology (at least 60% or more) as possible to the amino acid sequence of the FR of VH or VL of the original antibody is selected.

Next, the amino acid sequences of the CDRs of the original antibody are inserted to the respective selected amino acid sequences of the FRs of VH and VL of the human antibody, and the amino acid sequences of VH and VL of a humanized antibody are designed. By converting the designed amino acid sequences into DNA sequences in consideration of the use frequency of codons found in the nucleotide sequences of the antibody genes [Sequences of Proteins of Immunological Interest, US Dept. Health and Human Services (1991)], DNA sequences encoding the amino acid sequences of VH and VL of a humanized antibody are designed.

Based on the designed DNA sequences, several synthetic DNAs having lengths of around 100 bases are synthesized, and the PCR reaction is carried out using the DNAs. In this case, due to the reaction efficiency of the PCR reaction and the synthesizable lengths of DNAs, 6 synthetic DNAs are preferably designed for each of the VH and VL.

Furthermore, by introducing an appropriate restriction enzyme recognition sequence at the 5′ or 3′ terminus of the synthetic DNAs located at both ends, cDNA that encodes VH or VL of a humanized antibody can be easily cloned into the expression vector for a genetically recombinant antibody obtained in (1).

After the PCR reaction, the amplified products are each cloned into a plasmid such as pBluescript SK (−) (manufactured by Stratagene), and the nucleotide sequences are determined by the same method as the method described in (2). A plasmid having the DNA sequence that encodes the amino acid sequence of VH or VL of a desired humanized antibody is thus obtained.

Alternatively, the entire VH and the entire VL each synthesized as a long chain DNA based on the designed DNA sequences can also be used instead of the PCR amplified products. Moreover, by introducing an appropriate restriction enzyme recognition sequence at both ends of the synthesized long chain DNAs, cDNAs that encode VH and VL of the humanized antibody can be easily cloned into the expression vector for a genetically recombinant antibody obtained in (1).

### (5) Modification of Amino Acid Sequence of V Region of Humanized Antibody

When only the CDRs of VH and VL of a non-human antibody are merely inserted into the FRs of VH and VL of a human antibody, the antigen binding activity of the humanized antibody is lower than that of the original non-human antibody [BIO/TECHNOLOGY, 9, 266 (1991)].

In a humanized antibody, by identifying the amino acid residues directly related to antigen binding, the amino acid residues interacting with the amino acid residues of the CDRs and the amino acid residues which maintain the three-dimensional structure of the antibody and which are indirectly related to antigen binding, in the amino acid sequences of the FRs of VH and VL of a human antibody, and by substituting these amino acid residues with the amino acid residues of the original non-human antibody, the lowered antigen binding activity can be increased.

In order to identify the amino acid residues of FR related to the antigen binding activity, the three-dimensional structure of the antibody can be constructed and analyzed using X-ray crystallography [J. Mol. Biol., 112, 535 (1977)], computer modeling [Protein Engineering, 7, 1501 (1994)] or the like. Furthermore, a humanized antibody having necessary antigen binding activity can be obtained by producing various types of variants for each antibody and repeatedly examining their correlation with the antigen binding activities and through trial and error.

Amino acid residues of the FRs of VH and VL of a human antibody can be modified by carrying out the PCR reaction described in (4) using synthetic DNA for the modification. The nucleotide sequence of the amplified product after the PCR reaction is determined, and whether the intended modification has been carried out is confirmed by the method described in (2).

### (6) Construction of Expression Vector for Humanized Antibody

By cloning the cDNAs that encode VH and VL of the constructed genetically recombinant antibody in the upstream of the respective genes that encode CH and CL of the human antibody in the expression vector for a genetically recombinant antibody obtained in (1), an expression vector for a humanized antibody can be constructed.

For example, the cDNAs are cloned in the upstream of the respective genes that encode CH and CL of the human antibody in the expression vector for a genetically recombinant antibody obtained in (1) in a manner that the cDNAs are expressed in an appropriate form by introducing an appropriate restriction enzyme recognition sequence at the 5′ or 3′ terminus of the synthetic DNAs located at both ends of the synthetic DNAs used for constructing the VH and VL of the humanized antibody obtained in (4) and (5).

### (7) Transient Expression of Genetically Recombinant Antibody

By transiently expressing genetically recombinant antibodies using the expression vectors of a genetically recombinant antibody obtained in (3) and (6) or modified expression vectors thereof, the antigen binding activities of the produced various human chimeric antibodies and humanized antibodies can be efficiently evaluated.

As a host cell into which an expression vector is introduced, any cell can be used as long as it is a host cell capable of expressing a genetically recombinant antibody, but for example, COS-7 cells [American Type Culture Collection (ATCC) number: CRL1651] are used [Methods in Nucleic Acids Res., CRC press, 283 (1991)].

For introduction of an expression vector into COS-7 cells, the DEAE-dextran method [Methods in Nucleic Acids Res., CRC press (1991)], the lipofection method [Proc. Natl. Acad. Sci. USA, 84, 7413 (1987)] or the like is used.

After the introduction of the expression vector, the expression level and the antigen binding activity of the genetically recombinant antibody in a culture supernatant are measured using the enzyme immunoassay method [Monoclonal Antibodies-Principles and practice, Third edition, Academic Press (1996), Antibodies-A Laboratory Manual, Cold Spring Harbor Laboratory (1988) and A manual for monoclonal antibody experiments, Kodansha scientific books (1987)] or the like.

### (8) Acquisition of Stable Expression Transformant of Genetically Recombinant Antibody and Preparation of Genetically Recombinant Antibody

By introducing the expression vector for a genetically recombinant antibody obtained in (3) or (6) into an appropriate host cell, a transformant stably expressing the genetically recombinant antibody can be obtained.

For the introduction of the expression vector into a host cell, the electroporation method [JP-A-H2-257891 and Cytotechnology, 3, 133 (1990)] or the like is used.

As the host cell into which the expression vector for a genetically recombinant antibody is introduced, any cell can be used as long as it is a host cell capable of expressing the genetically recombinant antibody. For example, CHO-K1 (ATCC CCL-61), DUKXB11 (ATCC CCL-9096), Pro-5 (ATCC CCL-1781), CHO—S(Life Technologies, Cat #11619), rat myeloma cells YB2/3HL.P2.G11.16Ag.20 (ATCC No. CRL1662, also called YB2/0), mouse myeloma cells NS0, mouse myeloma cells SP2/0-Ag14 (ATCC No. CRL1581), mouse P3X63-Ag8.653 cells (ATCC No. CRL1580), CHO cells in which the dihydrofolate reductase gene (referred to as dhfr below) is deficient (CHO/DG44 cells) [Proc. Natl. Acad. Sci. USA, 77, 4216 (1980)] and the like are used.

In addition, a host cell in which the activity of a protein such as enzymes related to intracellular synthesis of sugar nucleotide GDP-fucose, a protein such as enzymes related to glycosylation modification in which the 1-position of fucose is α-bonded to the 6-position of N-acetylglucosamine at the reducing terminus of a N-glycoside-linked complex type sugar chain, a protein related to intracellular transport of sugar nucleotide GDP-fucose to the Golgi body or the like is reduced or lost, for example, CHO cells in which the α1,6-fucosyltransferase gene is deficient (International Publication No. 2005/035586 and International Publication No. 02/31140), Lec13 having lectin resistance [Somatic Cell and Molecular genetics, 12, 55 (1986)] and the like can also be used.

After the introduction of the expression vector, a transformant stably expressing a genetically recombinant antibody is selected by culturing the transformant in a medium for animal cell culture containing a drug such as G418 sulfate (referred to as G418 below) (JP-A-H2-257891).

As the medium for animal cell culture, RPMI 1640 medium (manufactured by Invitrogen), GIT medium (manufactured by Nippon Pharmaceutical Co., Ltd.), EX-CELL 301 medium (manufactured by Jay Earl H., Inc.), EVIDM medium (manufactured by Invitrogen), Hybridoma-SFM medium (manufactured by Invitrogen), a medium in which various additives such as FBS are added to any of these media or the like is used.

A genetically recombinant antibody is expressed and accumulated in a culture supernatant by culturing the obtained transformant in the medium. The expression level and the antigen binding activity of the genetically recombinant antibody in the culture supernatant can be measured by the ELISA method or the like. In addition, the expression level of the genetically recombinant antibody produced by the transformant can be increased using the dhfr gene amplification system (JP-A-H2-257891) or the like.

The genetically recombinant antibody is purified using a protein A-column from the culture supernatant of the transformant [Monoclonal Antibodies—Principles and practice, Third edition, Academic Press (1996) and Antibodies—A Laboratory Manual, Cold Spring Harbor Laboratory (1988)]. In addition, a method used for purifying proteins, such as gel filtration, ion exchange chromatography and ultrafiltration, can also be combined.

The molecular weights of the H chains, the L chains or the whole antibody molecule of the purified genetically recombinant antibody can be measured using polyacrylamide gel electrophoresis [Nature, 227, 680 (1970)], western blotting method [Monoclonal Antibodies—Principles and practice, Third edition, Academic Press (1996) and Antibodies—A Laboratory Manual, Cold Spring Harbor Laboratory (1988)] or the like.

### (9) Production Method of Antibody Fragment

The antibody fragment of the invention can be produced according to a known method. The antibody fragment of the invention may be produced by cleaving an antibody produced according to the method described in (1) to (8) using an enzyme or the like or may be produced by the genetic engineering technique after preparing a nucleotide sequence which encodes a desired antibody fragment.

### (10) Production Method of Monovalent Antibody

In the invention, a monovalent antibody can be produced by the method described in International Publication No. 2014/054804, International Publication No. 2011/090754, International Publication No. 2007/048037, International Publication No. 2012/116927 or the like or another method.

### (11) Production Method of Bispecific Antibody or Multispecific Antibody

The bispecific antibody or the multispecific antibody of the invention can be produced according to the production method of the antibody described above. For example, the bispecific antibody or the multispecific antibody can be produced using the method described in International Publication No. 2009/131239, International Publication No. 2014/054804, International Publication No. 01/077342, US Patent Application Publication No. 2007/0071675, International Publication No. 2007/024715, Wu et al., [Nature Biotechnology, 2007, 25(11), p. 1290-1297], Labrijn et al., [PNAS 2013, vol. 110, no. 13, p 5145-5150], Jong et al., [http://dx.doi.org/10.1371/journal.pbio.1002344], Kontermann et al., [mAbs 2012, vol. 4, issue2, p 182-197], Spiess et al., [Molecular Immunology 67 (2015) 95-106], Ridgway et al., [Protein engineering, 1996 vol. 9 no. 7 pp 617-621, International Publication No. 2009/080251, International Publication No. 2010/151792, International Publication No. 2014/033074 or the like.

For example, an expression vector of a bispecific antibody in which scFv that binds to MOG is fused to the C-terminus of an IgG antibody that binds to an antigen that is present in the brain can be produced by the method described below, and the bispecific antibody can be produced according to the expression method of the antibody and the purification method of the antibody described above. In addition, a bispecific antibody in which an antibody fragment is fused to the C-terminus of an antibody can also be produced by the same methods.

A gene fragment of a CH1-Hinge-CH2-CH3-linker domain is amplified by the PCR method using a synthetic gene of a heavy chain constant region of an IgG antibody which binds to an antigen that is present in the brain as a template. Next, using the nucleotide sequence of an antibody which binds to MOG as a template, the nucleotide sequence of a scFv domain in which VH and VL of the antibody are linked with an appropriate linker is prepared using the PCR method or the like. The two domains are linked by the PCR method or the like, and the obtained gene fragment is inserted to an appropriate vector such as pCI vector.

Moreover, a gene fragment of a light chain domain (VL and CL) of an IgG antibody which binds to an antigen that is present in the brain and a gene fragment of VH of the antibody are amplified by the PCR method using appropriate templates and are inserted into the appropriate position of the vector.

In addition, the bispecific antibody of the invention can also be produced by binding an antigen binding site containing an antibody fragment to an IgG antibody by a chemical method.

### 3. Evaluation of Activity of Antibody or Antibody Fragment Thereof

In the invention, the activity of an antibody or an antibody fragment thereof can be evaluated as follows.

### (1) Binding Activity to MOG

The binding activity of the antibody or the antibody fragment of the invention to MOG is measured using flow cytometry, ELISA or surface plasmon resonance detection described in 1-(6) above or the like. Moreover, the binding activity can also be measured using a fluorescent antibody method [Cancer Immunol. Immunother., 36, 373 (1993)].

Also when the antibody or the antibody fragment of the invention is a monovalent antibody which binds to MOG, the binding activity of the monovalent antibody to MOG can be measured by the same method. Also when the antibody or the antibody fragment of the invention is a bispecific antibody or a multispecific antibody which binds to MOG and an antigen that is present in the brain, the binding activity of the bispecific antibody or the multispecific antibody to MOG or the antigen that is present in the brain can be measured by the same method.

### (2) Measurement Method of Property of Accumulating in Brain

The property of the antibody or the antibody fragment of the invention of accumulating in the brain can be measured by the method described below.

A method of collecting brain tissues several days after administering the antibody or the antibody fragment to an animal, homogenizing the brain tissues, measuring the concentration of the antibody or the antibody fragment in the supernatant after centrifugation and calculating the amount of the antibody or the antibody fragment per unit brain weight, a method of detecting the presence of the antibody or the antibody fragment by a known immunological method using the collected brain tissues or the like is used. Moreover, a method of administering the antibody or the antibody fragment to which a pharmacologically acceptable label has been attached to an animal and detecting the presence of the antibody or the antibody fragment by in vivo imaging system sequentially or the like is used.

As the animal used, an animal suitable for the use of the antibody or the antibody fragment of the invention can be selected.

### (3) Measurement Method of ADCC and CDC

The CDC or the ADCC of the antibody or the antibody fragment of the invention to human MOG-expressing cells or cells in which MOG and the antigen that is present in the brain are expressed can be measured by a known measurement method [Cancer Immunol. Immunother., 36, 373(1993); and Current protocols in Immunology, Chapter 7. Immunologic studies in humans, Editor, John E, Coligan et al., John Wiley & Sons, Inc., (1993)].

### 4. Method for Controlling Effector Activity of Antibody or Antibody Fragment

As the method for controlling the effector activity of the antibody or the antibody fragment of the invention, a method for controlling the amount of α1,6-fucose (also called a core fucose) binding to N-acetylglucosamine (GlcNAc) present on the reducing terminal of the N-linked complex sugar chain that bind to the 297^{th} asparagine (Asn) in the Fc region of the antibody or the antibody fragment containing Fc (International Publication No. 2005/035586, International Publication No. 2002/31140 and International Publication No. 00/61739), a method for controlling by modifying an amino acid residue in the Fc region of the antibody or the antibody fragment and the like are known. The effector activity of the antibody or the antibody fragment of the invention can be controlled using any of the methods.

The effector activity refers to the antibody-dependent activity that is caused through the Fc region of the antibody or the antibody fragment, and ADCC, CDC, Antibody-dependent phagocytosis (ADP) that is caused by phagocytes such as macrophages or dendritic cells and the like are known.

As the method for measuring the effector activity, for example, the effector activity can be measured by mixing the target cells, human peripheral blood mononuclear cells (PBMCs) as the effector and a target cell-specific antibody or an antibody fragment thereof, incubating the mixture for around four hours and then measuring the released lactate dehydrogenase (LDH) as an index of the cytotoxicity. In addition, the effector activity can also be measured by ⁵¹Cr-release method, flow cytometry method or the like.

The effector activity of an antibody or an antibody fragment containing Fc can be increased or decreased by controlling the core fucose content of the N-linked complex sugar chain of Fc of the antibody. Regarding the method for reducing the amount of fucose that binds to the N-linked complex sugar chain binding to Fc of the antibody or the antibody fragment, an antibody or an antibody fragment thereof to which fucose is not bound can be obtained by expressing the antibody or the antibody fragment using CHO cells in which the α1,6-fucosyltransferase gene is deficient. An antibody or an antibody fragment thereof to which fucose is not bound has high ADCC.

On the other hand, as the method for increasing the amount of fucose that binds to the N-linked complex sugar chain binding to Fc of the antibody or the antibody fragment, an antibody or an antibody fragment thereof to which fucose is bound can be obtained by expressing the antibody or the antibody fragment using host cells into which the α1,6-fucosyltransferase gene has been introduced. An antibody or an antibody fragment thereof to which fucose is bound has lower ADCC than that of an antibody or an antibody fragment thereof to which fucose is not bound.

Moreover, by modifying an amino acid residue in the Fc region of the antibody or the antibody fragment, the ADCC or the CDC can be increased or reduced. For example, the CDC of the antibody or the antibody fragment can be increased using the amino acid sequence of the Fc region described in US Patent Application Publication No. 2007/0148165.

Furthermore, the ADCC or the CDC can be increased or decreased by the amino acid modifications described in U.S. Pat. Nos. 6,737,056, 7,297,775 or U.S. Pat. No. 7,317,091.

The antibody or the antibody fragment of the invention also includes an antibody or an antibody fragment thereof whose half-life in the blood is controlled by controlling the reactivity with Fc receptor, for example through the amino acid modifications described in JP-A-2013-165716, JP-A-2012-021004 or the like in accordance with the amino acid modifications or the sugar chain modifications in the constant region contained in the antibody or the antibody fragment.

Moreover, when a combination of the above methods is applied to one antibody or an antibody fragment thereof, an antibody or an antibody fragment thereof whose effector activity and the half-life in the blood are controlled can be obtained.

### 5. Method for Treating Disease Using Antibody or Antibody Fragment of Invention

The antibody or the antibody fragment of the invention can be used for treating a brain disease of an animal in which MOG is expressed in the brain.

Examples of the brain disease include Alzheimer's disease, the prodromal stage of Alzheimer's disease, Huntington disease, Parkinson's disease, brain tumors, multiple sclerosis, muscular dystrophy, amyotrophic lateral sclerosis, multiple system atrophy, progressive supranuclear palsy, nigrostriatal degeneration, olivopontocerebellar atrophy, bulbospinal muscular atrophy, spinocerebellar degeneration, cerebrovascular disease, epilepsy, migraine, hyperactivity disorder, Creutzfeldt-Jakob disease, corticobasal degeneration, lysosomal storage disease, depression, dystonia and the like.

The brain disease that the antibody or the antibody fragment of the invention can treat differs with the antigen to which the antibody or the antibody fragment of the invention binds, the kind of the molecule which modifies the antibody or the antibody fragment in the fused antibody or the fused antibody fragment of the invention and the like.

The therapeutic agent containing the antibody or the antibody fragment of the invention may contain only the antibody or the antibody fragment as an active ingredient, but the agent is generally mixed with one or more pharmacologically acceptable carriers and provided as medicinal formulation that is produced by a method known in the technical field of pharmaceutical science.

Examples of the route of administration include oral administration or parenteral administration such as intraoral, airway, intrarectal, subcutaneous, intramuscular, intraventricular, intraperitoneal, intradermal, intranasal, intrathecal or intravenous administration. Examples of the form of administration include a spray, a capsule, a tablet, a powder, a granule, a syrup, an emulsion, a suppository, an injection, an ointment, a tape and the like.

Formulations suitable for oral administration are emulsions, syrups, capsules, tablets, powders, granules and the like.

Liquid preparations such as emulsions or syrups are produced using water, sugars such as sucrose, sorbitol or fructose, glycols such as polyethylene glycol or propylene glycol, oils such as sesame oil, olive oil or soybean oil, preservatives such as p-hydroxybenzoic acid esters, flavors such as strawberry flavor or peppermint or the like as an additive.

The capsules, the tablets, the powders, the granules and the like are produced using excipients such as lactose, glucose, sucrose or mannitol, disintegrating agents such as starch or sodium alginate, lubricants such as magnesium stearate or talc, binders such as polyvinyl alcohol, hydroxypropyl cellulose or gelatin, surfactants such as a fatty acid ester, plasticizers such as glycerin or the like as an additive.

Formulations suitable for parenteral administration are injections, suppositories, sprays and the like. The injections are produced using a salt solution, a glucose solution, a carrier formed of a mixture of these solutions or the like. The suppositories are produced using carriers such as cocoa butter, hydrogenated fats or carboxylic acids.

The sprays are produced using a carrier which does not stimulate the oral and respiratory mucosa of a recipient and which enables dispersion of the antibody or the antibody fragment of the invention as fine particles and easy absorption or the like. As the carrier, for example, lactose, glycerin or the like is used. In addition, it can also be produced as an aerosol or a dry powder. Furthermore, also for the above parenteral preparations, the components exemplified as the additives for the formulations suitable for oral administration can also be added.

### 6. Method for Detecting or Measuring Antigen Present in Brain or Method for Diagnosing Disease Using Antibody or Antibody Fragment of Invention

Using the antibody or the antibody fragment of the invention, MOG or MOG and an antigen that is present in the brain can be detected or measured. Moreover, by detecting or measuring MOG or MOG and an antigen that is present in the brain, a brain disease of an animal in which MOG is expressed in the brain can be diagnosed.

Examples of the brain disease include Alzheimer's disease, the prodromal stage of Alzheimer's disease, Huntington disease, Parkinson's disease, brain tumors, multiple sclerosis, muscular dystrophy, amyotrophic lateral sclerosis, multiple system atrophy, progressive supranuclear palsy, nigrostriatal degeneration, olivopontocerebellar atrophy, bulbospinal muscular atrophy, spinocerebellar degeneration, cerebrovascular disease, epilepsy, migraine, hyperactivity disorder, Creutzfeldt-Jakob disease, corticobasal degeneration, lysosomal storage disease, depression, dystonia and the like. The brain disease that the antibody or the antibody fragment of the invention can diagnose differs with the antigen to which the antibody or the antibody fragment of the invention binds, the kind of the molecule which modifies the antibody or the antibody fragment in the fused antibody or the fused antibody fragment of the invention and the like.

The brain disease of an animal in which MOG is expressed in the brain can be diagnosed for example by detecting or measuring MOG that is present in the brain of the patient or the patient animal by an immunological method. Moreover, the brain disease can be diagnosed by detecting MOG that is expressed or present in the cells in the brain of the patient or the patient animal using an immunological method such as flow cytometry.

When a monovalent antibody which binds to MOG is used as the antibody or the antibody fragment of the invention, MOG in the brain can be measured by the same method as that described above. When a bispecific antibody or a multispecific antibody which binds to MOG and an antigen that is present in the brain is used as the antibody or the antibody fragment of the invention, MOG in the brain or the antigen that is present in the brain can be detected or measured by the same method as that described above.

The immunological method is a method of detecting or measuring the amount of an antibody or the amount of an antigen using a labeled antigen, antibody or the like. For example, the radioactive material labeled immune antibody method, the enzyme immunoassay method, the fluorescence immunoassay method, the luminescence immunoassay method, the western blotting method, the physicochemical method or the like is used.

In the radioactive material labeled immune antibody method, for example, the antibody or the antibody fragment of the invention is reacted with an antigen, cells expressing an antigen or the like and then reacted with an anti-immunoglobulin antibody or an antibody fragment thereof subjected to radiolabeling, followed by measurement with a scintillation counter or the like.

In the enzyme immunoassay method, for example, the antibody or the antibody fragment of the invention is reacted with an antigen, cells expressing an antigen or the like and then reacted with an anti-immunoglobulin antibody or an antibody fragment thereof subjected to labeling with an enzyme or the like, followed by addition of a substrate and measurement of the absorbance of the reaction solution with an absorptiometer. For example, a sandwich ELISA method or the like is used. As a labeling substance used in the enzyme immunoassay method, a known [Enzyme Immunoassay Method, Igaku-Shoin Ltd. (1987)] enzyme label can be used.

For example, alkaline phosphatase label, peroxidase label, luciferase label, biotin label or the like is used. The sandwich ELISA method is a method in which after binding an antibody to a solid phase, a target antigen to be detected or to be measured is trapped, and then a second antibody is reacted with the trapped antigen.

In the ELISA method, two kinds of antibodies which recognize the antigen to be detected or measured and which have different antigen recognition sites are prepared, and among these, a first antibody is adsorbed on a plate (for example, a 96-well plate) in advance, followed by labeling the second antibody with a fluorescent substance such as FITC, an enzyme such as peroxidase, biotin or the like.

The plate on which the first antibody is adsorbed is allowed to react with cells or a lysate thereof, tissues or a lysate thereof, a cell culture supernatant, serum, pleural effusion, ascites, intraocular fluid or the like separated from the living body and then to react with the second antibody, followed by the detection reaction according to the labeling material. From a calibration curve prepared by serially diluting the antigen of a known concentration, the antigen concentration in the test sample is calculated.

As the antibodies used in the sandwich ELISA method, either a polyclonal antibody or a monoclonal antibody may be used. Antibody fragments such as Fab, Fab′ and F(ab)₂ may be used instead of the antibodies. The combination of the two kinds of antibodies used in the sandwich ELISA method may be a combination of monoclonal antibodies or antibody fragments thereof which recognize different epitopes or may be a combination of a polyclonal antibody, a monoclonal antibody and antibody fragments thereof.

In the fluorescence immunoassay method, measurement is carried out by the method described in documents [Monoclonal Antibodies-Principles and practice, Third edition, Academic Press (1996) and A manual for monoclonal antibody experiments, Kodansha scientific books (1987)] or the like. As the labeling substance used in the fluorescence immunoassay method, a known [Fluorescent Antibody Method, Soft Science (1983)]fluorescent label can be used. For example, FITC, RITC or the like is used.

In the luminescence immunoassay method, measurement is carried out by the method described in a document [Bioluminescence and Chemiluminescence, Clinical Test 42, Hirokawa-Shoten Ltd. (1998)] or the like. As the labeling substance used in the luminescence immunoassay method, a known luminescent label is used, and an acridinium ester, a lophine or the like is used.

In the western blotting method, measurement is carried out by after fractionating antigens, cells expressing an antigen or the like by SDS (sodium dodecyl sulfate)—PAGE (polyacrylamide gel) [Antibodies—A Laboratory Manual Cold Spring Harbor Laboratory (1988)], blotting the gel on a polyvinylidene fluoride (PVDF) membrane or a nitrocellulose membrane, reacting an antibody or an antibody fragment that recognizes the antigen with the membrane, further reacting it with an anti-mouse IgG antibody or a binding fragment subjected to labeling with a fluorescent substance such as FITC, labeling with an enzyme such as peroxidase, biotin labeling or the like and then visualizing the label. An example is shown below.

Cells or tissues expressing a polypeptide having the amino acid sequence of MOG are lysed, and 0.1 to 30 μg as a protein amount per lane is subjected to electrophoresis by the SDS-PAGE method under reducing conditions. The electrophoresed proteins are transferred to a PVDF membrane and reacted with PBS containing 1 to 10% BSA (referred to as BSA-PBS below) for 30 minutes at room temperature to perform blocking operation.

The antibody or the antibody fragment of the invention is reacted therewith, and the membrane is washed with PBS containing 0.05 to 0.1% Tween-20 (referred to as Tween-PBS below) and reacted with a goat anti-mouse IgG labeled with peroxidase for 2 hours at room temperature.

By washing with Tween-PBS and detecting a band to which the antibody or the antibody fragment of the invention is bound using ECL Western Blotting Detection Reagents (manufactured by Amersham) or the like, the polypeptide having the amino acid sequence of MOG is detected.

As the antibody or the antibody fragment used for detection by western blotting, an antibody or an antibody fragment thereof capable of binding to a polypeptide that does not retain the natural three-dimensional structure is used.

The physicochemical method is carried out, for example, by binding MOG, which is the antigen, with the antibody or the antibody fragment of the invention to form an aggregate and detecting the aggregate. As another physicochemical method, a capillary tube method, a one-dimensional immunodiffusion method, an immunoturbidimetric method, a latex immunoturbidimetric method [Outline of Clinical Examination Method, KANEHARA & Co., LTD. (1998)] or the like can also be used.

In the latex immunoturbidimetric method, when a carrier such as a polystyrene latex having a particle size of approximately 0.1 to 1 μm sensitized with an antibody or an antigen is used to cause the antigen-antibody reaction with a corresponding antigen or antibody, the scattered light is increased in a reaction solution, and the transmitted light is decreased. The antigen concentration and the like in the test sample are measured by detecting this change as absorbance or integrating sphere turbidity.

For detection or measurement of cells expressing MOG, a known immunological detection method can be used, but of known methods, the immunoprecipitation method, the immunocytostaining method, the immunohistochemical staining method, the fluorescent antibody staining method or the like is preferably used.

In the immunoprecipitation method, after reacting cells expressing MOG or the like with the antibody or the antibody fragment of the invention, a carrier having specific binding ability to an immunoglobulin such as Protein G-Sepharose is added thereto, and therefore an antigen-antibody complex is precipitated. Alternatively, the method can also be carried out by the following method.

The antibody or the antibody fragment of the invention described above is immobilized on a 96-well plate for ELISA and then blocked with BSA-PBS. When the antibody is an antibody which is not purified such as a hybridoma culture supernatant for example, the hybridoma culture supernatant is dispensed and bound after immobilizing anti-mouse immunoglobulin, anti-rat immunoglobulin, protein-A, protein-G or the like on a 96-well plate for ELISA in advance and blocking the plate with BSA-PBS.

Next, after discarding BSA-PBS and thoroughly washing with PBS, lysates of cells or tissues expressing human MOG are reacted therewith. Immunoprecipitates are extracted from the plate after thoroughly washing with a sample buffer for SDS-PAGE and detected by the above western blotting.

The immunocytostaining method or the immunohistochemical staining method is a method in which cells, tissues or the like expressing an antigen are treated with a surfactant, methanol or the like in order to improve passing of the antibody in some cases, then reacted with the antibody of the invention and further reacted with an anti-immunoglobulin antibody or a binding fragment thereof subjected to fluorescent labeling with FITC or the like, labeling with an enzyme such as peroxidase, biotin labeling or the like and in which the label is then visualized and observed with a microscope.

In addition, detection can be carried out by the fluorescent antibody staining method in which a fluorescently-labeled antibody is reacted with cells and analyzed with a flow cytometer [Monoclonal Antibodies—Principles and practice, Third edition, Academic Press (1996) and A manual for monoclonal antibody experiments, Kodansha scientific books (1987)]. In particular, the antibody or the antibody fragment of the invention can detect cells in which the antigen is expressed and retains the natural three-dimensional structure by the fluorescent antibody staining method.

In addition, when the FMAT 8100 HTS system (manufactured by Applied Biosystems) or the like of the fluorescent antibody staining methods is used, the amount of an antigen or the amount of an antibody can be measured without separating the formed antibody-antigen complex from the free antibody or antigen that is not involved in formation of the antibody-antigen complex.

Hereinafter, the invention will be explained in more detail by Examples, but the invention is not limited to the following Examples.

### EXAMPLES

### [Example 1] Acquisition of Anti-MOG Antibodies

### (1) Acquisition of Antibodies Using Human Antibody Phage Libraries

A VH gene fragment and a VL gene fragment were amplified from human PBMC-derived cDNA by PCR. The VH gene fragment and the VL gene fragment were inserted to a phagemid vector pCANTAB 5E (manufactured by Amersham Pharmacia Biotech), and a plasmid was obtained by transforming Escherichia coli TG1 (manufactured by Lucigen).

The obtained plasmid was infected with M13KO7 Helper Phage (manufactured by Invitrogen), and a human antibody M13 phage library of the VH gene and the VL gene was thus obtained.

Using the human antibody M13 phage libraries, anti-rat MOG (rMOG) monoclonal antibodies were obtained using the phage display method described below. rMOG-FLAG_Fc of Example 4 described below was immobilized on a MAXISORP STARTUBE (manufactured by NUNC), and the sites to which rMOG-FLAG_Fc was not bound were blocked using SuperBlock Blocking Buffer (manufactured by Thermo Fisher Scientific Inc.).

A human antibody M13 phage library was reacted with the tube at room temperature for an hour, and the phage was eluted with 0.1 M Gly-HCl (pH 2.2) after washing with PBS or PBS containing 0.1% Tween 20 (referred to as PBS-T below). The eluate was neutralized by adding Tris-HCl (pH 8.5). TG1 competent cells were infected with the eluted phage, and the phage was amplified.

Then, the reaction with rMOG-FLAG Fc immobilized on a MAXISORP STARTUBE was conducted again, and washing and elution were conducted. This procedure was repeated, and phages displaying scFv which specifically binds to rMOG-FLAG_Fc were concentrated. The concentrated phages were monocloned, and three clones having affinity to rMOG-FLAG_Fc were selected by ELISA.

In ELISA, rMOG-FLAG_Fc was immobilized on MAXISORP (manufactured by NUNC), and the sites to which rMOG-FLAG_Fc was not bound were blocked using SuperBlock Blockig Buffer (manufactured by Thermo Fisher Scientific Inc.). As the negative control, a plate in which FLAG_Fc was immobilized was also prepared.

The phage clones were added to separate wells and reacted at room temperature for 30 minutes, and then the wells were washed with PBS-T. Subsequently, a solution obtained by diluting horseradish peroxidase-labeled anti-M13 antibody (manufactured by GE Healthcare) with PBS-T containing 10% Block Ace (manufactured by Dainippon Pharma Co., Ltd.) was added to the wells, and the plates were incubated at room temperature for 30 minutes.

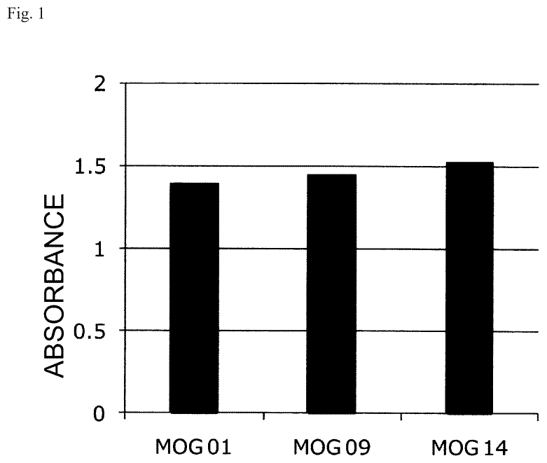

After washing the microplates three times with PBS-T, a TMB chromogenic substrate solution (manufactured by DAKO) was added, and the plates were incubated at room temperature. The chromogenic reaction was stopped by adding 0.5 M sulfuric acid to the wells, and the absorbances at the wavelength of 450 nm (reference wavelength of 570 nm) were measured with a microplate reader (manufactured by Molecular Devices). The results obtained are shown in FIG. 1.

As shown in FIG. 1, it could be confirmed that the three phage clones all bind to rMOG-FLAG Fc. On the other hand, none of the phage clones bound to FLAG Fc (data not shown).

The sequences of the clones which bound to rMOG-FLAG_Fc were analyzed, and anti-MOG antibody phagemid vectors, pCANTAB_MOG01, pCANTAB_MOG09 and pCANTAB_MOG14 were obtained.

In the following paragraphs, the names of the anti-MOG scFv antibodies displayed by the phages expressed using pCANTAB_MOG01, pCANTAB_MOG09 and pCANTAB_MOG14 are referred to as MOG01 antibody, MOG09 antibody and MOG14 antibody, respectively. The nucleotide sequences which encode VH or VL of the anti-MOG scFv antibodies, and the amino acid sequences deduced from the nucleotide sequences are shown in Table 1.

TABLE 1
Sequence Information of Anti-MOG scFv Antibodies (MOG01
Antibody, MOG09 Antibody and MOG14 Antibody)

| Clone Name | MOG01 | MOG09 | MOG14 |
|---|---|---|---|
| Nucleotide | SEQ ID | SEQ ID NO: 13 | SEQ ID NO: 25 |
| sequence encoding | NO: 1 | | |
| VH (including | | | |
| signal sequence) | | | |
| Amino acid | SEQ ID | SEQ ID NO: 14 | SEQ ID NO: 26 |
| sequence of VH | NO: 2 | | |
| (including signal | | | |
| sequence) | | | |
| Amino acid | SEQ ID | SEQ ID NO: 15 | SEQ ID NO: 27 |
| sequence of VH | NO: 3 | | |
| (excluding | | | |
| signal sequence) | | | |
| Amino acid | SEQ ID | SEQ ID NO: 16 | SEQ ID NO: 28 |
| sequence of HCDR1 | NO: 4 | | |
| Amino acid | SEQ ID | SEQ ID NO: 17 | SEQ ID NO: 29 |
| sequence of HCDR2 | NO: 5 | | |
| Amino acid | SEQ ID | SEQ ID NO: 18 | SEQ ID NO: 30 |
| sequence of HCDR3 | NO: 6 | | |
| Nucleotide | SEQ ID | SEQ ID NO: 19 | SEQ ID NO: 31 |
| sequence | NO: 7 | | |
| encoding VL | | | |
| (including | | | |
| signal sequence) | | | |
| Amino acid | SEQ ID | SEQ ID NO: 20 | SEQ ID NO: 32 |
| sequence of VL | NO: 8 | | |
| (including | | | |
| signal sequence) | | | |
| Amino acid | SEQ ID | SEQ ID NO: 21 | SEQ ID NO: 33 |
| sequence of VL | NO: 9 | | |
| (excluding | | | |
| signal sequence) | | | |
| Amino acid | SEQ ID | SEQ ID NO: 22 | SEQ ID NO: 34 |
| sequence of LCDR1 | NO: 10 | | |
| Amino acid | SEQ ID | SEQ ID NO: 23 | SEQ ID NO: 35 |
| sequence of LCDR2 | NO: 11 | | |
| Amino acid | SEQ ID | SEQ ID NO: 24 | SEQ ID NO: 36 |
| sequence of LCDR3 | NO: 12 | | |

### (2) Acquisition of Antibodies Using Alpaca Antibody Libraries

An emulsion of rMOG-FLAG_Fc and complete adjuvant for the first immunization and an emulsion of rMOG-FLAG_Fc and incomplete adjuvant for the second and third immunization were produced as immunogens, and an alpaca was immunized.

Lymphocytes (2×10⁷ cells) were collected from the blood (50 mL) of the immunized alpaca, and RNA was extracted from the obtained cells using RNA IsoPlus (manufactured by TAKARA). After synthesizing cDNA by reverse transcription reaction using SuperScript (registered trademark) III First-Strand Synthesis System for RT-PC (manufactured by Invitrogen), VHH gene was amplified using primers specific to alpaca IgG2 (Short hinge-heavy chain antibody) and IgG3 (Long hinge-heavy chain antibody).

The VHH gene fragment was inserted to a phagemid vector pKSTV-02 (described in Miyazaki et al, J. Biochem. 2015; 1), and Escherichia coli TG1 was transformed by electroporation using a MicroPulser electroporator (manufactured by BioRad) (the IgG2 titer of the transformant was 2.6×10⁷, and the IgG3 titer was 3.2×10⁷).

The obtained transformant was infected with M13KO7 Helper Phage (manufactured by Invitrogen), and an alpaca antibody M13 phage library of the VHH gene was thus obtained.

Using the alpaca antibody M13 phage libraries, anti-MOG antibodies were obtained using the biopanning method described below. rMOG-GST (4 μg/2 mL) was immobilized on an immuno tube, and the sites to which rMOG-GST was not bound were blocked using 0.5% BSA.

The alpaca antibody M13 phage library was reacted with the tube at room temperature for an hour, and the phage was eluted with 0.1 M Gly-HCl (pH 2.7) after washing with PBS-T. The eluant was neutralized by adding Tris-HCl (pH 9.1). Escherichia coli TG1 was infected with the eluted phage, and then the phage was amplified. Then, the reaction with rMOG-GST immobilized on an immuno tube was conducted again, and washing and elution were conducted.

The procedure was repeated three times for IgG2 and twice for IgG3, and phages displaying VHH which specifically binds to rMOG-GST were concentrated. From the concentrated phages, 96 phage clones displaying VHH of IgG2 and 96 phage clones displaying VHH of IgG3 were monocloned, and clones having affinity to rMOG-GST were selected by ELISA.

In ELISA, rMOG-GST was immobilized (50 ng/50 μL) on MAXISORP (manufactured by NUNC), and the sites to which rMOG-GST was not bound were blocked using 0.5% BSA. The phage clones were added to separate wells and reacted at room temperature for an hour, and then the wells were washed five times with PBS-T.

Subsequently, 50 μL of a biotinylated anti-M13 phage antibody (manufactured by Abcam) and horseradish peroxidase-labeled streptavidin (manufactured by Vector) were added to the wells, and the plate was incubated at room temperature for an hour.

After washing the microplate with PBS-T, a TMB chromogenic substrate solution (manufactured by CALBIOCHEM) was added to the wells, and the plate was incubated at room temperature. The chromogenic reaction was stopped by adding 1 M hydrochloric acid to the wells, and the absorbances at the wavelength of 450 nm (reference wavelength of 570 nm) were measured with a microplate reader (Model 680XR, manufactured by BioRad).

The sequences of the clones which bound to rMOG-GST were analyzed, and an anti-MOG VHH antibody, iMOG-3Rim1-S32 antibody was obtained. The nucleotide sequence encoding VHH of iMOG-3Rim1-S32 antibody and the amino acid sequence deduced from the nucleotide sequence are shown in Table 2.

TABLE 2
Sequence Information of Anti-MOG VHH Antibody
(iMOG-3Rim1-S32 Antibody)

| Clone Name | iMOG-3Rim1-S32 |
|---|---|
| Nucleotide sequence encoding | SEQ ID NO: 37 |
| VHH (including signal sequence) | |
| Amino acid sequence of VHH | SEQ ID NO: 38 |
| (including signal sequence) | |
| Amino acid sequence of VHH | SEQ ID NO: 39 |
| (excluding signal sequence) | |
| Amino acid sequence of CDR1 | SEQ ID NO: 40 |
| Amino acid sequence of CDR2 | SEQ ID NO: 41 |
| Amino acid sequence of CDR3 | SEQ ID NO: 42 |

### [Example 2] Construction of Antibody Expression Vectors

### (1) Construction of Anti-MOG Antibody Expression Vectors

To produce anti-MOG antibodies of human IgG type, expression vectors for anti-MOG antibodies in which the DNA sequences encoding the amino acid sequences of the variable regions of the human antibody phage library-derived anti-MOG scFv antibodies obtained in Example 1 were incorporated into a nucleotide sequence encoding the amino acid sequence of a constant region of human IgG antibody were produced by the method described below.

A nucleotide sequence encoding the lambda chain constant region of human IgG was synthesized and inserted to the BglII-EcoRI site of N5KG4PE vector (described in International Publication No. 2002/088186), and N5LG4PE vector was thus produced.

Expression vectors obtained by inserting nucleotide sequences encoding the amino acid sequences of VH and VL of MOG01 antibody and MOG09 antibody into N5LG4PE were named N5LG4PE_MOG01 and N5LG4PE_MOG09, respectively. Moreover, an expression vector obtained by inserting nucleotide sequences encoding the amino acid sequences of VH and VL of MOG14 antibody into N5KG4PE vector was named N5KG4PE_MOG14.

### (1-1) MOG01 Antibody Expression Vector N5LG4PE_MOG01

Using phagemid vector pCANTAB_MOG01 as a template and using primer 1 (SEQ ID NO: 43) and primer 2 (SEQ ID NO: 44) and KOD plus DNA Polymerase (manufactured by Toyobo Co., Ltd.), a gene fragment of the VL region was amplified by PCR. In the PCR, 30 cycles of reaction of 30 seconds at 94° C., 30 seconds at 58° C. and 45 seconds at 68° C. were conducted. The PCR described in Example 2 was conducted under the conditions unless particularly described.

Using the PCR product as a template and using primer 3 (SEQ ID NO: 45) and primer 2 (SEQ ID NO: 44) and KOD plus DNA Polymerase (manufactured by Toyobo Co., Ltd.), a signal sequence was added to the gene fragment of the VL region by PCR.

The obtained gene fragment was inserted to the BglII-BlpI site of N5LG4PE vector, and N5LG4PE_MOG01VL was obtained. Next, using pCANTAB_MOG01 as a template and using primer 4 (SEQ ID NO: 46) and primer 5 (SEQ ID NO: 47) and KOD plus DNA Polymerase (manufactured by Toyobo Co., Ltd.), a gene fragment of the VH region was amplified by PCR.

Using the PCR product as a template and using primer 6 (SEQ ID NO: 48) and primer 5 (SEQ ID NO: 47) and KOD plus DNA Polymerase (manufactured by Toyobo Co., Ltd.), a signal sequence was added to the gene fragment of the VH region by PCR. The obtained gene fragment was inserted to the SalI-NheI site of N5LG4PE_MOG01VL vector, and N5LG4PE_MOG01 was obtained.

### (1-2) MOG09 Antibody Expression Vector N5LG4PE_MOG09

N5LG4PE_MOG09 was produced by the same method as that of (1-1). A phagemid vector pCANTAB_MOG09 was used as a template. Primer 7 (SEQ ID NO: 49) and primer 8 (SEQ ID NO: 50) were used to amplify a gene fragment of the VL region, and primer 3 (SEQ ID NO: 45) and primer 8 (SEQ ID NO: 50) were used to add a signal sequence to the gene fragment of the VL region. Primer 9 (SEQ ID NO: 51) and primer 10 (SEQ ID NO: 52) were used to amplify a gene fragment of the VH region, and primer 6 (SEQ ID NO: 48) and primer 10 (SEQ ID NO: 52) were used to add a signal sequence to the gene fragment of the VH region.

### (1-3) MOG14 Antibody Expression Vector N5KG4PE_MOG14

N5KG4PE_MOG14 was produced by the same method as that of (1-1). A phagemid vector pCANTAB_MOG14 was used as a template. Primer 11 (SEQ ID NO: 53) and primer 12 (SEQ ID NO: 54) were used to amplify a gene fragment of the VL region, and primer 3 (SEQ ID NO: 45) and primer 12 (SEQ ID NO: 54) were used to add a signal sequence to the gene fragment of the VL region. The obtained gene fragment of the VL region to which the signal sequence was added was inserted to the BglII-BsiWI site of N5KG4PE vector, and N5KG4PE_MOG14VL was obtained.

Next, pCANTAB_MOG14 was used as a template. Primer 13 (SEQ ID NO: 55) and primer 14 (SEQ ID NO: 56) were used to amplify a gene fragment of the VH region, and primer 6 (SEQ ID NO: 48) and primer 14 (SEQ ID NO: 56) were used to add a signal sequence to the gene fragment of the VH region. The obtained gene fragment of the VH region to which the signal sequence was added was inserted to the SalI-NheI site of N5KG4PE_MOG14VL, and N5KG4PE_MOG14 was obtained.

(1-4) iMOG-3Rim1-S32 Antibody Expression Vector N5G4PEFc_iMOG-3Rim1-S32

A sequence obtained by adding a signal sequence to the gene encoding the Fc region of human IgG4PE was synthesized, and a gene fragment of human Fc region was amplified by PCR using primer 25 (SEQ ID NO: 79) and primer 26 (SEQ ID NO: 80) and KOD plus DNA Polymerase (manufactured by Toyobo Co., Ltd.).

In the PCR, 30 cycles of reaction of 30 seconds at 94° C., 30 seconds at 58° C. and 60 seconds at 68° C. were conducted. The obtained Fc gene fragment was inserted to the BglII-BamHI site of N5KG4PE vector, and N5G4PEFc vector was produced.

An expression vector obtained by inserting a nucleotide sequence encoding the amino acid sequence of VHH of iMOG-3Rim1-532 to N5G4PEFc was named N5G4PEFc_iMOG-3Rim1-S32. The VHH-Fc expression vector was produced by the method described below.

The nucleotide sequence of VHH of iMOG-3Rim1-S32 was synthesized, and a gene fragment of the VHH region was amplified by PCR using primer 15 (SEQ ID NO: 57) and primer 16 (SEQ ID NO: 58) and KOD plus DNA Polymerase (manufactured by Toyobo Co., Ltd.). In the PCR, 30 cycles of reaction of 30 seconds at 94° C., 30 seconds at 58° C. and 60 seconds at 68° C. were conducted. The obtained VHH gene fragment was inserted to the EcoRI-BglII site of N5G4PEFc vector, and N5G4PEFc_iMOG-3Rim1-S32 was obtained.

### (2) Anti-Avermectin Antibody Expression Vector N5LG4PE_AVM

As a negative control antibody, a chimeric anti-Avermectin (AVM) antibody was produced by the same method as that of (1-1). An expression vector obtained by inserting nucleotide sequences encoding the amino acid sequences of VH and VL of an AVM antibody to N5LG4PE was named N5LG4PE_AVM.

An SD rat was immunized with AVM, and an anti-AVM antibody-producing hybridoma was established by a general method. The variable region of the anti-AVM antibody derived from the hybridoma was used as a template. Primer 29 (SEQ ID NO: 83) and primer 30 (SEQ ID NO: 84) were used to amplify a gene fragment of the VL region, and primer 3 (SEQ ID NO: 45) and primer 30 (SEQ ID NO: 84) were used to add a signal sequence to the gene fragment of the VL region.

Primer 31 (SEQ ID NO: 85) and primer 32 (SEQ ID NO: 86) were used to amplify a gene fragment of the VH region, and primer 6 (SEQ ID NO: 48) and primer 32 (SEQ ID NO: 86) were used to add a signal sequence to the gene fragment of the VH region.

### (3) Anti-Rat Transferrin Receptor Antibody OX26 Antibody Expression Vector N5KG4PE(R409K)_OX26

As a positive control antibody of an anti-rat transferrin receptor antibody, the anti-rat transferrin receptor antibody, OX26 antibody described in [Protein Engineering, 12, 787-796, 1999] was produced. An expression vector obtained by inserting nucleotide sequences encoding the amino acid sequences of VH and VL of OX26 antibody to N5KG4PE (R409K) (described in International Publication No. 2002/088186) was produced by the same method as that of (1-1) and named N5KG4PE(R409K)_OX26.

The gene encoding the amino acid sequence of VL of OX26 antibody was synthesized and used as a template. Primer 40 (SEQ ID NO: 94) and primer 41 (SEQ ID NO: 95) were used to amplify a gene fragment of the VL region, and primer 42 (SEQ ID NO: 96) and primer 43 (SEQ ID NO: 97) were used to amplify a gene fragment of the VH region.

### [Example 3] Construction of Bispecific Antibody Expression Vectors

### (l) Production of Vector Expressing Bispecific Antibody Binding to Her2 and MOG

A vector expressing a bispecific antibody binding to HER2 and MOG, pCI-Trastuzumab-hKG4PE(R409K)_MOG01scFv was produced by the following method. In the bispecific antibody, scFv of an anti-MOG antibody is fused to the C-terminuses of the two H chains of IgG of an anti-HER2 antibody.

Using a synthetic gene of the heavy chain constant region as a template and using primer 17 (SEQ ID NO: 59) and primer 18 (SEQ ID NO: 60) and KOD plus DNA Polymerase (manufactured by Toyobo Co., Ltd.), a gene fragment of the CH1-Hinge-CH2-CH3-linker region was amplified by PCR.

In the PCR, 30 cycles of reaction of 30 seconds at 94° C., 30 seconds at 58° C. and two minutes at 68° C. were conducted. Using a phagemid vector pCANTAB_MOG01 as a template and using primer 19 (SEQ ID NO: 61) and primer 20 (SEQ ID NO: 62) and KOD plus DNA Polymerase (manufactured by Toyobo Co., Ltd.), a gene fragment of the scFv region (referred to as MOG01scFv below) was amplified by PCR.

In the PCR, 30 cycles of reaction of 30 seconds at 94° C., 30 seconds at 58° C. and 90 seconds at 68° C. were conducted. Next, using the CH1-Hinge-CH2-CH3 region and the MOG01scFv region as templates and using primer 17 (SEQ ID NO: 59) and primer 20 (SEQ ID NO: 62) and KOD plus DNA Polymerase (manufactured by Toyobo Co., Ltd.), CH1-Hinge-CH2-CH3-MOG01scFv was amplified.

In the PCR, 30 cycles of reaction of 30 seconds at 94° C., 30 seconds at 58° C. and two minutes at 68° C. were conducted. The obtained gene fragment was inserted to pCI vector (manufactured by Promega Corporation), and pCI-hG4PE(R409K)_MOG01scFv vector was produced.

The gene encoding the amino acid sequence of VL of an anti-HER2 antibody (Trastuzumab) (described in International Publication No. 1999/57134) was synthesized and used as a template, and a gene fragment of the VL region was amplified by PCR using primer 21 (SEQ ID NO: 63) and primer 22 (SEQ ID NO: 64) and KOD plus DNA Polymerase (manufactured by Toyobo Co., Ltd.).

In the PCR, 30 cycles of reaction of 30 seconds at 94° C., 30 seconds at 58° C. and 45 seconds at 68° C. were conducted. Using N5KG4PE vector (described in International Publication No. 2002/088186) as a template and using primer 27 (SEQ ID NO: 81) and primer 28 (SEQ ID NO: 82) and KOD plus DNA Polymerase (manufactured by Toyobo Co., Ltd.), a gene fragment of the CL region was amplified by PCR.

In the PCR, 30 cycles of reaction of 30 seconds at 94° C., 30 seconds at 58° C. and 45 seconds at 68° C. were conducted. Using the obtained gene fragments VL and CL as templates and using primer 21 (SEQ ID NO: 63) and primer 28 (SEQ ID NO: 82) and KOD plus DNA Polymerase (manufactured by Toyobo Co., Ltd.), a gene fragment was amplified by PCR.

In the PCR, 30 cycles of reaction of 30 seconds at 94° C., 30 seconds at 58° C. and 90 seconds at 68° C. were conducted. The obtained gene fragment was inserted to pCI-hG4PE(R409K)_MOG01scFv, and pCI-TrastuzumabVL-hKG4PE(R409K)_MOG01scFv was obtained.

Next, the gene encoding the amino acid sequence of VH of Trastuzumab was synthesized and used as a template, and a gene fragment of the VH region was amplified by PCR using primer 23 (SEQ ID NO: 65) and primer 24 (SEQ ID NO: 66) and KOD plus DNA Polymerase (manufactured by Toyobo Co., Ltd.). In the PCR, 30 cycles of reaction of 30 seconds at 94° C., 30 seconds at 58° C. and 45 seconds at 68° C. were conducted.

The obtained gene fragment was inserted to pCI-TrastuzumabVL-hKG4PE(R409K)_MOG01scFv, and pCI-Trastuzumab-hKG4PE(R409K)_MOG01scFv was obtained.

### (2) Production of Vector Expressing Bispecific Antibody Binding to AVM and MOG

Moreover, a vector expressing a bispecific antibody binding to AVM and MOG, pCI-AVM-hLG4PE(R409K)_MOG01scFv was produced by the method described below. In the bispecific antibody, scFv of an anti-MOG antibody is fused to the C-terminus of IgG of an anti-AVM antibody.

Using N5LG4PE_AVM as a template and using primer 33 (SEQ ID NO: 87) and primer 34 (SEQ ID NO: 88) and KOD plus DNA Polymerase (manufactured by Toyobo Co., Ltd.), a gene fragment of the AVM light chain region was amplified by PCR. In the PCR, 30 cycles of reaction of 30 seconds at 94° C., 30 seconds at 58° C. and 60 seconds at 68° C. were conducted.

Using N5LG4PE_AVM as a template and using primer 35 (SEQ ID NO: 89) and primer 32 (SEQ ID NO: 86) and KOD plus DNA Polymerase (manufactured by Toyobo Co., Ltd.), a gene fragment of the AVM VH region was amplified by PCR.

In the PCR, 30 cycles of reaction of 30 seconds at 94° C., 30 seconds at 58° C. and 45 seconds at 68° C. were conducted. The obtained gene fragment was inserted to pCI-hG4PE(R409K)_MOG01scFv produced above, and pCI-AVM-hLG4PE(R409K)_MOG01scFv was obtained.

(3) Production of Vector Expressing Antibody in which scFv of Anti-AVM Antibody is Fused to C-Terminus of IgG of Anti-AVM Antibody

As a negative control antibody, a vector expressing an antibody in which scFv of an anti-AVM antibody is fused to the C-terminus of IgG of an anti-AVM antibody was named pCI-AVM-hLG4PE(R409K)_AVM scFv.

Using a synthetic gene of the heavy chain constant region as a template and using primer 36 (SEQ ID NO: 90) and primer 37 (SEQ ID NO: 91) and KOD plus DNA Polymerase (manufactured by Toyobo Co., Ltd.), a gene fragment of the CH1-Hinge-CH2-CH3-linker region was amplified by PCR.

In the PCR, 30 cycles of reaction of 30 seconds at 94° C., 30 seconds at 58° C. and two minutes at 68° C. were conducted. Using a synthetic gene of AVM scFv as a template and using primer 38 (SEQ ID NO: 92) and primer 39 (SEQ ID NO: 93) and KOD plus DNA Polymerase (manufactured by Toyobo Co., Ltd.), a gene fragment of the scFv region was amplified by PCR.

In the PCR, 30 cycles of reaction of 30 seconds at 94° C., 30 seconds at 58° C. and 90 seconds at 68° C. were conducted. Next, using the CH1-Hinge-CH2-CH3 region and the AVM scFv region as templates and using primer 36 (SEQ ID NO: 90) and primer 39 (SEQ ID NO: 93) and KOD plus DNA Polymerase (manufactured by Toyobo Co., Ltd.), CH1-Hinge-CH2-CH3-AVM scFv was amplified. In the PCR, 30 cycles of reaction of 30 seconds at 94° C., 30 seconds at 58° C. and two minutes at 68° C. were conducted.

The obtained gene fragment was inserted to the NheI-BamHI site of pCI-AVM-hLG4PE(R409K)_MOG01scFv, and pCI-AVM-hLG4PE(R409K)_AVM scFv was obtained.

### Example 4

### Production of Soluble MOG Antigen and Soluble HER2 Antigen

(1) Production of Extracellular Domain Protein of Rat MOG to which FLAG-Fc is Bound

As a soluble antigen of rat MOG, an extracellular domain protein of MOG to which FLAG-Fc was added at the C-terminus was produced by the method described below. The nucleotide sequence encoding rMOG is shown in SEQ ID NO: 67, and the amino acid sequence deduced from the nucleotide sequence is shown in SEQ ID NO: 68.

A gene sequence of the extracellular domain of MOG was synthesized and inserted to the BglII-XbaI site of INPEP4 (manufactured by IDEC) vector to which FLAG-Fc had been inserted, and a plasmid vector INPEP4_rMOG-FLAG-Fc expressing the extracellular domain of MOG to which FLAG-Fc was added at the C-terminus was thus produced. The nucleotide sequence of rMOG-FLAG-Fc is shown in SEQ ID NO: 69, and the amino acid sequence deduced from the nucleotide sequence is shown in SEQ ID NO: 70.

INPEP4_rMOG-FLAG-Fc was introduced to suspension 293 cells using Expi293 (trademark) Expression System (manufactured by Thermo Fisher Scientific Inc.), and the cells were cultured to express the protein in a transient expression system. The culture supernatant was collected four days after the introduction of the vector and filtered through a membrane filter having a pore size of 0.22 μm (manufactured by Millipore Corporation.).

The MOG-FLAG-Fc protein in the culture supernatant was affinity-purified using Protein A resin (MabSelect SuRe, manufactured by GE Healthcare BioSciences). A phosphate buffer solution was used as a washing solution.

The protein adsorbed on the Protein A was eluted with 20 mM sodium citrate and 50 mM NaCl buffer solution (pH 3.4) and collected in a tube containing 1 M Tris-HCl Buffer Solution (pH 8.0).

Next, the solvent of the eluate was replaced with PBS by ultrafiltration using VIVASPIN (manufactured by Sartrius stealin) and a NAP column (manufactured by GE Healthcare BioSciences), and then filtration sterilization with a membrane filter having a pore size of 0.22 μm (Millex-GV, manufactured by Millipore Corporation) was conducted. The concentration of the purified MOG-FLAG-Fc protein in the solution was measured from the absorbance at 280 nm.

(2) Production of Extracellular Domain Protein of MOG to which GST is Bound

As a soluble antigen of rat MOG, an extracellular domain protein of MOG to which GST was added at the C-terminus was produced by the method described below.

A gene sequence of the extracellular domain of MOG was synthesized and inserted to the BglII-KpnI site of N5 vector (manufactured by IDEC) to which GST had been inserted, and a plasmid vector N5_rMOG-GST expressing the extracellular domain of MOG to which GST was added at the C-terminus was thus produced. The nucleotide sequence of rMOG-GST is shown in SEQ ID NO: 71, and the amino acid sequence deduced from the nucleotide sequence is shown in SEQ ID NO: 72.

As a soluble antigen of human HER2, an extracellular domain protein of HER2 to which GST was added at the C-terminus was produced by the method described below. A gene sequence of the extracellular domain of HER2 was synthesized and inserted to the BglII-KpnI site of N5 vector (manufactured by IDEC) to which GST had been inserted, and a plasmid vector N5_hHER2-GST expressing the extracellular domain of HER2 to which GST was added at the C-terminus was thus produced. The nucleotide sequence of hHER2-GST is shown in SEQ ID NO: 71, and the amino acid sequence deduced from the nucleotide sequence is shown in SEQ ID NO: 72.

N5_rMOG-GST and N5_hHER2-GST were introduced to suspension 293 cells using Expi293 (trademark) Expression System (manufactured by Thermo Fisher Scientific Inc.), and the cells were cultured to express the proteins in a transient expression system. The culture supernatants were collected four days after the introduction of the vectors and filtered through a membrane filter having a pore size of 0.22 μm (manufactured by Millipore Corporation).

The proteins in the culture supernatants were affinity-purified using Glutathione Sepharose 4B (manufactured by GE Healthcare BioSciences). A phosphate buffer solution was used as a washing solution. The proteins adsorbed on the Glutathione Sepharose 4B were eluted with 50 mM Tris-HCl and 10 mM reduced glutatione (pH 8.0).

Next, the solvents of the solutions were replaced with PBS by ultrafiltration using VIVASPIN (manufactured by Sartrius stealin) and a NAP column (manufactured by GE Healthcare BioSciences), and then filtration sterilization with a membrane filter having a pore size of 0.22 μm (Millex-GV, manufactured by Millipore Corporation) was conducted. The concentrations of the purified rMOG-GST protein and the hHER2-GST protein in the solutions were measured from the absorbances at 280 nm.

### [Example 5] Production of Membrane MOG Antigen Expression Vectors

The entire gene sequences of rat MOG (rMOG), mouse MOG (mMOG), monkey MOG (cMOG) and human MOG (hMOG) were synthesized, and the gene sequences were each inserted to the BamHI-NotI site of pEF6/V5-His (manufactured by Thermo Fisher Scientific Inc.) vector. Plasmid vectors for expressing MOG in the membrane, pEF6_rMOG, pEF6_mMOG, pEF6_cMOG and pEF6_hMOG were thus produced.

The nucleotide sequence encoding mMOG is shown in SEQ ID NO: 73, and the amino acid sequence deduced from the nucleotide sequence is shown in SEQ ID NO: 74. The nucleotide sequence encoding cMOG is shown in SEQ ID NO: 75, and the amino acid sequence deduced from the nucleotide sequence is shown in SEQ ID NO: 76. The nucleotide sequence encoding hMOG is shown in SEQ ID NO: 77, and the amino acid sequence deduced from the nucleotide sequence is shown in SEQ ID NO: 78.

### [Example 6] Preparation of Antibodies

The antibody expression plasmid vectors produced in Example 2 and Example 3 were introduced to suspension 293 cells using Expi293™ Expression System (manufactured by Thermo Fisher Scientific Inc.), and the cells were cultured to express the antibodies in a transient expression system.

The culture supernatants were collected four days after the introduction of the vectors and filtered through a membrane filter having a pore size of 0.22 μm (manufactured by Millipore Corporation). The proteins in the culture supernatants were affinity-purified using Protein A resin (MabSelect SuRe, manufactured by GE Healthcare BioSciences). A phosphate buffer solution was used as a washing solution. The antibodies adsorbed on the Protein A were eluted with 20 mM sodium citrate and 50 mM NaCl buffer solution (pH 3.4) and collected in tubes containing 1 M Tris-HCl Buffer Solution (pH 8.0).

Next, the solvents of the eluates were replaced with PBS by ultrafiltration using VIVASPIN (manufactured by Sartrius stealin) and a NAP column (manufactured by GE Healthcare BioSciences), and then filtration sterilization with a membrane filter having a pore size of 0.22 μm (Millex-GV, manufactured by Millipore Corporation) was conducted. The absorbances of the antibody solutions at 280 nm were measured, and the concentrations of the purified antibodies were calculated by converting the concentration 1 mg/mL to 1.40 Optimal density.

The anti-MOG human IgG antibodies expressed using the anti-MOG antibody expression vectors, N5LG4PE_MOG01, N5LG4PE_MOG09, N5KG4PE_MOG14 and N5G4PEFc_iMOG-3Rim1-S32 described in Example 2 are referred to as MOG01 antibody, MOG09 antibody, MOG14 antibody and iMOG-3Rim1-S32 antibody, respectively.

The antibodies obtained by expressing using the bispecific antibody expression vectors, pCI-AVM-hLG4PE(R409K)_AVM scFv, pCI-AVM-hLG4PE(R409K)_MOG01scFv and pCI-Trastuzumab-hKG4PE(R409K)_MOG01scFv produced in Example 3 were named AVM IgG4PE(R409K)_AVM dscFv antibody, AVM IgG4PE(R409K)_MOG01dscFv antibody and Trastuzumab IgG4PE(R409K)_MOG01scFv antibody, respectively.

### [Example 7] Evaluation of Affinities of Anti-MOG Antibodies to MOG Using Flow Cytometer

Binding of the anti-MOG antibodies, MOG01 antibody, MOG09 antibody, MOG14 antibody and iMOG-3Rim1-S32 antibody obtained in Example 6 to MOG were evaluated by the fluorescence activated cell sorting (FACS) method according to the following procedures.

The membrane MOG antigen expression vectors produced in Example 5 were introduced to suspension 293 cells using FreeStyle (trademark) 293 Expression System (manufactured by Thermo Fisher Scientific Inc.), and the cells were cultured to express the membrane antigens in a transient expression system. Using the cells, the reactivities of the 25 anti-MOG antibodies were analyzed by the method described below.

rMOG/HEK293F, mMOG/IEK293F, cMOG/HEK293 and hMOG/HEK293 cells were suspended in a Staining Buffer (SB) of PBS containing 0.1% NaN₃ and 1% FBS each at a concentration of 5×10⁵ cells/mL and dispensed to a round-bottom 96-well plate (manufactured by Becton Dickinson).

After centrifugation (2000 rpm, 4° C., two minutes), the supernatants were removed, and the antibodies obtained in Example 6 at 10 μg/mL were added to the pellets. After suspending the pellets, the plate was left to stand at ice temperature for 30 minutes. The supernatants were removed after further centrifugation (2000 rpm, 4° C., two minutes), and the pellets were washed with SB. Then, 1 μg/mL RPE fluorescently labeled goat anti-human antibody (manufactured by Southern Bioblot) was added, and the plate was incubated at ice temperature for 30 minutes.

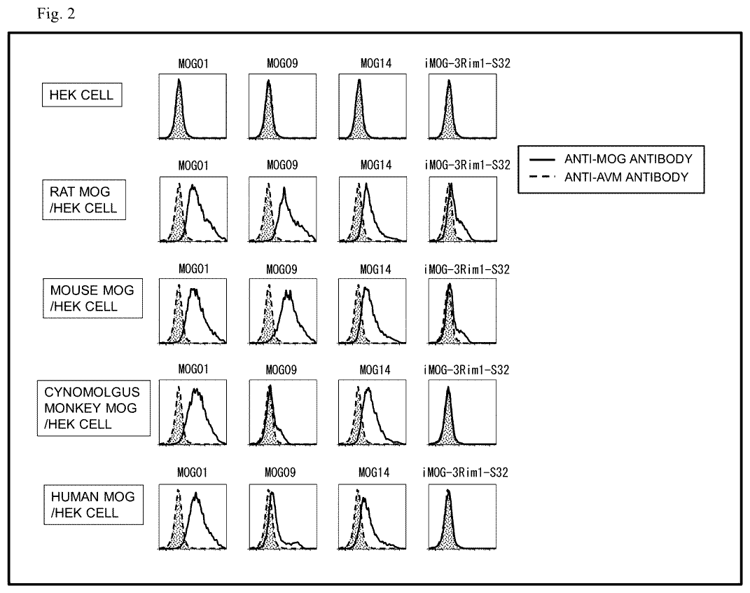

After washing with SB, the cells were suspended in SB, and the fluorescence intensities of the cells were measured using a flow cytometer FACS CANTO II (manufactured by Becton Dickinson). The results obtained are shown in FIG. 2. As the negative control, an anti-AVM antibody was used.

As shown in FIG. 2, MOG01 antibody, MOG09 antibody, MOG14 antibody and iMOG-3Rim1-S32 antibody, which are anti-MOG antibodies, all showed binding activity to rMOG/HEK293F cells and mMOG/HEK293F cells. Moreover, MOG01 antibody and MOG14 antibody both showed binding activity also to cMOG/HEK293 cells and hMOG/HEK293 cells.

Accordingly, it was elucidated that anti-MOG human IgG antibodies, MOG01 and MOG14 recognize and bind to not only rat and mouse MOG but also cynomolgus monkey and human MOG.

### [Example 8] Evaluation of Affinities of Anti-MOG Antibodies to MOG by Surface Plasmon Resonance Detection

The affinities of the anti-MOG antibodies, MOG01 antibody, MOG09 antibody, MOG14 antibody and iMOG-3Rim1-S32 antibody obtained in Example 6 to rat MOG were measured using Biacore T-100 (GE Healthcare).

The antibodies were immobilized on CM5 sensor chips using a Human antibody Capture kit, and the binding abilities were evaluated using rMOG-GST produced in Example 4 as an analyte. The obtained sensorgrams were analyzed with BIA evaluation software, and the dissociation constants (KD values) were thus calculated. The results obtained are shown in Table 3.

TABLE 3

| | ka (1/Ms) | kd (1/s) | KD (M) |
|---|---|---|---|
| MOG01 | 1.4E+05 | 9.5E−04 | 6.6E−09 |
| MOG09 | 9.0E+03 | 1.9E−07 | 2.1E−11 |
| MOG14 | 4.7E+03 | 1.9E−04 | 4.0E−08 |
| iMOG-3Rim1-S32 | 1.4E+06 | 8.4E−04 | 6.2E−10 |

As shown in Table 3, the dissociation constants (KD values) of the anti-MOG antibodies were 2.1×10⁻¹¹ (M) to 4.0×10⁻⁸ (M), and it was elucidated that all the antibodies show excellent affinity. The dissociation rate constant kd of MOG09 antibody was outside the measurement range of the device, and the KD value could not be determined as a unique value.

### [Example 9] Evaluation of Rat Brain Migration Properties of Anti-MOG Antibodies

An antibody was administered to the tail vein (i.v.) of a rat, and then blood was collected from the tail vein. On the same day of the blood collection, the brain tissues were collected after whole body perfusion under anesthesia with pentobarbital, and the weight was measured. A buffer solution was added to the collected brain tissues, and the brain tissues were homogenized. After centrifugation, the antibody solution eluted in the supernatant was collected. While the volume was measured, the antibody concentration was measured with AlphaLISA (manufactured by PerkinElmer, Inc.). The antibody amount per unit brain weight was calculated.

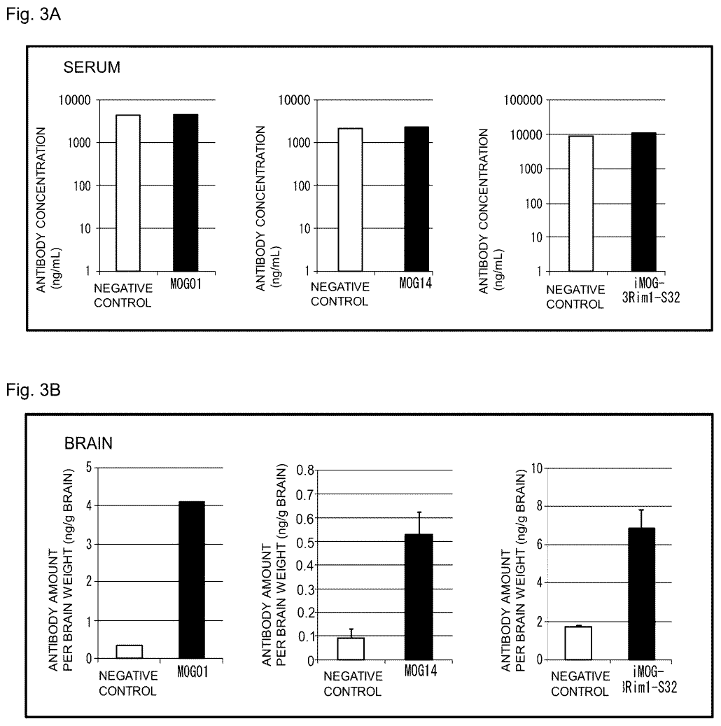

With respect to the anti-MOG antibodies, MOG01 antibody, MOG14 antibody and iMOG-3Rim1-S32 antibody and the anti-AVM antibody as a negative control, MOG01 antibody and MOG14 antibody were administered at an amount of 1 mg/kg body weight, and iMOG-3Rim1-S32 antibody was administered at an amount of 5 mg/kg body weight. The antibody concentrations in the serum and the antibody amounts per unit brain weight in the brain tissues four days after the administration of the antibodies are shown in FIGS. 3A and 3B.

As shown in FIGS. 3A and 3B, it was shown that the antibody concentrations in the serum of all the anti-MOG antibodies did not change as compared to that of the negative control (AVM) but that the antibody amounts in the brain increased to 5-10 times.

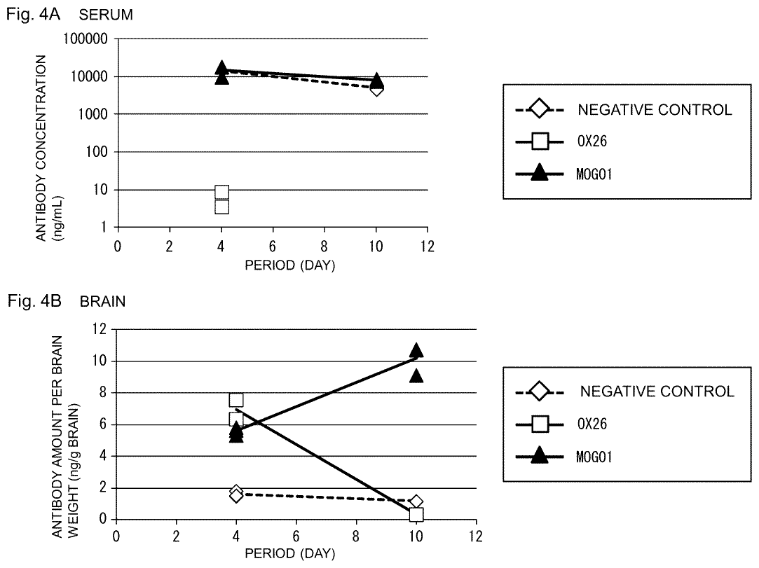

With respect to the anti-MOG antibody, MOG01 antibody, the anti-transferrin receptor antibody, OX26 antibody and the anti-AVM antibody as a negative control, the antibody concentrations in the serum and the antibody amounts per unit brain weight in the brain tissues four days and 10 days after the administration of the antibodies at an amount of 5 mg/kg body weight are shown in FIGS. 4A and 4B.

As shown in FIG. 4A, the antibody concentration of OX26 antibody in the serum was the lowest of the evaluated antibodies after four days and was equal to or lower than the detection sensitivity after 10 days, and thus the dynamics of the antibody in the blood was poor. The antibody concentration in the serum of MOG01 antibody, which is an anti-MOG antibody, did not change largely four days and 10 days after the administration, and the antibody concentration was equivalent to that of the negative control. This suggests that the half-life of MOG01 antibody in the blood is equivalent to that of the negative control.

Moreover, as shown in FIG. 4B, with respect to the antibody amounts in the brain, the antibody amount of the negative control was the lowest of the evaluated antibodies four days after the administration, and the antibody amount further decreased after 10 days although it was a slight decrease.

The antibody amount of OX26 antibody rapidly decreased between four days and 10 days after the administration, and the antibody amount 10 days after the administration was not higher than that of the negative control. On the other hand, the antibody amount of MOG01 antibody increased between four days and 10 days after the administration. The antibody amount four days after the administration was about 2.5 times the amount of the negative control, and the antibody amount 10 days after the administration was about 10 times the amount of the negative control.

The above results show that, while the anti-MOG antibody, MOG01 antibody shows an antibody concentration equal to that of the negative control in the serum, MOG01 antibody can increase its amount in the brain to about 2.5 times the amount of the negative control four days after the administration and to about 10 times the amounts of the negative control and OX26 antibody 10 days after the administration.

### [Example 10] Evaluation of Affinities of Bispecific Antibodies of MOG to MOG or HER2 Using Flow Cytometer

Binding to MOG or HER2 of the bispecific antibody binding to MOG and Her2, Trastuzumab IgG4PE(R409K)_MOG01 dscFv antibody, the bispecific antibody binding to MOG and AVM, AVM IgG4PE(R409K)_MOG01 dscFv antibody and the antibody binding to AVM, AVM IgG4PE(R409K)_AVM dscFv antibody obtained in Example 6 was evaluated by the fluorescence activated cell sorting (FACS) method according to the following procedures.

The membrane MOG antigen expression vectors produced in Example 5 were introduced to suspension 293 cells using FreeStyle (trademark) 293Expression System (manufactured by Thermo Fisher Scientific Inc.), and the cells were cultured to express the membrane antigens in a transient expression system.

HEK293F cells, rMOG/HEK293F cells, hMOG/HEK293F cells and human breast cancer cell line SK-BR-3 cells were suspended in a Staining Buffer (SB) of PBS containing 0.1% NaN₃ and 1% FBS each at a concentration of 5×10⁵ cells/mL and dispensed to a round-bottom 96-well plate (manufactured by Becton Dickinson).

After centrifugation (2000 rpm, 4° C., two minutes), the supernatants were removed, and the antibodies obtained in Example 6 at 10 μg/mL were added to the pellets. After suspending the pellets, the plate was left to stand at ice temperature for 30 minutes. The supernatants were removed after further centrifugation (2000 rpm, 4° C., two minutes), and the pellets were washed with SB. Then, 1 μg/mL RPE fluorescently labeled goat anti-human antibody (manufactured by Southern Bioblot) was added, and the plate was incubated at ice temperature for 30 minutes.

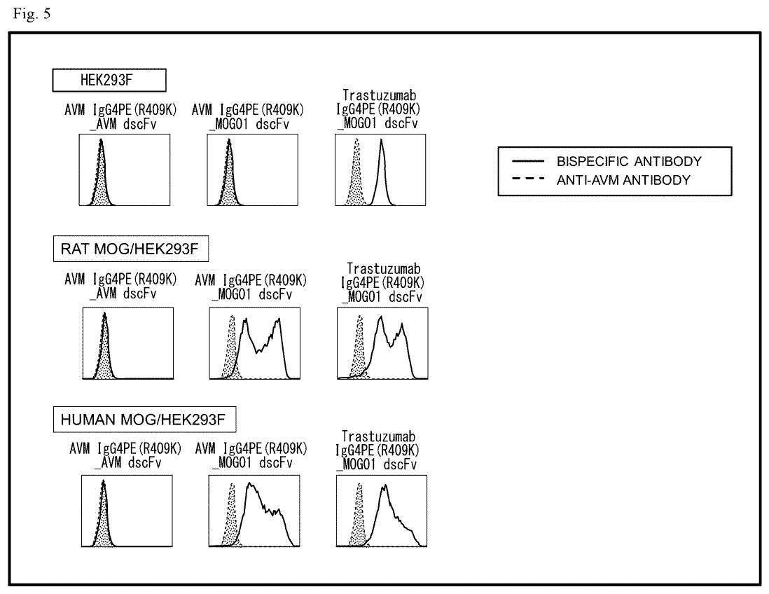

After washing with SB, the cells were suspended in SB, and the fluorescence intensities of the cells were measured with a flow cytometer FACS CANTO II (manufactured by Becton Dickinson). As the negative control, 10 μg/mL anti-AVM antibody was used. The results of the analysis of the affinities to HEK293F cells, rMOG/HEK293F cells and hMOG/HEK293 cells are shown in FIG. 5.

From FIG. 5, binding of AVM IgG4PE(R409K)_MOG01 dscFv antibody and Trastuzumab IgG4PE(R409K)_MOG01 dscFv antibody to rMOG/HEK293F cells and hMOG/HEK293 cells can be observed. Thus, it was shown that the antibodies maintain the affinities to rat MOG and human MOG also in the form of bispecific antibody.

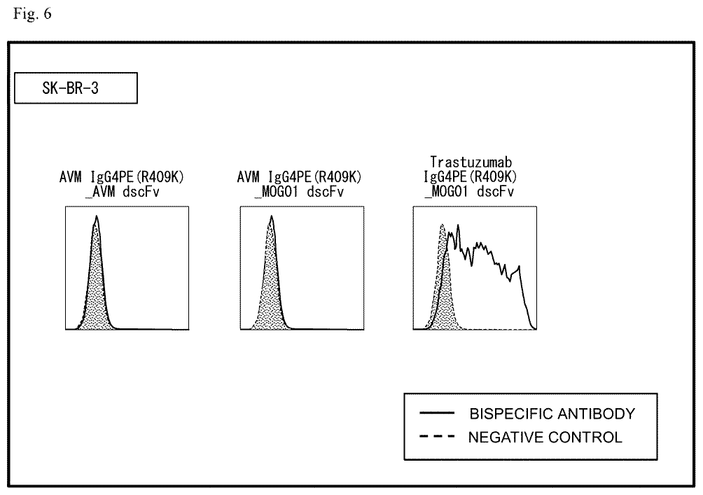

The results of the analysis of the affinities to human breast cancer cell line SK-BR-3 cells are shown in FIG. 6. It is known that HER2 is expressed in the cells.

From FIG. 6, it was shown that Trastuzumab IgG4PE(R409K)_MOG01 dscFv antibody maintains the affinity to HER2 also in the form of bispecific antibody.

### [Example 11] Evaluation of Affinities of Bispecific Antibodies of MOG to MOG by Surface Plasmon Resonance Detection

The affinities of the bispecific antibodies of MOG to MOG were measured by the same method as that of Example 8, and the results are shown in Table 4.

TABLE 4

| Antibody Name | ka (1/Ms) | kd (1/s) | KD (M) |
|---|---|---|---|
| AVM IgG4PE(R409K)_MOG01 dscFv | 2.3E+04 | 4.6E−03 | 2.0E−07 |
| Trastuzumab IgG4PE(R409K)_MOG01 | 2.4E+08 | 2.5E+01 | 1.0E−07 |
| dscFv | | | |

As shown in Table 4, the dissociation constant (KD value) of the bispecific antibody, AVM IgG4PE(R409K)_MOG01 dscFv antibody was 2.0×10⁻⁷ (M), and that of Trastuzumab IgG4PE(R409K)_MOG01 dscFv antibody was 1.0×10⁻⁷ (M). It was elucidated that both bispecific antibodies of MOG show excellent affinity.

The association rate constant ka and the dissociation rate constant kd of Trastuzumab IgG4PE(R409K)_MOG01 dscFv antibody were outside the measurement ranges of the device, and the KD value could not be determined as a unique value.

### [Example 12] Evaluation of Affinity of Bispecific Antibody of MOG to HER2 by Surface Plasmon Resonance Detection

The affinity of the bispecific antibody binding to MOG and HER2, Trastuzumab IgG4PE(R409K)_MOG01 dscFv antibody to HER2 was measured using Biacore T-100 (GE Healthcare).

The antibody was immobilized on a CM5 sensor chip using a Human antibody Capture kit, and the binding ability of the MOG-Her2 bispecific antibody was evaluated using HER2-GST produced in Example 4 as an analyte. The obtained sensorgram was analyzed with BIA evaluation software, and the dissociation constant (KD value) was thus calculated. The results are shown in Table 5.

TABLE 5

| Antibody Name | ka (1/Ms) | kd (1/s) | KD (M) |
|---|---|---|---|
| Trastuzumab IgG4PE(R409K)_MOG01 | 4.7E+04 | 1.8E−04 | 3.7E−09 |
| dscFv | | | |

As shown in Table 5, the dissociation constant (KD value) of Trastuzumab IgG4PE(R409K)_MOG01 dscFv antibody to HER2 was 3.7×10−⁹ (M), and it was elucidated that this is an antibody showing excellent affinity.

### [Example 13] Evaluation of Rat Brain Migration Properties of Bispecific Antibodies of MOG

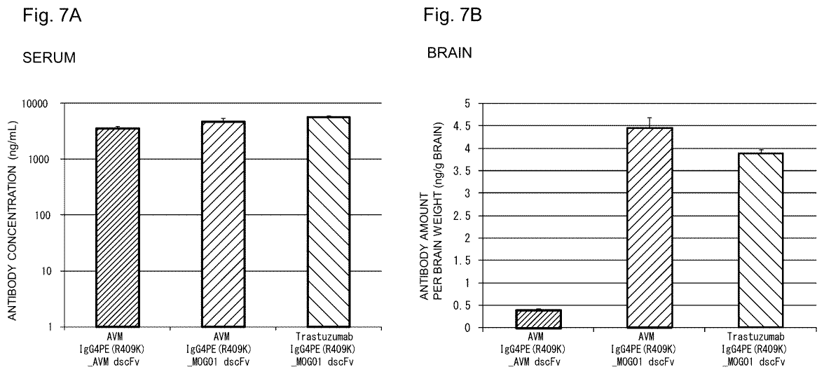

The rat brain migration properties of the bispecific antibodies, AVM IgG4PE(R409K)_MOG01 dscFv antibody, Trastuzumab IgG4PE(R409K)_MOG01 dscFv antibody and AVM IgG4PE(R409K)_AVM dscFv antibody was evaluated by the same method as that of Example 9. The antibody concentrations in the serum and the antibody amounts per unit brain weight in the brain tissues 10 days after the administration of the antibodies at an amount of 5 mg/kg body weight are shown in FIGS. 7A and 7B.

As shown in FIG. 7A, as compared to AVM IgG4PE(R409K)_AVM dscFv antibody, which is the negative control of the bispecific antibodies, the antibody concentrations in the serum of AVM IgG4PE(R409K)_MOG01 dscFv antibody and Trastuzumab IgG4PE(R409K)_MOG01 dscFv antibody were not different.

On the other hand, as shown in FIG. 7B, it was shown that, as compared to AVM IgG4PE(R409K)_AVM dscFv antibody, which is the negative control of the bispecific antibodies, the antibody amounts of AVM IgG4PE(R409K)_MOG01 dscFv antibody and Trastuzumab IgG4PE(R409K)_MOG01 dscFv antibody in the brain increased to about 10 times.

The above results show that while the bispecific antibodies which bind to MOG can increase the antibody amount in the brain to about 10 times the value of the bispecific antibody which does not bind to MOG, the half-lives in the blood do not change.

### [Example 14] Evaluation of Mouse Brain Migration Property of Anti-MOG01 Antibody

### (1) Measurement of Antibody Amount

Several days after administering the antibody to the tail vein (i.v.) of a mouse at 35 nmol/kg, blood was collected from the tail vein. On the same day of the blood collection, the brain tissues were collected after whole body perfusion under anesthesia with pentobarbital, and the weight was measured. A buffer solution was added to the collected brain tissues, and the brain tissues were homogenized. After centrifugation, the antibody solution eluted in the supernatant was collected. While the volume was measured, the antibody concentration was measured with AlphaLISA (manufactured by PerkinElmer, Inc.). The antibody amount per unit brain weight was calculated.

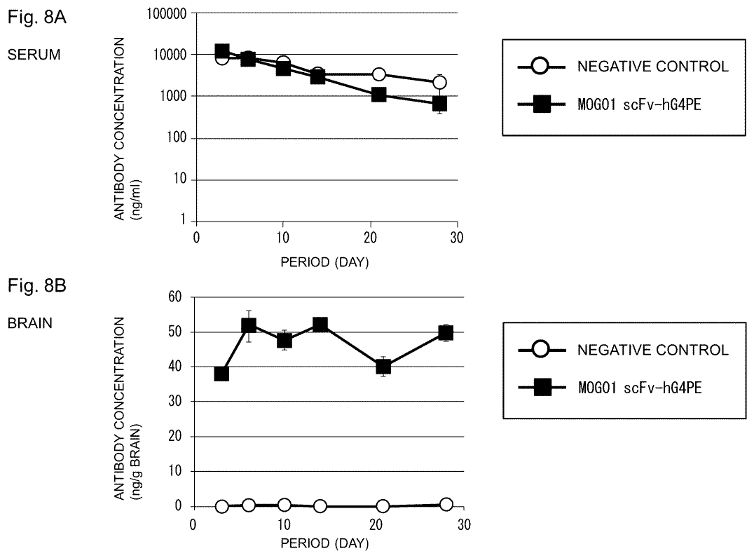

With respect to the anti-MOG01 human IgG antibody and an anti-AVM human IgG antibody as the negative control, the antibody concentrations in the serum and the antibody amounts per unit brain weight in the brain tissues 3, 6, 10, 14, 21 and 28 days after the administration of the antibodies are shown in FIGS. 8A and 8B, respectively.

As shown in FIG. 8A, the antibody concentrations of the anti-MOG01 human IgG antibody in the serum were not different from those of the negative control. On the other hand, as shown in FIG. 8B, it was shown that the antibody amount in the brain can be increased to several ten times over 28 days.

### (2) Imaging Analysis

The anti-MOG01 human IgG antibody and the anti-AVM human IgG antibody as the negative control were labeled with Alexa FluorR 488 Protein Labeling Kit (manufactured by Molecular Probes). The labeled antibodies are referred to as AF488-MOG01 IgG4PE antibody and AF488-AVM IgG4PE antibody.

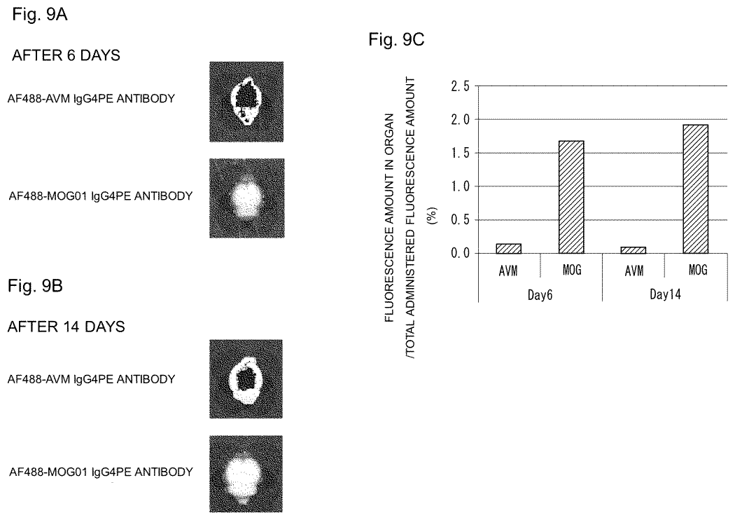

Several days after administering the labeled antibodies to the tail veins (i.v.) of mice at 10 mg/kg, Tomato lectin was administered, and the blood was collected from the cheeks of the mice. The brain tissues were collected after the blood collection and after whole body perfusion under anesthesia with pentobarbital, and the fluorescence intensities were measured with IVIS Spectrum (manufactured by PerkinElmer, Inc.). The brain images after six days are shown in FIG. 9A, and the brain images after 14 days are shown in FIG. 9B. The fluorescence amounts in the brain corrected with the fluorescence intensities of the administered antibodies are shown in FIG. 9C.

As shown in FIGS. 9A to 9C, it was shown that the anti-MOG01 antibody can increase the antibody amount to several ten times over the entire brain as compared to the negative control.

### [Example 15] Construction of Bispecific Antibody Expression Vectors

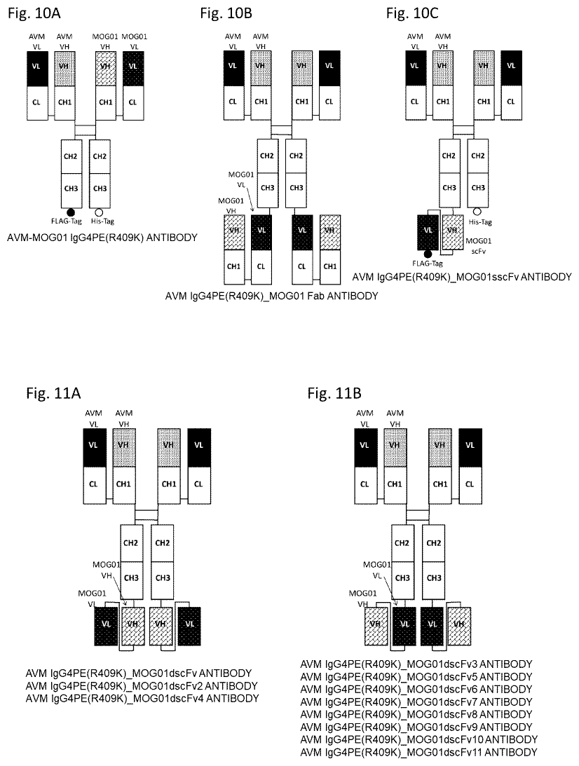

Vectors expressing bispecific antibodies which have any of the structures described in FIGS. 10A to 10C and FIGS. 11A and 11B and which bind to AVM and MOG were produced by the following method. The names of the bispecific antibodies and the names of the antibody expression vectors are shown in Table 6, and the names of the antibody expression vectors, the nucleotide sequences of the antibodies and the amino acid sequences deduced from the nucleotide sequences are shown in Table 7.

TABLE 6

| Name of Bispecific Antibody | Name of Antibody Expression Vector |
|---|---|
| AVM-MOG01 | pCI-AVM-hLG4PE(R409K/ |
| IgG4PE(R409K) antibody | S354C/T366W)-FLAG tag |
| | pCI-MOG01-hLG4PE(R409K/Y349C/ |
| | T366S/L368A/Y407V)-His tag |
| AVM IgG4PE(R409K)_ | pCI-AVM-hLG4PE(R409K)- |
| MOG01 Fab antibody | linker-MOG01VL-CL |
| | pCI-MOG01VH-CH |
| AVM IgG4PE(R409K)_ | pCI-AVM-hLG4PE(R409K/S354C/ |
| MOG01 sscFv antibody | T366W)-linker-MOG01scFv-FLAG tag |
| | pCI-AVM-hLG4PE(R409K/Y349C/ |
| | T366S/L368A/Y407V)-His tag |
| AVM IgG4PE(R409K)_ | pCI-AVM-hLG4PE(R409K)_ |
| MOG01dscFv2 antibody | MOG01scFv2 |
| AVM IgG4PE(R409K)_ | pCI-AVM-hLG4PE(R409K)_ |
| MOG01dscFv3 antibody | MOG01scFv3 |
| AVM IgG4PE(R409K)_ | pCI-AVM-hLG4PE(R409K)_ |
| MOG01dscFv4 antibody | MOG01scFv4 |
| AVM IgG4PE(R409K)_ | pCI-AVM-hLG4PE(R409K)_ |
| MOG01dscFv5 antibody | MOG01scFv5 |
| AVM IgG4PE(R409K)_ | pCI-AVM-hLG4PE(R409K)_ |
| MOG01dscFv6 antibody | MOG01scFv6 |
| AVM IgG4PE(R409K)_ | pCI-AVM-hLG4PE(R409K)_ |
| MOG01dscFv7 antibody | MOG01scFv7 |
| AVM IgG4PE(R409K)_ | pCI-AVM-hLG4PE(R409K)_ |
| MOG01dscFv8 antibody | MOG01scFv8 |
| AVM IgG4PE(R409K)_ | pCI-AVM-hLG4PE(R409K)_ |
| MOG01dscFv9 antibody | MOG01scFv9 |
| AVM IgG4PE(R409K)_ | pCI-AVM-hLG4PE(R409K)_ |
| MOG01dscFv10 antibody | MOG01scFv10 |
| AVM IgG4PE(R409K)_ | pCI-AVM-hLG4PE(R409K)_ |
| MOG01dscFv11 antibody | MOG01scFv11 |

TABLE 7

| | | Nucleotide Sequence | |
|---|---|---|---|
| | | of Heavy Chain | Amino Acid Sequence of |
| | Light Chain | Antibody Sequence | Heavy Chain Antibody |
| | Antibody | (excluding signal | Sequence (excluding |
| Name of Antibody Expression Vector | Sequence | sequence) | signal sequence) |
| pCI-AVM-hLG4PE(R409K/S354C/T366W)-FLAG tag | AVM | SEQ ID NO: 108 | SEQ ID NO: 109 |
| pCI-MOG01-hLG4PE(R409K/Y349C/ | MOG01 | SEQ ID NO: 110 | SEQ ID NO: 111 |
| T366S/L368A/Y407V)-His tag | | | |
| pCI-AVM-hLG4PE(R409K)-linker-MOG01VL-CL | AVM | SEQ ID NO: 112 | SEQ ID NO: 113 |
| pCI-MOG01VH-CH | None | SEQ ID NO: 114 | SEQ ID NO: 115 |
| pCI-AVM-hLG4PE(R409K/S354C/T366W)- | AVM | SEQ ID NO: 116 | SEQ ID NO: 117 |
| linker-MOG01scFv-FLAG | | | |
| pCI-AVM-hLG4PE(R409K/Y349C/T366S/ | AVM | SEQ ID NO: 118 | SEQ ID NO: 119 |
| L368A/Y407V)-His tag | | | |
| pCI-AVM-hLG4PE(R409K)_MOG01scFv2 | AVM | SEQ ID NO: 120 | SEQ ID NO: 121 |
| pCI-AVM-hLG4PE(R409K)_MOG01scFv3 | AVM | SEQ ID NO: 122 | SEQ ID NO: 123 |
| pCI-AVM-hLG4PE(R409K)_MOG01scFv4 | AVM | SEQ ID NO: 124 | SEQ ID NO: 125 |
| pCI-AVM-hLG4PE(R409K)_MOG01scFv5 | AVM | SEQ ID NO: 126 | SEQ ID NO: 127 |
| pCI-AVM-hLG4PE(R409K)_MOG01scFv6 | AVM | SEQ ID NO: 128 | SEQ ID NO: 129 |
| pCI-AVM-hLG4PE(R409K)_MOG01scFv7 | AVM | SEQ ID NO: 130 | SEQ ID NO: 131 |
| pCI-AVM-hLG4PE(R409K)_MOG01scFv8 | AVM | SEQ ID NO: 132 | SEQ ID NO: 133 |
| pCI-AVM-hLG4PE(R409K)_MOG01scFv9 | AVM | SEQ ID NO: 134 | SEQ ID NO: 135 |
| pCI-AVM-hLG4PE(R409K)_MOG01scFv10 | AVM | SEQ ID NO: 136 | SEQ ID NO: 137 |
| pCI-AVM-hLG4PE(R409K)_MOG01scFv11 | AVM | SEQ ID NO: 138 | SEQ ID NO: 139 |

### (1) Construction of Bispecific Antibody Expression Vectors Related to Structure in FIG. 10A

(1-1) Construction of pCI-AVM-hLG4PE(R409K/S354C/T366W)-FLAG tag Vector

A gene fragment of the CH1-Hinge-CH2-CH3(R409K/S354C/T366W) region was amplified by PCR using a synthetic gene as a template and inserted to the NheI-BamHI site of pCI-AVM-hLG4PE(R409K)_AVMscFv, and pCI-AVM-hLG4PE(R409K/S354C/T366W)-FLAG tag vector was produced.

(1-2) Construction of pCI-MOG01-hLG4PE(R409K/Y349C/T366S/L368A/Y407V)-His Tag Vector

A gene fragment of the CH1-Hinge-CH2-CH3(R409K/Y349C/T366S/L368A/Y407V)-His tag region was amplified by PCR using a synthetic gene as a template. Moreover, a gene fragment of the MOG01 light chain region and a gene fragment of the MOG01 VH region were amplified by PCR using N5LG4PE_MOG01 as a template. The obtained gene fragments were inserted to pCI vector (manufactured by Promega Corporation), and pCI-MOG01-hLG4PE(R409K/Y349C/T366S/L368A/Y407V)-His tag vector was produced.

### (2) Construction of Bispecific Antibody Expression Vectors Related to Structure in FIG. 10B

(2-1) Construction of pCI-AVM-hLG4PE(R409K)-Linker-MOG01VL-CL Vector

A gene fragment of the CH1-Hinge-CH2-CH3-linker-MOG01VL-CL region was amplified by PCR using a synthetic gene as a template and inserted to the NheI-BamHI site of pCI-AVM-hLG4PE(R409K)_AVMscFv, and pCI-AVM-hLG4PE(R409K)-linker-MOG01VL-CL vector was produced.

(2-2) Construction of pCI-MOG01VH-CH Vector

A gene fragment of the MOG01VH-CH region was amplified by PCR using a synthetic gene as a template and inserted to pCI vector (manufactured by Promega Corporation), and pCI-MOG01VH-CH vector was produced.

### (3) Construction of Bispecific Antibody Expression Vectors Related to Structure in FIG. 10C

(3-1) Construction of pCI-AVM-hLG4PE(R409K/S354C/T366W)-Linker-MOG01scFv-FLAG Tag Vector

A gene fragment of the CH1-Hinge-CH2-CH3(R409K/S354C/T366W)-linker region was amplified by PCR using a synthetic gene as a template. Moreover, a gene fragment of the linker-MOG01scFv region was amplified by PCR using MOG01scFv as a template. Furthermore, a gene fragment of the linker-MOG01scFv-FLAG tag region was amplified by PCR using the PCR product as a template. The gene fragments of the CH1-Hinge-CH2-CH3(R409K/S354C/T366W)-linker region and the linker-MOG01scFv-FLAG tag region were inserted to the NheI-BamHI site of pCI-AVM-hLG4PE(R409K)_AVM scFv, and pCI-AVM-hLG4PE(R409K/S354C/T366W)-linker-MOG01scFv-FLAG tag vector was produced.

(3-2) Construction of pCI-AVM-hLG4PE(R409K/Y349C/T366S/L368A/Y407V)-His Tag Vector

A gene fragment of the CH1-Hinge-CH2-CH3(R409K/Y349C/T366S/L368A/Y407V)-His tag region was amplified by PCR using a synthetic gene as a template and inserted to the NheI-BamHI site of pCI-AVM-hLG4PE(R409K)_AVMscFv, and pCI-AVM-hLG4PE(R409K/Y349C/T366S/L368A/Y407V)-His tag vector was produced.

### (4) Construction of Vectors Expressing Bispecific Antibodies Having Structures in FIGS. 11A and 11B

(4-1) Construction of pCI-AVM-hLG4PE(R409K)_MOG01scFv Vector

A gene fragment of the CH1-Hinge-CH2-CH3-linker region was amplified by PCR using a synthetic gene as a template. Moreover, a gene fragment of the VH region and the VL region of MOG01 was amplified by PCR using MOG01scFv as a template. The gene fragment of the CH1-Hinge-CH2-CH3-linker region and the gene fragment of the VH region and the VL region of MOG01 were inserted to the NheI-BamHI site of pCI-AVM-hLG4PE(R409K)_AVMscFv, and pCI-AVM-hLG4PE(R409K)_MOG01scFv2 vector was produced.

In the same manner, pCI-AVM-hLG4PE(R409K)_MOG01scFv3 vector, pCI-AVM-hLG4PE(R409K)_MOG01scFv4 vector, pCI-AVM-hLG4PE(R409K)_MOG01scFv5 vector, pCI-AVM-hLG4PE(R409K)_MOG01scFv6 vector, pCI-AVM-hLG4PE(R409K)_MOG01scFv7 vector, pCI-AVM-hLG4PE(R409K) MOG01scFv8 vector, pCI-AVM-hLG4PE(R409K)_MOG01scFv9 vector, pCI-AVM-hLG4PE(R409K)_MOG01scFv10 vector and pCI-AVM-hLG4PE(R409K)_MOG01scFv11 vector were produced.

### (5) Construction of Vectors Expressing Antibodies as Negative Controls

The antibodies as the negative controls were produced by the following method. The names of the antibodies and the names of the antibody expression vectors are shown in Table 8, and the names of the antibody expression vectors, the nucleotide sequences of the antibodies and the amino acid sequences deduced from the nucleotide sequences are shown in Table 9.

TABLE 8

| Name of Negative | Name of Antibody |
|---|---|
| Control Antibody | Expression Vector |
| AVM IgG4PE(R409K) antibody | pCI-AVM-hLG4PE(R409K) |
| AVM IgG4PE(R409K)_AVM | pCI-AVM-hLG4PE(R409K)- |
| Fab antibody | linker-AVMVL-CL |
| | pCI-AVMVH-CH |
| AVM IgG4PE(R409K)_ | pCI-AVM-hLG4PE(R409K/ |
| AVMsscFv antibody | S354C/T366W)-linker- |
| | AVMscFv-FLAG tag |
| | pCI-AVM-hLG4PE(R409K/ |
| | Y349C/T366S/L368A/ |
| | Y407V)-His tag |
| AVM IgG4PE(R409K)_ | pCI-AVM-hLG4PE(R409K)_ |
| AVMdscFv3 antibody | AVMscFv3 |
| AVM IgG4PE(R409K)_ | pCI-AVM-hLG4PE(R409K)_ |
| AVMdscFv5 antibody | AVMscFv5 |

TABLE 9

| | | Nucleotide | Amino Acid |
|---|---|---|---|
| | | Sequence of | Sequence of |
| | | Heavy Chain | Heavy Chain |
| | | Antibody | Antibody |
| | Light | Sequence | Sequence |
| | Chain | (excluding | (excluding |
| Name of Antibody | Antibody | signal | signal |
| Expression Vector | Sequence | sequence) | sequence) |
| pCI-AVM-hLG4PE(R409K)- | AVM | SEQ ID | SEQ ID |
| linker-AVMVL-CL | | NO: 140 | NO: 141 |
| pCI-AVMVH-CH | None | SEQ ID | SEQ ID |
| | | NO: 142 | NO: 143 |
| pCI-AVM- | AVM | SEQ ID | SEQ ID |
| hLG4PE(R409K/S354C/ | | NO: 144 | NO: 145 |
| T366W)-linker- | | | |
| AVMscFv-FLAG tag | | | |
| pCI-AVM-hLG4PE(R409K)_ | AVM | SEQ ID | SEQ ID |
| AVMscFv3 | | NO: 146 | NO: 147 |
| pCI-AVM-hLG4PE(R409K)_ | AVM | SEQ ID | SEQ ID |
| AVMscFv5 | | NO: 148 | NO: 149 |

(2-1) Production of pCI-AVM-hLG4PE(R409K) Vector

A gene fragment of the VH region, the VL region and the antibody constant region of AVM was amplified by PCR using a synthetic gene as a template and inserted to pCI vector (manufactured by Promega Corporation), and pCI-AVM-hLG4PE(R409K) vector was produced.

(2-2) Production of pCI-AVM-hLG4PE(R409K)-Linker-AVMVL-CL Vector

A gene fragment of the CH1-Hinge-CH2-CH3-linker-AVMVL-CL region was amplified by PCR using a synthetic gene as a template and inserted to the NheI-BamHI site of pCI-AVM-hLG4PE(R409K)_AVM scFv, and pCI-AVM-hLG4PE(R409K)-linker-AVMVL-CL vector was produced.

(2-3) Construction of pCI-AVMVH-CH Vector

A gene fragment of the AVMVH-CH region was amplified by PCR using a synthetic gene as a template and inserted to pCI vector (manufactured by Promega Corporation), and pCI-AVMVH-CH vector was produced.

(2-4) Construction of pCI-AVM-hLG4PE(R409K/S354C/T366W)-Linker-AVMscFv-FLAG Tag Vector

A gene fragment of the CH1-Hinge-CH2-CH3(R409K/S354C/T366W)-linker region was amplified by PCR using a synthetic gene as a template. Moreover, a gene fragment of the linker-AVMscFv-FLAG tag region was amplified by PCR using N5LG4PE_AVM as a template. The gene fragments of the CH1-Hinge-CH2-CH3(R409K/S354C/T366W)-linker region and the linker-AVMscFv-FLAG tag region were inserted to the NheI-BamHI site of pCI-AVM-hLG4PE(R409K)_AVM scFv, and pCI-AVM-hLG4PE(R409K/S354C/T366W)-linker-AVMscFv-FLAG tag vector was produced.

(2-5) Construction of pCI-AVM-hLG4PE(R409K)_AVMscFv Vector

A gene fragment of the CH1-Hinge-CH2-CH3-linker region was amplified by PCR using a synthetic gene as a template. Moreover, a gene fragment of the VH region and the VL region of AVM was amplified by PCR using N5LG4PE_AVM as a template. The gene fragment of the CH1-Hinge-CH2-CH3-linker region and the gene fragment of the VH region and the VL region of AVM were inserted to the NheI-BamHI site of pCI-AVM-hLG4PE(R409K)_AVMscFv, and pCI-AVM-hLG4PE(R409K)_AVMscFv3 vector and pCI-AVM-hLG4PE(R409K)_AVMscFv5 vector were produced.

### [Example 16] Preparation of Bispecific Antibodies

By the method described in Example 6, AVM IgG4PE(R409K)_MOG01 Fab antibody, AVM IgG4PE(R409K)_MOG01dscFv2 antibody, AVM IgG4PE(R409K)_MOG01dscFv3 antibody, AVM IgG4PE(R409K)_MOG01dscFv4 antibody, AVM IgG4PE(R409K)_MOG01dscFv5 antibody, AVM IgG4PE(R409K)_MOG01dscFv6 antibody, AVM IgG4PE(R409K) MOG01dscFv7 antibody, AVM IgG4PE(R409K)_MOG01dscFv8 antibody, AVM IgG4PE(R409K)_MOG01dscFv9 antibody, AVM IgG4PE(R409K)_MOG01dscFv10 antibody, AVM IgG4PE(R409K)_MOG01dscFv11 antibody, AVM IgG4PE(R409K)_AVM Fab antibody, AVM IgG4PE(R409K)_AVMdscFv3 antibody and AVM IgG4PE(R409K)_AVMdscFv5 antibody were prepared.

AVM-MOG01 IgG4PE(R409K) antibody, AVM IgG4PE(R409K)_MOG01sscFv antibody and AVM IgG4PE(R409K)_AVMsscFv antibody were prepared by the method described below. The antibody expression plasmid vectors were introduced to suspension 293 cells using Expi293 (trademark) Expression System (manufactured by Thermo Fisher Scientific Inc.), and the cells were cultured to express the antibodies in a transient expression system.

The culture supernatants were collected four days after the introduction of the vectors and filtered through a membrane filter having a pore size of 0.22 μm (manufactured by Millipore Corporation). The proteins in the culture supernatants were affinity-purified with His tag using Ni Sepharose resin (manufactured by GE Healthcare BioSciences). A 20 mM Imidazole-phosphate buffer solution was used as a washing solution.

The antibodies adsorbed on the Ni Sepharose resin were eluted with a 500 mM Imidazole-phosphate buffer solution. Next, the solvents of the eluates were replaced with PBS by ultrafiltration using VIVASPIN (manufactured by Sartrius stealin) and a NAP column (manufactured by GE Healthcare BioSciences).

The proteins after the His tag purification were affinity-purified using FLAG antibody affinity gel (manufactured by Sigma-Aldrich Co. LLC.). A phosphate buffer solution was used as a washing solution. The antibodies adsorbed on the FLAG antibody affinity gel were eluted with 20 mM sodium citrate and 50 mM NaCl buffer solution (pH 3.4) and collected in tubes containing 1 M Tris-HCl Buffer Solution (pH 8.0).

Next, the solvents of the eluates were replaced with PBS by ultrafiltration using VIVASPIN (manufactured by Sartrius stealin) and a NAP column (manufactured by GE Healthcare BioSciences), and then filtration sterilization with a membrane filter having a pore size of 0.22 um (Millex-GV, manufactured by Millipore Corporation) was conducted. The absorbances of the antibody solutions at 280 nm were measured, and the concentrations of the purified antibodies were calculated.

### [Example 17] Evaluation of Affinities of Bispecific Antibodies to MOG Using Flow Cytometer

Binding of the bispecific antibodies and the negative control antibodies obtained in Example 6 and Example 16 to MOG was evaluated by the fluorescence activated cell sorting (FACS) method according to the following procedures.

pEF6_hMOG obtained in Example 5 was introduced to mouse connective tissue-derived fibroblast L929 cells [American Type Culture Collection (ATCC) No.: CCL-1] using HilyMax (manufactured by Dojindo Laboratories). The gene-transfected cells were selected using an antibiotic substance, Blasticidin (manufactured by Invitrogen) and then cloned by the limiting dilution method. Using the L929 cells expressing hMOG on the cell surface (abbreviated as hMOG/L929 below), the reactivities of the bispecific antibodies were analyzed by the method described below.

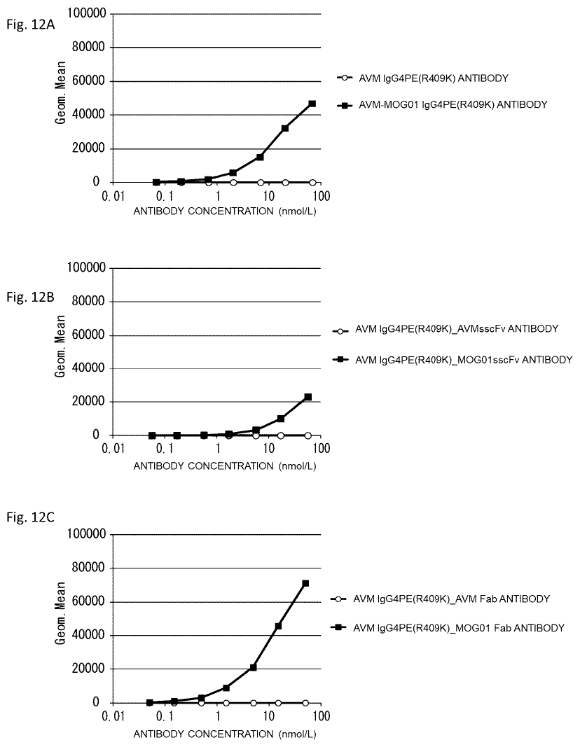

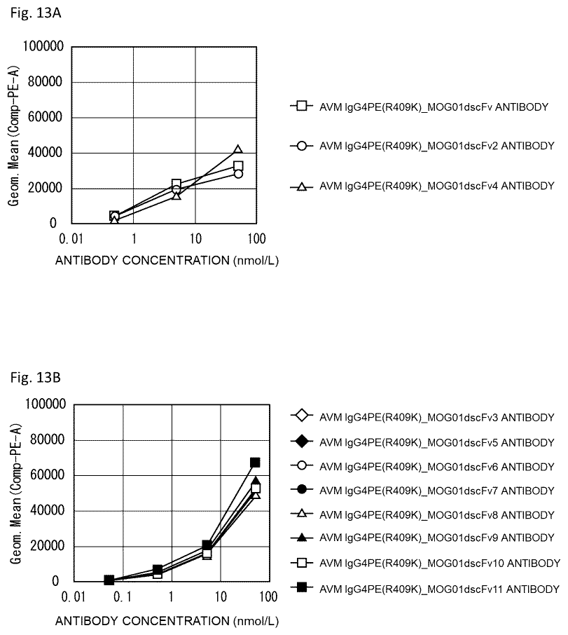

The hMOG/L929 cells were suspended in a Staining Buffer (SB) of PBS containing 0.1% NaN₃ and 1% FBS and dispensed to a round-bottom 96-well plate (manufactured by Becton Dickinson). After centrifugation (2000 rpm, 4° C., two minutes), the supernatants were removed, and the MOG01 bispecific antibodies obtained in Example 6 and Example 16 were added to the pellets. After suspending the pellets, the plate was left to stand at ice temperature for 30 minutes. The supernatants were removed after further centrifugation (2000 rpm, 4° C., two minutes), and the pellets were washed with SB. Then, 1 μg/mL RPE fluorescently labeled goat anti-human antibody (manufactured by Southern Biotech) was added, and the plate was incubated at ice temperature for 30 minutes. After washing with SB, the cells were suspended in SB, and the fluorescence intensities of the cells were measured with a flow cytometer FACS CANTO II (manufactured by Becton Dickinson). The results are shown in FIGS. 12A to 12C and FIGS. 13A and 13B.

As shown in FIGS. 12A to 12C and FIGS. 13A and 13B, it was confirmed that all the bispecific antibodies have affinity to MOG. In particular, it was elucidated that the affinities of AVM IgG4PE(R409K)_MOG01Fab antibody [FIG. 10B and FIG. 12C], AVM IgG4PE(R409K)_MOG01dscFv3 antibody, AVM IgG4PE(R409K)_MOG01dscFv5 antibody, AVM IgG4PE(R409K)_MOG01dscFv6 antibody, AVM IgG4PE(R409K)_MOG01dscFv7 antibody, AVM IgG4PE(R409K)_MOG01dscFv8 antibody, AVM IgG4PE(R409K)_MOG01dscFv9 antibody, AVM IgG4PE(R409K)_MOG01dscFv10 antibody and AVM IgG4PE(R409K)_MOG01dscFv11 antibody [FIG. 11B and FIG. 13B] are high.

### [Example 18] Evaluation of Affinities of Bispecific Antibodies to MOG by Surface Plasmon Resonance Detection

Binding of the bispecific antibodies obtained in Example 6 and Example 16 to MOG was evaluated by the same method as that of Example 8. The results obtained are shown in Table 10 and Table 11.

TABLE 10

| Antibody Name | ka (1/Ms) | kd (1/s) | KD (M) |
|---|---|---|---|
| AVM-MOG01 IgG4PE(R409K) | 1.3E+05 | 1.6E−03 | 1.2E−08 |
| antibody | | | |
| AVM IgG4PE(R409K)_MOG01sscFv | 5.1E+04 | 1.0E−02 | 2.0E−07 |
| antibody | | | |
| AVM IgG4PE(R409K)_MOG01 Fab | 1.0E+05 | 3.5E−03 | 3.4E−08 |
| antibody | | | |

TABLE 11

| Antibody Name | ka (1/Ms) | kd (1/s) | KD (M) |
|---|---|---|---|
| AVM IgG4PE(R409K)_MOG01dscFv | 2.4E+04 | 4.9E−03 | 2.0E−07 |
| antibody | | | |
| AVM IgG4PE(R409K)_MOG01dscFv3 | 1.7E+05 | 2.2E−03 | 1.3E−08 |
| antibody | | | |
| AVM IgG4PE(R409K)_MOG01dscFv5 | 2.0E+05 | 4.8E−03 | 2.4E−08 |
| antibody | | | |

As shown in Table 10 and Table 11, the dissociation constants (KD values) of the bispecific antibodies of MOG were 1.2×10−⁸ (M) to 2.0×10−⁷ (M), and it was elucidated that all the antibodies show excellent affinity.

In particular, it was elucidated that the affinities of AVM-MOG01 IgG4PE(R409K) antibody [FIG. 10A], AVM IgG4PE(R409K)_MOG01Fab antibody [FIG. 10B], AVM IgG4PE(R409K)_MOG01dscFv3 antibody and AVM IgG4PE(R409K)_MOG01dscFv5 antibody [FIG. 11B] are high.

### [Example 19] Evaluation of Mouse Brain Migration Properties of Bispecific Antibodies

The mouse brain migration properties of the bispecific antibodies and the negative control antibodies obtained in Example 6 and Example 16 were evaluated by the method of Example 14.

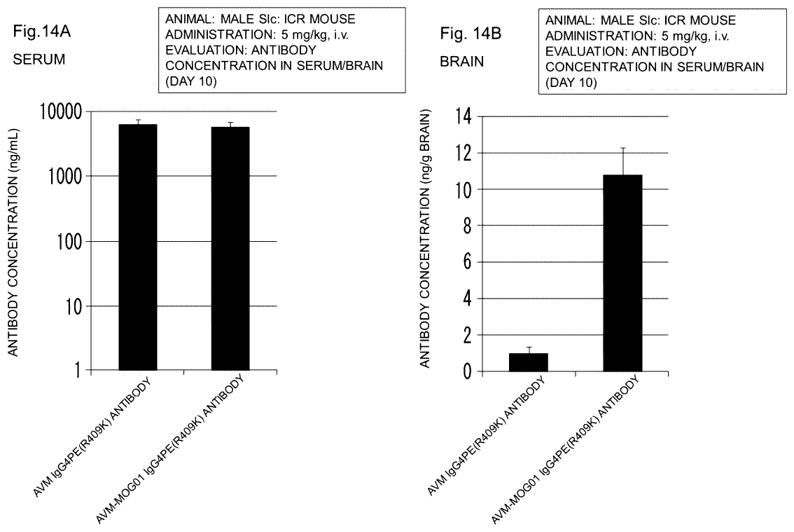

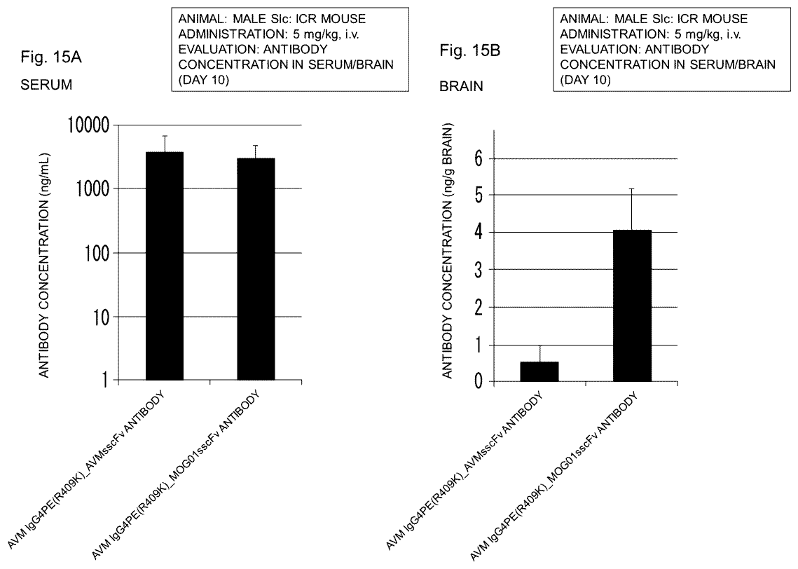

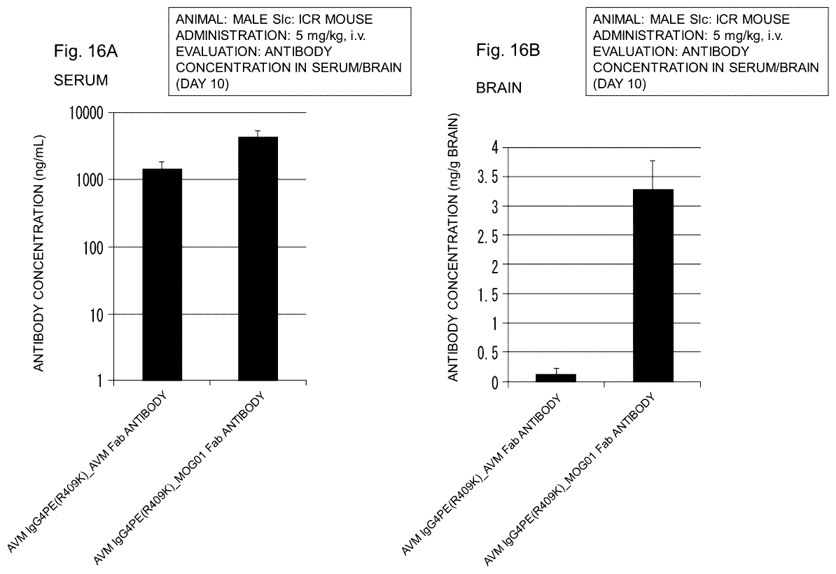

AVM-MOG01 IgG4PE(R409K) antibody, AVM IgG4PE(R409K)_MOG01sscFv antibody and AVM IgG4PE(R409K)_MOG01 Fab antibody were administered at 5 mg/kg, and the antibody concentrations in the serum and the antibody amounts per unit brain weight in the brain tissues after 10 days are shown in FIG. 14A to FIG. 16B.

As shown in FIG. 14A, FIG. 15A and FIG. 16A, the antibody concentrations of all the MOG01 modified antibodies in the serum were not different from those of the negative controls. On the other hand, as shown in FIG. 14B, FIG. 15B and FIG. 16B, it was shown that the antibody amounts in the brain increase to about eight times in the case of AVM-MOG01 IgG4PE(R409K) antibody, about 12 times in the case of AVM IgG4PE(R409K)_MOG01sscFv antibody and about 30 times in the case of AVM IgG4PE(R409K)_MOG01 Fab antibody as compared to those of the negative controls.

The above results show that while the bispecific antibodies which bind to MOG can increase the antibody amount in the brain as compared to those of the negative control antibodies which do not bind to MOG, the half-lives in the blood do not change.

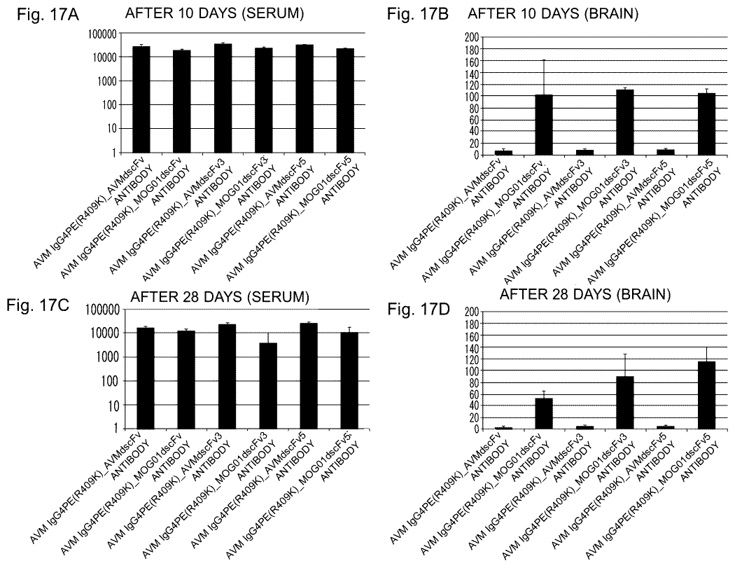

AVM IgG4PE(R409K)_MOG01dscFv antibody, AVM IgG4PE(R409K)_MOG01dscFv3 antibody and AVM IgG4PE(R409K)_MOG01dscFv5 antibody were administered at 5 mg/kg, and the antibody concentrations in the serum and the antibody amounts per unit brain weight in the brain tissues after 10 days and after 28 days are shown in FIGS. 17A to 17D.

As shown in FIGS. 17A and 17C, the antibody concentrations of all the bispecific antibodies in the serum were not different from those of the negative controls. On the other hand, as shown in FIGS. 17B and 17D, it was shown that the antibody amounts in the brain of AVM IgG4PE(R409K)_MOG01dscFv antibody, AVM IgG4PE(R409K)_MOG01dscFv3 antibody and AVM IgG4PE(R409K) MOG01dscFv5 antibody can be increased to several ten times over 28 days. Moreover, as shown in FIG. 17D, when the antibody amounts in the brain were high after 28 days, the affinities of the bispecific antibodies to MOG were also high (Table 11). It was elucidated that there is a correlation between the MOG binding activity and the antibody amount in the brain.

### [Example 20] Acquisition of Novel MOG Antibodies Showing Higher Affinity to MOG than Anti-MOG01 Antibody

### (1) Production of Extracellular Domain Proteins of Soluble Human MOG Antigen and Soluble Mouse MOG Antigen to Which FLAG-Fc is Bound

Plasmid vectors, INPEP4_hMOG-FLAG-Fc and INPEP4_mMOG-FLAG-Fc, which each express an extracellular domain protein of MOG to which FLAG-Fc was added at the C-terminus, as soluble antigens of human MOG and mouse MOG were produced by the method described in Example 4. The nucleotide sequence of hMOG-FLAG-Fc is shown in SEQ ID NO: 100, and the amino acid sequence deduced from the nucleotide sequence is shown in SEQ ID NO: 101. The nucleotide sequence of mMOG-FLAG-Fc is shown in SEQ ID NO: 102, and the amino acid sequence deduced from the nucleotide sequence is shown in SEQ ID NO: 103. The extracellular domain proteins of MOG to which FLAG-Fc was bound were obtained by transiently expressing and purifying the proteins by the method described in Example 4.

(2) Production of Extracellular Domain Proteins of MOG to which GST is Bound

Plasmid vectors, N5_hMOG-GST and N5_mMOG-GST, which each express an extracellular domain protein of MOG to which GST was added at the C-terminus, as soluble antigens of human MOG and mouse MOG were produced by the method described in Example 4. The nucleotide sequence of hMOG-GST is shown in SEQ ID NO: 104, and the amino acid sequence deduced from the nucleotide sequence is shown in SEQ ID NO: 105. The nucleotide sequence of mMOG-GST is shown in SEQ ID NO: 106, and the amino acid sequence deduced from the nucleotide sequence is shown in SEQ ID NO: 107. The extracellular domain proteins of MOG to which GST was bound were obtained by transiently expressing and purifying the proteins by the method described in Example 4.

(3) Acquisition of Anti-MOG Antibodies from Human Antibody-Producing Mice

hMOG-GST and mMOG-GST were mixed with pertussis vaccine and Alumgel and intraperitoneally or intradermally administered to human antibody-producing mice (Ishida & Lonberg, IBC's 11th Antibody Engineering, Abstract 2000; Ishida, I. et al., Cloning & Stem Cells 4, 85-96 (2002) and Ishida Isao (2002) Experimental Medicine 20, 6, 846-851).

After the first immunization, the mice were immunized with hMOG-GST and mMOG-GST three times. The individuals immunized by intraperitoneal administration were dissected four days after the final immunization, and the spleens were taken. After removing the red blood cells using a reagent for removing red blood cells (manufactured by Sigma Co. LLC.), the spleens were frozen with CELLBANKER 1 (manufactured by Nippon Zenyaku Kogyo Co., Ltd.). The individuals immunized by intradermal administration were dissected, and the axillary lymph nodes were taken. After removing the red blood cells using a reagent for removing red blood cells, the axillary lymph nodes were frozen with CELLBANKER 1. RNAs were extracted from the obtained spleen cells and the cells of the axillary lymph nodes using an RNeasy Plus Mini kit (manufactured by QIAGEN), and cDNAs were synthesized with a SMARTer RACE cDNA amplification kit (manufactured by Clontech Laboratories, Inc.). Human antibody-producing mouse-derived phage libraries were produced using the synthesized cDNAs by the method described in Example 1.

Anti-human MOG monoclonal antibodies were obtained using the human antibody-producing mouse-derived phage libraries by the phage display method. The phage display method and cloning ELISA were conducted using hMOG-FLAG_Fc and mMOG-FLAG_Fc by the methods described in Example 1.

The sequences of the clones which bound to hMOG-FLAG_Fc, mMOG-FLAG_Fc and hMOG/Expi293F cells were analyzed, and anti-MOG antibody phagemid vectors, pCANTAB_MOG301, pCANTAB_MOG303, pCANTAB_MOG307, pCANTAB_MOG310, pCANTAB_MOG312, pCANTAB_MOG326, pCANTAB_MOG329, pCANTAB_MOG446, pCANTAB_MOG456 and pCANTAB_MOG473 were obtained.

In the following paragraphs, the names of the anti-MOG scFv antibodies displayed by the phages expressed using pCANTAB_MOG301, pCANTAB_MOG303, pCANTAB_MOG307, pCANTAB_MOG310, pCANTAB_MOG312, pCANTAB_MOG326, pCANTAB_MOG329, pCANTAB_MOG446, pCANTAB_MOG456 and pCANTAB_MOG473 are referred to as MOG301 antibody, MOG303 antibody, MOG307 antibody, MOG310 antibody, MOG312 antibody, MOG326 antibody, MOG329 antibody, MOG446 antibody, MOG456 antibody and MOG473 antibody, respectively.

The nucleotide sequences which encode VH or VL of the anti-MOG antibodies and the amino acid sequences deduced from the nucleotide sequences are shown in Table 12.

TABLE 12

| Clone Name | MOG301 | MOG303 | MOG307 | MOG310 | MOG312 | MOG326 | MOG329 | MOG446 | MOG456 | MOG473 |
|---|---|---|---|---|---|---|---|---|---|---|
| Nucleotide | SEQ ID | SEQ ID | SEQ ID | SEQ ID | SEQ ID | SEQ ID | SEQ ID | SEQ ID | SEQ ID | SEQ ID |
| sequence | NO: 151 | NO: 161 | NO: 171 | NO: 181 | NO: 191 | NO: 201 | NO: 211 | NO: 221 | NO: 231 | NO: 241 |
| encoding | | | | | | | | | | |
| VH (excluding | | | | | | | | | | |
| signal sequence) | | | | | | | | | | |
| Amino acid | SEQ ID | SEQ ID | SEQ ID | SEQ ID | SEQ ID | SEQ ID | SEQ ID | SEQ ID | SEQ ID | SEQ ID |
| sequence | NO: 152 | NO: 162 | NO: 172 | NO: 182 | NO: 192 | NO: 202 | NO: 212 | NO: 222 | NO: 232 | NO: 242 |
| of VH (excluding | | | | | | | | | | |
| signal sequence) | | | | | | | | | | |
| Amino acid | SEQ ID | SEQ ID | SEQ ID | SEQ ID | SEQ ID | SEQ ID | SEQ ID | SEQ ID | SEQ ID | SEQ ID |
| sequence of | NO: 153 | NO: 163 | NO: 173 | NO: 183 | NO: 193 | NO: 203 | NO: 213 | NO: 223 | NO: 233 | NO: 243 |
| HCDR1 | | | | | | | | | | |
| Amino acid | SEQ ID | SEQ ID | SEQ ID | SEQ ID | SEQ ID | SEQ ID | SEQ ID | SEQ ID | SEQ ID | SEQ ID |
| sequence of | NO: 154 | NO: 164 | NO: 174 | NO: 184 | NO: 194 | NO: 204 | NO: 214 | NO: 224 | NO: 234 | NO: 244 |
| HCDR2 | | | | | | | | | | |
| Amino acid | SEQ ID | SEQ ID | SEQ ID | SEQ ID | SEQ ID | SEQ ID | SEQ ID | SEQ ID | SEQ ID | SEQ ID |
| sequence of | NO: 155 | NO: 165 | NO: 175 | NO: 185 | NO: 195 | NO: 205 | NO: 215 | NO: 225 | NO: 235 | NO: 245 |
| HCDR3 | | | | | | | | | | |
| Nucleotide | SEQ ID | SEQ ID | SEQ ID | SEQ ID | SEQ ID | SEQ ID | SEQ ID | SEQ ID | SEQ ID | SEQ ID |
| sequence | NO: 156 | NO: 166 | NO: 176 | NO: 186 | NO: 196 | NO: 206 | NO: 216 | NO: 226 | NO: 236 | NO: 246 |
| encoding | | | | | | | | | | |
| VL (excluding | | | | | | | | | | |
| signal sequence) | | | | | | | | | | |
| Amino acid | SEQ ID | SEQ ID | SEQ ID | SEQ ID | SEQ ID | SEQ ID | SEQ ID | SEQ ID | SEQ ID | SEQ ID |
| sequence | NO: 157 | NO: 167 | NO: 177 | NO: 187 | NO: 197 | NO: 207 | NO: 217 | NO: 227 | NO: 237 | NO: 247 |
| of VL (excluding | | | | | | | | | | |
| signal sequence) | | | | | | | | | | |
| Amino acid | SEQ ID | SEQ ID | SEQ ID | SEQ ID | SEQ ID | SEQ ID | SEQ ID | SEQ ID | SEQ ID | SEQ ID |
| sequence of | NO: 158 | NO: 168 | NO: 178 | NO: 188 | NO: 198 | NO: 208 | NO: 218 | NO: 228 | NO: 238 | NO: 248 |
| LCDR1 | | | | | | | | | | |
| Amino acid | SEQ ID | SEQ ID | SEQ ID | SEQ ID | SEQ ID | SEQ ID | SEQ ID | SEQ ID | SEQ ID | SEQ ID |
| sequence of | NO: 159 | NO: 169 | NO: 179 | NO: 189 | NO: 199 | NO: 209 | NO: 219 | NO: 229 | NO: 239 | NO: 249 |
| LCDR2 | | | | | | | | | | |
| Amino acid | SEQ ID | SEQ ID | SEQ ID | SEQ ID | SEQ ID | SEQ ID | SEQ ID | SEQ ID | SEQ ID | SEQ ID |
| sequence of | NO: 160 | NO: 170 | NO: 180 | NO: 190 | NO: 200 | NO: 210 | NO: 220 | NO: 230 | NO: 240 | NO: 250 |
| LCDR3 | | | | | | | | | | |

Moreover, clones having similar sequences having homology of 91 to 93% to that of MOG301 antibody (MOG426 and MOG428), clones having similar sequences having homology of 85 to 95% to that of MOG303 antibody (MOG313, MOG314, MOG315, MOG331, MOG357 and MOG476), clones having similar sequences having homology of 97 to 99% to that of MOG307 antibody (MOG323, MOG341, MOG354 and MOG355), clones having similar sequences having homology of 85 to 98% to that of MOG310 antibody (MOG308, MOG316, MOG319, MOG320, MOG338, MOG352, MOG359 and MOG478), a clone having a similar sequence having homology of 85% to that of MOG329 antibody (MOG470) and a clone having a similar sequence having homology of 84% to that of MOG456 antibody (MOG418) were obtained by the phage display method using MOG affinity as an index. Because it was confirmed that these similar clones bind to hMOG-FLAG_Fc, mMOG-FLAG_Fc and hMOG/Expi293F cells, it was elucidated that antibody clones having high homology to the amino acid sequences of the antibody clones are also antibodies having MOG binding activity.

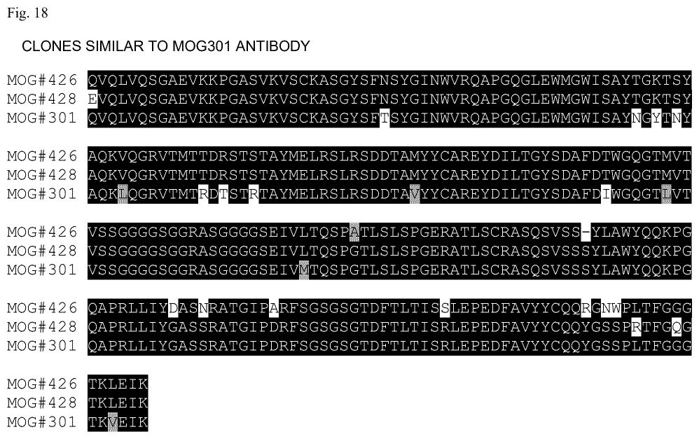

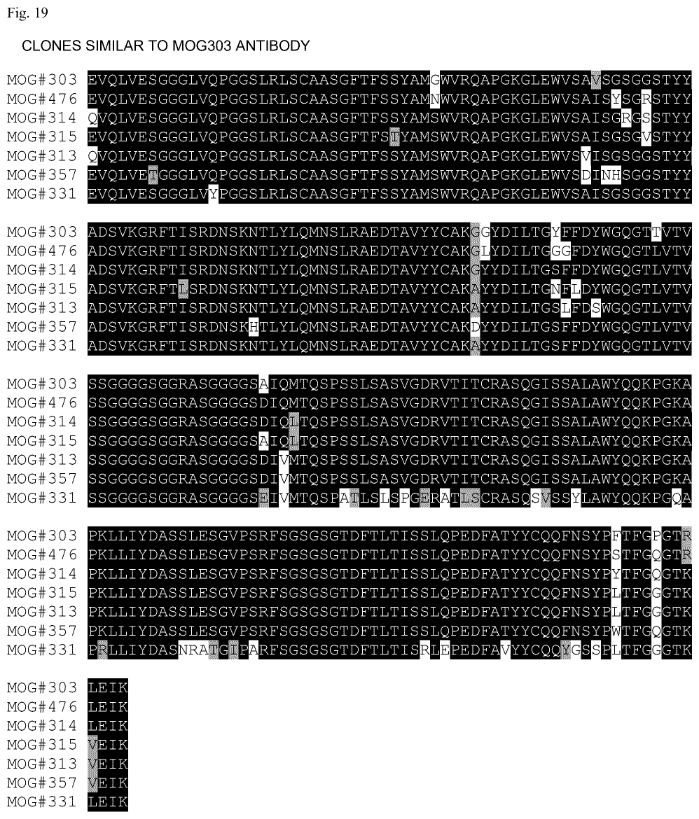

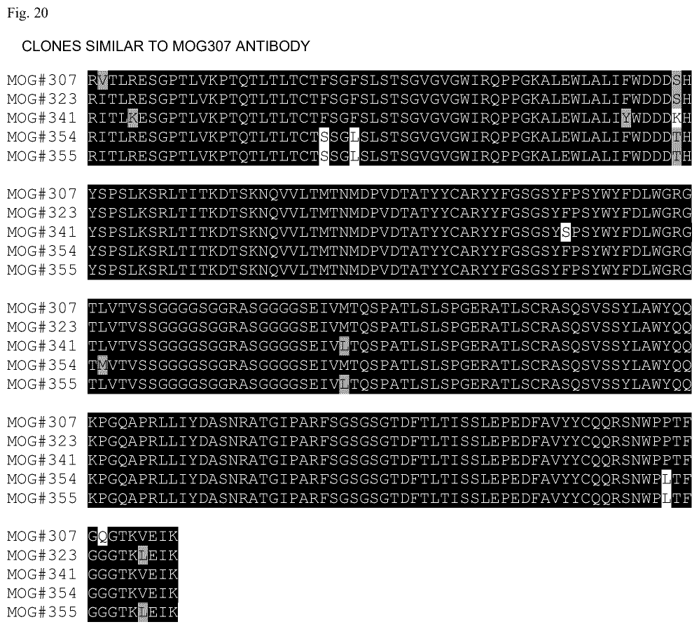

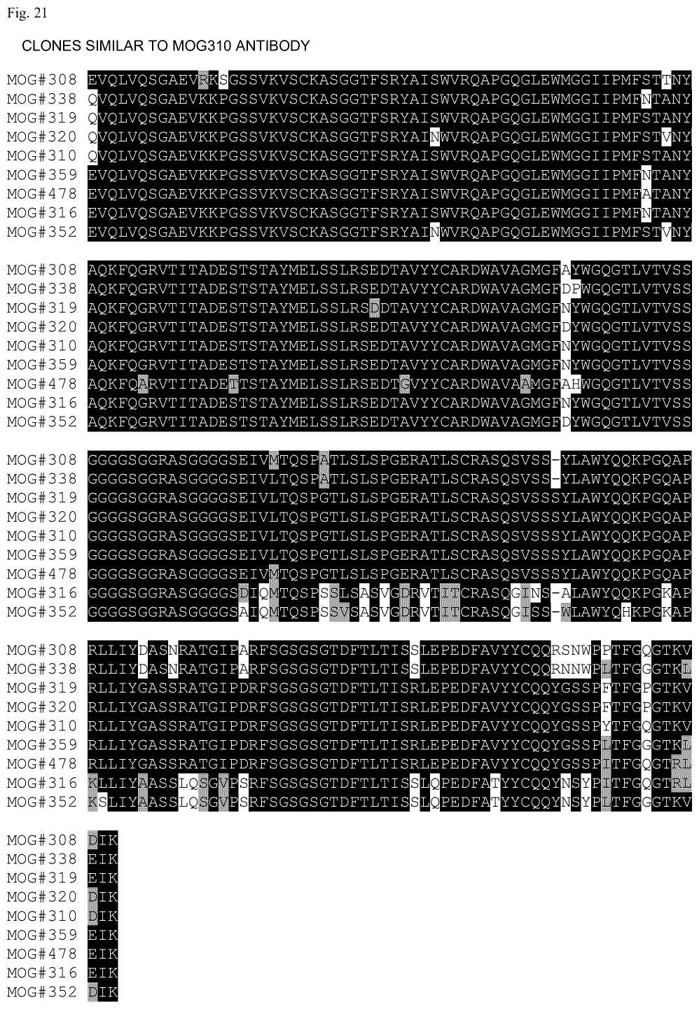

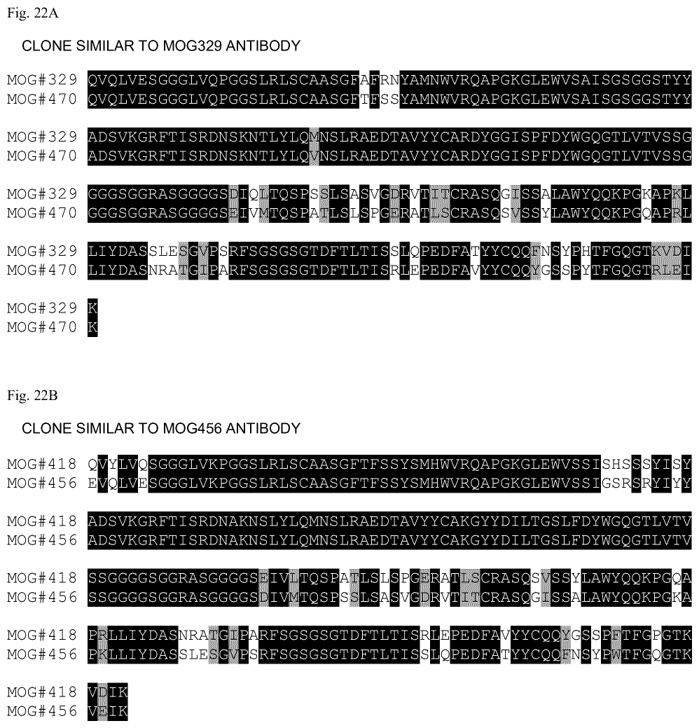

The nucleotide sequences which encode VH or VL of the similar clones and the amino acid sequences deduced from the nucleotide sequences are shown in Table 13, and comparisons of the amino acid sequences of the similar clones are shown in FIG. 18 to FIG. 22B.

TABLE 13

| Name of | | Nucleotide sequence | Amino acid sequence of | Nucleotide sequence | Amino acid sequence |
|---|---|---|---|---|---|
| Representative | Name of | encoding VH (excluding | VH (excluding signal | encoding VL (excluding | of VL (excluding signal |
| Clone | Similar Clone | signal sequence) | sequence) | signal sequence) | sequence) |
| MOG301 | MOG426 | SEQ ID NO: 251 | SEQ ID NO: 252 | SEQ ID NO: 253 | SEQ ID NO: 254 |
| | MOG428 | SEQ ID NO: 255 | SEQ ID NO: 256 | SEQ ID NO: 257 | SEQ ID NO: 258 |
| MOG303 | MOG313 | SEQ ID NO: 259 | SEQ ID NO: 260 | SEQ ID NO: 261 | SEQ ID NO: 262 |
| | MOG314 | SEQ ID NO: 263 | SEQ ID NO: 264 | SEQ ID NO: 265 | SEQ ID NO: 266 |
| | MOG315 | SEQ ID NO: 267 | SEQ ID NO: 268 | SEQ ID NO: 269 | SEQ ID NO: 270 |
| | MOG331 | SEQ ID NO: 271 | SEQ ID NO: 272 | SEQ ID NO: 273 | SEQ ID NO: 274 |
| | MOG357 | SEQ ID NO: 275 | SEQ ID NO: 276 | SEQ ID NO: 277 | SEQ ID NO: 278 |
| | MOG476 | SEQ ID NO: 279 | SEQ ID NO: 280 | SEQ ID NO: 281 | SEQ ID NO: 282 |
| MOG307 | MOG323 | SEQ ID NO: 283 | SEQ ID NO: 284 | SEQ ID NO: 285 | SEQ ID NO: 286 |
| | MOG341 | SEQ ID NO: 287 | SEQ ID NO: 288 | SEQ ID NO: 289 | SEQ ID NO: 290 |
| | MOG354 | SEQ ID NO: 291 | SEQ ID NO: 292 | SEQ ID NO: 293 | SEQ ID NO: 294 |
| | MOG355 | SEQ ID NO: 295 | SEQ ID NO: 296 | SEQ ID NO: 297 | SEQ ID NO: 298 |
| MOG310 | MOG308 | SEQ ID NO: 299 | SEQ ID NO: 300 | SEQ ID NO: 301 | SEQ ID NO: 302 |
| | MOG316 | SEQ ID NO: 303 | SEQ ID NO: 304 | SEQ ID NO: 305 | SEQ ID NO: 306 |
| | MOG319 | SEQ ID NO: 307 | SEQ ID NO: 308 | SEQ ID NO: 309 | SEQ ID NO: 310 |
| | MOG320 | SEQ ID NO: 311 | SEQ ID NO: 312 | SEQ ID NO: 313 | SEQ ID NO: 314 |
| | MOG338 | SEQ ID NO: 315 | SEQ ID NO: 316 | SEQ ID NO: 317 | SEQ ID NO: 318 |
| | MOG352 | SEQ ID NO: 319 | SEQ ID NO: 320 | SEQ ID NO: 321 | SEQ ID NO: 322 |
| | MOG359 | SEQ ID NO: 323 | SEQ ID NO: 324 | SEQ ID NO: 325 | SEQ ID NO: 326 |
| | MOG478 | SEQ ID NO: 327 | SEQ ID NO: 328 | SEQ ID NO: 329 | SEQ ID NO: 330 |
| MOG329 | MOG470 | SEQ ID NO: 331 | SEQ ID NO: 332 | SEQ ID NO: 333 | SEQ ID NO: 334 |
| MOG456 | MOG418 | SEQ ID NO: 335 | SEQ ID NO: 336 | SEQ ID NO: 337 | SEQ ID NO: 338 |

### [Example 21] Production of Anti-MOG scFv-Fc Antibodies

A gene fragment of the scFv region was amplified by PCR using a phagemid vector pCANTAB_MOG01 as a template. A gene fragment of the Hinge-CH2-CH3 region was amplified by PCR using a synthetic gene of the heavy chain constant region as a template. The obtained gene fragments were inserted to N5KG4PE vector (described in International Publication No. 2002/088186), and N5-MOG01 scFv-hG4PE vector was produced.

A gene fragment of the scFv region was amplified by PCR using a phagemid vector pCANTAB_MOG301 as a template. A gene fragment of the Hinge-CH2-CH3 region was amplified by PCR using a synthetic gene of the heavy chain constant region as a template. The obtained gene fragments were inserted to pCI vector (manufactured by Promega Corporation), and pCI-MOG301 scFv-hG4PE(R409K) vector was produced.

By the same method, antibody expression vectors to which gene fragments of the scFv regions of the anti-MOG antibodies shown in Table 12 were inserted were produced and named pCI-MOG303 scFv-hG4PE(R409K), pCI-MOG307 scFv-hG4PE(R409K), pCI-MOG310 scFv-hG4PE(R409K), pCI-MOG312 scFv-hG4PE(R409K), pCI-MOG326 scFv-hG4PE(R409K), pCI-MOG329 scFv-hG4PE(R409K), pCI-MOG446 scFv-hG4PE(R409K), pCI-MOG456 scFv-hG4PE(R409K) and pCI-MOG473 scFv-hG4PE(R409K).

The produced anti-MOG antibody expression vectors were prepared by the method described in Example 6. Antibodies were expressed using the anti-MOG antibody expression vectors, pCI-MOG301 scFv-hG4PE(R409K), pCI-MOG303 scFv-hG4PE(R409K), pCI-MOG307 scFv-hG4PE(R409K), pCI-MOG310 scFv-hG4PE(R409K), pCI-MOG312 scFv-hG4PE(R409K), pCI-MOG326 scFv-hG4PE(R409K), pCI-MOG329 scFv-hG4PE(R409K), pCI-MOG446 scFv-hG4PE(R409K), pCI-MOG456 scFv-hG4PE(R409K) and pCI-MOG473 scFv-hG4PE(R409K), and MOG301 scFv-hG4PE(R409K) antibody, MOG303 scFv-hG4PE(R409K) antibody, MOG307 scFv-hG4PE(R409K) antibody, MOG310 scFv-hG4PE(R409K) antibody, MOG312 scFv-hG4PE(R409K) antibody, MOG326 scFv-hG4PE(R409K) antibody, MOG329 scFv-hG4PE(R409K) antibody, MOG446 scFv-hG4PE(R409K) antibody, MOG456 scFv-hG4PE(R409K) antibody and MOG473 scFv-hG4PE(R409K) antibody were obtained, respectively.

### [Example 22] Evaluation of Affinities of Anti-MOG Antibodies to MOG Using Flow Cytometer

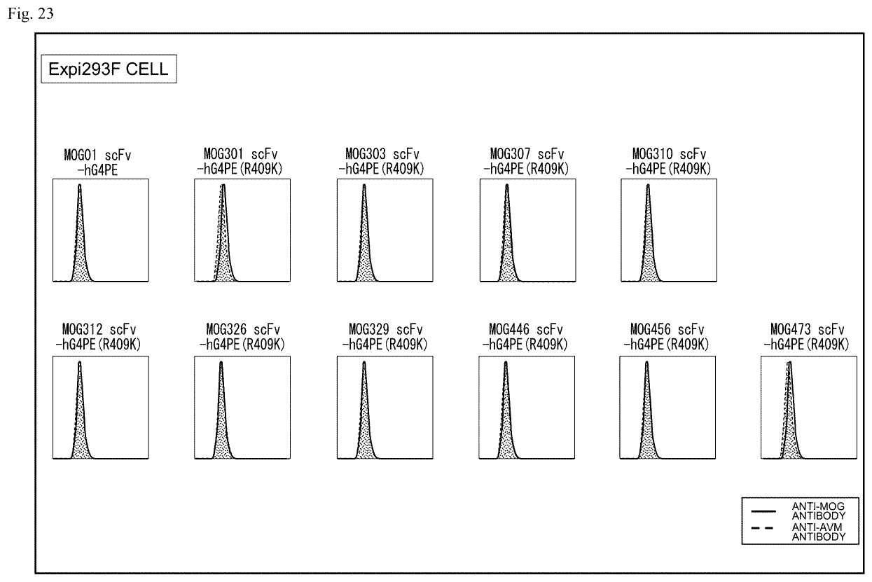

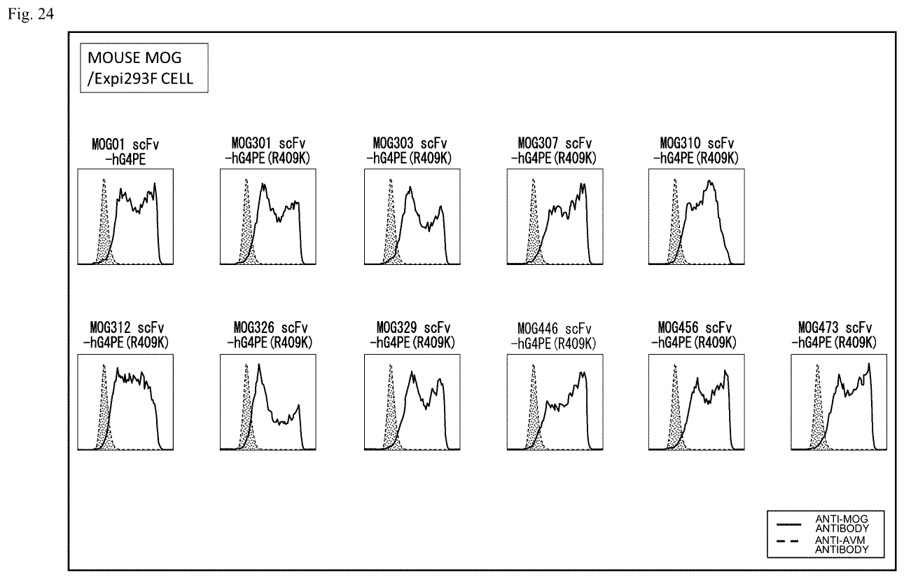

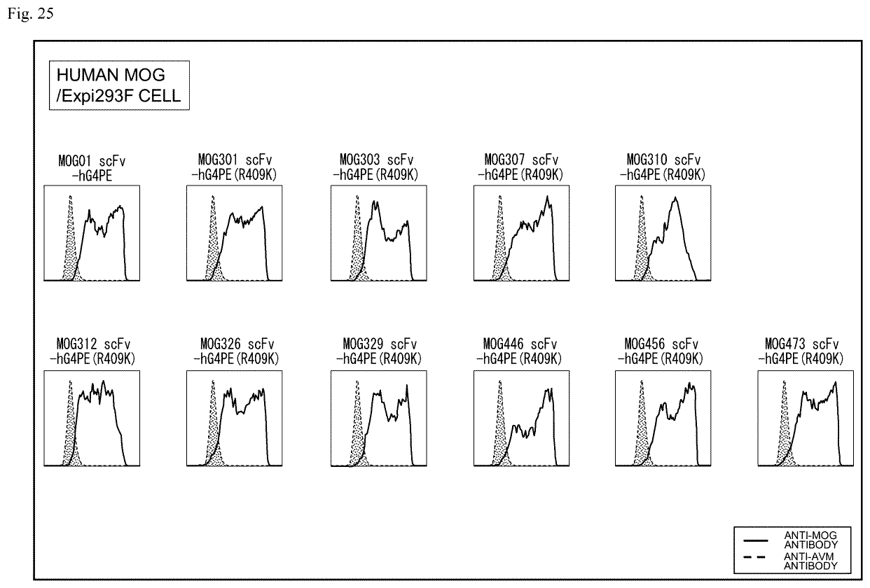

Binding of the anti-MOG antibodies obtained in Example 21 to MOG was evaluated by the same method as that of Example 7. The results are shown in FIGS. 23 to 25.

As shown in FIGS. 23 to 25, MOG01 scFv-hG4PE, MOG301 scFv-hG4PE(R409K), MOG303 scFv-hG4PE(R409K), MOG307 scFv-hG4PE(R409K), MOG310 scFv-hG4PE(R409K), MOG312 scFv-hG4PE(R409K), MOG326 scFv-hG4PE(R409K), MOG329 scFv-hG4PE(R409K), MOG446 scFv-hG4PE(R409K), MOG456 scFv-hG4PE(R409K) and MOG473 scFv-hG4PE(R409K) all showed binding activity to hMOG/Expi293F cells and mMOG/Expi293F cells.

### [Example 23] Evaluation of Affinities of Anti-MOG Antibodies to MOG by Surface Plasmon Resonance Detection

Binding of MOG01 scFv-hG4PE, MOG301 scFv-hG4PE(R409K), MOG303 scFv-hG4PE(R409K), MOG307 scFv-hG4PE(R409K), MOG329 scFv-hG4PE(R409K), MOG446 scFv-hG4PE(R409K), MOG456 scFv-hG4PE(R409K) and MOG473 scFv-hG4PE(R409K) obtained in Example 21 to human MOG and mouse MOG was evaluated by the same method as that of Example 8. hMOG-GST and mMOG-GST were used as analytes. The results of evaluation of the affinities to human MOG are shown in Table 14, and the results of evaluation of the affinities to mouse MOG are shown in Table 15.

TABLE 14
Reactivity to human MOG

| Antibody Name | ka (1/Ms) | kd (1/s) | KD (M) |
|---|---|---|---|
| MOG01 scFv-hG4PE | 4.1E+06 | 1.5E−02 | 3.6E−09 |
| MOG301 scFv-hG4PE(R409K) | 1.1E+06 | 1.8E−04 | 1.8E−10 |
| MOG303 scFv-hG4PE(R409K) | 9.1E+05 | 1.6E−04 | 1.7E−10 |
| MOG307 scFv-hG4PE(R409K) | 1.6E+05 | 1.4E−04 | 8.9E−10 |
| MOG329 scFv-hG4PE(R409K) | 1.6E+06 | 2.1E−04 | 1.3E−10 |
| MOG446 scFv-hG4PE(R409K) | 1.9E+05 | 1.7E−04 | 8.7E−10 |
| MOG456 scFv-hG4PE(R409K) | 1.0E+06 | 2.6E−04 | 2.5E−10 |
| MOG473 scFv-hG4PE(R409K) | 1.5E+06 | 1.5E−04 | 1.0E−10 |

ka of MOG01 was outside the measurement range.

TABLE 15
Reactivity to mouse MOG

| Antibody Name | ka (1/Ms) | kd (1/s) | KD (M) |
|---|---|---|---|
| MOG01 scFv-hG4PE | 5.9E+06 | 4.0E−02 | 6.9E−09 |
| MOG301 scFv-hG4PE(R409K) | 4.9E+05 | 2.1E−04 | 4.3E−10 |
| MOG303 scFv-hG4PE(R409K) | 1.1E+06 | 2.1E−04 | 1.9E−10 |
| MOG307 scFv-hG4PE(R409K) | 1.6E+05 | 1.5E−04 | 9.6E−10 |
| MOG329 scFv-hG4PE(R409K) | 1.1E+06 | 2.3E−04 | 2.2E−10 |
| MOG446 scFv-hG4PE(R409K) | 1.2E+05 | 3.7E−04 | 3.2E−09 |
| MOG456 scFv-hG4PE(R409K) | 7.6E+05 | 3.9E−04 | 5.2E−10 |
| MOG473 scFv-hG4PE(R409K) | 7.6E+05 | 3.7E−04 | 4.9E−10 |

As shown in Table 14 and Table 15, the dissociation constants (KD values) of the anti-MOG antibodies to human MOG were 1.0⁻¹⁰ (M) to 3.6×10⁻⁹ (M), and the dissociation constants (KD values) to mouse MOG were 1.9×10−¹⁰ (M) to 6.9×10−⁹ (M). It was thus elucidated that all the antibodies show excellent affinity. The association rate constant ka of MOG01 scFv-hG4PE was outside the measurement range of the device, and the KD value could not be determined as a unique value.

### [Example 24] Production of Enzyme-Fused Antibodies

Enzyme-fused antibodies in which acid sphingomyelinase (ASM) was fused to the C-terminus of anti-MOG01IgG antibody or anti-AVMIgG antibody were produced by the method described below. The vector expressing the antibody in which ASM was fused to the C-terminus of anti-MOG01IgG antibody was named pCI-MOG01-hLG4PE(R409K)_ASM, and the vector expressing the antibody in which ASM was fused to the C-terminus of anti-AVMIgG antibody was named pCI-AVM-hLG4PE(R409K)_ASM.

A gene fragment of the linker-ASM region was amplified by PCR using a synthetic gene of ASM shown in SEQ ID NO: 150 as a template. Moreover, a gene fragment of the CH1-Hinge-CH2-CH3(R409K) region was synthesized by PCR using a synthetic gene as a template. A gene fragment of the MOG01 light chain region and a gene fragment of the MOG01 VH region were amplified by PCR using N5LG4PE_MOG01 as a template.

The obtained gene fragments were inserted to pCI vector (manufactured by Promega Corporation), and pCI-MOG01-hLG4PE(R409K)_ASM vector was produced. A gene fragment of the CH2-CH3 region was amplified by PCR using a synthetic gene as a template. The gene fragments of the CH2-CH3 region and the linker-ASM region were inserted to the PmlI-BamHI site of pCI-AVM-hLG4PE(R409K) vector, and pCI-AVM-hLG4PE(R409K)_ASM was produced.

pCI-MOG01-hLG4PE(R409K)_ASM and pCI-AVM-hLG4PE(R409K)_ASM were expressed and purified by the method shown in Example 6. The antibody obtained by expression using pCI-MOG01-hLG4PE(R409K)_ASM was named MOG01 IgG4PE(R409K)-ASM, and the antibody obtained by expression using pCI-AVM-hLG4PE(R409K)_ASM was named AVM IgG4PE(R409K)-ASM.

### [Example 25] Evaluation of Activities of Enzyme-Fused Antibodies

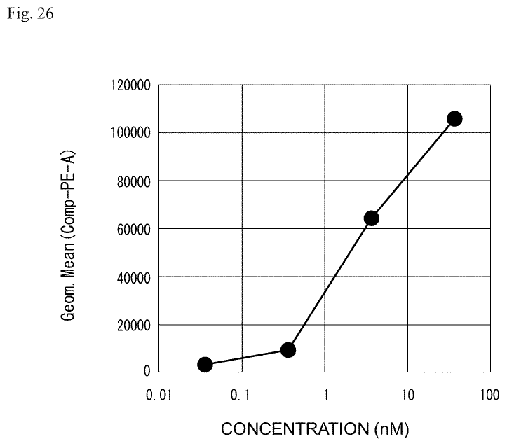

The affinity of MOG01 IgG4PE(R409K)-ASM to MOG-expressing cells was examined by the same method as that of Example 23, and the results are shown in FIG. 26. Moreover, the affinity to MOG soluble antigen was examined by the same method as that of Example 8. As a result, the dissociation constant (KD value) of MOG01 IgG4PE(R409K)-ASM was 2.9×10−⁹ (M), and excellent affinity was observed.

That an anti-ASM antibody (manufactured by LSBio) binds to produced MOG01 IgG4PE(R409K)-ASM and AVM IgG4PE(R409K)-ASM was confirmed by the ELISA method shown below.

In the ELISA, MOG01 IgG4PE(R409K)-ASM and AVM IgG4PE(R409K)-ASM were immobilized (100 ng/50 μL) on MAXISORP (manufactured by NUNC), and the sites to which MOG01 IgG4PE(R409K)-ASM and AVM IgG4PE(R409K)-ASM were not bound were blocked using SuperBlock Blocking Buffer (manufactured by Thermo Fisher Scientific Inc.). As a negative control, a plate on which anti-MOG01IgG antibody and anti-AVMIgG antibody were immobilized (50 ng/50 μL) was also prepared. The anti-ASM antibody which was diluted to a concentration of 0.2, 1 or 5 μg/mL with PBS-T was added to the wells and reacted at room temperature for an hour, and then the wells were washed with PBS-T.

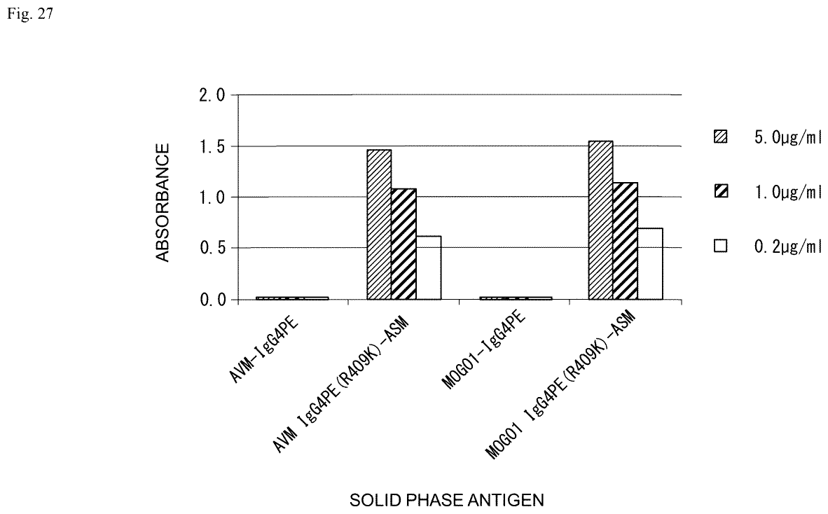

Next, a solution obtained by diluting horseradish peroxidase-labeled anti-Mouse Immunoglobulins antibody (manufactured by Dako) with PBS-T was added to the wells and reacted at room temperature for an hour. A TMB chromogenic substrate solution (manufactured by DAKO) was added, and the plates were incubated at room temperature. The chromogenic reaction was stopped by adding 2 M hydrochloric acid to the wells, and the absorbances at the wavelength of 450 nm (reference wavelength of 570 nm) were measured. The results obtained are shown in FIG. 27.

As shown in FIG. 27, it was shown that the anti-ASM antibody recognized and bound to the produced ASM-fused antibodies. Moreover, as a result of measurement of the sphingomyelinase activities of produced MOG01 IgG4PE(R409K)-ASM and AVM IgG4PE(R409K)-ASM using a sphingomyelinase activity measurement kit (manufactured by Echelon Biosciences), it was confirmed that the produced ASM-fused antibodies had enzymatic activities.

From the above results, it was confirmed that the enzyme-fused antibody obtained by fusing an enzyme to a MOG antibody maintains both the antigen binding activity and the enzymatic activity.

### [Example 26] Evaluation of Mouse Brain Migration Properties of Enzyme-Fused Antibodies

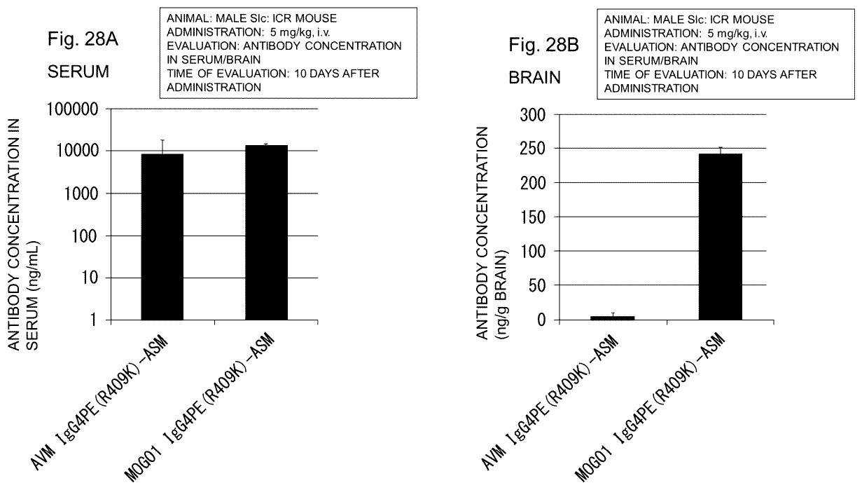

The mouse brain migration properties of the ASM-fused antibodies obtained in Example 24 were evaluated by the same method as that of Example 14. The antibody concentrations in the serum and the antibody amounts per unit brain weight in the brain tissues 10 days after administering the ASM-fused antibodies at 5 mg/kg are shown in FIGS. 28A and 28B.

As shown in FIGS. 28A and 28B, the antibody concentration of MOG01 IgG4PE(R409K)-ASM in the serum was not different from that of AVM IgG4PE(R409K)-ASM. On the other hand, it was shown that the antibody amount of MOG01 IgG4PE(R409K)-ASM in the brain increased to about 58 times the amount of AVM IgG4PE(R409K)-ASM.

The invention has been explained in detail using the specific aspects, but it is obvious for those skilled in the art that various changes and modifications can be made without departing from the spirit and scope of the invention. The present application is based on a Japanese patent application filed on Dec. 26, 2016 (patent application No. 2016-251106), which is incorporated by reference in its entirety.

### SEQUENCE LISTING FREE TEXT

Definition of SEQ ID NO: 3-artificial sequence: amino acid sequence of VH of MOG01 excluding signal sequence
Definition of SEQ ID NO: 4-artificial sequence: amino acid sequence of HCDR1 of MOG01
Definition of SEQ ID NO: 5-artificial sequence: amino acid sequence of HCDR2 of MOG01
Definition of SEQ ID NO: 6-artificial sequence: amino acid sequence of HCDR3 of MOG01
Definition of SEQ ID NO: 9-artificial sequence: amino acid sequence of VL of MOG01 excluding signal sequence
Definition of SEQ ID NO: 10-artificial sequence: amino acid sequence of LCDR1 of MOG01
Definition of SEQ ID NO: 11-artificial sequence: amino acid sequence of LCDR2 of MOG01
Definition of SEQ ID NO: 12-artificial sequence: amino acid sequence of LCDR3 of MOG01
Definition of SEQ ID NO: 15-artificial sequence: amino acid sequence of VH of MOG09 excluding signal sequence
Definition of SEQ ID NO: 16-artificial sequence: amino acid sequence of HCDR1 of MOG09
Definition of SEQ ID NO: 17-artificial sequence: amino acid sequence of HCDR2 of MOG09
Definition of SEQ ID NO: 18-artificial sequence: amino acid sequence of HCDR3 of MOG09
Definition of SEQ ID NO: 21-artificial sequence: amino acid sequence of VL of MOG09 excluding signal sequence
Definition of SEQ ID NO: 22-artificial sequence: amino acid sequence of LCDR1 of MOG09
Definition of SEQ ID NO: 23-artificial sequence: amino acid sequence of LCDR2 of MOG09
Definition of SEQ ID NO: 24-artificial sequence: amino acid sequence of LCDR3 of MOG09
Definition of SEQ ID NO: 27-artificial sequence: amino acid sequence of VH of MOG14 excluding signal sequence
Definition of SEQ ID NO: 28-artificial sequence: amino acid sequence of HCDR1 of MOG14
Definition of SEQ ID NO: 29-artificial sequence: amino acid sequence of HCDR2 of MOG14
Definition of SEQ ID NO: 30-artificial sequence: amino acid sequence of HCDR3 of MOG14
Definition of SEQ ID NO: 33-artificial sequence: amino acid sequence of VL of MOG14 excluding signal sequence
Definition of SEQ ID NO: 34-artificial sequence: amino acid sequence of LCDR1 of MOG14
Definition of SEQ ID NO: 35-artificial sequence: amino acid sequence of LCDR2 of MOG14
Definition of SEQ ID NO: 36-artificial sequence: amino acid sequence of LCDR3 of MOG14
Definition of SEQ ID NO: 37-artificial sequence: nucleotide sequence of VHH of iMOG_3Rim1_S32 including signal sequence
Definition of SEQ ID NO: 38-artificial sequence: amino acid sequence of synthetic construct
Definition of SEQ ID NO: 39-artificial sequence: amino acid sequence of VHH of iMOG_3Rim1_S32 excluding signal sequence
Definition of SEQ ID NO: 40 (Thr Met Gly)-artificial sequence: amino acid sequence of CDR1 of iMOG_3Rim1_S32
Definition of SEQ ID NO: 41-artificial sequence: amino acid sequence of CDR2 of iMOG_3Rim1_S32
Definition of SEQ ID NO: 42-artificial sequence: amino acid sequence of CDR3 of iMOG 3Rim1 S32
Definition of SEQ ID NO: 43-artificial sequence: nucleotide sequence of primer 1
Definition of SEQ ID NO: 44-artificial sequence: nucleotide sequence of primer 2
Definition of SEQ ID NO: 45-artificial sequence: nucleotide sequence of primer 3
Definition of SEQ ID NO: 46-artificial sequence: nucleotide sequence of primer 4
Definition of SEQ ID NO: 47-artificial sequence: nucleotide sequence of primer 5
Definition of SEQ ID NO: 48-artificial sequence: nucleotide sequence of primer 6
Definition of SEQ ID NO: 49-artificial sequence: nucleotide sequence of primer 7
Definition of SEQ ID NO: 50-artificial sequence: nucleotide sequence of primer 8
Definition of SEQ ID NO: 51-artificial sequence: nucleotide sequence of primer 9
Definition of SEQ ID NO: 52-artificial sequence: nucleotide sequence of primer 10
Definition of SEQ ID NO: 53-artificial sequence: nucleotide sequence of primer 11
Definition of SEQ ID NO: 54-artificial sequence: nucleotide sequence of primer 12
Definition of SEQ ID NO: 55-artificial sequence: nucleotide sequence of primer 13
Definition of SEQ ID NO: 56-artificial sequence: nucleotide sequence of primer 14
Definition of SEQ ID NO: 57-artificial sequence: nucleotide sequence of primer 15
Definition of SEQ ID NO: 58-artificial sequence: nucleotide sequence of primer 16
Definition of SEQ ID NO: 59-artificial sequence: nucleotide sequence of primer 17
Definition of SEQ ID NO: 60-artificial sequence: nucleotide sequence of primer 18
Definition of SEQ ID NO: 61-artificial sequence: nucleotide sequence of primer 19
Definition of SEQ ID NO: 62-artificial sequence: nucleotide sequence of primer 20
Definition of SEQ ID NO: 63-artificial sequence: nucleotide sequence of primer 21
Definition of SEQ ID NO: 64-artificial sequence: nucleotide sequence of primer 22
Definition of SEQ ID NO: 65-artificial sequence: nucleotide sequence of primer 23
Definition of SEQ ID NO: 66-artificial sequence: nucleotide sequence of primer 24
Definition of SEQ ID NO: 69-artificial sequence: nucleotide sequence of rMOG-FLAG-Fc
Definition of SEQ ID NO: 70-artificial sequence: amino acid sequence of synthetic construct
Definition of SEQ ID NO: 71-artificial sequence: nucleotide sequence of rMOG-GST
Definition of SEQ ID NO: 72-artificial sequence: amino acid sequence of synthetic construct
Definition of SEQ ID NO: 79-artificial sequence: nucleotide sequence of primer 25
Definition of SEQ ID NO: 80-artificial sequence: nucleotide sequence of primer 26
Definition of SEQ ID NO: 81-artificial sequence: nucleotide sequence of primer 27
Definition of SEQ ID NO: 82-artificial sequence: nucleotide sequence of primer 28
Definition of SEQ ID NO: 83-artificial sequence: nucleotide sequence of primer 29
Definition of SEQ ID NO: 84-artificial sequence: nucleotide sequence of primer 30
Definition of SEQ ID NO: 85-artificial sequence: nucleotide sequence of primer 31
Definition of SEQ ID NO: 86-artificial sequence: nucleotide sequence of primer 32
Definition of SEQ ID NO: 87-artificial sequence: nucleotide sequence of primer 33
Definition of SEQ ID NO: 88-artificial sequence: nucleotide sequence of primer 34
Definition of SEQ ID NO: 89-artificial sequence: nucleotide sequence of primer 35
Definition of SEQ ID NO: 90-artificial sequence: nucleotide sequence of primer 36
Definition of SEQ ID NO: 91-artificial sequence: nucleotide sequence of primer 37
Definition of SEQ ID NO: 92-artificial sequence: nucleotide sequence of primer 38
Definition of SEQ ID NO: 93-artificial sequence: nucleotide sequence of primer 39
Definition of SEQ ID NO: 94-artificial sequence: nucleotide sequence of primer 40
Definition of SEQ ID NO: 95-artificial sequence: nucleotide sequence of primer 41
Definition of SEQ ID NO: 96-artificial sequence: nucleotide sequence of primer 42
Definition of SEQ ID NO: 97-artificial sequence: nucleotide sequence of primer 43
Definition of SEQ ID NO: 98-artificial sequence: nucleotide sequence of hHER2-GST
Definition of SEQ ID NO: 99-artificial sequence: amino acid sequence of synthetic construct
Definition of SEQ ID NO: 100-artificial sequence: nucleotide sequence of hMOG-FLAG-Fc (including signal sequence)
Definition of SEQ ID NO: 101-artificial sequence: amino acid sequence of hMOG-FLAG-Fc (including signal sequence)
Definition of SEQ ID NO: 102-artificial sequence: nucleotide sequence of mMOG-FLAG-Fc (including signal sequence)
Definition of SEQ ID NO: 103-artificial sequence: amino acid sequence of mMOG-FLAG-Fc (including signal sequence)
Definition of SEQ ID NO: 104-artificial sequence: nucleotide sequence of hMOG-GST (including signal sequence)
Definition of SEQ ID NO: 105-artificial sequence: amino acid sequence of hMOG-GST (including signal sequence)
Definition of SEQ ID NO: 106-artificial sequence: nucleotide sequence of mMOG-GST (including signal sequence)
Definition of SEQ ID NO: 107-artificial sequence: amino acid sequence of mMOG-GST (including signal sequence)
Definition of SEQ ID NO: 108-artificial sequence: nucleotide sequence of antibody sequence of pCI-AVM-hLG4PE(R409K/S354C/T366W)-FLAG tag (excluding signal sequence)
Definition of SEQ ID NO: 109-artificial sequence: amino acid sequence of antibody sequence of pCI-AVM-hLG4PE(R409K/S354C/T366W)-FLAG tag (excluding signal sequence)
Definition of SEQ ID NO: 110-artificial sequence: nucleotide sequence of antibody sequence of pCI-MOG01-hLG4PE(R409K/Y349C/T366S/L368A/Y407V)-His tag (excluding signal sequence)
Definition of SEQ ID NO: 111-artificial sequence: amino acid sequence of antibody sequence of pCI-MOG01-hLG4PE(R409K/Y349C/T366S/L368A/Y407V)-His tag (excluding signal sequence)
Definition of SEQ ID NO: 112-artificial sequence: nucleotide sequence of antibody sequence of pCI-AVM-hLG4PE(R409K)-linker-MOG01VL-CL (excluding signal sequence)
Definition of SEQ ID NO: 113-artificial sequence: amino acid sequence of antibody sequence of pCI-AVM-hLG4PE(R409K)-linker-MOG01VL-CL (excluding signal sequence)
Definition of SEQ ID NO: 114-artificial sequence: nucleotide sequence of antibody sequence of pCI-MOG01VH-CH (excluding signal sequence)
Definition of SEQ ID NO: 115-artificial sequence: amino acid sequence of antibody sequence of pCI-MOG01VH-CH (excluding signal sequence)
Definition of SEQ ID NO: 116-artificial sequence: nucleotide sequence of antibody sequence of pCI-AVM-hLG4PE(R409K/S354C/T366W)-linker-MOG01scFv-FLAG tag (excluding signal sequence)
Definition of SEQ ID NO: 117-artificial sequence: amino acid sequence of antibody sequence of pCI-AVM-hLG4PE(R409K/S354C/T366W)-linker-MOG01scFv-FLAG tag (excluding signal sequence)
Definition of SEQ ID NO: 118-artificial sequence: nucleotide sequence of antibody sequence of pCI-AVM-hLG4PE(R409K/Y349C/T366S/L368A/Y407V)-His tag (excluding signal sequence)
Definition of SEQ ID NO: 119-artificial sequence: amino acid sequence of antibody sequence of pCI-AVM-hLG4PE(R409K/Y349C/T366S/L368A/Y407V)-His tag (excluding signal sequence)
Definition of SEQ ID NO: 120-artificial sequence: nucleotide sequence of antibody sequence of pCI-AVM-hLG4PE(R409K)_MOG01scFv2 (excluding signal sequence)
Definition of SEQ ID NO: 121-artificial sequence: amino acid sequence of antibody sequence of pCI-AVM-hLG4PE(R409K)_MOG01scFv2 (excluding signal sequence)
Definition of SEQ ID NO: 122-artificial sequence: nucleotide sequence of antibody sequence of pCI-AVM-hLG4PE(R409K)_MOG01scFv3 (excluding signal sequence)
Definition of SEQ ID NO: 123-artificial sequence: amino acid sequence of antibody sequence of pCI-AVM-hLG4PE(R409K)_MOG01scFv3 (excluding signal sequence)
Definition of SEQ ID NO: 124-artificial sequence: nucleotide sequence of antibody sequence of pCI-AVM-hLG4PE(R409K)_MOG01scFv4 (excluding signal sequence)
Definition of SEQ ID NO: 125-artificial sequence: amino acid sequence of antibody sequence of pCI-AVM-hLG4PE(R409K)_MOG01scFv4 (excluding signal sequence)
Definition of SEQ ID NO: 126-artificial sequence: nucleotide sequence of antibody sequence of pCI-AVM-hLG4PE(R409K)_MOG01scFv5 (excluding signal sequence)
Definition of SEQ ID NO: 127-artificial sequence: amino acid sequence of antibody sequence of pCI-AVM-hLG4PE(R409K)_MOG01scFv5 (excluding signal sequence)
Definition of SEQ ID NO: 128-artificial sequence: nucleotide sequence of antibody sequence of pCI-AVM-hLG4PE(R409K)_MOG01scFv6 (excluding signal sequence)
Definition of SEQ ID NO: 129-artificial sequence: amino acid sequence of antibody sequence of pCI-AVM-hLG4PE(R409K)_MOG01scFv6 (excluding signal sequence)
Definition of SEQ ID NO: 130-artificial sequence: nucleotide sequence of antibody sequence of pCI-AVM-hLG4PE(R409K)_MOG01scFv7 (excluding signal sequence)
Definition of SEQ ID NO: 131-artificial sequence: amino acid sequence of antibody sequence of pCI-AVM-hLG4PE(R409K)_MOG01scFv7 (excluding signal sequence)
Definition of SEQ ID NO: 132-artificial sequence: nucleotide sequence of antibody sequence of pCI-AVM-hLG4PE(R409K)_MOG01scFv8 (excluding signal sequence)
Definition of SEQ ID NO: 133-artificial sequence: amino acid sequence of antibody sequence of pCI-AVM-hLG4PE(R409K)_MOG01scFv8 (excluding signal sequence)
Definition of SEQ ID NO: 134-artificial sequence: nucleotide sequence of antibody sequence of pCI-AVM-hLG4PE(R409K)_MOG01scFv9 (excluding signal sequence)
Definition of SEQ ID NO: 135-artificial sequence: amino acid sequence of antibody sequence of pCI-AVM-hLG4PE(R409K)_MOG01scFv9 (excluding signal sequence)
Definition of SEQ ID NO: 136-artificial sequence: nucleotide sequence of antibody sequence of pCI-AVM-hLG4PE(R409K)_MOG01scFv10 (excluding signal sequence)
Definition of SEQ ID NO: 137-artificial sequence: amino acid sequence of antibody sequence of pCI-AVM-hLG4PE(R409K)_MOG01scFv10 (excluding signal sequence)
Definition of SEQ ID NO: 138-artificial sequence: nucleotide sequence of antibody sequence of pCI-AVM-hLG4PE(R409K)_MOG01scFv11 (excluding signal sequence)
Definition of SEQ ID NO: 139-artificial sequence: amino acid sequence of antibody sequence of pCI-AVM-hLG4PE(R409K)_MOG01scFv11 (excluding signal sequence)
Definition of SEQ ID NO: 140-artificial sequence: nucleotide sequence of antibody sequence of pCI-AVM-hLG4PE(R409K)-linker-AVMVL-CL (excluding signal sequence)
Definition of SEQ ID NO: 141-artificial sequence: amino acid sequence of antibody sequence of pCI-AVM-hLG4PE(R409K)-linker-AVMVL-CL (excluding signal sequence)
Definition of SEQ ID NO: 142-artificial sequence: nucleotide sequence of antibody sequence of pCI-AVMVH-CH (excluding signal sequence)
Definition of SEQ ID NO: 143-artificial sequence: amino acid sequence of antibody sequence of pCI-AVMVH-CH (excluding signal sequence)
Definition of SEQ ID NO: 144-artificial sequence: nucleotide sequence of antibody sequence of pCI-AVM-hLG4PE(R409K/S354C/T366W)-linker-AVMscFv-FLAG tag (excluding signal sequence)
Definition of SEQ ID NO: 145-artificial sequence: amino acid sequence of antibody sequence of pCI-AVM-hLG4PE(R409K/S354C/T366W)-linker-AVMscFv-FLAG tag (excluding signal sequence)
Definition of SEQ ID NO: 146-artificial sequence: nucleotide sequence of antibody sequence of pCI-AVM-hLG4PE(R409K)_AVMscFv3 (excluding signal sequence)
Definition of SEQ ID NO: 147-artificial sequence: amino acid sequence of antibody sequence of pCI-AVM-hLG4PE(R409K)_AVMscFv3 (excluding signal sequence)
Definition of SEQ ID NO: 148-artificial sequence: nucleotide sequence of antibody sequence of pCI-AVM-hLG4PE(R409K)_AVMscFv5 (excluding signal sequence)
Definition of SEQ ID NO: 149-artificial sequence: amino acid sequence of antibody sequence of pCI-AVM-hLG4PE(R409K) AVMscFv5 (excluding signal sequence)
Definition of SEQ ID NO: 150-artificial sequence: nucleotide sequence of Acid Sphingomyelinase (ASM)
Definition of SEQ ID NO: 151-artificial sequence: nucleotide sequence encoding VH (excluding signal sequence) of MOG301
Definition of SEQ ID NO: 152-artificial sequence: amino acid sequence of VH (excluding signal sequence) of MOG301
Definition of SEQ ID NO: 153-artificial sequence: amino acid sequence of HCDR1 of MOG301
Definition of SEQ ID NO: 154-artificial sequence: amino acid sequence of HCDR2 of MOG301
Definition of SEQ ID NO: 155-artificial sequence: amino acid sequence of HCDR3 of MOG301
Definition of SEQ ID NO: 156-artificial sequence: nucleotide sequence encoding VL (excluding signal sequence) of MOG301
Definition of SEQ ID NO: 157-artificial sequence: amino acid sequence of VL (excluding signal sequence) of MOG301
Definition of SEQ ID NO: 158-artificial sequence: amino acid sequence of LCDR1 of MOG301
Definition of SEQ ID NO: 159-artificial sequence: amino acid sequence of LCDR2 of MOG301
Definition of SEQ ID NO: 160-artificial sequence: amino acid sequence of LCDR3 of MOG301
Definition of SEQ ID NO: 161-artificial sequence: nucleotide sequence encoding VH (excluding signal sequence) of MOG303
Definition of SEQ ID NO: 162-artificial sequence: amino acid sequence of VH (excluding signal sequence) of MOG303
Definition of SEQ ID NO: 163-artificial sequence: amino acid sequence of HCDR1 of MOG303
Definition of SEQ ID NO: 164-artificial sequence: amino acid sequence of HCDR2 of MOG303
Definition of SEQ ID NO: 165-artificial sequence: amino acid sequence of HCDR3 of MOG303
Definition of SEQ ID NO: 166-artificial sequence: nucleotide sequence encoding VL (excluding signal sequence) of MOG303
Definition of SEQ ID NO: 167-artificial sequence: amino acid sequence of VL (excluding signal sequence) of MOG303
Definition of SEQ ID NO: 168-artificial sequence: amino acid sequence of LCDR1 of MOG303
Definition of SEQ ID NO: 169-artificial sequence: amino acid sequence of LCDR2 of MOG303
Definition of SEQ ID NO: 170-artificial sequence: amino acid sequence of LCDR3 of MOG303
Definition of SEQ ID NO: 171-artificial sequence: nucleotide sequence encoding VH (excluding signal sequence) of MOG307
Definition of SEQ ID NO: 172-artificial sequence: amino acid sequence of VH (excluding signal sequence) of MOG307
Definition of SEQ ID NO: 173-artificial sequence: amino acid sequence of HCDR1 of MOG307
Definition of SEQ ID NO: 174-artificial sequence: amino acid sequence of HCDR2 of MOG307
Definition of SEQ ID NO: 175-artificial sequence: amino acid sequence of HCDR3 of MOG307
Definition of SEQ ID NO: 176-artificial sequence: nucleotide sequence encoding VL (excluding signal sequence) of MOG307
Definition of SEQ ID NO: 177-artificial sequence: amino acid sequence of VL (excluding signal sequence) of MOG307
Definition of SEQ ID NO: 178-artificial sequence: amino acid sequence of LCDR1 of MOG307
Definition of SEQ ID NO: 179-artificial sequence: amino acid sequence of LCDR2 of MOG307
Definition of SEQ ID NO: 180-artificial sequence: amino acid sequence of LCDR3 of MOG307
Definition of SEQ ID NO: 181-artificial sequence: nucleotide sequence encoding VH (excluding signal sequence) of MOG310
Definition of SEQ ID NO: 182-artificial sequence: amino acid sequence of VH (excluding signal sequence) of MOG310
Definition of SEQ ID NO: 183-artificial sequence: amino acid sequence of HCDR1 of MOG310
Definition of SEQ ID NO: 184-artificial sequence: amino acid sequence of HCDR2 of MOG310
Definition of SEQ ID NO: 185-artificial sequence: amino acid sequence of HCDR3 of MOG310
Definition of SEQ ID NO: 186-artificial sequence: nucleotide sequence encoding VL (excluding signal sequence) of MOG310
Definition of SEQ ID NO: 187-artificial sequence: amino acid sequence of VL (excluding signal sequence) of MOG310
Definition of SEQ ID NO: 188-artificial sequence: amino acid sequence of LCDR1 of MOG310
Definition of SEQ ID NO: 189-artificial sequence: amino acid sequence of LCDR2 of MOG310
Definition of SEQ ID NO: 190-artificial sequence: amino acid sequence of LCDR3 of MOG310
Definition of SEQ ID NO: 191-artificial sequence: nucleotide sequence encoding VH (excluding signal sequence) of MOG312
Definition of SEQ ID NO: 192-artificial sequence: amino acid sequence of VH (excluding signal sequence) of MOG312
Definition of SEQ ID NO: 193-artificial sequence: amino acid sequence of HCDR1 of MOG312
Definition of SEQ ID NO: 194-artificial sequence: amino acid sequence of HCDR2 of MOG312
Definition of SEQ ID NO: 195-artificial sequence: amino acid sequence of HCDR3 of MOG312
Definition of SEQ ID NO: 196-artificial sequence: nucleotide sequence encoding VL (excluding signal sequence) of MOG312
Definition of SEQ ID NO: 197-artificial sequence: amino acid sequence of VL (excluding signal sequence) of MOG312
Definition of SEQ ID NO: 198-artificial sequence: amino acid sequence of LCDR1 of MOG312
Definition of SEQ ID NO: 199-artificial sequence: amino acid sequence of LCDR2 of MOG312
Definition of SEQ ID NO: 200-artificial sequence: amino acid sequence of LCDR3 of MOG312
Definition of SEQ ID NO: 201-artificial sequence: nucleotide sequence encoding VH (excluding signal sequence) of MOG326
Definition of SEQ ID NO: 202-artificial sequence: amino acid sequence of VH (excluding signal sequence) of MOG326
Definition of SEQ ID NO: 203-artificial sequence: amino acid sequence of HCDR1 of MOG326
Definition of SEQ ID NO: 204-artificial sequence: amino acid sequence of HCDR2 of MOG326
Definition of SEQ ID NO: 205-artificial sequence: amino acid sequence of HCDR3 of MOG326
Definition of SEQ ID NO: 206-artificial sequence: nucleotide sequence encoding VL (excluding signal sequence) of MOG326
Definition of SEQ ID NO: 207-artificial sequence: amino acid sequence of VL (excluding signal sequence) of MOG326
Definition of SEQ ID NO: 208-artificial sequence: amino acid sequence of LCDR1 of MOG326
Definition of SEQ ID NO: 209-artificial sequence: amino acid sequence of LCDR2 of MOG326
Definition of SEQ ID NO: 210-artificial sequence: amino acid sequence of LCDR3 of MOG326
Definition of SEQ ID NO: 211-artificial sequence: nucleotide sequence encoding VH (excluding signal sequence) of MOG329
Definition of SEQ ID NO: 212-artificial sequence: amino acid sequence of VH (excluding signal sequence) of MOG329
Definition of SEQ ID NO: 213-artificial sequence: amino acid sequence of HCDR1 of MOG329
Definition of SEQ ID NO: 214-artificial sequence: amino acid sequence of HCDR2 of MOG329
Definition of SEQ ID NO: 215-artificial sequence: amino acid sequence of HCDR3 of MOG329
Definition of SEQ ID NO: 216-artificial sequence: nucleotide sequence encoding VL (excluding signal sequence) of MOG329
Definition of SEQ ID NO: 217-artificial sequence: amino acid sequence of VL (excluding signal sequence) of MOG329
Definition of SEQ ID NO: 218-artificial sequence: amino acid sequence of LCDR1 of MOG329
Definition of SEQ ID NO: 219-artificial sequence: amino acid sequence of LCDR2 of MOG329
Definition of SEQ ID NO: 220-artificial sequence: amino acid sequence of LCDR3 of MOG329
Definition of SEQ ID NO: 221-artificial sequence: nucleotide sequence encoding VH (excluding signal sequence) of MOG446
Definition of SEQ ID NO: 222-artificial sequence: amino acid sequence of VH (excluding signal sequence) of MOG446
Definition of SEQ ID NO: 223-artificial sequence: amino acid sequence of HCDR1 of MOG446
Definition of SEQ ID NO: 224-artificial sequence: amino acid sequence of HCDR2 of MOG446
Definition of SEQ ID NO: 225-artificial sequence: amino acid sequence of HCDR3 of MOG446
Definition of SEQ ID NO: 226-artificial sequence: nucleotide sequence encoding VL (excluding signal sequence) of MOG446
Definition of SEQ ID NO: 227-artificial sequence: amino acid sequence of VL (excluding signal sequence) of MOG446
Definition of SEQ ID NO: 228-artificial sequence: amino acid sequence of LCDR1 of MOG446
Definition of SEQ ID NO: 229-artificial sequence: amino acid sequence of LCDR2 of MOG446
Definition of SEQ ID NO: 230-artificial sequence: amino acid sequence of LCDR3 of MOG446
Definition of SEQ ID NO: 231-artificial sequence: nucleotide sequence encoding VH (excluding signal sequence) of MOG456
Definition of SEQ ID NO: 232-artificial sequence: amino acid sequence of VH (excluding signal sequence) of MOG456
Definition of SEQ ID NO: 233-artificial sequence: amino acid sequence of HCDR1 of MOG456
Definition of SEQ ID NO: 234-artificial sequence: amino acid sequence of HCDR2 of MOG456
Definition of SEQ ID NO: 235-artificial sequence: amino acid sequence of HCDR3 of MOG456
Definition of SEQ ID NO: 236-artificial sequence: nucleotide sequence encoding VL (excluding signal sequence) of MOG456
Definition of SEQ ID NO: 237-artificial sequence: amino acid sequence of VL (excluding signal sequence) of MOG456
Definition of SEQ ID NO: 238-artificial sequence: amino acid sequence of LCDR1 of MOG456
Definition of SEQ ID NO: 239-artificial sequence: amino acid sequence of LCDR2 of MOG456
Definition of SEQ ID NO: 240-artificial sequence: amino acid sequence of LCDR3 of MOG456
Definition of SEQ ID NO: 241-artificial sequence: nucleotide sequence encoding VH (excluding signal sequence) of MOG473
Definition of SEQ ID NO: 242-artificial sequence: amino acid sequence of VH (excluding signal sequence) of MOG473
Definition of SEQ ID NO: 243-artificial sequence: amino acid sequence of HCDR1 of MOG473
Definition of SEQ ID NO: 244-artificial sequence: amino acid sequence of HCDR2 of MOG473
Definition of SEQ ID NO: 245-artificial sequence: amino acid sequence of HCDR3 of MOG473
Definition of SEQ ID NO: 246-artificial sequence: nucleotide sequence encoding VL (excluding signal sequence) of MOG473
Definition of SEQ ID NO: 247-artificial sequence: amino acid sequence of VL (excluding signal sequence) of MOG473
Definition of SEQ ID NO: 248-artificial sequence: amino acid sequence of LCDR1 of MOG473
Definition of SEQ ID NO: 249-artificial sequence: amino acid sequence of LCDR2 of MOG473
Definition of SEQ ID NO: 250-artificial sequence: amino acid sequence of LCDR3 of MOG473
Definition of SEQ ID NO: 251-artificial sequence: nucleotide sequence encoding VH (excluding signal sequence) of MOG426
Definition of SEQ ID NO: 252-artificial sequence: amino acid sequence of VH (excluding signal sequence) of MOG426
Definition of SEQ ID NO: 253-artificial sequence: nucleotide sequence encoding VL (excluding signal sequence) of MOG426
Definition of SEQ ID NO: 254-artificial sequence: amino acid sequence of VL (excluding signal sequence) of MOG426
Definition of SEQ ID NO: 255-artificial sequence: nucleotide sequence encoding VH (excluding signal sequence) of MOG428
Definition of SEQ ID NO: 256-artificial sequence: amino acid sequence of VH (excluding signal sequence) of MOG428
Definition of SEQ ID NO: 257-artificial sequence: nucleotide sequence encoding VL (excluding signal sequence) of MOG428
Definition of SEQ ID NO: 258-artificial sequence: amino acid sequence of VL (excluding signal sequence) of MOG428
Definition of SEQ ID NO: 259-artificial sequence: nucleotide sequence encoding VH (excluding signal sequence) of MOG313
Definition of SEQ ID NO: 260-artificial sequence: amino acid sequence of VH (excluding signal sequence) of MOG313
Definition of SEQ ID NO: 261-artificial sequence: nucleotide sequence encoding VL (excluding signal sequence) of MOG313
Definition of SEQ ID NO: 262-artificial sequence: amino acid sequence of VL (excluding signal sequence) of MOG313
Definition of SEQ ID NO: 263-artificial sequence: nucleotide sequence encoding VH (excluding signal sequence) of MOG314
Definition of SEQ ID NO: 264-artificial sequence: amino acid sequence of VH (excluding signal sequence) of MOG314
Definition of SEQ ID NO: 265-artificial sequence: nucleotide sequence encoding VL (excluding signal sequence) of MOG314
Definition of SEQ ID NO: 266-artificial sequence: amino acid sequence of VL (excluding signal sequence) of MOG314
Definition of SEQ ID NO: 267-artificial sequence: nucleotide sequence encoding VH (excluding signal sequence) of MOG315
Definition of SEQ ID NO: 268-artificial sequence: amino acid sequence of VH (excluding signal sequence) of MOG315
Definition of SEQ ID NO: 269-artificial sequence: nucleotide sequence encoding VL (excluding signal sequence) of MOG315
Definition of SEQ ID NO: 270-artificial sequence: amino acid sequence of VL (excluding signal sequence) of MOG315
Definition of SEQ ID NO: 271-artificial sequence: nucleotide sequence encoding VH (excluding signal sequence) of MOG331
Definition of SEQ ID NO: 272-artificial sequence: amino acid sequence of VH (excluding signal sequence) of MOG331
Definition of SEQ ID NO: 273-artificial sequence: nucleotide sequence encoding VL (excluding signal sequence) of MOG331
Definition of SEQ ID NO: 274-artificial sequence: amino acid sequence of VL (excluding signal sequence) of MOG331
Definition of SEQ ID NO: 275-artificial sequence: nucleotide sequence encoding VH (excluding signal sequence) of MOG357
Definition of SEQ ID NO: 276-artificial sequence: amino acid sequence of VH (excluding signal sequence) of MOG357
Definition of SEQ ID NO: 277-artificial sequence: nucleotide sequence encoding VL (excluding signal sequence) of MOG357
Definition of SEQ ID NO: 278-artificial sequence: amino acid sequence of VL (excluding signal sequence) of MOG357
Definition of SEQ ID NO: 279-artificial sequence: nucleotide sequence encoding VH (excluding signal sequence) of MOG476
Definition of SEQ ID NO: 280-artificial sequence: amino acid sequence of VH (excluding signal sequence) of MOG476
Definition of SEQ ID NO: 281-artificial sequence: nucleotide sequence encoding VL (excluding signal sequence) of MOG476
Definition of SEQ ID NO: 282-artificial sequence: amino acid sequence of VL (excluding signal sequence) of MOG476
Definition of SEQ ID NO: 283-artificial sequence: nucleotide sequence encoding VH (excluding signal sequence) of MOG323
Definition of SEQ ID NO: 284-artificial sequence: amino acid sequence of VH (excluding signal sequence) of MOG323
Definition of SEQ ID NO: 285-artificial sequence: nucleotide sequence encoding VL (excluding signal sequence) of MOG323
Definition of SEQ ID NO: 286-artificial sequence: amino acid sequence of VL (excluding signal sequence) of MOG323
Definition of SEQ ID NO: 287-artificial sequence: nucleotide sequence encoding VH (excluding signal sequence) of MOG341
Definition of SEQ ID NO: 288-artificial sequence: amino acid sequence of VH (excluding signal sequence) of MOG341
Definition of SEQ ID NO: 289-artificial sequence: nucleotide sequence encoding VL (excluding signal sequence) of MOG341
Definition of SEQ ID NO: 290-artificial sequence: amino acid sequence of VL (excluding signal sequence) of MOG341
Definition of SEQ ID NO: 291-artificial sequence: nucleotide sequence encoding VH (excluding signal sequence) of MOG354
Definition of SEQ ID NO: 292-artificial sequence: amino acid sequence of VH (excluding signal sequence) of MOG354
Definition of SEQ ID NO: 293-artificial sequence: nucleotide sequence encoding VL (excluding signal sequence) of MOG354
Definition of SEQ ID NO: 294-artificial sequence: amino acid sequence of VL (excluding signal sequence) of MOG354
Definition of SEQ ID NO: 295-artificial sequence: nucleotide sequence encoding VH (excluding signal sequence) of MOG355
Definition of SEQ ID NO: 296-artificial sequence: amino acid sequence of VH (excluding signal sequence) of MOG355
Definition of SEQ ID NO: 297-artificial sequence: nucleotide sequence encoding VL (excluding signal sequence) of MOG355
Definition of SEQ ID NO: 298-artificial sequence: amino acid sequence of VL (excluding signal sequence) of MOG355
Definition of SEQ ID NO: 299-artificial sequence: nucleotide sequence encoding VH (excluding signal sequence) of MOG308
Definition of SEQ ID NO: 300-artificial sequence: amino acid sequence of VH (excluding signal sequence) of MOG308
Definition of SEQ ID NO: 301-artificial sequence: nucleotide sequence encoding VL (excluding signal sequence) of MOG308
Definition of SEQ ID NO: 302-artificial sequence: amino acid sequence of VL (excluding signal sequence) of MOG308
Definition of SEQ ID NO: 303-artificial sequence: nucleotide sequence encoding VH (excluding signal sequence) of MOG316
Definition of SEQ ID NO: 304-artificial sequence: amino acid sequence of VH (excluding signal sequence) of MOG316
Definition of SEQ ID NO: 305-artificial sequence: nucleotide sequence encoding VL (excluding signal sequence) of MOG316
Definition of SEQ ID NO: 306-artificial sequence: amino acid sequence of VL (excluding signal sequence) of MOG316
Definition of SEQ ID NO: 307-artificial sequence: nucleotide sequence encoding VH (excluding signal sequence) of MOG319
Definition of SEQ ID NO: 308-artificial sequence: amino acid sequence of VH (excluding signal sequence) of MOG319
Definition of SEQ ID NO: 309-artificial sequence: nucleotide sequence encoding VL (excluding signal sequence) of MOG319
Definition of SEQ ID NO: 310-artificial sequence: amino acid sequence of VL (excluding signal sequence) of MOG319
Definition of SEQ ID NO: 311-artificial sequence: nucleotide sequence encoding VH (excluding signal sequence) of MOG320
Definition of SEQ ID NO: 312-artificial sequence: amino acid sequence of VH (excluding signal sequence) of MOG320
Definition of SEQ ID NO: 313-artificial sequence: nucleotide sequence encoding VL (excluding signal sequence) of MOG320
Definition of SEQ ID NO: 314-artificial sequence: amino acid sequence of VL (excluding signal sequence) of MOG320
Definition of SEQ ID NO: 315-artificial sequence: nucleotide sequence encoding VH (excluding signal sequence) of MOG338
Definition of SEQ ID NO: 316-artificial sequence: amino acid sequence of VH (excluding signal sequence) of MOG338
Definition of SEQ ID NO: 317-artificial sequence: nucleotide sequence encoding VL (excluding signal sequence) of MOG338
Definition of SEQ ID NO: 318-artificial sequence: amino acid sequence of VL (excluding signal sequence) of MOG338
Definition of SEQ ID NO: 319-artificial sequence: nucleotide sequence encoding VH (excluding signal sequence) of MOG352
Definition of SEQ ID NO: 320-artificial sequence: amino acid sequence of VH (excluding signal sequence) of MOG352
Definition of SEQ ID NO: 321-artificial sequence: nucleotide sequence encoding VL (excluding signal sequence) of MOG352
Definition of SEQ ID NO: 322-artificial sequence: amino acid sequence of VL (excluding signal sequence) of MOG352
Definition of SEQ ID NO: 323-artificial sequence: nucleotide sequence encoding VH (excluding signal sequence) of MOG359
Definition of SEQ ID NO: 324-artificial sequence: amino acid sequence of VH (excluding signal sequence) of MOG359
Definition of SEQ ID NO: 325-artificial sequence: nucleotide sequence encoding VL (excluding signal sequence) of MOG359
Definition of SEQ ID NO: 326-artificial sequence: amino acid sequence of VL (excluding signal sequence) of MOG359
Definition of SEQ ID NO: 327-artificial sequence: nucleotide sequence encoding VH (excluding signal sequence) of MOG478
Definition of SEQ ID NO: 328-artificial sequence: amino acid sequence of VH (excluding signal sequence) of MOG478
Definition of SEQ ID NO: 329-artificial sequence: nucleotide sequence encoding VL (excluding signal sequence) of MOG478
Definition of SEQ ID NO: 330-artificial sequence: amino acid sequence of VL (excluding signal sequence) of MOG478
Definition of SEQ ID NO: 331-artificial sequence: nucleotide sequence encoding VH (excluding signal sequence) of MOG470
Definition of SEQ ID NO: 332-artificial sequence: amino acid sequence of VH (excluding signal sequence) of MOG470
Definition of SEQ ID NO: 333-artificial sequence: nucleotide sequence encoding VL (excluding signal sequence) of MOG470
Definition of SEQ ID NO: 334-artificial sequence: amino acid sequence of VL (excluding signal sequence) of MOG470
Definition of SEQ ID NO: 335-artificial sequence: nucleotide sequence encoding VH (excluding signal sequence) of MOG418
Definition of SEQ ID NO: 336-artificial sequence: amino acid sequence of VH (excluding signal sequence) of MOG418
Definition of SEQ ID NO: 337-artificial sequence: nucleotide sequence encoding VL (excluding signal sequence) of MOG418
Definition of SEQ ID NO: 338-artificial sequence: amino acid sequence of VL (excluding signal sequence) of MOG418

1. An antibody which binds to myelin oligodendrocyte glycoprotein (referred to as MOG below) or an antibody fragment thereof.

2. The antibody or the antibody fragment according to claim 1, wherein the antibody has a property of accumulating in a brain.

3. The antibody or the antibody fragment according to claim 1, wherein the antibody is selected from the group consisting of (a) to (q) below,
(a) an antibody in which the amino acid sequences of complementarity determining regions (CDRs) 1 to 3 of a heavy chain variable region (VH) comprise the amino acid sequences of SEQ ID NOs: 16, 17 and 18, respectively, and in which the amino acid sequences of CDRs 1 to 3 of a light chain variable region (VL) comprise the amino acid sequences of SEQ ID NOs: 22, 23 and 24, respectively,
(b) an antibody in which the amino acid sequences of CDRs 1 to 3 of VH comprise the amino acid sequences of SEQ ID NOs: 28, 29 and 30, respectively, and in which the amino acid sequences of CDRs 1 to 3 of VL comprise the amino acid sequences of SEQ ID NOs: 34, 35 and 36, respectively,
(c) an antibody fragment in which the amino acid sequences of CDRs 1 to 3 of a heavy chain variable region of a heavy chain antibody (VHH) comprise the amino acid sequences of SEQ ID NOs: 40, 41 and 42, respectively,
(d) an antibody in which the amino acid sequences of CDRs 1 to 3 of VH comprise the amino acid sequences of SEQ ID NOs: 153, 154 and 155, respectively, and in which the amino acid sequences of CDRs 1 to 3 of VL comprise the amino acid sequences of SEQ ID NOs: 158, 159 and 160, respectively,
(e) an antibody in which the amino acid sequences of CDRs 1 to 3 of VH comprise the amino acid sequences of SEQ ID NOs: 163, 164 and 165, respectively, and in which the amino acid sequences of CDRs 1 to 3 of VL comprise the amino acid sequences of SEQ ID NOs: 168, 169 and 170, respectively,
(f) an antibody in which the amino acid sequences of CDRs 1 to 3 of VH comprise the amino acid sequences of SEQ ID NOs: 173, 174 and 175, respectively, and in which the amino acid sequences of CDRs 1 to 3 of VL comprise the amino acid sequences of SEQ ID NOs: 178, 179 and 180, respectively,
(g) an antibody in which the amino acid sequences of CDRs 1 to 3 of VH comprise the amino acid sequences of SEQ ID NOs: 183, 184 and 185, respectively, and in which the amino acid sequences of CDRs 1 to 3 of VL comprise the amino acid sequences of SEQ ID NOs: 188, 189 and 190, respectively,
(h) an antibody in which the amino acid sequences of CDRs 1 to 3 of VH comprise the amino acid sequences of SEQ ID NOs: 193, 194 and 195, respectively, and in which the amino acid sequences of CDRs 1 to 3 of VL comprise the amino acid sequences of SEQ ID NOs: 198, 199 and 200, respectively,
(i) an antibody in which the amino acid sequences of CDRs 1 to 3 of VH comprise the amino acid sequences of SEQ ID NOs: 203, 204 and 205, respectively, and in which the amino acid sequences of CDRs 1 to 3 of VL comprise the amino acid sequences of SEQ ID NOs: 208, 209 and 210, respectively,
(j) an antibody in which the amino acid sequences of CDRs 1 to 3 of VH comprise the amino acid sequences of SEQ ID NOs: 213, 214 and 215, respectively, and in which the amino acid sequences of CDRs 1 to 3 of VL comprise the amino acid sequences of SEQ ID NOs: 218, 219 and 220, respectively,
(k) an antibody in which the amino acid sequences of CDRs 1 to 3 of VH comprise the amino acid sequences of SEQ ID NOs: 223, 224 and 225, respectively, and in which the amino acid sequences of CDRs 1 to 3 of VL comprise the amino acid sequences of SEQ ID NOs: 228, 229 and 230, respectively,
(l) an antibody in which the amino acid sequences of CDRs 1 to 3 of VH comprise the amino acid sequences of SEQ ID NOs: 233, 234 and 235, respectively, and in which the amino acid sequences of CDRs 1 to 3 of VL comprise the amino acid sequences of SEQ ID NOs: 238, 239 and 240, respectively,
(m) an antibody in which the amino acid sequences of CDRs 1 to 3 of VH comprise the amino acid sequences of SEQ ID NOs: 243, 244 and 245, respectively, and in which the amino acid sequences of CDRs 1 to 3 of VL comprise the amino acid sequences of SEQ ID NOs: 248, 249 and 250, respectively,
(n) an antibody which competes in binding to MOG with at least one of the antibodies described in (a) to (m),
(o) an antibody which binds to an epitope comprising an epitope to which any one of the antibodies described in (a) to (m) binds,
(p) an antibody which binds to the same epitope as an epitope to which any one of the antibodies described in (a) to (m) binds, and
(q) an antibody which comprises an amino acid sequence having homology of 85% or higher to the amino acid sequence of any one of the antibodies described in (a) to (m).

4. The antibody or the antibody fragment according to claim 1, wherein the antibody is selected from the group consisting of (a) to (m), (o1) to (o22) and (p) below,
(a) an antibody in which the amino acid sequence of VH comprises the amino acid sequence of SEQ ID NO: 15 and in which the amino acid sequence of VL comprises the amino acid sequence of SEQ ID NO: 21,
(b) an antibody in which the amino acid sequence of VH comprises the amino acid sequence of SEQ ID NO: 27 and in which the amino acid sequence of VL comprises the amino acid sequence of SEQ ID NO: 33,
(c) an antibody fragment in which the amino acid sequence of VHH comprises the amino acid sequence of SEQ ID NO: 39,
(d) an antibody in which the amino acid sequence of VH comprises the amino acid sequence of SEQ ID NO: 152 and in which the amino acid sequence of VL comprises the amino acid sequence of SEQ ID NO: 157,
(e) an antibody in which the amino acid sequence of VH comprises the amino acid sequence of SEQ ID NO: 162 and in which the amino acid sequence of VL comprises the amino acid sequence of SEQ ID NO: 167,
(f) an antibody in which the amino acid sequence of VH comprises the amino acid sequence of SEQ ID NO: 172 and in which the amino acid sequence of VL comprises the amino acid sequence of SEQ ID NO: 177,
(g) an antibody in which the amino acid sequence of VH comprises the amino acid sequence of SEQ ID NO: 182 and in which the amino acid sequence of VL comprises the amino acid sequence of SEQ ID NO: 187,
(h) an antibody in which the amino acid sequence of VH comprises the amino acid sequence of SEQ ID NO: 192 and in which the amino acid sequence of VL comprises the amino acid sequence of SEQ ID NO: 197,
(i) an antibody in which the amino acid sequence of VH comprises the amino acid sequence of SEQ ID NO: 202 and in which the amino acid sequence of VL comprises the amino acid sequence of SEQ ID NO: 207,
(j) an antibody in which the amino acid sequence of VH comprises the amino acid sequence of SEQ ID NO: 212 and in which the amino acid sequence of VL comprises the amino acid sequence of SEQ ID NO: 217,
(k) an antibody in which the amino acid sequence of VH comprises the amino acid sequence of SEQ ID NO: 222 and in which the amino acid sequence of VL comprises the amino acid sequence of SEQ ID NO: 227,
(l) an antibody in which the amino acid sequence of VH comprises the amino acid sequence of SEQ ID NO: 232 and in which the amino acid sequence of VL comprises the amino acid sequence of SEQ ID NO: 237,
(m) an antibody in which the amino acid sequence of VH comprises the amino acid sequence of SEQ ID NO: 242 and in which the amino acid sequence of VL comprises the amino acid sequence of SEQ ID NO: 247,
(o1) an antibody in which the amino acid sequence of VH comprises the amino acid sequence of SEQ ID NO: 252 and in which the amino acid sequence of VL comprises the amino acid sequence of SEQ ID NO: 254,
(o2) an antibody in which the amino acid sequence of VH comprises the amino acid sequence of SEQ ID NO: 256 and in which the amino acid sequence of VL comprises the amino acid sequence of SEQ ID NO: 258,
(o3) an antibody in which the amino acid sequence of VH comprises the amino acid sequence of SEQ ID NO: 260 and in which the amino acid sequence of VL comprises the amino acid sequence of SEQ ID NO: 262,
(o4) an antibody in which the amino acid sequence of VH comprises the amino acid sequence of SEQ ID NO: 264 and in which the amino acid sequence of VL comprises the amino acid sequence of SEQ ID NO: 266,
(o5) an antibody in which the amino acid sequence of VH comprises the amino acid sequence of SEQ ID NO: 268 and in which the amino acid sequence of VL comprises the amino acid sequence of SEQ ID NO: 270,
(o6) an antibody in which the amino acid sequence of VH comprises the amino acid sequence of SEQ ID NO: 272 and in which the amino acid sequence of VL comprises the amino acid sequence of SEQ ID NO: 274,
(o7) an antibody in which the amino acid sequence of VH comprises the amino acid sequence of SEQ ID NO: 276 and in which the amino acid sequence of VL comprises the amino acid sequence of SEQ ID NO: 278,
(o8) an antibody in which the amino acid sequence of VH comprises the amino acid sequence of SEQ ID NO: 280 and in which the amino acid sequence of VL comprises the amino acid sequence of SEQ ID NO: 282,
(o9) an antibody in which the amino acid sequence of VH comprises the amino acid sequence of SEQ ID NO: 284 and in which the amino acid sequence of VL comprises the amino acid sequence of SEQ ID NO: 286,
(o10) an antibody in which the amino acid sequence of VH comprises the amino acid sequence of SEQ ID NO: 288 and in which the amino acid sequence of VL comprises the amino acid sequence of SEQ ID NO: 290,
(o11) an antibody in which the amino acid sequence of VH comprises the amino acid sequence of SEQ ID NO: 292 and in which the amino acid sequence of VL comprises the amino acid sequence of SEQ ID NO: 294,
(o12) an antibody in which the amino acid sequence of VH comprises the amino acid sequence of SEQ ID NO: 296 and in which the amino acid sequence of VL comprises the amino acid sequence of SEQ ID NO: 298,
(o13) an antibody in which the amino acid sequence of VH comprises the amino acid sequence of SEQ ID NO: 300 and in which the amino acid sequence of VL comprises the amino acid sequence of SEQ ID NO: 302,
(o14) an antibody in which the amino acid sequence of VH comprises the amino acid sequence of SEQ ID NO: 304 and in which the amino acid sequence of VL comprises the amino acid sequence of SEQ ID NO: 306,
(o15) an antibody in which the amino acid sequence of VH comprises the amino acid sequence of SEQ ID NO: 308 and in which the amino acid sequence of VL comprises the amino acid sequence of SEQ ID NO: 310,
(o16) an antibody in which the amino acid sequence of VH comprises the amino acid sequence of SEQ ID NO: 312 and in which the amino acid sequence of VL comprises the amino acid sequence of SEQ ID NO: 314,
(o17) an antibody in which the amino acid sequence of VH comprises the amino acid sequence of SEQ ID NO: 316 and in which the amino acid sequence of VL comprises the amino acid sequence of SEQ ID NO: 318,
(o18) an antibody in which the amino acid sequence of VH comprises the amino acid sequence of SEQ ID NO: 320 and in which the amino acid sequence of VL comprises the amino acid sequence of SEQ ID NO: 322,
(o19) an antibody in which the amino acid sequence of VH comprises the amino acid sequence of SEQ ID NO: 324 and in which the amino acid sequence of VL comprises the amino acid sequence of SEQ ID NO: 326,
(o20) an antibody in which the amino acid sequence of VH comprises the amino acid sequence of SEQ ID NO: 328 and in which the amino acid sequence of VL comprises the amino acid sequence of SEQ ID NO: 330,
(o21) an antibody in which the amino acid sequence of VH comprises the amino acid sequence of SEQ ID NO: 332 and in which the amino acid sequence of VL comprises the amino acid sequence of SEQ ID NO: 334,
(o22) an antibody in which the amino acid sequence of VH comprises the amino acid sequence of SEQ ID NO: 336 and in which the amino acid sequence of VL comprises the amino acid sequence of SEQ ID NO: 338, and
(p) an antibody which comprises an amino acid sequence having homology of 85% or higher to the amino acid sequence of any one of the antibodies described in (a) to (m) and (o1) to (o22).

5. The antibody or the antibody fragment according to claim 1, wherein the antibody or the antibody fragment is a bispecific antibody.

6. The bispecific antibody according to claim 5, wherein the bispecific antibody binds to MOG and an antigen that is present in a brain.

7. The bispecific antibody according to claim 5, wherein the bispecific antibody comprises an antigen binding site which binds to MOG and an antigen binding site which binds to an antigen that is present in a brain.

8. The antibody fragment according to claim 1 which is selected from the group consisting of Fab, Fab′, F(ab′)₂, a single chain antibody (scFv), a dimerized V region (diabody), a disulfide-stabilized V region (dsFv), VHH, and a peptide comprising CDR.

9. The antibody or the antibody fragment according to claim 1, wherein the antibody is a genetically recombinant antibody.

10. The antibody or the antibody fragment according to claim 1, wherein the antibody is selected from the group consisting of a mouse antibody, a rat antibody, a rabbit antibody, an alpaca antibody, a camel antibody, a llama antibody, a chimeric antibody, a humanized antibody, and a human antibody.

11. A fused antibody or a fused antibody fragment which is obtained by binding at least one selected from the group consisting of (a) to (c) below to the antibody which binds to MOG according to claim 1 or the antibody fragment thereof,
(a) a hydrophilic polymer,
(b) an amphipathic polymer, and
(c) a functional molecule.

12. A hybridoma which produces the antibody according to claim 1.

13. A nucleic acid which comprises a nucleotide sequence which encodes the antibody according to claim 1.

14. A transformant cell which comprises a vector comprising the nucleic acid according to claim 13.

15. A method for producing the antibody or the antibody fragment according to claim 1, comprising:
culturing a hybridoma which produces the antibody according to claim 1 or a transformant cell which comprises a vector comprising a nucleic acid which comprises a nucleotide sequence which encodes the antibody according to claim 1, and
collecting the antibody or the antibody fragment according to claim 1 from a culture solution.

16. A composition which comprises the antibody or the antibody fragment according to claim 1.

17. The composition according to claim 16, wherein the composition is a composition for detecting or measuring an antigen that is present in a brain.

18. The composition according to claim 16, wherein the composition is a composition for diagnosing or treating a brain disease.

19. A method for detecting or measuring an antigen that is present in a brain using the antibody or the antibody fragment according to claim 1 or a composition which comprises the antibody or the antibody fragment according to claim 1.

20. A method for diagnosing or treating a brain disease using the antibody or the antibody fragment according to claim 1 or a composition which comprises the antibody or the antibody fragment according to claim 1.

21. A method for improving the property of accumulating in a brain of an antibody, an antibody fragment thereof, a fused antibody or a fused antibody fragment using the antibody, the antibody fragment, the fused antibody or the fused antibody fragment according to claim 1 or a composition which comprises the antibody or the antibody fragment according to claim 1.

22. A method for increasing the amount of an antibody, the amount of an antibody fragment thereof, the amount of a fused antibody or the amount of a fused antibody fragment in a brain using the antibody, the antibody fragment, the fused antibody or the fused antibody fragment according to claim 1 or a composition which comprises the antibody or the antibody fragment according to claim 1.
